(19) United States
(12) Patent Application Publication
(10) Pub. No.: US 2024/0317892 A1
(43) Pub. Date: Sep. 26, 2024

(54) ANTI-ADAM12 ANTIBODIES AND CHIMERIC ANTIGEN RECEPTORS, AND COMPOSITIONS AND METHODS COMPRISING

(71) Applicant: JAVELIN ONCOLOGY, INC, LEBANON, NH (US)

(72) Inventor: Anthony B. Cooper, Lebanon, NH (US)

(21) Appl. No.: 17/440,299

(22) PCT Filed: Mar. 20, 2020
(86) PCT No.: PCT/US2020/023859

§ 371 Date: May 6, 2022

Related U.S. Application Data
(60) Provisional application No. 62/821,257, filed on Mar. 20, 2019.

(51) Int. Cl. C07K 16/40; A61K 39/00; A61P 35/00; C12N 5/0783

(52) U.S. Cl. CPC C07K 16/40; A61K 39/4611; A61K 39/4631; A61K 39/464458; A61P 35/00; C12N 5/0636; C07K 2317/24; C12N 2510/00

(57) ABSTRACT

Anti-ADAM12 agents are provided such as anti-ADAM12 antibodies (Abs), antigen-binding Ab fragments, multi-specific Abs and antigen-binding Ab fragments, antibody-drug conjugates (ADCs), and chimeric antigen receptors (CARs). Also, nucleic acid sequences and vectors are provided encoding, cells and pharmaceutical compositions comprising such anti-ADAM12 agents and methods for expanding such cells. Methods of treating, preventing, or diagnosing a disease such as cancer and methods of stimulating an immune response using such materials are also provided.

### RELATED APPLICATIONS

This application claims priority to U.S. Provisional Application No. 62/821,257 filed on Mar. 20, 2019, entitled “ANTI-ADAM12 ANTIBODIES AND CHIMERIC ANTIGEN RECEPTORS, AND COMPOSITIONS AND METHODS COMPRISING”, the contents of which are incorporated by reference in their entirety herein.

### SEQUENCE LISTING DISCLOSURE

This application includes as part of its disclosure a biological sequence listing which is being concurrently submitted through EFS-Web. Said biological sequence listing is contained in a file named “1156867o001613.txt” which was created on Feb. 25, 2020, and has a size of 204,342 bytes, and is hereby incorporated by reference in its entirety.

### FIELD OF INVENTION

The present disclosure relates to anti-ADAM12 agents such as anti-ADAM12 antibodies (Abs), antigen-binding Ab fragments, multi-specific Abs and antigen-binding Ab fragments, antibody-drug conjugates (ADCs), and chimeric antigen receptors (CARs). The disclosure also relates to nucleic acid sequences and vectors encoding, cells and pharmaceutical compositions comprising such anti-ADAM12 agents and methods for expanding such cells. The present disclosure further relates to methods of treating a subject using such anti-ADAM12 agents and compositions, and to methods of treating, preventing, or diagnosing a disease such as cancer and methods of stimulating an immune response. The invention also relates to methods of producing such an anti-ADAM12 agent or composition.

### BACKGROUND OF THE INVENTION

Immunotherapy is a growing field, enabling treatment of a range of diseases that did not have an effective treatment option before. Many examples of immunotherapy have been employed in oncology, including antibody therapies targeting CD20 (Non-Hodgkin's lymphoma), HER2 (HER2-positive breast cancer), and immune checkpoints such as PD-1, PD-L1, and CTLA-4 (various cancers). Various immunotherapies are also being developed, tested, and/or marketed for non-cancer disease indications, such as autoimmune diseases (Wraith D. C. et al, Front Immunol. 2017 Nov. 28; 8:1668. doi: 10.3389/fimmu.2017.01668. eCollection 2017).

Chimeric antigen receptor (CAR) T cell therapy is an emerging type of immunotherapy whereby patient lymphocytes are genetically modified to express a receptor that allows recognition of a specific antigen. Upon antigen recognition, these modified T cells are activated via signaling domains, converting them into potent cell killers. In 2017, an anti-CD19 CAR T cell product received FDA approval for B cell lymphoma, illustrating the potential of this therapeutic approach in cancer, e.g., hematological cancer (Leyfman Y, et la., Cancer Cell Int. 2018 Nov. 14; 18:182. doi: 10.1186/s12935-018-0685-x. eCollection 2018). While also showing great promise for the treatment of solid tumors (Louis C. U. et al., Blood. 2011 Dec. 1; 118(23): 6050-6056. Prepublished online 2011 Oct. 7. doi: 10.1182/blood-2011-05-354449), CAR T cell therapy has encountered additional challenges in these indications (Yong C. S. M. et al., Immunol Cell Biol. 2017 April; 95(4):356-363. doi: 10.1038/icb.2016.128. Epub 2016 Dec. 22): the presence of an immunosuppressive tumor microenvironment; the issue of access to tumors; and a lack of the tumor-selective targeting required to minimize “off-tumor” toxicity.

The ADAM family (a disintegrin and metalloproteinase family) of enzymes are multifunctional, generally membrane-bound, zinc proteases (Nyren-Erickson E. K. Biochim Biophys Acta. 2013 October; 1830(10):4445-55. doi: 10.1016/j.bbagen.2013.05.011. Epub 2013 May 13). ADAM12 (also known as ADAM metallopeptidase domain 12, a disintegrin and metalloproteinase-12, a disintegrin and metalloproteinase domain-containing protein 12, ADAM12-OT1, CAR10, MCMP, MCMPMltna, Meltrin-a, MLTN, or MLTNA) is a member of the ADAM family. In humans, ADAM12 is encoded by the ADAM12 gene on chromosome 10, with gene location 10q26.2 (NCBI), and two naturally occurring ADAM12 splice variants named ADAM12-L and ADAM12-S exist (Kveiborg M. et al., Int J Biochem Cell Biol. 2008; 40(9):1685-702. doi: 10.1016/j.biocel.2008.01.025. Epub 2008 Feb. 1). The ADAM12-L domain composition resembles the prototypical transmembrane ADAM protein, containing extracellular pro-, metalloprotease, disintegrin-like, cysteine-rich, and epidermal growth factor (EGF)-like domains, followed by a transmembrane domain and a cytoplasmic tail domain. ADAM12-S, the soluble splice variant, contains the same domains as ADAM12-L, except that the transmembrane and cytoplasmic domains are replaced by a unique stretch of 33 amino acids in the C-terminus.

While the ADAM12 expression in healthy tissues is low, the general biological roles of ADAM12 are in cell adhesion and fusion, extracellular matrix restructuring, and cell signaling (Nyren-Erickson E. K. Biochim Biophys Acta. 2013 October; 1830(10):4445-55. doi: 10.1016/j.bbagen.2013.05.011. Epub 2013 May 13). ADAM12 has been implicated in the pathogenesis of various diseases, including many different types of cancer.

### SUMMARY OF THE INVENTION

The present invention relates to anti-ADAM12 agents.

In some embodiments, the anti-ADAM12 anent is an antibody (Ab) or antigen-binding Ab fragment. The Ab or antigen-binding Ab fragment may bind to ADAM12 and comprise: (a) a heavy chain (HC) variable domain (VH); and (b) a light chain (LC) variable domain (VL). The VH may comprise: a HC complementarity determining region (CDR) 1 (also referred to as CDR-H1); a HC CDR 2 (also referred to as CDR-H2); a HC CDR 3 (also referred to as CDR-H3); and human-like HC framework regions. The VL may comprise: a LC CDR 1 (CDR-L1); a LC CDR 2 (CDR-L2); a LC CDR 3 (CDR-L3); and human-like LC framework regions.

In some aspects, the HC CDR 1, HC CDR 2, and HC CDR 3 may comprise the amino acid sequence set forth in SEQ ID NOs: 132, 133, and 134, respectively, and the LC CDR 1, LC CDR 2, and LC CDR 3 may comprise the amino acid sequence set forth in SEQ ID NOs: 136, 137, and 138, respectively. The HC CDR 1, HC CDR 2, and HC CDR 3 may comprise the amino acid sequence encoded by SEQ ID NOs: 232, 233, and 234, respectively, and the LC CDR 1, LC CDR 2, and LC CDR 3 may comprise the amino acid sequence encoded by SEQ ID NOs: 236, 237, and 238, respectively. Alternatively, the HC CDR 1, HC CDR 2, and HC CDR 3 may comprise the amino acid sequence set forth in SEQ ID NOs: 142, 143, and 144, respectively, and the LC CDR 1, LC CDR 2, and LC CDR 3 may comprise the amino acid sequence set forth in SEQ ID NOs: 146, 147, and 148, respectively. The HC CDR 1, HC CDR 2, and HC CDR 3 may comprise the amino acid sequence encoded by SEQ ID NOs: 242, 243, and 244, respectively, and the LC CDR 1, LC CDR 2, and LC CDR 3 may comprise the amino acid sequence encoded by SEQ ID NOs: 246, 247, and 248, respectively.

In some aspects, the human-like HC framework may be at least 80%, at least 85%, at least 90%, at least 92%, at least 94%, at least 95%, at least 96%, at least 97%, at least 98%, at least 99%, or 100% identical to a human HC framework. The human-like LC framework may be at least 80%, at least 85%, at least 90%, at least 92%, at least 94%, at least 95%, at least 96%, at least 97%, at least 98%, at least 99%, or 100% identical to a human LC framework.

In certain aspects, the HC variable domain may comprise an amino acid sequence at least 80%, at least 85%, at least 90%, at least 92%, at least 94%, at least 95%, at least 96%, at least 97%, at least 98%, at least 99%, or 100% identical to SEQ ID NO: 131, and the LC variable domain may comprise an amino acid sequence at least 80%, at least 85%, at least 90%, at least 92%, at least 94%, at least 95%, at least 96%, at least 97%, at least 98%, at least 99%, or 100% identical to SEQ ID NO: 135. The HC variable domain may comprise an amino acid sequence at least 80%, at least 85%, at least 90%, at least 92%, at least 94%, at least 95%, at least 96%, at least 97%, at least 98%, at least 99%, or 100% identical to the amino acid sequence encoded by SEQ ID NO: 231, and the LC variable domain may comprise an amino acid sequence at least 80%, at least 85%, at least 90%, at least 92%, at least 94%, at least 95%, at least 96%, at least 97%, at least 98%, at least 99%, or 100% identical to the amino acid sequence encoded by SEQ ID NO: 235. Alternatively, the HC variable domain may comprise an amino acid sequence at least 80%, at least 85%, at least 90%, at least 92%, at least 94%, at least 95%, at least 96%, at least 97%, at least 98%, at least 99%, or 100% identical to SEQ ID NO: 141, and the LC variable domain may comprise an amino acid sequence at least 80%, at least 85%, at least 90%, at least 92%, at least 94%, at least 95%, at least 96%, at least 97%, at least 98%, at least 99%, or 100% identical to SEQ ID NO: 145. The HC variable domain may comprise an amino acid sequence at least 80%, at least 85%, at least 90%, at least 92%, at least 94%, at least 95%, at least 96%, at least 97%, at least 98%, at least 99%, or 100% identical to the amino acid sequence encoded by SEQ ID NO: 241, and the LC variable domain may comprise an amino acid sequence at least 80%, at least 85%, at least 90%, at least 92%, at least 94%, at least 95%, at least 96%, at least 97%, at least 98%, at least 99%, or 100% identical to the amino acid sequence encoded by SEQ ID NO: 245.

In some aspects, the Ab or antigen-binding Ab fragment may be for example, but not limited to, a monoclonal Ab, a monospecific Ab, a bispecific Ab, a multispecific Ab, a humanized Ab, a tetrameric Ab, a tetravalent Ab, a single chain Ab, a domain-specific Ab, a domain-deleted Ab, an scFc fusion protein, a chimeric Ab, a synthetic Ab, a recombinant Ab, a hybrid Ab, a mutated Ab, CDR-grafted Ab, a fragment antigen-binding (Fab), an F(ab′)2, an Fab′ fragment, a variable fragment (Fv), a single-chain Fv (scFv) fragment, an Fd fragment, a diabody, or a minibody.

In certain aspects, the antibody or antigen-binding Ab fragment may comprise the amino acid sequence at least 80%, at least 85%, at least 90%, at least 92%, at least 94%, at least 95%, at least 96%, at least 97%, at least 98%, at least 99%, or 100% identical to: (i) the amino acid sequence set forth in SEQ ID NOs: 139, 140, 149, or 150 or (ii) the amino acid sequence encoded by SEQ ID NOs: 239, 240, 249, or 250.

In some aspects, the Ab or antigen-binding Ab fragment may comprise two or more binding specificities. The first specificity may be to an epitope in ADAM12. In one aspect, the second specificity may to another epitope in ADAM12. In another aspect, the second specificity may be to an epitope in a second antigen other than ADAM12. In certain aspects, the second antigen may be for example, but not limited to, CD3, NKG2D, or 4-1BB.

In some aspects, the Ab or antigen-binding Ab fragment may comprise a human-like fragment crystallizable (Fc) region. The human-like Fc region may be at least 80%, at least 85%, at least 90%, at least 92%, at least 94%, at least 95%, at least 96%, at least 97%, at least 98%, at least 99%, or 100% identical to a human Fc region. The human-like Fc region may bind to an Fc receptor (FcR). The FcR may eb for example, but not limited to, Fc gamma receptor (FcgR), FcgRI, FcgRIIA, FcgRIIB1, FcgRIIB2, FcgRIIIA, FcgRIIIB, Fc epsilon receptor (FceR), FceRI, FceRII, Fc alpha receptor (FcaR), FcaRI, Fc alpha/mu receptor (Fca/mR), and neonatal Fc receptor (FcRn).

In some embodiments, the anti-ADAM12 anent of the present invention is an antibody-drug conjugate (ADC). The ADC may comprise: (a) any Ab or antigen-binding Ab fragment described above; and (b) an drug conjugated to the Ab or antigen-binding Ab fragment.

In some aspects, the drug may be for example, but not limited to, an anti-cancer drug, an anti-proliferative drug, a cytotoxic drug, an anti-angiogenic drug, an apoptotic drug, an immunostimulatory drug, an anti-microbial drug, an antibiotic drug, an antiviral drug, an anti-inflammatory drug, an anti-fibrotic drug, an immunosuppressive drug, a steroid, a bronchodilator, a beta blocker, a matrix metalloproteinase inhibitor, an ADAM12 inhibitor, an ADAM12 signaling inhibitor, an enzyme, a hormone, a neurotransmitter, a toxin, a radioisotope, a compound, a small molecule, a small molecule inhibitor, a protein, a peptide, a vector, a plasmid, a viral particle, a nanoparticle, a DNA molecule, an RNA molecule, an siRNA, an shRNA, a micro RNA, an oligonucleotide, or an imaging drug.

In some embodiments, the ADC may comprise a drug selected from the group consisting of doxorubicin, daunorubicin, cucurbitacin, chaetocin, chaetoglobosin, chlamydocin, calicheamicin, nemorubicin, cryptophyscin, mensacarcin, ansamitocin, mitomycin C, geldanamycin, mechercharmycin, rebeccamycin, safracin, okilactomycin, oligomycin, actinomycin, sandramycin, hypothemycin, polyketomycin, hydroxyellipticine, thiocolchicine, methotrexate, triptolide, taltobulin, lactacystin, dolastatin, auristatin, monomethyl auristatin E (MMAE), monomethyl auristatin F (MMAF), telomestatin, tubastatin A, combretastatin, maytansinoid, MMAD, MMAF, DM1, DM4, DTT, 16-GMB-APA-GA, 17-DMAP-GA, JW 55, pyrrolobenzodiazepine, SN-38, Ro 5-3335, puwainaphycin, duocarmycin, bafilomycin, taxoid, tubulysin, ferulenol, lusiol A, fumagillin, hygrolidin, glucopiericidin, amanitin, ansatrienin, cinerubin, phallacidin, phalloidin, phytosphongosine, piericidin, poronetin, phodophyllotoxin, gramicidin A, sanguinarine, sinefungin, herboxidiene, microcolin B, microcystin, muscotoxin A, tolytoxin, tripolin A, myoseverin, mytoxin B, nocuolin A, psuedolaric acid B, pseurotin A, cyclopamine, curvulin, colchicine, aphidicolin, englerin, cordycepin, apoptolidin, epothilone A, limaquinone, isatropolone, isofistularin, quinaldopeptin, ixabepilone, aeroplysinin, arruginosin, agrochelin, epothilone, and a derivative of any one of the foregoing.

In some embodiments, the anti-ADAM12 agent of the present invention is a chimeric antigen receptor (CAR). The CAR may comprise: (a) an AB domain that binds to ADAM12, (b) a transmembrane (TM) domain,

(c) an intracellular signaling (ICS) domain, (d) optionally a hinge that joins said AB domain and said TM domain, and (e) optionally one or more costimulatory (CS) domains.

In some aspects, the AB domain may be any Ab or antigen-binding Ab fragment described above.

In one aspect, the AB domain may comprise an amino acid sequence at least 80%, at least 85%, at least 90%, at least 92%, at least 94%, at least 95%, at least 96%, at least 97%, at least 98%, at least 99%, or 100% identical to the amino acid sequence: (i) set forth in SEQ ID NOs: 139, 140, 149, or 150, or (ii) encoded by SEQ ID NOs: 239, 240, 249, or 250.

In yet another aspect, the AB domain may compete for binding to ADAM12 with a scFv comprising an amino acid sequence at least 80%, at least 85%, at least 90%, at least 92%, at least 94%, at least 95%, at least 96%, at least 97%, at least 98%, at least 99%, or 100% identical to the amino acid sequence: (i) set forth in SEQ ID NOs: 139, 140, 149, or 150, or (ii) encoded by SEQ ID NOs: 239, 240, 249, or 250.

In certain aspects, the AB domain may comprise an amino acid sequence at least 80%, at least 85%, at least 90%, at least 92%, at least 94%, at least 95%, at least 96%, at least 97%, at least 98%, at least 99%, or 100% identical to the ADAM12-binding domain of a natural ADAM12-binding molecule. In one aspect, the natural ADAM12-binding molecule may be for example, but not limited to alpha actinin 2 (ACTN2), insulin-like growth factor-binding protein 3 (IGFBP3), IGFBP-5, phosphatidylinositol 3 kinase regulatory subunit alpha (PIK3R1), heparin-binding epidermal growth factor (HB-EGF), epidermal growth factor (EGF), Betacellulin, Delta-like 1, placental leucine aminopeptidase (P-LAP), and matrix metalloprotease 14 (MMP-14)

In some aspects, the TM domain may be derived from the TM region, or a membrane-spanning portion thereof, of for example, but not limited to, CD28, CD3e, CD4, CD5, CD8, CD9, CD16, CD22, CD33, CD37, CD45, CD64, CD80, CD86, CD134, CD137, CD154, TCRa, TCRb, or CD3z.

In certain aspects, the TM domain may be derived from the TM region of CD28, or a membrane-spanning portion thereof. The TM domain may optionally comprise an amino acid sequence at least 80%, at least 85%, at least 90%, at least 95%, at least 98% at least 99%, or 100% identical to the amino acid sequence: (i) set forth in SEQ ID NO: 161, or (ii) encoded by SEQ ID NO: 261.

In some aspects, the ICS domain may be derived from a cytoplasmic signaling sequence, or a functional fragment thereof, of for example, but not limited to, CD3z, a lymphocyte receptor chain, a TCR/CD3 complex protein, an Fc receptor (FcR) subunit, an IL-2 receptor subunit, FcRg, FcRb, CD3g, CD3d, CD3e, CD5, CD22, CD66d, CD79a, CD79b, CD278 (ICOS), FceRI, DAP10, or DAP12.

In certain aspects, the ICS domain may be derived from a cytoplasmic signaling sequence of CD3z, or a functional fragment thereof. The ICS domain may optionally comprise an amino acid sequence at least 80%, at least 85%, at least 90%, at least 95%, at least 98% at least 99%, or 100% identical to the amino acid sequence: (i) set forth in SEQ ID NO: 162, or (ii) encoded by SEQ ID NO: 262.

In some aspects, the hinge may be derived from CD28. The hinge may optionally comprise an amino sequence at least 80%, at least 85%, at least 90%, at least 95%, at least 98% at least 99%, or 100% identical to the amino acid sequence: (i) set forth in SEQ ID NO: 163, or (ii) encoded by SEQ ID NO: 263.

In some aspects, at least one of the one or more CS domains may be derived from a cytoplasmic signaling sequence, or functional fragment thereof, of for example, but not limited to, CD28, DAP10, 4-1BB (CD137), CD2, CD4, CD5, CD7, CD8a, CD8b, CD11a, CD11b, CD11c, CD11d, CD18, CD19, CD27, CD29, CD30, CD40, CD49d, CD49f, CD69, CD84, CD96 (Tactile), CD100 (SEMA4D), CD103, OX40 (CD134), SLAM (SLAMFI, CD150, IPO-3), CD160 (BY55), SELPLG (CD162), DNAM1 (CD226), Ly9 (CD229), SLAMF4 (CD244, 2B4), ICOS (CD278), B7-H3, BAFFR, BTLA, BLAME (SLAMF8), CEACAMI, CDS, CRTAM, GADS, GITR, HVEM (LIGHTER), IA4, ICAM-1, IL2Rb, IL2Rg, IL7Ra, ITGA4, ITGA6, ITGAD, ITGAE, ITGAL, ITGAM, ITGAX, ITGB1, ITGB2, ITGB7, KIRDS2, LAT, LFA-1, LIGHT, LTBR, NKG2C, NKG2D, NKp30, NKp44, NKp46, NKp80 (KLRF1), PAG/Cbp, PD-1, PSGL1, SLAMF6 (NTB-A, Ly108), SLAMF7, SLP-76, TNFR2, TRANCE/RANKL, VLA1, VLA-6, or CD83 ligand.

In certain aspects, the CS domain may be derived from a cytoplasmic signaling sequence of CD28, 4-1BB, or DAP10, or functional fragment thereof. The CS domain may optionally comprise an amino sequence at least 80%, at least 85%, at least 90%, at least 95%, at least 98% at least 99%, or 100% identical to the amino acid sequence: (i) set forth in SEQ ID NO: 164, (ii) encoded by SEQ ID NO: 264, (iii) set forth in SEQ ID NO: 165,

(iv) encoded by SEQ ID NO: 265, (v) set for the in SEQ ID NO: 166, or (vi) encoded by SEQ ID NO: 266.

In certain aspects, the CAR may comprise an amino acid sequence at least 80%, at least 85%, at least 90%, at least 95%, at least 98% at least 99%, or 100% identical to the amino acid sequence of (i) h6E6scFvHL-CD28H-CD28TM-CD28CS-CD3zICS (SEQ ID NO: 171), (ii) h6E6scFvHL-CD28H-CD28TM-41BBCS-CD3zICS (SEQ ID NO: 172), (iii) h6E6scFvHL-CD28H-CD28TM-DAP10CS-CD3zICS (SEQ ID NO: 173), (iv) h6E6scFvLH-CD28H-CD28TM-CD28CS-CD3zICS (SEQ ID NO: 174), (v) h6E6scFvLH-CD28H-CD28TM-41BBCS-CD3zICS (SEQ ID NO: 175), (vi) h6E6scFvLH-CD28H-CD28TM-DAP10CS-CD3zICS (SEQ ID NO: 176), (vii) h6C10scFvHL-CD28H-CD28TM-CD28CS-CD3zICS (SEQ ID NO: 177), (viii) h6C10scFvHL-CD28H-CD28TM-41BBCS-CD3zICS (SEQ ID NO: 178), (ix) h6C10scFvHL-CD28H-CD28TM-DAP10CS-CD3zICS (SEQ ID NO: 179), (x) h6C10scFvLH-CD28H-CD28TM-CD28CS-CD3zICS (SEQ ID NO: 180), (xi) h6C10scFvLH-CD28H-CD28TM-41BBCS-CD3zICS (SEQ ID NO: 181), (xii) h6C10scFvLH-CD28H-CD28TM-DAP10CS-CD3zICS (SEQ ID NO: 182), or

(xiii) to the amino acid sequence encoded by any one of SEQ ID NOs 271-282.

In some aspects, the CAR may further comprise a cytotoxic drug conjugated to the AB domain.

The present invention relates to isolated nucleic acid sequences encoding any anti-ADAM12 agents described above.

In some embodiments, the isolated nucleic acid sequence may encode an antibody (Ab) or antigen-binding Ab fragment. The Ab or antigen-binding Ab fragment may bind to ADAM12 and comprise: a HC variable domain (VH) and a LC variable domain (VL). The VH may comprise: a HC complementarity determining region (CDR) 1; a HC CDR 2; a HC CDR 3, and a human-like HC framework. The VL may comprise: a LC CDR 1; a LC CDR 2; a LC CDR 3; and a human-like LC framework.

In some aspects, the HC CDR 1, HC CDR 2, and HC CDR3 may comprise the amino acid sequence set forth in SEQ ID NOs: 132, 133, and 134, respectively, and the LC CDR 1, LC CDR 2, and LC CDR 3 may comprise the amino acid sequence set forth in SEQ ID NOs: 136, 137, and 138, respectively. The HC CDR 1, HC CDR 2, and HC CDR 3 may comprise the amino acid sequence encoded by SEQ ID NOs: 232, 233, and 234, respectively, and the LC CDR 1, LC CDR 2, and LC CDR 3 may comprise the amino acid sequence encoded by SEQ ID NOs: 236, 237, and 238, respectively. Alternatively, the HC CDR 1, HC CDR 2, and HC CDR 3 may comprise the amino acid sequence set forth in SEQ ID NOs: 142, 143, and 144, respectively, and the LC CDR 1, LC CDR 2, and LC CDR 3 may comprise the amino acid sequence set forth in SEQ ID NOs: 146, 147, and 148, respectively. The HC CDR 1, HC CDR 2, and HC CDR 3 may comprise the amino acid sequence encoded by SEQ ID NOs: 242, 243, and 244, respectively, and the LC CDR 1, LC CDR 2, and LC CDR 3 may comprise the amino acid sequence encoded by SEQ ID NOs: 246, 247, and 248, respectively.

In some aspects, the human-like HC framework may be at least 80%, at least 85%, at least 90%, at least 92%, at least 94%, at least 95%, at least 96%, at least 97%, at least 98%, at least 99%, or 100% identical to a human HC framework. The human-like LC framework may be at least 80%, at least 85%, at least 90%, at least 92%, at least 94%, at least 95%, at least 96%, at least 97%, at least 98%, at least 99%, or 100% identical to a human LC framework.

In certain aspects, the isolated nucleic acid sequence may encode an Ab or antigen-binding Ab fragment and the VH may comprise an amino acid sequence at least 80%, at least 85%, at least 90%, at least 92%, at least 94%, at least 95%, at least 96%, at least 97%, at least 98%, at least 99%, or 100% identical to SEQ ID NO: 131, and the VL may comprise an amino acid sequence at least 80%, at least 85%, at least 90%, at least 92%, at least 94%, at least 95%, at least 96%, at least 97%, at least 98%, at least 99%, or 100% identical to SEQ ID NO: 135. The VH may compriss an amino acid sequence at least 80%, at least 85%, at least 90%, at least 92%, at least 94%, at least 95%, at least 96%, at least 97%, at least 98%, at least 99%, or 100% identical to the amino acid sequence encoded by SEQ ID NO: 231, and the VL may comprise an amino acid sequence at least 80%, at least 85%, at least 90%, at least 92%, at least 94%, at least 95%, at least 96%, at least 97%, at least 98%, at least 99%, or 100% identical to the amino acid sequence encoded by SEQ ID NO: 235. Alternatively, the VH may comprise an amino acid sequence at least 80%, at least 85%, at least 90%, at least 92%, at least 94%, at least 95%, at least 96%, at least 97%, at least 98%, at least 99%, or 100% identical to SEQ ID NO: 141, and the VL may comprise an amino acid sequence at least 80%, at least 85%, at least 90%, at least 92%, at least 94%, at least 95%, at least 96%, at least 97%, at least 98%, at least 99%, or 100% identical to SEQ ID NO: 145. The VH may comprise an amino acid sequence at least 80%, at least 85%, at least 90%, at least 92%, at least 94%, at least 95%, at least 96%, at least 97%, at least 98%, at least 99%, or 100% identical to the amino acid sequence encoded by SEQ ID NO: 241, and the VL may comprise an amino acid sequence at least 80%, at least 85%, at least 90%, at least 92%, at least 94%, at least 95%, at least 96%, at least 97%, at least 98%, at least 99%, or 100% identical to the amino acid sequence encoded by SEQ ID NO: 245.

In some aspects, the isolated nucleic acid sequence may encode an Ab or antigen-binding Ab fragment that is for example, but not limited to, a monoclonal Ab, a monospecific Ab, a bispecific Ab, a multispecific Ab, a humanized Ab, a tetrameric Ab, a tetravalent Ab, a single chain Ab, a domain-specific Ab, a domain-deleted Ab, an scFc fusion protein, a chimeric Ab, a synthetic Ab, a recombinant Ab, a hybrid Ab, a mutated Ab, CDR-grafted Ab, a fragment antigen-binding (Fab), an F(ab′)2, an Fab′ fragment, a variable fragment (Fv), a single-chain Fv (scFv) fragment, an Fd fragment, a diabody, or a minibody.

In certain aspects, the isolated nucleic acid sequence may encode an Ab or antigen-binding Ab fragment that comprise the amino acid at least 80%, at least 85%, at least 90%, at least 92%, at least 94%, at least 95%, at least 96%, at least 97%, at least 98%, at least 99%, or 100% identical to the amino acid sequence: (i) set forth in SEQ ID NOs: 139, 140, 149, or 150, or (ii) encoded by SEQ ID NOs: 239, 240, 249, or 250.

In some aspects, the isolated nucleic acid sequence may encode an Ab or antigen-binding Ab fragment that comprise two or more binding specificities. The first specificity may be to an epitope in ADAM12. In one aspect, the second specificity may be to another epitope in ADAM12. In another aspect, the second specificity may be to an epitope in a second antigen other than ADAM12. In certain aspects, the second antigen may be for example, but not limited to, CD3, NKG2D, or 4-1BB.

In some aspects, the isolated nucleic acid may encode an Ab or antigen-binding Ab fragment that comprises a human-like fragment crystallizable (Fc) region. In certain aspects, the human-like Fc region may be at least 80%, at least 85%, at least 90%, at least 92%, at least 94%, at least 95%, at least 96%, at least 97%, at least 98%, at least 99%, or 100% identical to a human Fc region.

The human-like Fc region may bind to an Fc receptor (FcR). The FcR may be for example, but not limited to, Fc gamma receptor (FcgR), FcgRI, FcgRIIA, FcgRIIB1, FcgRIIB2, FcgRIIIA, FcgRIIIB, Fc epsilon receptor (FceR), FceRI, FceRII, Fc alpha receptor (FcaR), FcaRI, Fc alpha/mu receptor (Fca/mR), or neonatal Fc receptor (FcRn).

In some embodiments, the isolated nucleic acid sequence may encode any CAR described above. The CAR may comprise: (a) an AB domain that binds to ADAM12; (b) a transmembrane (TM) domain; (c) an intracellular signaling (ICS) domain; (d) optionally a hinge that joins said AB domain and said TM domain; and (e) optionally one or more costimulatory (CS) domains.

In some aspects, the isolated nucleic acid sequence may encode a CAR whose AB domain is encoded by any of the AB domain-encoding nucleic acid sequences described above.

In certain aspects, the isolated nucleic acid sequence may encode a CAR whose AB domain comprises an amino acid sequence at least 80%, at least 85%, at least 90%, at least 92%, at least 94%, at least 95%, at least 96%, at least 97%, at least 98%, at least 99%, or 100% identical to the amino acid sequence: (i) set forth in SEQ ID NOs: 139, 140, 149, or 150, or (ii) encoded by SEQ ID NOs: 239, 240, 249, or 250.

In certain aspects, the isolated nucleic acid sequence may encode a CAR whose AB domain competes for binding to ADAM12 with a scFv comprising an amino acid sequence at least 80%, at least 85%, at least 90%, at least 92%, at least 94%, at least 95%, at least 96%, at least 97%, at least 98%, at least 99%, or 100% identical to: (i) SEQ ID NOs: 139, 140, 149, or 150, or (ii) the amino acid sequence encoded by SEQ ID NOs: 239, 240, 249, or 250.

In certain aspects, the isolated nucleic acid sequence may encode a CAR whose AB domain comprises an amino acid sequence at least 80%, at least 85%, at least 90%, at least 92%, at least 94%, at least 95%, at least 96%, at least 97%, at least 98%, at least 99%, or 100% identical to the ADAM12-binding domain of a natural ADAM12-binding molecule. In certain embodiments, the natural ADAM12-binding molecule may be, for example, but not limited to, alpha actinin 2 (ACTN2), insulin-like growth factor-binding protein 3 (IGFBP3), IGFBP-5, phosphatidylinositol 3 kinase regulatory subunit alpha (PIK3R1), heparin-binding epidermal growth factor (HB-EGF), epidermal growth factor (EGF), Betacellulin, Delta-like 1, placental leucine aminopeptidase (P-LAP), and matrix metalloprotease 14 (MMP-14).

In some aspects, the isolated nucleic acid sequence may encode a CAR whose TM domain is derived from the TM region, or a membrane-spanning portion thereof, of, for example, but not limited to, CD28, CD3e, CD4, CD5, CD8, CD9, CD16, CD22, CD33, CD37, CD45, CD64, CD80, CD86, CD134, CD137, CD154, TCRa, TCRb, and CD3z. In certain aspects, the TM domain may be derived from the TM region of CD28, or a membrane-spanning portion thereof. The TM domain may optionally comprise an amino acid sequence at least 80%, at least 85%, at least 90%, at least 95%, at least 98% at least 99%, or 100% identical to the amino acid sequence: (i) set forth in SEQ ID NO: 161, or (ii) encoded by SEQ ID NO: 261.

In some aspects, the isolated nucleic acid sequence may encode a CAR whose ICS domain is derived from a cytoplasmic signaling sequence, or a functional fragment thereof, of, for example, but not limited to, CD3z, a lymphocyte receptor chain, a TCR/CD3 complex protein, an Fc receptor (FcR) subunit, an IL-2 receptor subunit, FcRg, FcRb, CD3g, CD3d, CD3e, CD5, CD22, CD66d, CD79a, CD79b, CD278 (ICOS), FceRI, DAP10, and DAP12. In certain aspects, the ICS domain may derived from a cytoplasmic signaling sequence of CD3z, or a functional fragment thereof. The ICS domain may optionally comprise an amino acid sequence at least 80%, at least 85%, at least 90%, at least 95%, at least 98% at least 99%, or 100% identical to the amino acid sequence: (i) set forth in SEQ ID NO: 162, or (ii) encoded by SEQ ID NO: 262.

In some aspects, the isolated nucleic acid sequence may encode a CAR whose hinge is derived from CD28. The hinge may optionally comprise an amino sequence at least 80%, at least 85%, at least 90%, at least 95%, at least 98% at least 99%, or 100% identical to the amino acid sequence: (i) set forth in SEQ ID NO: 163, or (ii) encoded by SEQ ID NO: 263.

In some aspects, the isolated nucleic acid sequence may encode a CAR whose at least one of the one or more CS domains is derived from a cytoplasmic signaling sequence, or functional fragment thereof, of, for example, but not limited to, CD28, DAP10, 4-1BB (CD137), CD2, CD4, CD5, CD7, CD8a, CD8b, CD11a, CD11b, CD11c, CD11d, CD18, CD19, CD27, CD29, CD30, CD40, CD49d, CD49f, CD69, CD84, CD96 (Tactile), CD100 (SEMA4D), CD103, OX40 (CD134), SLAM (SLAMFI, CD150, IPO-3), CD160 (BY55), SELPLG (CD162), DNAM1 (CD226), Ly9 (CD229), SLAMF4 (CD244, 2B4), ICOS (CD278), B7-H3, BAFFR, BTLA, BLAME (SLAMF8), CEACAMI, CDS, CRTAM, GADS, GITR, HVEM (LIGHTER), IA4, ICAM-1, IL2Rb, IL2Rg, IL7Ra, ITGA4, ITGA6, ITGAD, ITGAE, ITGAL, ITGAM, ITGAX, ITGB1, ITGB2, ITGB7, KIRDS2, LAT, LFA-1, LIGHT, LTBR, NKG2C, NKG2D, NKp30, NKp44, NKp46, NKp80 (KLRF1), PAG/Cbp, PD-1, PSGL1, SLAMF6 (NTB-A, Ly108), SLAMF7, SLP-76, TNFR2, TRANCE/RANKL, VLA1, VLA-6, or CD83 ligand. In certain aspects, the CS domain may be derived from a cytoplasmic signaling sequence of CD28, 4-1BB, or DAP10, or functional fragment thereof. The CS domain may optionally comprise an amino sequence at least 80%, at least 85%, at least 90%, at least 95%, at least 98% at least 99%, or 100% identical to the amino acid sequence: (i) set forth in SEQ ID NO: 164, (ii) encoded by SEQ ID NO: 264, (iii) set forth in SEQ ID NO: 165, (iv) encoded by SEQ ID NO: 265, (v) set for the in SEQ ID NO: 166, or (vi) encoded by SEQ ID NO: 266.

In some aspects, the isolated nucleic acid sequence may encode a CAR that comprises amino acid sequence at least 80%, at least 85%, at least 90%, at least 95%, at least 98% at least 99%, or 100% identical to the amino acid sequence of (i) h6E6scFvHL-CD28H-CD28TM-CD28CS-CD3zICS (SEQ ID NO: 171), (ii) h6E6scFvHL-CD28H-CD28TM-41BBCS-CD3zICS (SEQ ID NO: 172), (iii) h6E6scFvHL-CD28H-CD28TM-DAP10CS-CD3zICS (SEQ ID NO: 173), (iv) h6E6scFvLH-CD28H-CD28TM-CD28CS-CD3zICS (SEQ ID NO: 174), (v) h6E6scFvLH-CD28H-CD28TM-41BBCS-CD3zICS (SEQ ID NO: 175), (vi) h6E6scFvLH-CD28H-CD28TM-DAP10CS-CD3zICS (SEQ ID NO: 176), (vii) h6C10scFvHL-CD28H-CD28TM-CD28CS-CD3zICS (SEQ ID NO: 177), (viii) h6C10scFvHL-CD28H-CD28TM-41BBCS-CD3zICS (SEQ ID NO: 178), (ix) h6C10scFvHL-CD28H-CD28TM-DAP10CS-CD3zICS (SEQ ID NO: 179), (x) h6C10scFvLH-CD28H-CD28TM-CD28CS-CD3zICS (SEQ ID NO: 180), (xi) h6C10scFvLH-CD28H-CD28TM-41BBCS-CD3zICS (SEQ ID NO: 181), (xii) h6C10scFvLH-CD28H-CD28TM-DAP10CS-CD3zICS (SEQ ID NO: 182), or (xiii) to the amino acid sequence encoded by any one of SEQ ID NOs 271-282.

In some embodiments, the isolated nucleic acid sequence of any of the above may further comprise a leader sequence (LS). In one aspect, the LS is optionally at least 80%, at least 85%, at least 90%, at least 95%, at least 98% at least 99%, or 100% identical to SEQ ID NO: 260.

In some embodiments, the isolated nucleic acid sequence of any one of the above may further comprise T2A sequence and/or a sequence encoding truncated CD19 (trCD19). In one aspect, the T2A sequence is optionally at least 80%, at least 85%, at least 90%, at least 95%, at least 98% at least 99%, or 100% identical to SEQ ID NO: 269. In one aspect, the trCD19 may optionally comprise the amino acid sequence at least 85%, at least 90%, at least 95%, at least 98% at least 99%, or 100% identical to SEQ ID NO: 170. In certain aspect, the trCD19 may be encoded by the nucleic acid sequence at least 80%, at least 85%, at least 90%, at least 95%, at least 98% at least 99%, or 100% identical to SEQ ID NO: 270.

The present invention relates to vectors comprising any nucleic acid sequence encoding an anti-ADAM12 agent.

In some embodiments, the vector may comprise any nucleic acid sequence described above.

In some aspects, the vector may be for example, but not limited to, a DNA, an RNA, a plasmid, a cosmid, a viral vector, a lentiviral vector, an adenoviral vector, or a retroviral vector.

The present invention relates to recombinant or isolated cells.

In some embodiments, the recombinant or isolated cell may comprise: (i) any Ab or antigen-binding Ab fragment described above, (ii) any ADC described above, (iii) any CAR described above, (iv) any nucleic acid sequence described above, or (v) any vector described above.

In some aspects, the recombinant or isolated cell may be, for example, but not limited to, a non-mammalian cell, optionally a plant cell, a bacterial cell, a fungal cell, a yeast cell, a protozoa cell, or an insect cell.

In some aspects, the recombinant or isolated cell may be, for example, but not limited to, a mammalian cell, optionally a human cell, a rat cell, or a mouse cell.

In some aspects, the recombinant or isolated cell may be, for example, but not limited to, a stem cell.

In some aspects, the recombinant or isolated cell may be, for example, but not limited to, a primary cell, optionally a human primary cell or derived therefrom.

In some aspects, the recombinant or isolated cell may be, for example, but not limited to, a cell line, optionally a hybridoma cell line.

In some aspects, the recombinant or isolated cell may be, for example, but not limited to, an immune cell.

In some aspects, the recombinant or isolated cell may be MHC+ or MHC−.

In some aspects, the recombinant or isolated cell may be, for example, but not limited to, a cell line, a T cell, a T cell progenitor cell, a CD4+ T cell, a helper T cell, a regulatory T cell, a CD8+ T cell, a naïve T cell, an effector T cell, a memory T cell, a stem cell memory T (TSCM) cell, a central memory T (TCM) cell, an effector memory T (TEM) cell, a terminally differentiated effector memory T cell, a tumor-infiltrating lymphocyte (TIL), an immature T cell, a mature T cell, a cytotoxic T cell, a mucosa-associated invariant T (MAIT) cell, a TH1 cell, a TH2 cell, a TH3 cell, a TH17 cell, a TH9 cell, a TH22 cell, a follicular helper T cells, and a/b T cell, a g/d T cell, a Natural Killer T (NKT) cell, a cytokine-induced killer (CIK) cell, a lymphokine-activated killer (LAK) cell, a perforin-deficient cell, a granzyme-deficient cell, a B cell, a myeloid cell, a monocyte, a macrophage, or a dendritic cell.

In certain aspects, the recombinant or isolated cell may be a T cell or T cell progenitor cell.

In certain aspects, the recombinant or isolated cell may be a T cell which has been modified such that its endogenous T cell receptor (TCR) is (i) not expressed, (ii) not functionally expressed, or (iii) expressed at reduced levels compared to a wild-type T cell.

In certain aspects, the recombinant or isolated cell may be activated or stimulated to proliferate when the CAR binds to its target molecule.

In certain aspects, the recombinant or isolated cell may exhibit cytotoxicity against cells expressing the target molecule when the CAR binds to the target molecule.

In certain aspects, administration of the recombinant or isolated cell may ameliorate a disease, cancer, a cardiac condition, an autoimmune condition, an inflammatory condition, a fibrotic condition, when the CAR binds to its target molecule.

In certain aspects, the recombinant or isolated cell may increase expression of cytokines and/or chemokines when the CAR binds to its target molecule. The cytokine may be IFN-g.

In certain aspects, the recombinant or isolated cell may decrease expression of cytokines and/or chemokines when the CAR binds to its target. The cytokine may be TGF-b.

The present invention relates to populations of recombinant or isolated cells.

In some embodiments, the population may comprise at least any one recombinant or isolated cell described above.

The present invention relates to pharmaceutical compositions.

In some embodiments, the pharmaceutical composition may comprise: (a) (a-i) any Ab or antigen-binding Ab fragment described above, (a-ii) any ADC described above, (iii) any CAR described above, (iv) any nucleic acid sequence described above, (v) any vector described above, (vi) any cell described above, or (vii) any population of cells; and optionally (b) a pharmaceutically acceptable excipient or carrier.

The present invention relates to methods of treating a subject.

In some embodiments, the method may be a method of treating a subject, and the method may comprise administering to the subject in need thereof a therapeutically effective amount of: (i) any Ab or antigen-binding Ab fragment described above, (ii) any ADC described above, (iii) any CAR described above, (iv) any nucleic acid sequence described above, (v) any vector described above, (vi) any cell described above, (vii) any population of cells described above, or (viii) any pharmaceutical composition described above.

In some aspects, the method may be used in the treatment of, for example, but not limited to, cancer, fibrosis, autoimmunity, a cardiovascular condition, an allergic condition, a respiratory disease, a nephropathy, a neural disease, a muscular disease, a liver disease, metabolic syndrome, infection, or an inflammatory disorder. In certain aspects, the method may be used in the treatment of cancer, which is for example, but not limited to bladder cancer, bone cancer, brain cancer, breast cancer, colon cancer, colorectal cancer, desmoid tumor, esophageal cancer, fibromatosis, glioblastoma, head and neck cancer, liver cancer, lung cancer, melanoma, oesophago-gastric adenocarcinoma, oligodendroma, oral cancer, oral squamous cell carcinoma, osteosarcoma, ovarian cancer, pancreatic cancer, prostate cancer, skin cancer, small cell lung cancer, stomach cancer, or thyroid cancer.

In some embodiments, the method may be a method of treating a subject with anti-ADAM12 agent. The method may comprise the steps of: (a) obtaining or having obtained a biological sample from the subject; (b) measuring the expression level of ADAM12 in the biological sample; (c) determining whether the subject is an ADAM12 over-expresser; and (d) if the subject is an ADAM12 over-expresser, administering to the subject a therapeutically effective amount of (d-i) any Ab or antigen-binding Ab fragment described above, (d-ii) any ADC described above, (d-iii) any CAR described above, (d-iv) any nucleic acid sequence described above, (d-v) any vector described above, (d-vi) any cell described above, (d-vii) any population of cells described above, or (d-viii) any pharmaceutical composition described above. In certain aspects, an ADAM12 over-expresser is a subject whose ADAM12 expression is at least 1.5 times higher than the ADAM12 expression of normal or healthy subjects. In certain aspects, an ADAM12 over-expresser is a subject whose ADAM12 expression is at least 1.75 times higher than the ADAM12 expression of normal or healthy subjects. In certain aspects, an ADAM12 over-expresser is a subject whose ADAM12 expression is at least twice higher than the ADAM12 expression of normal or healthy subjects.

In some aspects, the subject may be suffering from cancer. The cancer may for example, but not limited to bladder cancer, bone cancer, brain cancer, breast cancer, colon cancer, colorectal cancer, desmoid tumor, esophageal cancer, fibromatosis, glioblastoma, head and neck cancer, liver cancer, lung cancer, melanoma, oesophago-gastric adenocarcinoma, oligodendroma, oral cancer, oral squamous cell carcinoma, osteosarcoma, ovarian cancer, pancreatic cancer, prostate cancer, skin cancer, small cell lung cancer, stomach cancer, or thyroid cancer.

The present invention relates to methods of immune stimulation.

In some embodiments, the method may be a method for stimulating an immune response in a subject, comprising administering to the subject a therapeutically effective amount of: (i) any Ab or antigen-binding Ab fragment described above, (ii) any ADC described above, (iii) any CAR described above, (iv) any nucleic acid sequence described above, (v) any vector described above, (vi) any cell described above, (vii) any population of cells described above, or (viii) any pharmaceutical composition described above.

The present invention relates to methods of treating a disease.

In some embodiments, the method may be a method of treating a disease in a subject, comprising administering to the subject in need thereof a therapeutically effective amount of: (i) any Ab or antigen-binding Ab fragment described above, (ii) any ADC described above, (iii) any CAR described above, (iv) any nucleic acid sequence described above, (v) any vector described above, (vi) any cell described above, (vii) any population of cells described above, or (viii) any pharmaceutical composition described above.

In some aspects, the disease may be, for example, but not limited to, cancer, fibrosis, autoimmunity, a cardiovascular condition, an allergic condition, a respiratory disease, a nephropathy, a neural disease, a muscular disease, a liver disease, metabolic syndrome, infection, or an inflammatory disorder.

In certain aspects, the disease may be cancer, which may optionally be bladder cancer, bone cancer, brain cancer, breast cancer, colon cancer, colorectal cancer, desmoid tumor, esophageal cancer, fibromatosis, glioblastoma, head and neck cancer, liver cancer, lung cancer, melanoma, oesophago-gastric adenocarcinoma, oligodendroma, oral cancer, oral squamous cell carcinoma, osteosarcoma, ovarian cancer, pancreatic cancer, prostate cancer, skin cancer, small cell lung cancer, stomach cancer, or thyroid cancer.

The present invention further relates to methods of expanding a population of cells.

In some aspects, the method may be a method of expanding a population of cells in a subject.

In some embodiments, the method may comprise administering to the subject: (i) any nucleic acid sequence described above; (ii) any vector described above; (iii) any cell described above; (iv) any population of cells described above; or (v) any pharmaceutical composition described above.

In some embodiments, the administration may lead to a population of cells including at least one desired cell, for example, a cell that may comprise any Ab or Ab fragment described above, any ADC described above, and/or any CAR described above. In certain embodiments, the cell may comprise a nucleic acid encoding such an Ab or Ab fragment, ADC, or CAR.

In some embodiments, the resulting population of cells may persist in the subject for at least three months, at least four months, at least five months, at least six months, at least seven months, at least eight months, at least nine months, at least ten months, at least eleven months, at least twelve months, at least eighteen months, at least two years, or at least three years after administration.

In some embodiments, the subject may be suffering from cancer.

In certain embodiments, the cancer may be bladder cancer, bone cancer, brain cancer, breast cancer, colon cancer, colorectal cancer, desmoid tumor, esophageal cancer, fibromatosis, glioblastoma, head and neck cancer, liver cancer, lung cancer, melanoma, oesophago-gastric adenocarcinoma, oligodendroma, oral cancer, oral squamous cell carcinoma, osteosarcoma, ovarian cancer, pancreatic cancer, prostate cancer, skin cancer, small cell lung cancer, stomach cancer, or thyroid cancer.

In some aspects, any of the methods described above may further comprise administering a second agent.

In certain aspects, the second agent may be, but is not limited to, an anti-cancer drug, an anti-proliferative drug, a cytotoxic drug, an anti-angiogenic drug, an apoptotic drug, an immunostimulatory drug, an anti-microbial drug, an antibiotic drug, an antiviral drug, an anti-inflammatory drug, an anti-fibrotic drug, an immunosuppressive drug, a steroid, a bronchodilator, a beta blocker, a matrix metalloproteinase inhibitor, an ADAM12 inhibitor, an ADAM12 signaling inhibitor, an anti-ADAM12 agent of the present invention, an enzyme, a hormone, a neurotransmitter, a toxin, a radioisotope, a compound, a small molecule, a small molecule inhibitor, a protein, a peptide, a vector, a plasmid, a viral particle, a nanoparticle, a DNA molecule, an RNA molecule, an siRNA, an shRNA, a micro RNA, an oligonucleotide, or an imaging drug.

The present invention further relates to methods of generating a cell comprising any CAR described above.

In some embodiments, the method may comprise: (i) introducing into a cell (i-a) a nucleic acid sequence encoding at least one CAR according to any one of the foregoing or (i-b) at least one nucleic acid sequence according to any one of the foregoing; or (ii) transducing a cell with the vector according to any one of the foregoing. Optionally, the method may further comprise (iii) isolating the cell based on expression of said CAR and/or a selectable marker as determined via flow cytometry or immunofluorescence assays.

### DETAILED DESCRIPTION OF THE SEVERAL VIEWS OF THE DRAWINGS

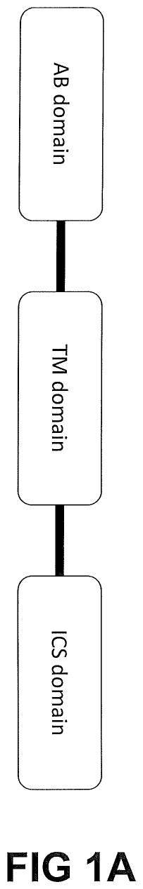

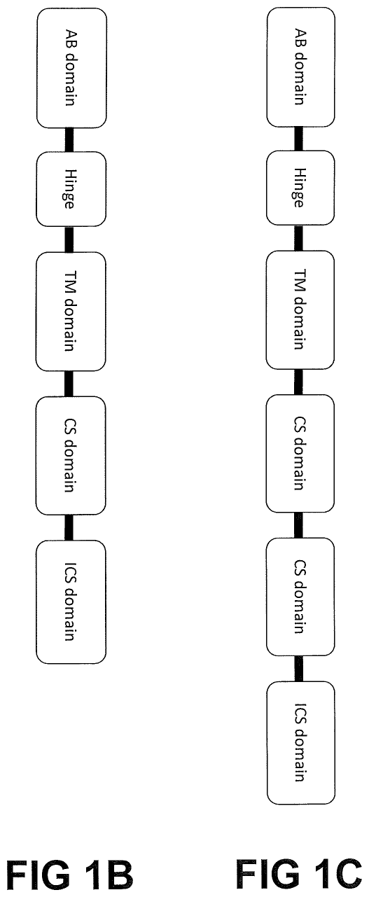

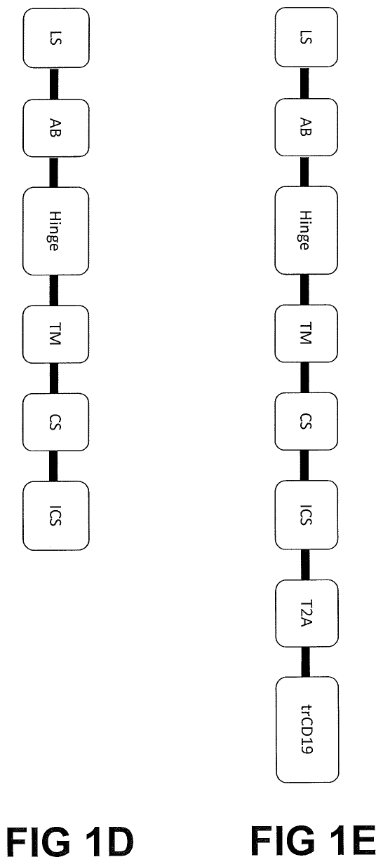

FIGS. 1A-E provide exemplary schematics of chimeric antigen receptors (CARs) according to the present disclosure. FIG. 1A shows a general schematic of chimeric antigen receptors (CARs) of the present invention. FIG. 1B shows an exemplary schematic of a CAR construct according to the present disclosure, wherein the CAR construct comprises an antigen-binding (AB) domain, a transmembrane (TM) domain, and an intracellular signaling (ICS) domain, and further comprises a hinge that joins the AB and TM domains, and one or two costimulatory (CS) domains. FIG. 1C shows an exemplary schematic of a vector construct encoding a CAR according to the present disclosure, wherein the vector comprises a leader sequence (LS) and an exemplary CAR construct as shown in FIG. 1B. FIG. 1E shows an exemplary schematic of a vector construct encoding a CAR according to the present disclosure, wherein the vector further comprises an exemplary ribosomal skip sequence (T2A) and an exemplary expression/purification marker, truncated CD19 (trCD19).

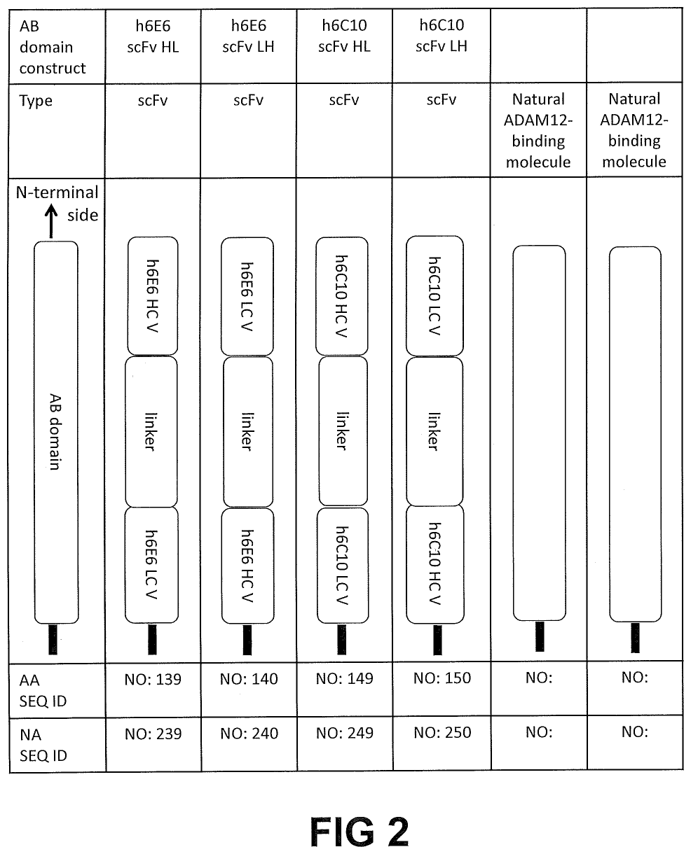

FIG. 2 illustrates a schematic showing various exemplary AB domain constructs of CARs of some embodiments. The first two examples are “h6E6scFvHL” (or “h6E6 scFv HL”) and “h6E6scFvLH” (or “h6E6 scFv LH”), which are scFvs derived from “h6E6”, the humanized version of mouse anti-ADAM12 antibody 6E6. The next two examples are “h6C10scFvHL” (or “h6C10 scFv HL”) and “h6C10scFvLH” (or “h6C10 scFv LH”), which are scFvs derived from “h6C10”, the humanized version of mouse anti-ADAM12 antibody 6C10. In the last two examples, a naturally existing molecule that binds to ADAM12 or the ADAM12-binding portion of such a molecule is used in an AB domain.

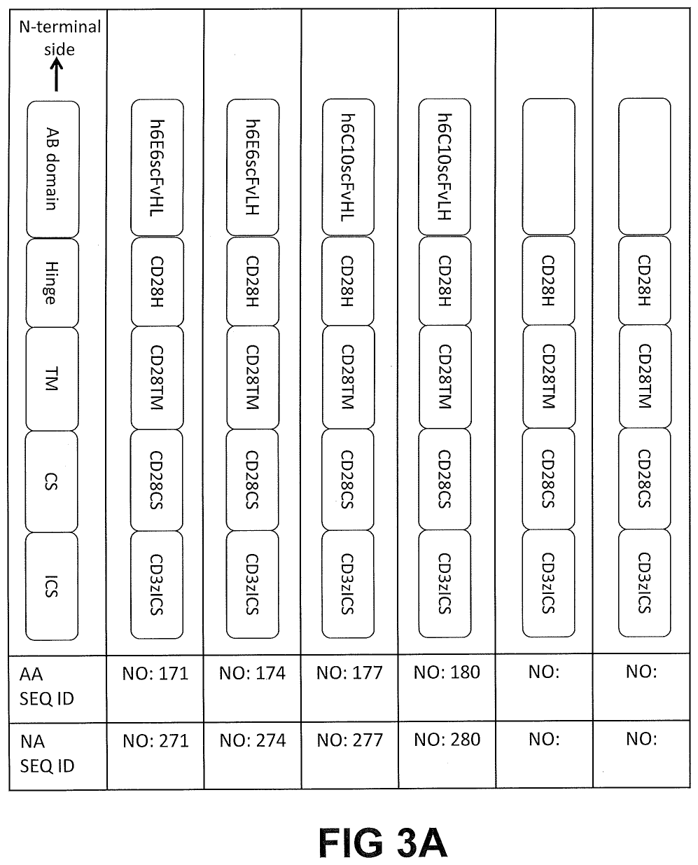

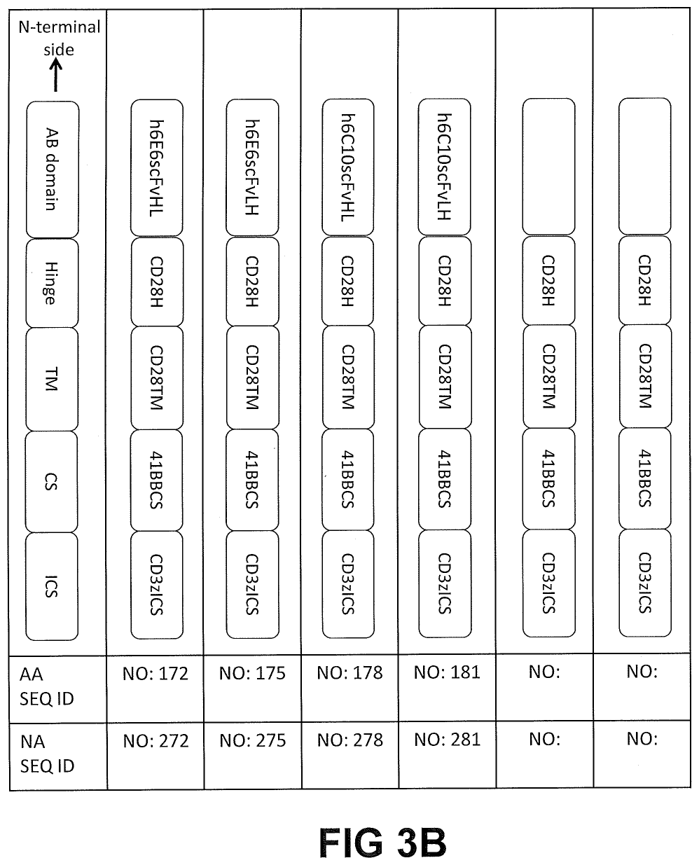

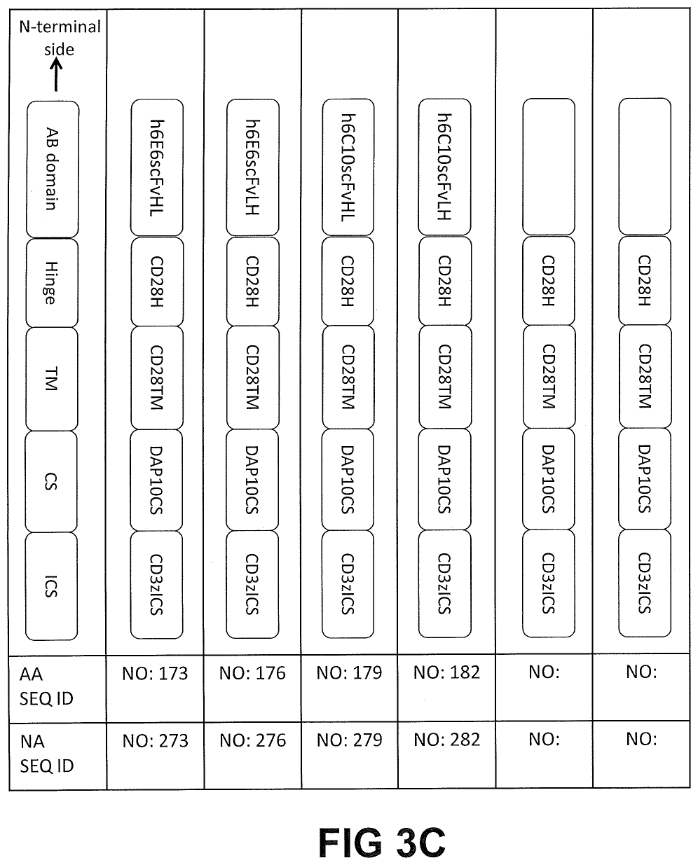

FIGS. 3A-C contain schematics of various exemplary CAR constructs of some embodiments of the invention. In the exemplary constructs in FIG. 3A, one of the AB domains shown in FIG. 2 is used as the AB domain, CD28H is used as the hinge, CD28TM is used as the TM domain, CD28CS is used as the CS domain, and CD3zICS is used as the ICS domain. In the exemplary constructs in FIG. 3B, one of the AB domains shown in FIG. 2 is used as the AB domain, CD28H is used as the hinge, CD28TM is used as the TM domain, 41BBCS is used as the CS domain, and CD3zICS is used as the ICS domain. In the exemplary constructs in FIG. 3C, one of the AB domains shown in FIG. 2 is used as the AB domain, CD28H is used as the hinge, CD28TM is used as the TM domain, DAP10CS is used as the CS domain, and CD3zICS is used as the ICS domain. CD28H is the hinge derived from human CD28. CD28TM is the TM domain derived from human CD28. CD28CS is the CS region derived from a cytoplasmic signaling sequence of human CD28. CD3zICS is the ICS domain derived from a human CD3 zeta. Any of the CAR constructs described in this figure or in this application may be used with LS, T2A, and/or trCD19, as shown in FIG. 1C.

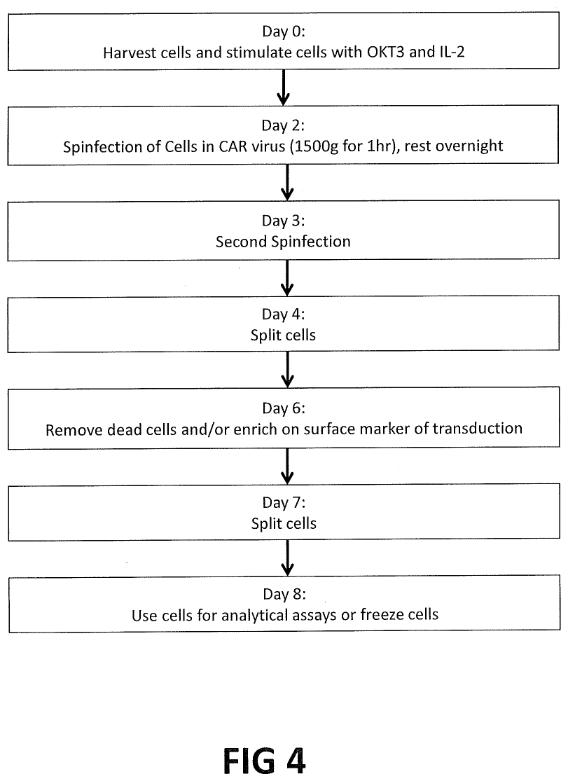

FIG. 4 shows a flow chart illustrating one of many possible methods for manufacturing isolated recombinant CAR-expressing cells that may be used for in vitro or in vivo assays.

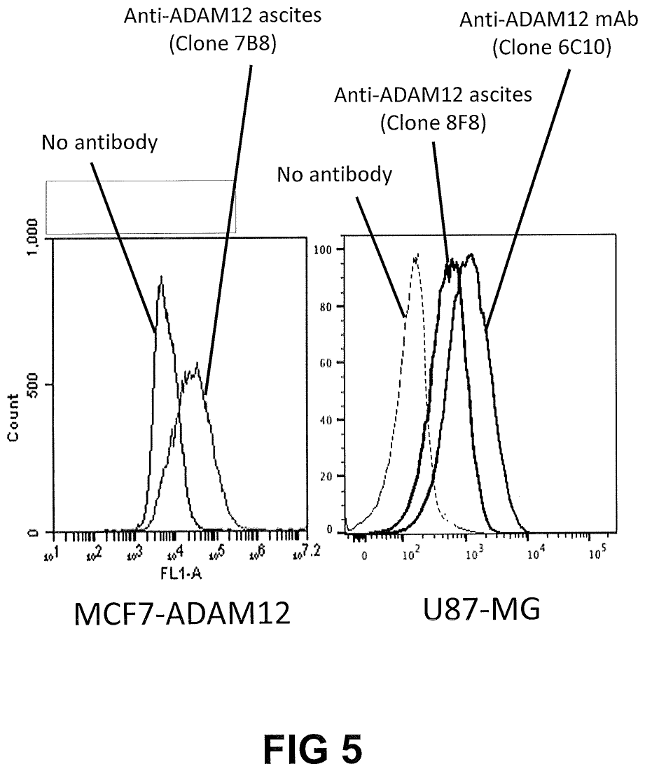

FIG. 5 contains histograms of ADAM12 staining of cancer cell lines analyzed by flow cytometry. MCF7-ADAM12 cells (human breast cancer cell line transfected with an ADAM12 expression vector) (left) were stained with an unpurified ascites sample harvested from mice harboring cells producing anti-human ADAM12 antibody (Clone 7B8), and U87-MG cells (glioblastoma cell line) (right) were stained with an unpurified ascites sample harvested from mice harboring cells producing anti-human ADAM12 antibody (Clone 8F8) or with mouse anti-human ADAM12 monoclonal antibody (Clone 6C10).

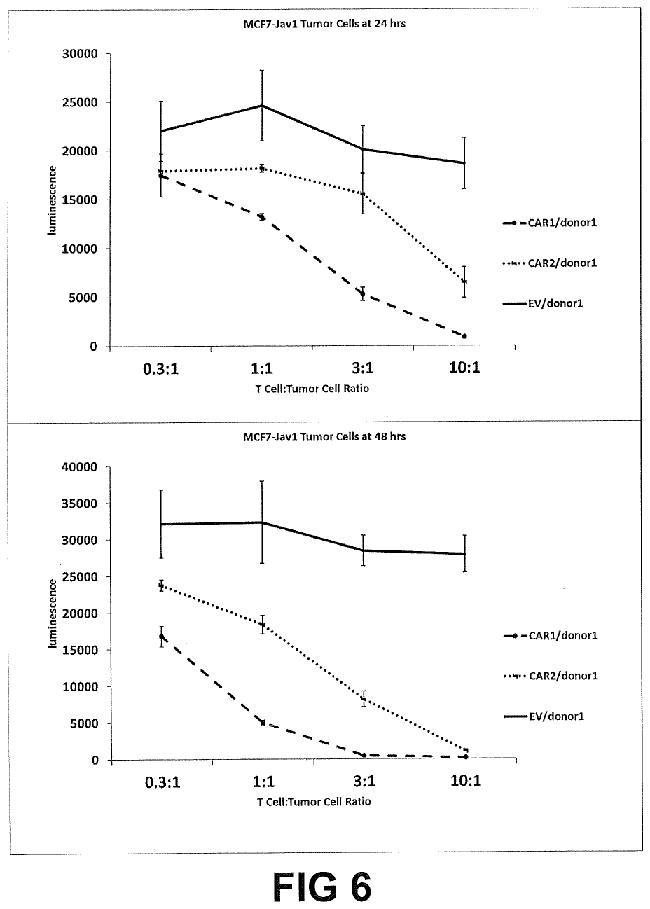

FIG. 6 contains graphs showing the cytotoxicity of anti-ADAM12 CAR-expressing cells of the present invention. The MCF7-ADAM12 cells were transduced with luciferase expression vector JC7 and used as target cells. Human T cells from Donor 1 were transduced with a vector encoding an anti-ADAM12 CAR (anti-ADAM12 CAR1 or anti-ADAM12 CAR2) or with an empty vector (EV, i.e., CD19 only), enriched for CD19-positive cells, expanded, and used as effector cells. Cells were co-cultured at various effector (CAR T cell): target (tumor cell) ratios, and cytotoxicity was evaluated using a luciferase plate assay after 24 (top) or 48 (bottom) hours of co-culture. Asterisks represent significance between anti-ADAM12 CAR and EV T cells using a student's T test (* p<0.05, ** p<0.01, *** p<0.001).

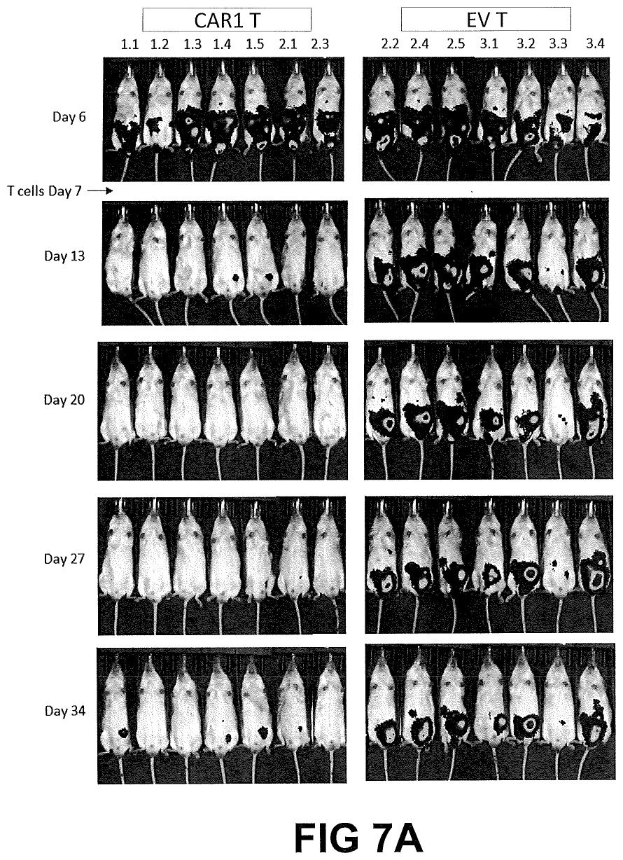

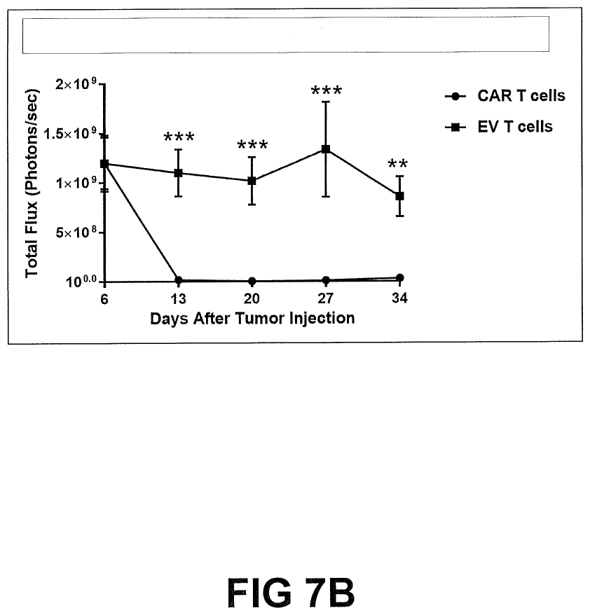

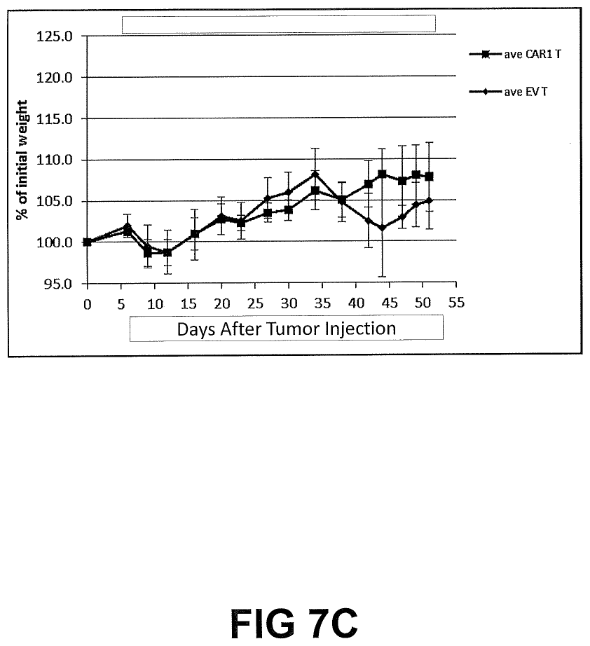

FIGS. 7 A-C contain the results from an in vivo efficacy test described in Example 6. NSG mice harboring intraperitoneal MCF7-ADAM12-Luc tumors were treated with human T cells expressing trCD19 but no anti-ADAM12 (EV T) or human T cells expressing anti-ADAM12 CAR1 (CAR1 T). FIG. 7A is a series of Xenogen-IVIS® images showing the changes in the tumor burden in each treatment group. FIG. 7B is a graph comparing the average tumor burden in the two treatment groups using the luminescent signal intensity (in radiance (photons/second)). Error bars: standard error of the mean (SEM). Statistical differences between the groups were analyzed using student T test with Mann-Whitney ranking (**p<0.01; and ***p<0.001). FIG. 7C is a graph comparing the average body weight in the two groups.

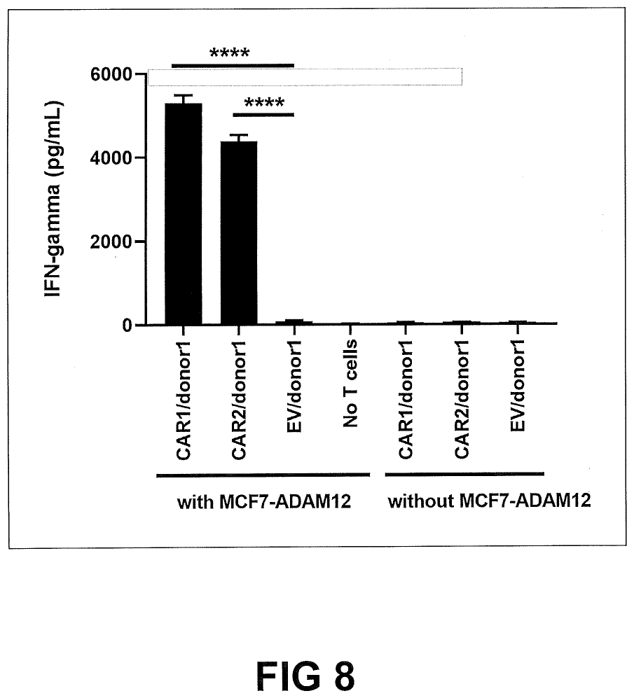

FIG. 8 contains the results from a cytokine production test described in Example 5. Anti-ADAM12 CAR1, anti-ADAM12 CAR2, or EV T cells were cultured with MCF7-ADAM12 for 24 hours, and the concentrations of IFN-g in the supernatant were compared. Error bars: standard error of the mean (SEM). Statistical differences in the IFN-g levels were calculated using Student's T test (**** p<0.0001).

### DETAILED DESCRIPTION

An aspect of the invention in general relates to the construction and use of novel ADAM12-binding agents.

In one aspect, the anti-ADAM12 agent is, for example, but not limited to, anti-ADAM12 antibodies (Abs), antigen-binding Ab fragments, multi-specific Abs, multi-specific antigen-binding Ab fragments, antibody-drug conjugates (ADCs), and chimeric antigen receptors (CARs).

In one aspect, the antigen-binding Ab fragment comprises an AB domain.

In one aspect, the antigen-binding Ab fragment may be an AB domain.

The invention also provides polynucleotides encoding such an Ab, antigen-binding Ab fragment, multi-specific Ab, multi-specific antigen-binding Ab fragment, ADC, or CAR that binds to ADAM12, vectors comprising such a polynucleotide, and cells comprising such an Ab, antigen-binding Ab fragment, multi-specific Ab, multi-specific antigen-binding Ab fragment, ADC, CAR, such a polynucleotide, or such a vector. The invention also provides compositions comprising such an Ab, antigen-binding Ab fragment, multi-specific Ab, multi-specific antigen-binding Ab fragment, ADC, CAR, such a polynucleotide, such a vector, or such a cell.

The invention further provides methods of making and using an ADAM12-binding Ab, antigen-binding Ab fragment, multi-specific Ab, multi-specific antigen-binding Ab fragment, ADC, or CAR, or cells expressing an ADAM12-binding Ab, antigen-binding Ab fragment, multi-specific Ab, multi-specific antigen-binding Ab fragment, ADC, or CAR. The invention also provides methods for treating a condition associated with ADAM12 expression in a subject, such as cancer. Such anti-ADAM12 Abs, antigen-binding Ab fragments, multi-specific Abs, multi-specific antigen-binding Ab fragments, ADCs, CARs, and cells comprising nucleic acid sequence encoding such an ADAM12-binding Ab, antigen-binding Ab fragment, multi-specific Ab, multi-specific antigen-binding Ab fragment, ADC, or CAR may be used to treat diseases, disorders, or conditions associated with the undesired proliferation of cells expressing ADAM12.

### Binding Target

In one aspect, the anti-ADAM12 agents of the present invention bind to ADAM12.

In one aspect, the target, or the binding target, of the anti-ADAM12 agents of the present invention is ADAM12.

In one aspect, the anti-ADAM12 antibody (Ab), anti-ADAM12 antigen-binding Ab fragment, anti-ADAM12 multi-specific Ab, anti-ADAM12 multi-specific antigen-binding Ab fragment, anti-ADAM12 antibody-drug conjugate (ADC), and anti-ADAM12 chimeric antigen receptor (CAR) of the present invention individually comprise an AB domain which binds to ADAM12 (a disintegrin and metalloproteinase-12), also known as ADAM metallopeptidase domain 12, a disintegrin, a disintegrin and metalloproteinase domain-containing protein 12, ADAM12-OT1, CAR10, MCMP, MCMPMltna, Meltrin-a, MLTN, or MLTNA.

In humans, ADAM12 is encoded by the ADAM12 gene on chromosome 10, with gene location 10q26.2 (NCBI), and two naturally occurring ADAM12 splice variants named ADAM12-L (L is for long) and ADAM12-S(S is for short) exist (Kveiborg M. et al., Int J Biochem Cell Biol. 2008; 40(9):1685-702. doi: 10.1016/j.biocel.2008.01.025. Epub 2008 Feb. 1).

The ADAM12-L domain composition resembles the prototypical transmembrane ADAM protein, containing extracellular pro-, metalloprotease, disintegrin-like, cysteine-rich, and epidermal growth factor (EGF)-like domains, followed by a transmembrane domain and a cytoplasmic tail domain. ADAM12-S, the soluble splice variant, contains the same domains as ADAM12-L, except that the transmembrane and cytoplasmic domains are replaced by a unique stretch of 33 amino acids in the C-terminus.

Human ADAM12-L has an amino acid sequence provided as GenBankAccession: AAC08702.2, or the equivalent residues from a non-human species, e.g., mouse, rodent, monkey, ape and the like. In one aspect, human ADAM12-L has the amino acid sequence provided as SEQ ID NO: 101 or the equivalent residues from a non-human species, e.g., mouse, rodent, monkey, ape and the like. Human ADAM12-S has an amino acid sequence provided as GenBankAccession: AAC08703.2, or the equivalent residues from a non-human species, e.g., mouse, rodent, monkey, ape and the like. In one aspect, human ADAM12-S has the amino acid sequence provided as SEQ ID NO: 102 or the equivalent residues from a non-human species, e.g., mouse, rodent, monkey, ape and the like.

In some aspect, the anti-ADAM12 agents of the present invention bind to ADAM12-L.

In some aspect, the anti-ADAM12 agents of the present invention bind to ADAM12-S. [127]. In some aspect, the anti-ADAM12 agents of the present invention bind to both ADAM12-L and ADAM12-S.

ADAM12 is upregulated and/or plays a pathological role in a wide variety of cancers, such as, but not limited to bladder cancer, bone cancer, brain cancer, breast cancer, colon cancer, colorectal cancer, desmoid tumor, esophageal cancer, fibromatosis, glioblastoma, head and neck cancer, liver cancer, lung cancer, melanoma, oesophago-gastric adenocarcinoma, oligodendrioma, oral cancer, oral squamous cell carcinoma, osteosarcoma, ovarian cancer, pancreatic cancer, prostate cancer, skin cancer, small cell lung cancer, stomach cancer, and thyroid cancer (Kveiborg M. et al., Int J Biochem Cell Biol. 2008; 40(9):1685-702. doi: 10.1016/j.biocel.2008.01.025. Epub 2008 Feb. 1; Le Pabic H. et l., Hepatology. 2003 May; 37(5):1056-66; Skubitz K. M. et al., J Lab Clin Med. 2004 February; 143(2):89-98.; Carl-McGrath S. et al., Int J Oncol. 2005 January; 26(1):17-24; Mochizuki S. et al., Cancer Sci. 2007 May; 98(5):621-8. Epub 2007 Mar. 9; Colombo C. et al., J Pathol. 2011 December; 225(4):574-82. doi: 10.1002/path.2951. Epub 2011 Aug. 8; Sookprasert A. et al., Asian Pac J Cancer Prev. 2012; 13 Suppl:3-6; Uehara E. et al., Int J Oncol. 2012 May; 40(5):1414-22. doi: 10.3892/ijo.2012.1339. Epub 2012 Jan. 20; Baren J. P. et al., Br J Cancer. 2012 Jun. 26; 107(1):143-9. doi: 10.1038/bjc.2012.239. Epub 2012 Jun. 7; Rao V. H. et al., Oncogene. 2012 Jun. 7; 31(23):2888-98. doi: 10.1038/onc.2011.460. Epub 2011 Oct. 10; Kanakis D. et al., Dis Markers. 2013; 34(2):81-91. doi: 10.3233/DMA-120953; Georges S. et al., Eur J Cancer. 2013 June; 49(9):2253-63. doi: 10.1016/j.ejca.2013.02.020. Epub 2013 Mar. 13; Cireap N. et al., Pathol Oncol Res. 2013 October; 19(4):755-62. doi: 10.1007/s12253-013-9639-8. Epub 2013 May 6; Bilgin Dogru E. et al., Tumour Biol. 2014 November; 35(11):11647-53. doi: 10.1007/s13277-014-2514-8. Epub 2014 Aug. 20; Cheon D. J. Et al., Carcinogenesis. 2015 July; 36(7):739-47. doi: 10.1093/carcin/bgv0S9. Epub 2015 Apr. 29; Rao V. H. et al., Mol Carcinog. 2015 October; 54(10):1026-36. doi: 10.1002/mc.22171. Epub 2014 May 5; Li Z. et al., Oncol Rep. 2015 December; 34(6):3231-7; Liu G. et al., Oncol Rep. 2016 November; 36(5):3005-3013. doi: 10.3892/or.2016.5064. Epub 2016 Sep. 5; Fröhlich C. et al., Clin Cancer Res. 2006 Dec. 15; 12(24):7359-68; Roy R. et al., Mol Cancer Res. 2017 November; 15(11):1608-1622. doi: 10.1158/1541-7786.MCR-17-0188. Epub 2017 Aug. 1; Xiong L. et al., J Proteomics. 2018 Jun. 30; 182:34-44. doi: 10.1016/j.jprot.2018.04.033. Epub 2018 May 2; Veenstra V. L. et al., Oncogenesis. 2018 Nov. 16; 7(11):87. doi: 10.1038/s41389-018-0096-9). Therefore, in some aspects, the anti-ADAM12 agents of the present invention may bind to or target ADAM12 on cancer cells of the above-mentioned cancer types.

In some embodiments, anti-ADAM12 agents according to the present disclosure may bind bladder cancer cells. In some embodiments, anti-ADAM12 agents may bind bone cancer cells. In some embodiments, anti-ADAM12 agents may bind brain cancer cells. In some embodiments, anti-ADAM12 agents may bind breast cancer cells. In some embodiments, anti-ADAM12 agents may bind colon cancer cells. In some embodiments, anti-ADAM12 agents may bind colorectal cancer cells. In some embodiments, anti-ADAM12 agents may bind desmoid tumor cells.

In some embodiments, anti-ADAM12 agents may bind esophageal cancer cells. In some embodiments, anti-ADAM12 agents may bind fibromatosis cells. In some embodiments, anti-ADAM12 agents may bind glioblastoma cells. In some embodiments, anti-ADAM12 agents may bind head and neck cancer cells. In some embodiments, anti-ADAM12 agents may bind liver cancer cells. In some embodiments, anti-ADAM12 agents may bind lung cancer cells. In some embodiments, anti-ADAM12 agents may bind melanoma cells. In some embodiments, anti-ADAM12 agents may bind oesophago-gastric adenocarcinoma cells.

In some embodiments, anti-ADAM12 agents may bind oligodendrioma cells. In some embodiments, anti-ADAM12 agents may bind oral cancer cells. In some embodiments, anti-ADAM12 agents may bind oral squamous cell carcinoma cells. In some embodiments, anti-ADAM12 agents may bind osteosarcoma cells. In some embodiments, anti-ADAM12 agents may bind ovarian cancer cells. In some embodiments, anti-ADAM12 agents may bind pancreatic cancer cells. In some embodiments, anti-ADAM12 agents may bind prostate cancer cells. In some embodiments, anti-ADAM12 agents may bind skin cancer cells. In some embodiments, anti-ADAM12 agents may bind small cell lung cancer cells. In some embodiments, anti-ADAM12 agents may bind stomach cancer cells. In some embodiments, anti-ADAM12 agents may bind thyroid cancer cells.

Of note, the overexpression of ADAM12 in various diseases is in stark contrast with its expression on normal tissues, which is extremely low and restricted to breast and ovary tissues. In addition, ADAM12 expression has been implicated in accelerated tumor growth, promotion of tumor angiogenesis, and poor prognosis (Kveiborg M. et al., Cancer Res. 2005 Jun. 1; 65(11):4754-61; Roy R. et al., Mol Cancer Res. 2017, November; 15(11):1608-1622. doi: 10.1158/1541-7786.MCR-17-0188. Epub 2017 Aug. 1). These observations particularly highlight the value of ADAM12 as a therapeutic target according to the present methods.

ADAM12 is also upregulated and/or plays a pathological role in many other diseases and conditions, such as, but not limited to, Alzheimer's disease, ostearthritis, muscular dystrophies, multiple sclerosis, fibrosis, cardiac hypertrophy, skin fibrosis and interstitial lung disease in systemic sclerosis, renal fibrosis, peripheral arterial disease, endometriosis, and Dupuytren's disease (Kveiborg M. et al., Int J Biochem Cell Biol. 2008; 40(9):1685-702. doi: 10.1016/j.biocel.2008.01.025. Epub 2008 Feb. 1; Harold D. et al., Am J Med Genet B Neuropsychiatr Genet. 2007 Jun. 5; 144B(4):448-52; Kerna I. et al., Rheumatol Int. 2012 February; 32(2):519-23. doi: 10.1007/s00296-010-1717-6. Epub 2011 Jan. 22; Dulauroy S. et al., Nat Med. 2012 August; 18(8):1262-70. doi: 10.1038/nm.2848. Epub 2012 Jul. 29; Berry E. et al., J Vasc Res. 2013; 50(1):52-68. doi: 10.1159/000345240. Epub 2012 Nov. 17; Taniguchi T. et al., J Eur Acad Dermatol Venereol. 2013 June; 27(6):747-53. doi: 10.1111/j.1468-3083.2012.04558.x. Epub 2012 Apr. 28; Ramdas V. et al., Am J Pathol. 2013 December; 183(6):1885-1896. doi: 10.1016/j.ajpath.2013.08.027. Epub 2013 Oct. 6; Dokun A. O. et al., Am J Physiol Heart Circ Physiol. 2015 September; 309(5):H790-803. doi: 10.1152/ajpheart.00803.2014. Epub 2015 Jul. 10; Miller M. A. et al., Sci Rep. 2015 Oct. 19; 5:15150. doi: 10.1038/srep15150; Sedic M. et al., (2012) Using Functional Genomics to Identify Drug Targets: A Dupuytren's Disease Example. In: Larson R. (eds) Bioinformatics and Drug Discovery. Methods in Molecular Biology (Methods and Protocols), vol 910. Humana Press, Totowa, NJ). Therefore, in some aspects, the anti-ADAM12 agents of the present invention may bind to or target ADAM12 on cells of the above-mentioned diseases or conditions.

In some embodiments, anti-ADAM12 agents or compositions according to the present disclosure may be used to treat Alzheimer's disease. In some embodiments, anti-ADAM12 agents or compositions may be used to treat ostearthritis. In some embodiments, anti-ADAM12 agents or compositions may be used to treat muscular dystrophies. In some embodiments, anti-ADAM12 agents or compositions may be used to treat multiple sclerosis. In some embodiments, anti-ADAM12 agents or compositions may be used to treat fibrosis. In some embodiments, anti-ADAM12 agents or compositions may be used to treat cardiac hypertrophy. In some embodiments, anti-ADAM12 agents or compositions may be used to treat skin fibrosis and interstitial lung disease in systemic sclerosis. In some embodiments, anti-ADAM12 agents or compositions may be used to treat renal fibrosis. In some embodiments, anti-ADAM12 agents or compositions may be used to treat peripheral arterial disease. In some embodiments, anti-ADAM12 agents or compositions may be used to treat endometriosis. In some embodiments, anti-ADAM12 agents or compositions may be used to treat Dupuytren's disease.

### Anti-ADAM12 Antibody, Antigen-Binding Fragment, Multi-Specific Antibody, Multi-Specific Antigen-Binding Fragment, and Antibody-Drug Conjugate

In some embodiments, the anti-ADAM12 antibody (Ab), anti-ADAM12 antigen-binding (AB) fragment, anti-ADAM12 multi-specific Ab, anti-ADAM12 multi-specific antigen-binding Ab fragment, and anti-ADAM12 antibody-drug conjugate (ADC) of the present invention, individually comprise at least one AB domain that binds to ADAM12.

The ADAM12-binding domain (i.e., the AB domain) may comprise the AB domain of a humanized version of mouse anti-ADAM12 monoclonal antibodies. In some embodiments, the ADAM12-binding domain (i.e., the AB domain) may comprise the AB domain of a humanized version of mouse anti-ADAM12 monoclonal antibody clones 6E6 and 6C10 or their variants.

The mouse anti-ADAM12 monoclonal antibody 6E6 comprises: (a) a heavy chain (HC) variable domain (VH) sequence as set forth in SEQ ID NO: 111, which may be encoded by SEQ ID NO: 211; and (b) a light chain (LC) variable domain (VL) sequence as set forth in SEQ ID NO: 115, which may be encoded by SEQ ID NO: 215. The complementarity determining regions 1, 2, and 3 (CDR1, CDR 2, and CDR 3) of the VH (i.e., CDR-H1, CDR-H2, and CDR-H3) comprise the amino acid sequence of SEQ ID NOs: 112, 113, and 114, respectively, which may be encoded by SEQ ID NOs: 212, 213, and 214, respectively. The CDR1, CDR 2, and CDR 3 of the VL (i.e., CDR-L1, CDR-L2, and CDR-L3) comprise the amino acid sequence of SEQ ID NOs: 116, 117, and 118, respectively, which may be encoded by SEQ ID NOs: 216, 217, and 218, respectively.

The mouse anti-ADAM12 monoclonal antibody clone 6C10 comprises: (a) a VH sequence as set forth in SEQ ID NO: 121, which may be encoded by SEQ ID NO: 221; and (b) VL sequence as set forth in SEQ ID NO: 125, which may be encoded by SEQ ID NO: 225. The CDR1, CDR 2, and CDR 3 of the VH (i.e., CDR-H1, CDR-H2, and CDR-H3) comprise the amino acid sequence of SEQ ID NOs: 122, 123, and 124, respectively, which may be encoded by SEQ ID NOs: 222, 223, and 224, respectively. The CDR1, CDR 2, and CDR 3 of the VL (i.e., CDR-L1, CDR-L2, and CDR-L3) comprise the amino acid sequence of SEQ ID NOs: 126, 127, and 128, respectively, which may be encoded by SEQ ID NOs: 226, 227, and 228, respectively.

Inventor performed humanization the 6E6 antibody as described in Example 1 herein. The humanized version of 6E6 (may be referred to as h6E6) comprises: (a) a VH sequence as set forth in SEQ ID NO: 131, which may be encoded by SEQ ID NO: 231; and (b) a VL sequence as set forth in SEQ ID NO: 135, which may be encoded by SEQ ID NO: 235. The CDR 1, CDR 2, and CDR 3 of the VH (i.e., CDR-H1, CDR-H2, and CDR-H3) may comprise the amino acid sequence of SEQ ID NOs: 132, 133, and 134, respectively, which may be encoded by SEQ ID NOs: 232, 233, and 234, respectively. The CDR1, CDR 2, and CDR 3 of the VL (i.e., CDR-L1, CDR-L2, and CDR-L3) may comprise the amino acid sequence of SEQ ID NOs: 136, 137, and 138, respectively, which may be encoded by SEQ ID NOs: 236, 237, and 238, respectively.

Inventor performed humanization the 6C10 antibody as described in Example 1 herein. The humanized version of 6C10 (may be referred to as h6C10) comprises: (a) a VH sequence as set forth in SEQ ID NO: 141, which may be encoded by SEQ ID NO: 241; and (b) a VL sequence as set forth in SEQ ID NO: 145, which may be encoded by SEQ ID NO: 245. The CDR 1, CDR 2, and CDR 3 of the VH (i.e., CDR-H1, CDR-H2, and CDR-H3) may comprise the amino acid sequence of SEQ ID NOs: 142, 143, and 144, respectively, which may be encoded by SEQ ID NOs: 242, 243, and 244, respectively. The CDR1, CDR 2, and CDR 3 of the VL (i.e., CDR-L1, CDR-L2, and CDR-L3) may comprise the amino acid sequence of SEQ ID NOs: 146, 147, and 148, respectively, which may be encoded by SEQ ID NOs: 246, 247, and 248, respectively.

Therefore, in some embodiments, the ADAM12-binding domain (i.e., the AB domain) of the anti-ADAM12 agent of the present invention may comprise (a) a CDR-H1, a CDR-H2, and a CDR-H3, and (b) a CDR-L1, a CDR-L2, and a CDR-L3.

In some aspects, the ADAM12-binding domain (i.e., the AB domain) may comprise (a) a VH comprising a CDR-H1, a CDR-H2, and a CDR-H3, and a human-like HC framework; and (b) a VL comprising a CDR-L1, a aCDR-L2, and CDR-L3, and a human-like LC framework.

In some aspects, in the AB binding domain of the anti-ADAM12 agent of the present invention, the CDR-H1, CDR-H2, and CDR-H3 may comprise the amino acid sequences set forth in SEQ ID NOs: 132, 133, and 134, respectively, and the CDR-L1, CDR-L2, and CDR-L3 may comprise the amino acid sequences set forth in SEQ ID NOs: 136, 137, and 138, respectively. The CDR-H1, CDR-H2, and CDR-H3 may comprise the amino acid sequences encoded by SEQ ID NOs: 232, 233, and 234, respectively, and the CDR-L1, CDR-L2, and CDR-L3 may comprise the amino acid sequences encoded by SEQ ID NOs: 236, 237, and 238, respectively.

In some aspects, the CDR-H1, CDR-H2, and CDR-H3 may comprise the amino acid sequences set forth in SEQ ID NOs: 142, 143, and 144, respectively, and the CDR-L1, CDR-L2, and CDR-L3 may comprise the amino acid sequences set forth in SEQ ID NOs: 146, 147, and 148, respectively. The CDR-H1, CDR-H2, and CDR-H3 may comprise the amino acid sequences encoded by SEQ ID NOs: 242, 243, and 244, respectively, and the CDR-L1, CDR-L2, and CDR-L3 may comprise the amino acid sequences encoded by SEQ ID NOs: 246, 247, and 248, respectively.

Generally, to humanize a mouse antibody, the CDRs from the mouse antibody may be grafted into the human antibody framework. Therefore, the human-like framework may be 100% identical to a human framework. Inventor used the Tabhu program (http://circe.med.uniroma1.it/tabhu/) to perform humanization, which involves four steps, which are: (i) loop grafting, (ii) estimation of the binding mode similarity between the native and human antibody, (iii) back-mutations and (iv) reevaluation of the binding mode similarity between input and humanized antibody (Olimpieri P. P. et al., Bioinformatics. 2015 Feb. 1; 31(3):434-5. doi: 10.1093/bioinformatics/btu667. Epub 2014 Oct. 9). Therefore, in some embodiments, the framework may not be 100% identical to a human framework but may still comprise significant sequence identity to a human framework.

In some aspects, (a) the human-like HC framework may be at least 80%, at least 85%, at least 90%, at least 92%, at least 94%, at least 95%, at least 96%, at least 97%, at least 98%, at least 99%, or 100% identical to a human HC framework, and (b) the human-like LC framework may at least 80%, at least 85%, at least 90%, at least 92%, at least 94%, at least 95%, at least 96%, at least 97%, at least 98%, at least 99%, or 100% identical to a human LC framework.

In some embodiments, the variable domain of an anti-ADAM12 agent disclosed herein may be altered without inhibiting ADAM12 binding. As long as the AB domain sufficiently binds to ADAM12, the sequence of the variable domain may be altered. Antigen-Ab interactions are largely determined by six CDRs, while a person of skilled in the art will appreciate that some deviation from the exact CDR sequences may be possible. Any suitable techniques such as affinity maturation can be used to alter the CDR sequence. Among the six CDRs, VH CDR 3 and VL CDR 3 are generally believed as the key determinant of specificity in antigen recognition. Particularly, diversity in the CDR 3 of VH (i.e., CDR-H3) may be particularly important for providing most antibody specificities (Xu J. L., Immunity. 2000 July; 13(1):37-45). Therefore, one or more mutations may be incorporated in the CDR 1 and/or CDR 2 without greatly decreasing the binding affinity while achieving a more desired property of an Ab. Abs or antigen-binding Ab fragments comprising CDR-H1, CDR-H2, CDR-L1, CDR-L2, and/or CDR-L3 with at least 80%, at least 85%, at least 90%, at least 92%, at least 94%, at least 95%, at least 96%, at least 97%, at least 98%, at least 99%, or 100% identical to the CDR-H1, CDR-H2, CDR-L1, CDR-L2, and/or CDR-L3 disclosed herein, respectively, are also within the scope of the present invention. Furthermore, one or more mutations in CDR-H3 may be incorporated to modify, increase, or fine tune the binding or any other properties of the AB domain. Alternatively, since any one mutation may alter biochemical properties such as thermodynamic stability or immunogenicity in addition to the affinity, all possible mutations in any of the six CDRs, and/or any combination thereof, and/or even in the framework sequence, may be tested to see if the sequence modification provides an improved or more desired overall property (Rajpal A. et al., Proc Natl Acad Sci USA. 2005 Jun. 14; 102(24): 8466-8471. doi: 10.1073/pnas.0503543102; Julian M. C. et al., Sci Rep. 2017; 7: 45259. Published online 2017 Mar. 28. doi: 10.1038/srep45259). To test the binding of a modified Ab to the target, any appropriate technique such as, but not limited to, ELISA, RIA, FACS, bioassay, or Western Blot assay may be used.

In some aspects, the HC variable domain (i.e., VH) may comprise an amino acid sequence at least 80%, at least 85%, at least 90%, at least 92%, at least 94%, at least 95%, at least 96%, at least 97%, at least 98%, at least 99%, or 100% identical to SEQ ID NO: 131, and the LC variable domain comprises an amino acid sequence at least 80%, at least 85%, at least 90%, at least 92%, at least 94%, at least 95%, at least 96%, at least 97%, at least 98%, at least 99%, or 100% identical to SEQ ID NO: 135. The HC variable domain may comprise an amino acid sequence at least 80%, at least 85%, at least 90%, at least 92%, at least 94%, at least 95%, at least 96%, at least 97%, at least 98%, at least 99%, or 100% identical to the amino acid sequence encoded by SEQ ID NO: 231, and the LC variable domain (i.e., VL) comprises an amino acid sequence at least 80%, at least 85%, at least 90%, at least 92%, at least 94%, at least 95%, at least 96%, at least 97%, at least 98%, at least 99%, or 100% identical to the amino acid sequence encoded by SEQ ID NO: 235.

In some aspects, the VH may comprise an amino acid sequence at least 80%, at least 85%, at least 90%, at least 92%, at least 94%, at least 95%, at least 96%, at least 97%, at least 98%, at least 99%, or 100% identical to SEQ ID NO: 141, and the VL may comprise an amino acid sequence at least 80%, at least 85%, at least 90%, at least 92%, at least 94%, at least 95%, at least 96%, at least 97%, at least 98%, at least 99%, or 100% identical to SEQ ID NO: 145. The VH may comprise an amino acid sequence at least 80%, at least 85%, at least 90%, at least 92%, at least 94%, at least 95%, at least 96%, at least 97%, at least 98%, at least 99%, or 100% identical to the amino acid sequence encoded by SEQ ID NO: 241, and the VL may comprise an amino acid sequence at least 80%, at least 85%, at least 90%, at least 92%, at least 94%, at least 95%, at least 96%, at least 97%, at least 98%, at least 99%, or 100% identical to the amino acid sequence encoded by SEQ ID NO: 245.

In some aspects, the anti-ADAM12 agent of the present invention, may be, for example, but not limited to, a monoclonal Ab, a monospecific Ab, a bispecific Ab, a multispecific Ab, a humanized Ab, a tetrameric Ab, a tetravalent Ab, a single chain Ab, a domain-specific Ab, a domain-deleted Ab, an scFc fusion protein, a chimeric Ab, a synthetic Ab, a recombinant Ab, a hybrid Ab, a mutated Ab, CDR-grafted Ab, a fragment antigen-binding (Fab), an F(ab′)2, an Fab′ fragment, a variable fragment (Fv), a single-chain Fv (scFv) fragment, an Fd fragment, a diabody, and a minibody.

In certain aspects, the AB domain of the anti-ADAM12 agent of the present invention may comprise an amino acid sequence at least 80%, at least 85%, at least 90%, at least 92%, at least 94%, at least 95%, at least 96%, at least 97%, at least 98%, at least 99%, or 100% identical to: (i) SEQ ID NOs: 139, 140, 149, or 150, or (ii) the amino acid sequence encoded by SEQ ID NOs: 239, 240, 249, or 250.

In some aspects, the anti-ADAM12 agent of the present invention may comprise two or more binding specificities (i.e., bispecific, trispecific, or multispecific generally). The first specificity is to an epitope in ADAM12 (first ADAM12 epitope).

In one aspect, the anti-ADAM12 agent of the present disclosure may have a second binding specificity to another epitope (i.e., second ADAM12 epitope) in ADAM12. The second ADAM12 epitope may or may not be overlapped with the first ADAM12 epitope.

In another aspect, the second specificity may be to an epitope in a second antigen other than ADAM12. A multi-specific ADAM12-binding agent according to the present disclosure may bind to ADAM12 and one or more other targets. In some embodiments, a multi-specific anti-ADAM12 agent binds to ADAM12 and a protein on an effector cell. In some embodiments, a multi-specific anti-ADAM12 agent binds to ADAM12 and a protein on a target (e.g., cancer) cell. In some embodiments, binding to a second antigen may improve functional characteristics of the anti-ADAM12 agent, e.g., recruitment, effector functions, lysis of target cells.

In certain aspects, the second antigen may be, for example without limitation, CD3, NKG2D, 4-1BB, or an Fc receptor (FcR), such as Fc gamma receptor (FcgR), FcgRI, FcgRIIA, FcgRIIB1, FcgRIIB2, FcgRIIIA, FcgRIIIB, Fc epsilon receptor (FceR), FceRI, FceRII, Fc alpha receptor (FcaR), FcaRI, Fc alpha/mu receptor (Fca/mR), or neonatal Fc receptor (FcRn). For anti-ADAM12 Abs and antigen-binding Ab fragments, having a specificity to an FcR allows FcR-mediated effects such as antibody-dependent cellular phagocytosis (ADCP) or antibody-dependnet cellular cytotoxicity (ADCC) of ADAM12-expressing cells or cytotoxic mediator release by Fc-expressing cells.

When the second epitope is in FcR, the FcR may be, but is not limited to, Fc gamma receptor (FcgR), FcgRI, FcgRIIA, FcgRIIB1, FcgRIIB2, FcgRIIIA, FcgRIIIB, Fc epsilon receptor (FceR), FceRI, FceRII, Fc alpha receptor (FcaR), FcaRI, Fc alpha/mu receptor (Fca/mR), or neonatal Fc receptor (FcRn).

When the anti-ADAM12 agent has two specificities, the agent may be called bispecific. Bispecific anti-ADAM12 agents include bispecific anti-ADAM12 Abs or antigen-binding Ab fragments. When the anti-ADAM agent has two or more specificities, the agent may be called multispecific. Multipecific anti-ADAM12 agents include multispecific anti-ADAM12 Abs or antigen-binding Ab fragments.

The present invention encompasses any types of bispecific Ab-like molecules (Abs or antigen-binding Ab fragments) such as reviewed in Brinkmann U. et al., MAbs. 2017 February-March; 9(2): 182-212. Published online 2017 Jan. 10. doi: 10.1080/19420862.2016.1268307; Klein C. et al., MAbs. 2016 August-September; 8(6):1010-20. doi: 10.1080/19420862.2016.1197457. In a bispecific embodiment according to the present disclosure, one of the AB domains is an anti-ADAM12 binding domain. General methods for designing and construction of bispecific or multispecific Abs or antigen-binding Ab fragments are known in the art (Brinkmann U. et al., MAbs. 2017 February-March; 9(2): 182-212. Published online 2017 Jan. 10. doi: 10.1080/19420862.2016.1268307; Dimasi N. et al. Methods. 2018 Aug. 11. pii: S1046-2023(18)30149-X. doi: 10.1016/j.ymeth.2018.08.004; Sedykh S. E. et al., Drug Des Devel Ther. 2018; 12: 195-208). Such methods include chemical conjugation, covalent attachment of fragments, and genetic engineering. For example, full-length bispecific Abs or antigen-binding Ab fragments may be generated by co-expressing two pairs of heavy and light chains, each pair having different specificities. The two pairs may be encoded in one vector, or encoded in separate vectors but expressed in the same hsot cell. Alternatively, antigen-binding Ab fragments or the AB domains having different specificities may be generated separately and then conjugated to one another, for example using sulfhydryl bonding (of, for example, the HC C-terminus hinge regions) and/or an appropriate coupling or crosslinking agent. Bispecific antigen-binding Ab fragments may also be generated, for example, by using leucine zippers or by using scFv dimers (see for example, Kosteln et al., J Immunol. 1992 Mar. 1; 148(5):1547-53). Binding of the bispecific agent of the present invention may be confirmed using any appropriate method, such as but not limited to, enzyme-linked immunosorbent assay (ELISA), radioimmunoassay (RIA), flow cytometry, bioassay, or Western blot.

In some embodiments, the anti-ADAM12 agents of the present invention may comprise a human-like fragment crystallizable (Fc) region.

In some aspects, the human-like Fc region may be at least 80%, at least 85%, at least 90%, at least 92%, at least 94%, at least 95%, at least 96%, at least 97%, at least 98%, at least 99%, or 100% identical to a human Fc region.

In certain aspects, the human-like Fc region may bind to an Fc receptor (FcR). The FcR may be, but is not limited to, Fc gamma receptor (FcgR), FcgRI, FcgRIIA, FcgRIIB1, FcgRIIB2, FcgRIIIA, FcgRIIIB, Fc epsilon receptor (FceR), FceRI, FceRII, Fc alpha receptor (FcaR), FcaRI, Fc alpha/mu receptor (Fca/mR), or neonatal Fc receptor (FcRn).

In some embodiments, when the anti-ADAM12 agent is an Ab, the Ab may be an IgM, IgD, IgG, IgE, or IgA isotype.

In some aspects, when the Ab is an IgG, the IgG may be an IgG1, IgG2, IgG3, or IgG4.

Certain amino acid modifications in the Fc region are known to modulate Ab effector functions and properties, such as, but not limited to, antibody-dependent cellular cytotoxicity (ADCC), antibody-dependent cellular phagocytosis (ADCP), complement dependent cytotoxicity (CDC), and half-life (Wang X. et al., Protein Cell. 2018 January; 9(1): 63-73; Dall'Acqua W. F. et al., J Biol Chem. 2006 Aug. 18; 281(33):23514-24. Epub 2006 Jun. 21; Monnet C. et al, Front Immunol. 2015 Feb. 4; 6:39. doi: 10.3389/fimmu.2015.00039. eCollection 2015). The mutation may be symmetrical or asymmetrical. In certain cases, antibodies with Fc regions that have asymmetrical mutation(s) (i.e., two Fc regions are not identical) may provide better functions such as ADCC (Liu Z. et al. J Biol Chem. 2014 Feb. 7; 289(6): 3571-3590).

When the Ab is an IgG1, the Fc region may comprise one or more amino acid substitutions. The substitution may be, for example, N297A, N297Q, D265A, L234A, L235A, C226S, C229S, P238S, E233P, L234V, G236-deleted, P238A, A327Q, A327G, P329A, K322A, L234F, L235E, P331S, T394D, A330L, P331S, F243L, R292P, Y300L, V305I, P396L, S239D, I332E, S298A, E333A, K334A, L234Y, L235Q, G236W, S239M, H268D, D270E, K326D, A330M, K334E, G236A, K326W, S239D, E333S, S267E, H268F, S324T, E345R, E430G, S440Y M428L, N434S, L328F, M252Y, S254T, T256E, and/or any combination thereof (the residue numbering is according to EU or Kabat numbering) (Dall'Acqua W. F. et al., J Biol Chem. 2006 Aug. 18; 281(33):23514-24. Epub 2006 Jun. 21; Wang X. et al., Protein Cell. 2018 January; 9(1): 63-73). The Fc region may further comprise one or more additional amino acid substitutions. The substitution may be, for example, but is not limited to, A330L, L234F, L235E, P3318, and/or any combination thereof (the residue numbering is according to EU or Kabat numbering).

When the Ab is an IgG2, the Fc region may comprise one or more amino acid substitutions. The substitution may be, for example, but is not limited to, P238S, V234A, G237A, H268A, H268Q, H268E, V309L, N297A, N297Q, A330S, P331S, C232S, C233S, M252Y, S254T, T256E, and/or any combination thereof (the residue numbering is according to EU or Kabat numbering). The Fc region may further comprise one or more additional amino acid substitutions. The substitution may be, for example, but is not limited to, M252Y, S254T, T256E, and/or any combination thereof (the residue numbering is according to EU or Kabat numbering).

When the Ab is an IgG3, the Fc region may comprise one or more amino acid substitutions. The substitution may be, for example, but is not limited to, E235Y (the residue numbering is according to EU or Kabat numbering).

When the Ab is an IgG4, the Fc region may comprise one or more amino acid substitutions. The substitution may be, for example, but is not limited to, E233P, F234V, L235A, G237A, E318A, S228P, L236E, S241P, L248E, T394D, M252Y, S254T, T256E, N297A, N297Q, and/or any combination thereof (the residue numbering is according to EU or Kabat numbering). The substitution may be, for example, S228P (the residue numbering is according to EU or Kabat numbering).

In some aspects, the glycan of the human-like Fc region may be engineered to modify the effector function (for example, see Li T. et al., Proc Natl Acad Sci USA. 2017 Mar. 28; 114(13):3485-3490. doi: 10.1073/pnas.1702173114. Epub 2017 Mar. 13).

In some embodiments, the anti-ADAM12 agent of the present invention may be an antibody-drug conjugate (ADC). The ADC may comprise: (a) any Ab or antigen-binding Ab fragment described herein; and (b) a drug conjugated to the Ab or antigen-binding Ab fragment.

In some aspects, the drug may be, but not limited to, an anti-cancer drug, an anti-proliferative drug, a cytotoxic drug, an anti-angiogenic drug, an apoptotic drug, an immunostimulatory drug, an anti-microbial drug, an antibiotic drug, an antiviral drug, an anti-inflammatory drug, an anti-fibrotic drug, an immunosuppressive drug, a steroid, a bronchodilator, a beta blocker, a matrix metalloproteinase inhibitor, an ADAM12 inhibitor, an ADAM12 signaling inhibitor, an enzyme, a hormone, a neurotransmitter, a toxin, a radioisotope, a compound, a small molecule, a small molecule inhibitor, a protein, a peptide, a vector, a plasmid, a viral particle, a nanoparticle, a DNA molecule, an RNA molecule, an siRNA, an shRNA, a micro RNA, an oligonucleotide, and an imaging drug.

The toxin may be a bacterial, fungal, plant, or animal toxin, or a fragment thereof. Examples include, but are not limited to, diphtheria A chain, diphtheria toxin, exotoxin A chain, ricin A chain, abrin A chain, modeccin A chain, alpha sarcin, Aleurites fordii protein, a dianthin protein, or a Phytolacca Americana protein.

The anti-cancer or anti-proliferative drug may be, for example, but is not limited to, doxorubicin, daunorubicin, cucurbitacin, chaetocin, chaetoglobosin, chlamydocin, calicheamicin, nemorubicin, cryptophyscin, mensacarcin, ansamitocin, mitomycin C, geldanamycin, mechercharmycin, rebeccamycin, safracin, okilactomycin, oligomycin, actinomycin, sandramycin, hypothemycin, polyketomycin, hydroxyellipticine, thiocolchicine, methotrexate, triptolide, taltobulin, lactacystin, dolastatin, auristatin, monomethyl auristatin E (MMAE), monomethyl auristatin F (MMAF), telomestatin, tubastatin A, combretastatin, maytansinoid, MMAD, MMAF, DM1, DM4, DTT, 16-GMB-APA-GA, 17-DMAP-GA, JW 55, pyrrolobenzodiazepine, SN-38, Ro 5-3335, puwainaphycin, duocarmycin, bafilomycin, taxoid, tubulysin, ferulenol, lusiol A, fumagillin, hygrolidin, glucopiericidin, amanitin, ansatrienin, cinerubin, phallacidin, phalloidin, phytosphongosine, piericidin, poronetin, phodophyllotoxin, gramicidin A, sanguinarine, sinefungin, herboxidiene, microcolin B, microcystin, muscotoxin A, tolytoxin, tripolin A, myoseverin, mytoxin B, nocuolin A, psuedolaric acid B, pseurotin A, cyclopamine, curvulin, colchicine, aphidicolin, englerin, cordycepin, apoptolidin, epothilone A, limaquinone, isatropolone, isofistularin, quinaldopeptin, ixabepilone, aeroplysinin, arruginosin, agrochelin, epothilone, or a derivative thereof (for example, see Polakis P. et al., Pharmacol Rev. 2016 January; 68(1):3-19. doi: 10.1124/pr.114.009373) (the drugs may be obtained from many vendors, including Creative Biolabs®).

The radioisotope may be for example, but is not limited to, At211, 1131, In131, 1125, Y90, Re186, Re188, Sm153, Bi212, P32, Pb212 and radioactive isotopes of Lu.

In certain aspects, the drug may be, but is not limited to, MMAE or MMAF.

In some aspects, the Ab or antigen-binding Ab fragment is directly conjugated to the drug to form an ADC.

In some aspects, the Ab or antigen-binding Ab fragment is indirectly conjugated to the drug to form an ADC.

Any appropriate conjugation method may be used to generate an ADC (for example, Nolting B. Methods Mol Biol. 2013; 1045:71-100. doi: 10.1007/978-1-62703-541-5_5; Jain N. et al., Pharm Res. 2015 November; 32(11):3526-40. doi: 10.1007/s11095-015-1657-7. Epub 2015 Mar. 11; Tsuchikama K. et al., Protein Cell. 2018 January; 9(1):33-46. doi: 10.1007/s13238-016-0323-0. Epub 2016 Oct. 14; Polakis P. et al., Pharmacol Rev. 2016 January; 68(1):3-19. doi: 10.1124/pr.114.009373). Examples of methods that may be used to perform conjugation include, but are not limited to, chemical conjugation and enzymatic conjugation.

Chemical conjugation may utilize, for example, but is not limited to, lysine amide coupling, cysteine coupling, and/or non-natural amino acid incorporation by genetic engineering. Enzymatic conjugation may utilize, for example, but is not limited to, transpeptidation using sortase, transpeptidation using microbial transglutaminase, and/or N-Glycan engineering.

In certain aspects, one or more of cleavable linkers may be used for conjugation. The cleavable linker may enable cleavage of the drug upon responding to, for example, but not limited to, an environmental difference between the extracellular and intracellular environments (pH, redox potential, etc.) or by specific lysosomal enzymes.

Examples of the cleavable linker include, but are not limited to, hydrazone linkers, peptide linkers including cathepsin B-responsive linkers, such as valin-citrulline (vc) linker, disulfide linkers such as

N-succinimidyl-4-(2-pyridyldithio) (SPP) linker or N-succinimidyl-4-(2-pyridyldithio)butanoate (SPDB) linker, and pyrophosphate diester linkers.

Alternatively or simultaneously, one or more of non-cleavable linkers may be used. Examples of non-cleavable linkers include thioether linkers, such as N-succinimidyl 4-(N-maleimidomethyl) cyclohexane-1-carboxylate (SMCC), and maleimidocaproyl (mc) linkers. Generally, non-cleavable linkers are more resistant to proteolytic degradation and more stable compared to cleavable linkers.

### Anti-ADAM12 Chimeric Antigen Receptors (CAR)

In some embodiments, an anti-ADAM12 agent according to the present disclosure may be a chimeric antigen receptor (CAR). In particular, the CARs of the present invention comprise an antigen binding (AB) domain that binds to ADAM12, a transmembrane (TM) domain, and an intracellular signaling (ICS) domain.

A schematic showing a generic CAR construct of the present invention is depicted in FIG. 1A.

The CAR may optionally comprise a hinge that joins the AB domain and said TM domain.

The CAR may optionally comprise one or more costimulatory (CS) domains.

Schematics showing two more generic CAR constructs of the present invention are depicted in FIG. 1B and FIG. 1C.

### AB Domain

The CAR of the present invention comprises an antigen-binding (AB) domain which binds to ADAM12.

In some embodiments, the AB domain of the CAR may comprise any of the anti-ADAM12 agents disclosed herein.

In some embodiments, the AB domain of the CAR may comprise any of the AB domain of any of the anti-ADAM12 agents disclosed herein.

In some embodiments, the AB domain of the CAR may comprise any of the anti-ADAM12 Abs, anti-ADAM12 antigen-binding Ab fragments, anti-ADAM12 multi-specific Abs, anti-ADAM12 multi-specific antigen-binding Ab fragments, and anti-ADAM12 ADCs disclosed herein, or the AB domain thereof.

In some aspects, the AB domain of the CAR may comprise an anti-ADAM12 scFv.

In some aspects, the AB domain may comprise an amino acid sequence at least 80%, at least 85%, at least 90%, at least 92%, at least 94%, at least 95%, at least 96%, at least 97%, at least 98%, at least 99%, or 100% identical to: (i) SEQ ID NOs: 139, 140, 149, or 150, or (ii) the amino acid sequence encoded by SEQ ID NOs: 239, 240, 249, or 250.

In some aspects, the AB domain may compete for binding to ADAM12 with a scFv comprising an amino acid sequence at least 80%, at least 85%, at least 90%, at least 92%, at least 94%, at least 95%, at least 96%, at least 97%, at least 98%, at least 99%, or 100% identical to: (i) SEQ ID NOs: 139, 140, 149, or 150, or (ii) the amino acid sequence encoded by SEQ ID NOs: 239, 240, 249, or 250.

Alternatively, or simultaneously, the AB domain may comprise the ADAM12-binding portion of a molecule that binds to ADAM12.

Examples of physiological ADAM12 substrates include, but are not limited to, alpha actinin 2 (ACTN2), insulin-like growth factor-binding protein 3 (IGFBP3), IGFBP-5, phosphatidylinositol 3 kinase regulatory subunit alpha (PIK3R1), heparin-binding epidermal growth factor (HB-EGF), epidermal growth factor (EGF), Betacellulin, Delta-like 1, and placental leucine aminopeptidase (P-LAP), and matrix metalloprotease (MMP-14) (Galliano M. F. et al., J Biol Chem. 2000 May 5; 275(18):13933-9; Kveiborg M. et al., Int J Biochem Cell Biol. 2008; 40(9):1685-702. doi: 10.1016/j.biocel.2008.01.025. Epub 2008 Feb. 1; Kang Q. et al., J Biol Chem. 2001 Jul. 6; 276(27):24466-72. Epub 2001 Apr. 19; Albrechtsen R. et al., J Cell Sci. 2013 Oct. 15; 126(Pt 20):4707-20. doi: 10.1242/jcs.129510. Epub 2013 Sep. 4). The amino acid sequences of the ADAM12 substrates described above are set forth in SEQ ID NOs: 151, 152, 153, 154, 155, 156, 157, 158, and 159, respectively.

Examples of the AB domain of the CAR, or any other anti-ADAM12 agents, of the present invention are depicted in FIG. 2.

### Hinge

In some embodiments, the CAR may comprise a hinge sequence between the AB domain and the TM domain. One of the ordinary skill in the art will appreciate that a hinge sequence is a short sequence of amino acids that facilitates flexibility (see, e.g. Woof J. M. et al., Nat. Rev. Immunol., 4(2): 89-99 (2004)). The hinge sequence can be any suitable sequence derived or obtained from any suitable molecule.

In some embodiments, the length of the hinge sequence may be optimized based on the desired length of the extracellular portion of the CAR, which may be based on the location of the epitope within the target molecule. For example, if the epitope is in the membrane proximal region within the target molecule, longer hinges may be optimal.

In some embodiments, the hinge may be derived from or include at least a portion of an immunoglobulin Fc region, for example, an IgG1 Fc region, an IgG2 Fc region, an IgG3 Fc region, an IgG4 Fc region, an IgE Fc region, an IgM Fc region, or an IgA Fc region. In certain embodiments, the hinge includes at least a portion of an IgG1, an IgG2, an IgG3, an IgG4, an IgE, an IgM, or an IgA immunoglobulin Fc region that falls within its CH2 and CH3 domains. In some embodiments, the hinge may also include at least a portion of a corresponding immunoglobulin hinge region. In some embodiments, the hinge is derived from or includes at least a portion of a modified immunoglobulin Fc region, for example, a modified IgG1 Fc region, a modified IgG2 Fc region, a modified IgG3 Fc region, a modified IgG4 Fc region, a modified IgE Fc region, a modified IgM Fc region, or a modified IgA Fc region. The modified immunoglobulin Fc region may have one or more mutations (e.g., point mutations, insertions, deletions, duplications) resulting in one or more amino acid substitutions, modifications, or deletions that cause impaired binding of the hinge to an Fc receptor (FcR). In some aspects, the modified immunoglobulin Fc region may be designed with one or more mutations which result in one ore more amino acid substitutions, modifications, or deletions that cause impaired binding of the hinge to one or more FcR including, but not limited to, FcγRI, FcγR2A, FcγR2B1, Fcγ2B2, Fcγ 3A, Fcγ 3B, FcεRI, FcεR2, FcαRI, Fcα/μR, or FcRn.

In some aspects, a portion of the immunoglobulin constant region serves as a hinge between the AB domain, for example scFv or nanobody, and the TM domain. The hinge can be of a length that provides for increased responsiveness of the CAR-expressing cell following antigen binding, as compared to in the absence of the hinge. In some examples, the hinge is at or about 12 amino acids in length or is no more than 12 amino acids in length. Exemplary hinges include those having at least about 10 to 229 amino acids, about 10 to 200 amino acids, about 10 to 175 amino acids, about 10 to 150 amino acids, about 10 to 125 amino acids, about 10 to 100 amino acids, about 10 to 75 amino acids, about 10 to 50 amino acids, about 10 to 40 amino acids, about 10 to 30 amino acids, about 10 to 20 amino acids, or about 10 to 15 amino acids, and including any integer between the endpoints of any of the listed ranges. In some embodiments, a hinge has about 12 amino acids or less, about 119 amino acids or less, or about 229 amino acids or less. Exemplary hinges include a CD28 hinge, IgG4 hinge alone, IgG4 hinge linked to CH2 and CH3 domains, or IgG4 hinge linked to the CH3 domain. Exemplary hinges include, but are not limited to, those described in Hudecek M. et al. (2013) Clin. Cancer Res., 19:3153, international patent application publication number WO2014031687, U.S. Pat. No. 8,822,647 or published App. No. US2014/0271635.

In some embodiments, the hinge sequence is derived from CD8α molecule or a CD28 molecule. In a preferred embodiment, the hinge sequence is derived from CD28. In one embodiment, the hinge comprises the amino acid sequence of human CD28 hinge (SEQ ID NO: 163) or the sequence encoded by SEQ ID NO: 263. In some embodiments, the hinge has an amino acid sequence at least 85%, 86%, 87%, 88%, 89%, 90%, 91%, 92%, 93%, 94%, 95%, 96%, 97%, 98%, 99%, or 100% identical to SEQ ID NO: 163.

### Transmembrane (TM) Domain

With respect to the TM domain, the CAR can be designed to comprise a TM domain that is fused to the AB domain of the CAR. A hinge sequence may be inserted between the AB domain and the TM domain. In one embodiment, the TM domain that naturally is associated with one of the domains in the CAR is used. In some instances, the TM domain can be selected or modified by amino acid substitution to avoid binding of such domains to the transmembrane domains of the same or different surface membrane proteins to minimize interactions with other members of the receptor complex.

The TM domain may be derived either from a natural or from a synthetic source. Where the source is natural, the domain may be derived from any membrane-bound or transmembrane protein. Typically, the TM domain denotes a single transmembrane a helix of a transmembrane protein, also known as an integral protein. TM domains of particular use in this invention may be derived from (i.e. comprise at least the transmembrane region(s) of) CD28, CD3 ε, CD4, CD5, CD8, CD9, CD16, CD22, CD33, CD37, CD45, CD64, CD80, CD86, CD134, CD137, CD154, TCR α, TCR β, or CD3 zeta and/or TM domains containing functional variants thereof such as those retaining a substantial portion of the structural, e.g., transmembrane, properties thereof.

Alternatively the TM domain may be synthetic, in which case the TM domain will comprise predominantly hydrophobic residues such as leucine and valine. Preferably a triplet of phenylalanine, tryptophan and valine will be found at each end of a synthetic TM domain. A TM domain of the invention is thermodynamically stable in a membrane. It may be a single α helix, a transmembrane β barrel, a β-helix of gramicidin A, or any other structure. Transmembrane helices are usually about 20 amino acids in length.

Preferably, the TM domain in the CAR of the invention is derived from the TM region of CD28. In one embodiment, the TM domain comprises the amino acid sequence of human CD28 TM (SEQ ID NO: 161) or the sequence encoded by SEQ ID NO: 261. In some embodiments, the TM domain comprises an amino acid sequence at least 85%, 86%, 87%, 88%, 89%, 90%, 91%, 92%, 93%, 94%, 95%, 96%, 97%, 98%, 99%, or 100% identical to SEQ ID NO: 161.

Optionally, a short oligo- or polypeptide spacer, preferably between 2 and 10 amino acids in length may form the linkage between the TM domain and the ICS domain(s) of the CAR. A glycine-serine doublet may provide a suitable spacer.

### Intracellular Signaling (ICS) Domain and Costimulatory (CS) Domain

The ICS domain or otherwise the cytoplasmic domain of the CAR of the invention triggers or elicits activation of at least one of the normal effector functions of the cell in which the CAR has been placed. The term “effector function” refers to a specialized function of a cell. Effector function of a T cell, for example, may be cytolytic activity or helper activity including the secretion of cytokines. Thus, the term “intracellular signaling domain” or “ICS domain” refers to the portion of a protein which transduces the effector function signal and directs the cell to perform a specialized function. While usually the entire ICS domain can be employed, in many cases it is not necessary to use the entire chain. To the extent that a truncated portion of the intracellular signaling domain is used, such truncated portion may be used in place of the intact chain as long as it transduces the effector function signal. The term “intracellular signaling domain” or “ICS domain” is thus meant to include any truncated portion of the ICS domain sufficient to transduce the effector function signal.

Preferred examples of ICS domains for use in the CAR of the invention include the cytoplasmic sequences of the T cell receptor (TCR) and co-receptors that act in concert to initiate signal transduction following antigen receptor engagement, as well as any derivative or variant of these sequences and any synthetic sequence that has the same functional capability.

Signals generated through one ICS domain alone may be insufficient for full activation of a cell, and a secondary or costimulatory signal may also be required. In such cases, a costimulatory domain (CS domain) may be included in the cytoplasmic portion of a CAR. A CS domain is a domain that transduces such a secondary or costimulatory signal. Optionally, the CAR of the present invention may comprise two or more CS domains. The CS domain(s) may be placed upstream of the ICS domain or downstream of the ICS domain. Two exemplary schematics of CAR constructs of the present invention containing at least one CS domain are illustrated in FIG. 1B and FIG. 1C.

In some embodiments, T cell activation can be said to be mediated by two distinct classes of cytoplasmic signaling sequence: those that initiate antigen-dependent primary activation through the TCR (primary cytoplasmic signaling sequences) and those that act in an antigen-independent manner to provide a secondary or costimulatory signal (secondary cytoplasmic signaling sequences). Primary cytoplasmic signaling sequences regulate primary activation of the TCR complex either in a stimulatory way, or in an inhibitory way. Primary cytoplasmic signaling sequences that act in a stimulatory manner may contain signaling motifs which are known as immunoreceptor tyrosine-based activation motifs or ITAMs. Such a cytoplasmic signaling sequence may be contained in the ICS or the CS domain of the CAR of the present invention.

Examples of ITAM-containing primary cytoplasmic signaling sequences that are of particular use in the invention include those derived from an ICS domain of a lymphocyte receptor chain, a TCR/CD3 complex protein, an Fc receptor subunit, an IL-2 receptor subunit, CD3 ζ, FcR γ, FcR β, CD3γ, CD3 δ, CD3 ε, CD5, CD22, CD66d, CD79a, CD79b, CD278 (ICOS), Fc ε RI, DAP10, and DAP12.

It is particularly preferred that the ICS domain in the CAR of the invention comprises a cytoplasmic signaling sequence derived from CD3 zeta. In one embodiment, the ICS domain comprises the amino acid sequence of human CD3 ζ ICS (SEQ ID NO: 162), or the sequence encoded by SEQ ID NO: 262. In some embodiments, the ICS domain comprises an amino acid sequence at least 85%, 86%, 87%, 88%, 89%, 90%, 91%, 92%, 93%, 94%, 95%, 96%, 97%, 98%, 99%, or 100% identical to SEQ ID NO: 162.

In a preferred embodiment, the cytoplasmic domain of the CAR may be designed to comprise the CD3 ζ ICS domain by itself. In another preferred embodiment, the CD3 ζ ICS domain may be combined with one or more of any other desired cytoplasmic domain(s) useful in the context of the CAR of the invention. For example, the cytoplasmic domain of the CAR can comprise a CD3 ζ ICS domain and a CS domain. The CS region refers to a portion of the CAR comprising the intracellular domain of a costimulatory molecule. A costimulatory molecule is a cell surface molecule other than an antigen receptor or their ligands that is required for an efficient response of lymphocytes to an antigen.

Various CS domains have been reported to confer differing properties. For example, the 4-1BB CS domain showed enhanced persistence in in vivo xenograph models (Milone M. C. et al. Mol Ther 2009; 17:1453-1464; Song D. G. et al. Cancer Res 2011; 71:4617-4627). Additionally, these different CS domains produce different cytokine profiles, which in turn, may produce effects on target cell-mediated cytotoxicity and the disease microenvironment. Indeed, DAP10 signaling in NK cells has been associated with an increase in Th1 and inhibition of Th2 type cytokine production in CD8+ T cells (Barber A. et al. Blood 2011; 117:6571-6581).

Examples of co-stimulatory molecules include an MHC class I molecule, TNF receptor proteins, Immunoglobulin-like proteins, cytokine receptors, integrins, signaling lymphocytic activation molecules (SLAM proteins), activating NK cell receptors, a Toll ligand receptor, B7-H3, BAFFR, BTLA, BLAME (SLAMF8), CD2, CD4, CD5, CD7, CD8α, CD8β, CD11a, LFA-1 (CD11a/CD18), CD11b, CD11c, CD11d, CD18, CD19, CD19a, CD27, CD28, CD29, CD30, CD40, CD49a, CD49D, CD49f, CD69, CD84, CD96 (Tactile), CD100 (SEMA4D), CD103, CRTAM, OX40 (CD134), 4-1BB (CD137), SLAM (SLAMFI, CD150, IPO-3), CD160 (BY55), SELPLG (CD162), DNAM1 (CD226), Ly9 (CD229), SLAMF4 (CD244, 2B4), ICOS (CD278), CEACAMI, CDS, CRTAM, DAP10, GADS, GITR, HVEM (LIGHTR), IA4, ICAM-1, IL2R β, IL2R γ, IL7R α, ITGA4, ITGA6, ITGAD, ITGAE, ITGAL, ITGAM, ITGAX, ITGB1, ITGB2, ITGB7, KIRDS2, LAT, LFA-1, LIGHT, LTBR, NKG2C, NKG2D, NKp30, NKp44, NKp46, NKp80 (KLRF1), PAG/Cbp, PD-1, PSGL1, SLAMF6 (NTB-A, Ly108), SLAMF7, SLP-76, TNFR2, TRANCE/RANKL, VLA1, VLA-6, a ligand that specifically binds with CD83, and the like. Thus, while the invention is exemplified primarily with regions of CD28, DAP10, and/or 4-1BB as the CS domain, other costimulatory elements are within the scope of the invention.

The ICS domain and the CS domain(s) of the CAR of the invention may be linked to each other in a random or specified order. Optionally, a short oligo- or polypeptide linker, preferably between 2 and 10 amino acids in length may form the linkage. A glycine-serine doublet provides a particularly suitable linker.

In one embodiment, the CAR is designed to comprise a cytoplasmic signaling sequence of CD3 ζ as the ICS domain and comprise a cytoplasmic signaling sequence of CD28 as the CS domain. In another embodiment, the CAR is designed to comprise a cytoplasmic signaling sequence of CD3 ζ as the ICS domain and comprise a cytoplasmic signaling sequence of DAP10 as the CS domain. In yet another embodiment, the CAR is designed to comprise a cytoplasmic signaling sequence of CD3 ζ as the ICS domain and comprise a cytoplasmic signaling sequence of 4-1BB as the CS domain. Such a cytoplasmic signaling sequence of CD3 ζ may be at least 85%, 86%, 87%, 88%, 89%, 90%, 91%, 92%, 93%, 94%, 95%, 96%, 97%, 98%, 99%, or 100% identical to the CD3 ζICS domain comprising the amino acid sequence of human CD3z ICS (SEQ ID NO: 162). Such a cytoplasmic signaling sequence of CD3 zeta may be encoded by a nucleic acid sequence at least 85%, 86%, 87%, 88%, 89%, 90%, 91%, 92%, 93%, 94%, 95%, 96%, 97%, 98%, 99%, or 100% identical to SEQ ID NO: 262.

Such a cytoplasmic signaling sequence of CD28 may be at least 85%, 86%, 87%, 88%, 89%, 90%, 91%, 92%, 93%, 94%, 95%, 96%, 97%, 98%, 99%, or 100% identical to the sequence of human CD28 CS domain (SEQ ID NO: 164). Such a cytoplasmic signaling sequence of CD28 may be encoded by a nucleic acid sequence at least 85%, 86%, 87%, 88%, 89%, 90%, 91%, 92%, 93%, 94%, 95%, 96%, 97%, 98%, 99%, or 100% identical to SEQ ID NO: 264. Such a cytoplasmic signaling sequence of DAP10 may be at least 85%, 86%, 87%, 88%, 89%, 90%, 91%, 92%, 93%, 94%, 95%, 96%, 97%, 98%, 99%, or 100% identical to the sequence of human 4-1BB CS domain (SEQ ID NO: 165). Such a cytoplasmic signaling sequence of 4-1BB may be encoded by a nucleic acid sequence at least 85%, 86%, 87%, 88%, 89%, 90%, 91%, 92%, 93%, 94%, 95%, 96%, 97%, 98%, 99%, or 100% identical to SEQ ID NO: 265. Such a cytoplasmic signaling sequence of DAP10 may be at least 85%, 86%, 87%, 88%, 89%, 90%, 91%, 92%, 93%, 94%, 95%, 96%, 97%, 98%, 99%, or 100% identical to the sequence of human DAP10 CS domain (SEQ ID NO: 166). Such a cytoplasmic signaling sequence of DAP10 may be encoded by a nucleic acid sequence at least 85%, 86%, 87%, 88%, 89%, 90%, 91%, 92%, 93%, 94%, 95%, 96%, 97%, 98%, 99%, or 100% identical to SEQ ID NO: 266.

Alternatively, when the AB domain comprises the ADAM12-binding portion of a molecule that binds to ADAM12 as described above, the TM domain of the CAR may be derived from the transmembrane portion of the molecule.

### Exemplary CAR Constructs

In the following CAR examples, the CAR construct is described as “AB domain—hinge—TM domain—CS domain—ICS domain.”

The CARs of the present invention may comprise an amino acid sequence at least 80%, at least 85%, at least 90%, at least 95%, at least 98% at least 99%, or 100% identical to any of the exemplary constructs below.

In one embodiment the CAR of the invention may be described as h6E6scFvHL-CD28H-CD28TM-CD28CS-CD3zICS, and may comprise the amino acid sequence as set forth in SEQ ID NO: 171. A nucleic acid sequence encoding such a CAR may comprise the sequence as set forth in SEQ ID NO: 271.

In one embodiment the CAR of the invention may be described as h6E6scFvHL-CD28H-CD28TM-41BBCS-CD3zlCS, and may comprise the amino acid sequence as set forth in SEQ ID NO: 172. A nucleic acid sequence encoding such a CAR may comprise the sequence as set forth in SEQ ID NO: 272.

In one embodiment the CAR of the invention may be described as h6E6scFvHL-CD28H-CD28TM-DAP10CS-CD3zICS, and may comprise the amino acid sequence as set forth in SEQ ID NO: 173. A nucleic acid sequence encoding such a CAR may comprise the sequence as set forth in SEQ ID NO: 273.

In one embodiment the CAR of the invention may be described as h6E6scFvLH-CD28H-CD28TM-CD28CS-CD3zICS, and may comprise the amino acid sequence as set forth in SEQ ID NO: 174. A nucleic acid sequence encoding such a CAR may comprise the sequence as set forth in SEQ ID NO: 274.

In one embodiment the CAR of the invention may be described as h6E6scFvLH-CD28H-CD28TM-41BBCS-CD3zICS, and may comprise the amino acid sequence as set forth in SEQ ID NO: 175. A nucleic acid sequence encoding such a CAR may comprise the sequence as set forth in SEQ ID NO: 275.

In one embodiment the CAR of the invention may be described as h6E6scFvLH-CD28H-CD28TM-DAP10CS-CD3zICS, and may comprise the amino acid sequence as set forth in SEQ ID NO: 176. A nucleic acid sequence encoding such a CAR may comprise the sequence as set forth in SEQ ID NO: 276.

In one embodiment the CAR of the invention may be described as h6C10scFvHL-CD28H-CD28TM-CD28CS-CD3zICS, and may comprise the amino acid sequence as set forth in SEQ ID NO: 177. A nucleic acid sequence encoding such a CAR may comprise the sequence as set forth in SEQ ID NO: 277.

In one embodiment the CAR of the invention may be described as h6C10scFvHL-CD28H-CD28TM-41BBCS-CD3zICS, and may comprise the amino acid sequence as set forth in SEQ ID NO: 178. A nucleic acid sequence encoding such a CAR may comprise the sequence as set forth in SEQ ID NO: 278.

In one embodiment the CAR of the invention may be described as h6C10scFvHL-CD28H-CD28TM-DAP10CS-CD3zICS, and may comprise the amino acid sequence as set forth in SEQ ID NO: 179. A nucleic acid sequence encoding such a CAR may comprise the sequence as set forth in SEQ ID NO: 279.

In one embodiment the CAR of the invention may be described as h6C10scFvLH-CD28H-CD28TM-CD28CS-CD3zICS, and may comprise the amino acid sequence as set forth in SEQ ID NO: 180. A nucleic acid sequence encoding such a CAR may comprise the sequence as set forth in SEQ ID NO: 280.

In one embodiment the CAR of the invention may be described as h6C10scFvLH-CD28H-CD28TM-41BBCS-CD3zICS, and may comprise the amino acid sequence as set forth in SEQ ID NO: 181. A nucleic acid sequence encoding such a CAR may comprise the sequence as set forth in SEQ ID NO: 281.

In one embodiment the CAR of the invention may be described as h6C10scFvLH-CD28H-CD28TM-DAP10CS-CD3zICS, and may comprise the amino acid sequence as set forth in SEQ ID NO: 182. A nucleic acid sequence encoding such a CAR may comprise the sequence as set forth in SEQ ID NO: 282.

Schematics showing examples of specific CAR constructs of some embodiments are illustrated in FIG. 3A-C.

In some embodiments, a leader sequence (LS) may be placed upstream of the polynucleotide sequences encoding the foregoing exemplary CARs. The leader sequence facilitates the expression of the CAR on the cell surface. The polynucleotide sequence of such a lead sequence may be as set forth in SEQ ID NO: 260, which encodes the amino acid sequence as set forth in SEQ ID NO: 160. Any other sequences that facilitate the expression of the CAR on the cell surface may be used.

A general exemplary schematic of a construct for a LS-containing CAR of the present invention is shown in FIG. 1D.

In some embodiments, the polynucleotide sequences for expressing the foregoing exemplary CARs further comprise a T2A ribosomal skip sequence (or also referred to as T2A) and/or a sequence encoding truncated CD19 (or also referred to as trCD19). The nucleic acid sequence for T2A may be as provided by SEQ ID NO: 269, which encodes the amino acid sequence provided by SEQ ID NO: 169. trCD19 may have the sequence as provided by SEQ ID NO: 170, which may be encoded by, for example, SEQ ID NO: 270.

A schematic showing such polynucleotide constructs are shown in FIG. 1E.

When the T2A and trCD19 sequences are placed downstream of the CAR sequence, the translation will be interrupted by the T2A sequence, resulting in two separate translation products, CAR protein and trCD19 protein.

The present disclosure encompasses the nucleic acid sequences encoding any of the CARs disclosed herein.

### Further Modification

The CARs of the present invention, nucleotide sequences encoding the same, vectors encoding the same, and cells comprising nucleotide sequences encoding said CARs may be further modified, engineered, optimized, or appended in order to provide or select for various features. These features may include, but are not limited to, efficacy, persistence, target specificity, reduced immunogenicity, multi-targeting, enhanced immune response, expansion, growth, reduced off-target effect, reduced subject toxicity, improved target cytotoxicity, improved attraction of disease alleviating immune cells, detection, selection, targeting, and the like. For example, the cells may be engineered to express another CAR, or to have a suicide mechanism, and may be modified to remove or modify expression of an endogenous receptor or molecule such as a TCR and/or MHC molecule.

In some embodiments, the vector or nucleic acid sequence encoding the CAR further encodes other genes. The vector or nucleic acid sequence may be constructed to allow for the co-expression of multiple genes using a multitude of techniques including co-transfection of two or more plasmids, the use of multiple or bidirectional promoters, or the creation of bicistronic or multicistronic vectors. The construction of multicistronic vectors may include the encoding of IRES elements or 2A peptides, such as T2A, P2A, E2A, or F2A (for example, see Kim, J. H., et al., “High cleavage efficiency of a 2A peptide derived from porcine teschovirus-1 in human cell lines, zebrafish and mice”, PLoS One. 2011; 6(4)). In a particular embodiment, the nucleic acid sequence or vector encoding the CAR further encodes trCD19 with the use of a T2A ribosomal skip sequence.

The CAR expressing cell may further comprise a disruption to one or more endogenous genes. In some embodiments, the endogenous gene encodes TCRα, TCRβ, CD52, glucocorticoid receptor (GR), deoxycytidine kinase (dCK), or an immune checkpoint protein such as, for example, programmed death-1 (PD-1).

### Efficacy

The CARs of the present invention and cells expressing these CARs may be further modified to improve efficacy against cells expressing the target molecule. The cells may be cells expressing ADAM12. The cells expressing ADAM12 may be cancer cells, vascular cells, or any other target disease-associated cells. In some embodiments, the improved efficacy may be measured by increased cytotoxicity against cells expressing the target molecule, for example cytotoxicity against cancer cells. In some embodiments, the improved efficacy may also be measured by increased production of cytotoxic mediators such as, but not limited to, IFN γ, perforin, and granzyme B. In some embodiments, the improved efficacy may be shown by reduction in the signature cytokines of the diseases, or alleviated symptoms of the disease when the CAR expressing cells are administered to a subject. Other cytokines that may be reduced include TGF-beta, IL-6, IL-4, IL-10, and/or IL-13. the improved efficacy may be shown by ADAM12-specific immune cell responses, such as T cell cytotoxicity. In case of cancer, improved efficacy may be shown by better tumor cytotoxicity, better infiltration into the tumor, reduction of immunosuppressive mediators, reduction in weight decrease, reduction in ascites, reduction in tumor burden, and/or increased lifespan. In case of autoimmune diseases, reduced responsiveness of autoreactive cells or decrease in autoreactive T cells, B cells, or Abs may represent improved efficacy. In some embodiments, gene expression profiles may be also investigated to evaluate the efficacy of the CAR.

In one aspect, the CAR expressing cells are further modified to evade or neutralize the activity of immunosuppressive mediators, including, but not limited to prostaglandin E2 (PGE2) and adenosine. In some embodiments, this evasion or neutralization is direct. In other embodiments, this evasion or neutralization is mediated via the inhibition of protein kinase A (PKA) with one or more binding partners, for example ezrin. In a specific embodiment, the CAR-expressing cells further express the peptide “regulatory subunit I anchoring disruptor” (RIAD). RIAD is thought to inhibit the association of protein kinase A (PKA) with ezrin, which thus prevents PKA's inhibition of TCR activation (Newick K. et al. Cancer Immunol Res. 2016 June; 4(6):541-51. doi: 10.1158/2326-6066.CIR-15-0263. Epub 2016 Apr. 4).

In some embodiments, the CAR expressing cells of the invention may induce a broad immune response, consistent with epitope spreading.

In some embodiments, the CAR expressing cells of the invention further comprise a homing mechanism. For example, the cell may transgenically express one or more stimulatory chemokines or cytokines or receptors thereof. In particular embodiments, the cells are genetically modified to express one or more stimulatory cytokines. In certain embodiments, one or more homing mechanisms are used to assist the inventive cells to accumulate more effectively to the disease site. In some embodiments, the CAR expressing cells are further modified to release inducible cytokines upon CAR activation, e.g., to attract or activate innate immune cells to a targeted cell (so-called fourth generation CARs or TRUCKS). In some embodiments, CARs may co-express homing molecules, e.g., CCR4 or CCR2b, to increase trafficking to the disease site.

### Controlling CAR Expression

In some instances, it may be advantageous to regulate the activity of the CAR or CAR expressing cells CAR. For example, inducing apoptosis using, e.g., a caspase fused to a dimerization domain (see, e.g., Di et al., N Engl. J. Med. 2011 Nov. 3; 365(18):1673-1683), can be used as a safety switch in the CAR therapy of the instant invention. In another example, CAR-expressing cells can also express an inducible Caspase-9 (iCaspase-9) molecule that, upon administration of a dimerizer drug (e.g., rimiducid (also called AP1903 (Bellicum Pharmaceuticals) or AP20187 (Ariad)) leads to activation of the Caspase-9 and apoptosis of the cells. The iCaspase-9 molecule contains a chemical inducer of dimerization (CID) binding domain that mediates dimerization in the presence of a CID. This results in inducible and selective depletion of CAR-expressing cells. In some cases, the iCaspase-9 molecule is encoded by a nucleic acid molecule separate from the CAR-encoding vector(s). In some cases, the iCaspase-9 molecule is encoded by the same nucleic acid molecule as the CAR-encoding vector. The iCaspase-9 can provide a safety switch to avoid any toxicity of CAR-expressing cells. See, e.g., Song et al. Cancer Gene Ther. 2008; 15(10):667-75; Clinical Trial Id. No. NCT02107963; and Di et al. N. Engl. J. Med. 2011; 365:1673-83.

Alternative strategies for regulating the CAR therapy of the instant invention include utilizing small molecules or antibodies that deactivate or turn off CAR activity, e.g., by deleting CAR-expressing cells, e.g., by inducing antibody dependent cell-mediated cytotoxicity (ADCC). For example, CAR-expressing cells described herein may also express an antigen that is recognized by molecules capable of inducing cell death, e.g., ADCC or compliment-induced cell death. For example, CAR expressing cells described herein may also express a receptor capable of being targeted by an antibody or antibody fragment. Examples of such receptors include EpCAM, VEGFR, integrins (e.g., integrins αVρ3, α4, α13/4β3, α4β7, α5β1, αvβ, αv), members of the TNF receptor superfamily (e.g., TRAIL-R1, TRAIL-R2), PDGF Receptor, interferon receptor, folate receptor, GPNMB, ICAM-1, HLA-DR, CEA, CA-125, MUC1, TAG-72, IL-6 receptor, 5T4, GD2, GD3, CD2, CD3, CD4, CDS, CD11, CD11a/LFA-1, CD15, CD18/ITGB2, CD19, CD20, CD22, CD23/lgE Receptor, CD25, CD28, CD30, CD33, CD38, CD40, CD41, CD44, CD51, CD52, CD62L, CD74, CD80, CD125, CD147/basigin, CD152/CTLA-4, CD154/CD40L, CD195/CCR5, CD319/SLAMF7, and EGFR, and truncated versions thereof (e.g., versions preserving one or more extracellular epitopes but lacking one or more regions within the cytoplasmic domain). For example, CAR-expressing cells described herein may also express a truncated epidermal growth factor receptor (EGFR) which lacks signaling capacity but retains the epitope that is recognized by molecules capable of inducing ADCC, e.g., cetuximab (ERBITUX®), such that administration of cetuximab induces ADCC and subsequent depletion of the CAR-expressing cells (see, e.g., WO2011/056894, and Jonnalagadda et al., “Gene Ther. 2013; 20(8)853-860).

In some embodiments, the CAR cell comprises a polynucleotide encoding a suicide polypeptide, such as for example RQR8. See, e.g., WO2013153391A, which is hereby incorporated by reference in its entirety. In CAR cells comprising the polynucleotide, the suicide polypeptide may be expressed at the surface of a CAR cell. The suicide polypeptide may also comprise a signal peptide at the amino terminus. Another strategy includes expressing a highly compact marker/suicide gene that combines target epitopes from both CD32 and CD20 antigens in the CAR-expressing cells described herein, which binds rituximab, resulting in selective depletion of the CAR-expressing cells, e.g., by ADCC (see, e.g., Philip et al., “Blood. 2014; 124(8)1277-1287). Other methods for depleting CAR-expressing cells described herein include administration of CAMPATH®, a monoclonal anti-CD52 antibody that selectively binds and targets mature lymphocytes, e.g., CAR-expressing cells, for destruction, e.g., by inducing ADCC. In other embodiments, the CAR-expressing cell can be selectively targeted using a CAR ligand, e.g., an anti-idiotypic antibody. In some embodiments, the anti-idiotypic antibody can cause effector cell activity, e.g., ADCC or ADC activities, thereby reducing the number of CAR-expressing cells. In other embodiments, the CAR ligand, e.g., the anti-idiotypic antibody, can be coupled to an agent that induces cell killing, e.g., a toxin, thereby reducing the number of CAR-expressing cells. Alternatively, the CAR molecules themselves can be configured such that the activity can be regulated, e.g., turned on and off, as described below.

In some embodiments, a regulatable CAR (RCAR) where the CAR activity can be controlled is desirable to optimize the safety and efficacy of a CAR therapy. In some embodiments, a RCAR comprises a set of polypeptides, typically two in the simplest embodiments, in which the components of a standard CAR described herein, e.g., an AB domain and an ICS domain, are partitioned on separate polypeptides or members. In some embodiments, the set of polypeptides include a dimerization switch that, upon the presence of a dimerization molecule, can couple the polypeptides to one another, e.g., can couple an AB domain to an ICS domain. Additional description and exemplary configurations of such regulatable CARs are provided herein and in International Publication No. WO 2015/090229, hereby incorporated by reference in its entirety.

In an aspect, an RCAR comprises two polypeptides or members: 1) an intracellular signaling member comprising an ICS domain, e.g., a primary ICS domain described herein, and a first switch domain; 2) an antigen binding member comprising an AB domain, e.g., that specifically binds a target molecule described herein, as described herein and a second switch domain. Optionally, the RCAR comprises a TM domain described herein. In an embodiment, a TM domain can be disposed on the intracellular signaling member, on the antigen binding member, or on both. Unless otherwise indicated, when members or elements of an RCAR are described herein, the order can be as provided, but other orders are included as well. In other words, in an embodiment, the order is as set out in the text, but in other embodiments, the order can be different. E.g., the order of elements on one side of a transmembrane region can be different from the example, e.g., the placement of a switch domain relative to an ICS domain can be different, e.g., reversed.

In some embodiments, the CAR expressing immune cell may only transiently express a CAR. For example, the cells of the invention may be transduced with mRNA comprising a nucleic acid sequence encoding an inventive CAR. In this vein, the present invention also includes an RNA construct that can be directly transfected into a cell. A method for generating mRNA for use in transfection involves in vitro transcription (IVT) of a template with specially designed primers, followed by polyA addition, to produce a construct containing 3′ and 5′ untranslated sequences (“UTRs”), a 5′ cap and/or Internal Ribosome Entry Site (IRES), the nucleic acid to be expressed, and a polyA tail, typically 50-2000 bases in length. RNA so produced can efficiently transfect different kinds of cells. In one embodiment, the template includes sequences for the CAR. In an embodiment, an RNA CAR vector is transduced into a cell by electroporation.

### Target Specificity

The CAR expressing cells of the present invention may further comprise one or more CARs, in addition to the first CAR. These additional CARs may or may not be specific for the target molecule of the first CAR. In some embodiments, the one or more additional CARs may act as inhibitory or activating CARs. In some aspects, the CAR of some embodiments is the stimulatory or activating CAR; in other aspects, it is the costimulatory CAR. In some embodiments, the cells further include inhibitory CARs (iCARs, see Fedorov et al., Sci. Transl. Medicine, 2013 December; 5(215): 215ra172), such as a CAR recognizing an antigen other than the target molecule of the first CAR, whereby an activating signal delivered through the first CAR is diminished or inhibited by binding of the inhibitory CAR to its ligand, e.g., to reduce off-target effects.

In some embodiments, the AB domain of the CAR is or is part of an immunoconjugate, in which the AB domain is conjugated to one or more heterologous molecule(s), such as, but not limited to, a cytotoxic agent, an imaging agent, a detectable moiety, a multimerization domain, or other heterologous molecule. Cytotoxic agents include, but are not limited to, radioactive isotopes (e.g., At211, I131, I125, Y90, Re186, Re188, Sm153, Bi212, P32, Pb212 and radioactive isotopes of Lu); chemotherapeutic agents; growth inhibitory agents; enzymes and fragments thereof such as nucleolytic enzymes; antibiotics; toxins such as small molecule toxins or enzymatically active toxins. In some embodiments, the AB domain is conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes.

In some embodiments, to enhance persistence, the cells of the invention may be further modified to overexpress pro-survival signals, reverse anti-survival signals, overexpress Bcl-xL, overexpress hTERT, lack Fas, or express a TGF-β dominant negative receptor. Persistence may also be facilitated by the administration of cytokines, e.g., IL-2, IL-7, and IL-15.

### Vectors

The present invention also provides vectors in which a polynucleotide encoding an anti-ADAM12 agent of the present invention is inserted.

The vector may be, for example, a DNA vector or a RNA vector. The vector may be, for example, but not limited to, a plasmid, a cosmid, or a viral vector. The viral vector may be a vector of a DNA virus, which may be an adenovirus, or an RNA virus, which may be a retrovirus. Types of vectors suite for Abs, antigen-binding Ab fragments, and/or CARs are well known in the art (for example, see Rita Costa A. et al., Eur J Pharm Biopharm. 2010 February; 74(2):127-38. doi: 10.1016/j.ejpb.2009.10.002. Epub 2009 Oct. 22; Frenzel A. et al. Front Immunol. 2013; 4: 217. Published online 2013 Jul. 29. doi: 10.3389/fimmu.2013.00217).

When the host cells are insect cells, such as for producing Abs or antigen-binding Ab fragments, insect-specific viruses may be used. Examples of the insect-specific viruses include, but are not limited to, the family of Baculoviridae, particularly the Autographa californica nuclear polyhedrosis virus (AcNPV). When the host cells are plant cells, plant-specific viruses and bacteria, such as Agrobacterium tumefaciens, may be used.

For expressing vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity. This would be particularly beneficial for expressing CAR constructs.

In brief summary, the expression of nucleic acids encoding anti-ADAM12 agents is typically achieved by operably linking a nucleic acid encoding the anti-ADAM12 agent polypeptide or portions thereof to a promoter, and incorporating the construct into an expression vector. The vectors can be suitable for replication and integration eukaryotes. Typical cloning vectors contain transcription and translation terminators, initiation sequences, and promoters useful for regulation of the expression of the desired nucleic acid sequence.

The expression constructs of the present invention may also be used for nucleic acid immunization and gene therapy, using standard gene delivery protocols. Methods for gene delivery are known in the art. See, e.g., U.S. Pat. Nos. 5,399,346, 5,580,859, 5,589,466, incorporated by reference herein in their entireties. In another embodiment, the invention provides a gene therapy vector.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses, which are useful as vectors include, but are not limited to, retroviruses, γ-retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

Various promoter sequences may be used, including, but not limited to the immediate early cytomegalovirus (CMV) promoter, the CMV-actin-globin hybrid (CAG) promotor, Elongation Growth Factor-1α (EF-1α), simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

In order to assess the expression of a CAR polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

In some embodiments, the selectable marker gene comprises a nucleic acid sequence encoding truncated CD19 (trCD19). When a marker such as trCD19, which can be expressed on the cell surface is used, the expression of the marker may be determined via any available technique including, but not limited to, flow cytometry or immunofluorescence assays. Expression of such a marker typically indicates successful introduction and expression of the transgene(s) introduced together with the marker gene. Therefore, cells expressing the anti-ADAM12 agent of the invention may be, for example, selected based on the expression of the marker.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, β-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

### Transfection/Transduction

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

For transduction of CAR constructs to obtain CAR-expressing cells, a flow chart illustrating a potential method for manufacturing isolated CAR-expressing cells is provided in FIG. 4.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York). A preferred method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20 degrees Celsius. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., “1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell or otherwise expose a cell to the inhibitor of the present invention, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

### Cells

Also provided are cells, cell populations, and compositions containing the cells, e.g., cells comprising a nucleic acid sequence encoding an anti-ADAM12 agent of the present invention. Cells expressing anti-ADAM12 Abs or antigen-binding Ab fragments may be used to harvest the Abs or antigen-binding Ab fragments. Cells expressing anti-ADAM12 CARs may be administered to a subject or may be incorporated in a composition to be administered to a subject. Among the compositions are pharmaceutical compositions and formulations for administration, such as for adoptive cell therapy.

Also provided are therapeutic methods for administering the Abs or Ab fragments or the cells and compositions to subjects, e.g., patients.

### Cell Types

Thus, also provided are cells expressing the anti-ADAM12 agents of the present invention.

For expressing an anti-ADAM12 Ab or antigen-binding Ab fragment, any appropriate cells may be used. For example, cells may be: (i) prokaryotic cells, such as gram-negative bacteria and gram-positive bacteria; or (ii) eukaryotic cells, such as yeast, filamentous fungi, protozoa, insect cells, plant cells, and mammalian cells (reviewed in Frenzel A. et al. Front Immunol. 2013; 4: 217. Published online 2013 Jul. 29. doi: 10.3389/fimmu.2013.00217).

Specific examples of gram-negative bacteria that are suited for production of Ab or antigen-binding Ab fragments include, but are not limited to, E. coli, Proteus mirabilis, and Pseudomonas putidas. Specific examples of gram-positive bacteria include, but are not limited to, Bacillus brevis, Bacillus subtilis, Bacillus megaterium, Lactobacillus zeae/casei, and Lactobacillus paracasei. Specific examples of yeast bacteria that are suited for production of Ab or antigen-binding Ab fragments include, but are not limited to, Pichia pastoris, Saccharomyces cerevisiae, Hansenula polymorpha, Schizosaccharomyces pombe, Schwanniomyces occidentalis, Kluyveromyces lactis, and Yarrowia lipolytica. Specific examples of filamentous fungi that are suited for production of Ab or antigen-binding Ab fragments include, but are not limited to, the genera Trichoderma and Aspergillus, A. niger (subgenus A. awamori), Aspergillus oryzae, and Chrysosporium lucknowense. Specific examples of protozoa that are suited for production of Ab or antigen-binding Ab fragments include, but are not limited to, Leishmania tarentolae. Specific examples of insect cells that are suited for production of Ab or antigen-binding Ab fragments include, but are not limited to, insect cell lines like Sf-9 and Sf-21 of Spodoptera frugiperda, DS2 cells of Drosophila melanogaster, High Five cells (BTI-TN-5B1-4) of Trichoplusia ni, or Schneider2 (S2) cells of D. melanogaster. They can be efficiently transfected with insect-specific viruses from the family of Baculoviridae, particularly the Autographa californica nuclear polyhedrosis virus (AcNPV). Specific examples of mammalian cells that are suited for production of Ab or antigen-binding Ab fragments include, but are not limited to, Chinese hamster ovary (CHO) cells, the human embryonic retinal cell line Per.C6 [Crucell, Leiden, Netherlands], CHO-derived cell lines such as K1−, DukXB11−, Lec13, and DG44− cell lines, mouse myeloma cells such as SP 2/0, YB 2/0, and NS0 cells, GS-NS0, hybridoma cells, baby hamster kidney (BHK) cells, and the human embryonic kidney cell line HEK293, HEK293T, HEK293E, and human neuronal precursor cell line AGE1.HN (Probiogen, Berlin, Germany).

Alternatively, genetically modified organisms such as transgenic plants and transgenic animals may be used. Exemplary plants that may be used include, but are not limited to, tobacco, maize, duckweed, Chlamydomonas reinhardtii, Nicotiana tabacum, Nicotianaben thamiana, and Nicotiana benthamiana. Exemplary animals that may be used include, but are not limited to mouse, rat, and chicken.

For expressing an anti-ADAM12 CAR, the cells generally are eukaryotic cells, such as mammalian cells, and typically are human cells, more typically primary human cells, e.g., allogeneic or autologous donor cells. The cells for introduction of the CAR may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered. In some embodiments, the cells are derived from the blood, bone marrow, lymph, or lymphoid organs, are cells of the immune system, such as cells of the innate or adaptive immunity, e.g., myeloid cells, including monocytes, macrophages, dendritic cells, neutrophils, eosinophils, basophils, or mast cells, or lymphoid cells, including lymphocytes, typically T cells and/or NK cells. Other exemplary cells include stem cells, such as multipotent and pluripotent stem cells, including induced pluripotent stem cells (iPSCs). The cells typically are primary cells, such as those isolated directly from a subject and/or isolated from a subject and frozen. In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation.

Alternatively, an immortalized cell or a cell line may be used for expressing a CAR of the present disclosure. Such examples include, but are not limited to, a T cell line, a CD4+ T cell line, a CD8+ T cell line, a regulatory T cell line, an NK-T cell line, an NK cell line (e.g., NK-92), a monocyte line, a macrophage line, a dendritic cell line, and a mast cell line. Furthermore, a desired cell type for CAR expression, for example T cells or NK cells may be generated from a stem cell, such as an embryonic stem cell, iPSCs, or hematopoietic stem cell.

With reference to the subject to be treated with cells expressing an anti-ADAM12 CAR, the cells may be allogeneic and/or autologous. Among the methods include off-the-shelf methods. In some aspects, such as for off-the-shelf technologies, the cells are pluripotent and/or multipotent, such as stem cells, such as induced pluripotent stem cells (iPSCs). In some embodiments, the methods include isolating cells from the subject, preparing, processing, culturing, and/or engineering them, as described herein, and re-introducing them into the same patient, before or after cryopreservation.

In some embodiments, the cells are T cells. Among the sub-types and subpopulations of T cells and/or of CD4+ and/or of CD8+ T cells are naive T (TN) cells, effector T cells (TEFF), memory T cells and sub-types thereof, such as stem cell memory T (TSCM), central memory T (TCM), effector memory T (TEM), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, α/β T cells, and δ/γ T cells.

In some embodiments, the cells are natural killer (NK) cells, Natural Killer T (NKT) cells, cytokine-induced killer (CIK) cells, tumor-infiltrating lymphocytes (TIL), lymphokine-activated killer (LAK) cells, or the like. In some embodiments, the cells are monocytes or granulocytes, e.g., myeloid cells, macrophages, neutrophils, dendritic cells, mast cells, eosinophils, and/or basophils. CAR-expressing phagocytic cells expressing may be able to bind to and phagocytose or nibble target cells (Morrissey M. A. et al., Elife. 2018 Jun. 4; 7. pii: e36688. doi: 10.7554/eLife.36688).

In some embodiments, the cells are derived from cell lines, e.g., T cell lines. The cells in some embodiments are obtained from a xenogeneic source, for example, from mouse, rat, non-human primate, and pig.

### Cell Acquisition for Anti-ADAM12 CAR Expression

For cells for expressing anti-ADAM12 CARs, prior to expansion and genetic modification, a source of cells can be obtained from a subject through a variety of non-limiting methods. Cells can be obtained from a number of non-limiting sources, including peripheral blood mononuclear cells, bone marrow, lymph node tissue, cord blood, thymus tissue, tissue from a site of infection, ascites, pleural effusion, spleen tissue, and disease sites such as the fibrotic sites or tumors. In some embodiments, any number of T cell lines available and known to those skilled in the art, may be used. In some embodiments, cells can be derived from a healthy donor, from a patient diagnosed with cancer or from a patient diagnosed with an infection. In some embodiments, cells can be part of a mixed population of cells which present different phenotypic characteristics.

Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g. transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an Cresis or leukapheresis product. Exemplary samples include whole blood, peripheral blood mononuclear cells (PBMCs), leukocytes, bone marrow, thymus, tissue biopsy, fibrotic tissue, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some examples, cells from the circulating blood of a subject are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects contains cells other than red blood cells and platelets.

Also provided herein are cell lines obtained from a transformed cell according to any of the above-described methods. Also provided herein are modified cells resistant to an immunosuppressive treatment. In some embodiments, an isolated cell according to the invention comprises a polynucleotide encoding a CAR.

### Cell Purification

In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, the wash solution lacks calcium and/or magnesium and/or many or all divalent cations. In some aspects, a washing step is accomplished a semi-automated “flow-through” centrifuge (for example, the Cobe 2991 cell processor, Baxter) according to the manufacturer's instructions. In some aspects, a washing step is accomplished by tangential flow filtration (TFF) according to the manufacturer's instructions. In some embodiments, the cells are resuspended in a variety of biocompatible buffers after washing, such as, for example, Ca++/Mg++ free PBS. In certain embodiments, components of a blood cell sample are removed and the cells directly resuspended in culture media.

In some embodiments, the isolation methods include the separation of different cell types based on the expression or presence in the cell of one or more specific molecules, such as surface markers, e.g., surface proteins, intracellular markers, or nucleic acid. This would be particularly useful for isolating CAR-expressing cells. In a specific embodiment, the surface maker is trCD19. In some embodiments, any known method for separation based on such markers may be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population.

In some embodiments, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

For example, in some aspects, specific subpopulations of T cells, such as cells positive or expressing high levels of one or more surface markers, e.g., CD28+, CD62L+, CCR7+, CD27+, CD127+, CD4+, CD8+, CD45RA+, and/or CD45RO+ T cells, are isolated by positive or negative selection techniques. For example, CD3+ T cells can be positively selected using CD3 conjugated magnetic beads (e.g., DYNABEADS® M-450 CD3/CD28 T Cell Expander).

In some embodiments, isolation is carried out by enrichment for a particular cell population by positive selection, or depletion of a particular cell population, by negative selection. In some embodiments, positive or negative selection is accomplished by incubating cells with one or more antibodies or other binding agent that specifically bind to one or more surface markers expressed or expressed (marker+) at a relatively higher level (markerhigh) on the positively or negatively selected cells, respectively.

In some embodiments, T cells are separated from a PBMC sample by negative selection of markers expressed on non-T cells, such as B cells, monocytes, or other white blood cells, such as CD14. In some aspects, a CD4+ or CD8+ selection step is used to separate CD4+ helper and CD8+ cytotoxic T cells. Such CD4+ and CD8+ populations can be further sorted into sub-populations by positive or negative selection for markers expressed or expressed to a relatively higher degree on one or more naive, memory, and/or effector T cell subpopulations.

In some embodiments, CD8+ cells are further enriched for or depleted of naive, central memory, effector memory, and/or central memory stem cells, such as by positive or negative selection based on surface antigens associated with the respective subpopulation. In some embodiments, enrichment for central memory T (TCM) cells is carried out to increase efficacy, such as to improve long-term survival, expansion, and/or engraftment following administration, which in some aspects is particularly robust in such sub-populations. See Terakura et al. (2012) Blood. 1:72-82; Wang et al. (2012) J Immunother. 35(9):689-701. In some embodiments, combining TCM-enriched CD8+ T cells and CD4+ T cells further enhances efficacy. In embodiments, memory T cells are present in both CD62L+ and CD62L-subsets of CD8+ peripheral blood lymphocytes. PBMC can be enriched for or depleted of CD62L-CD8+ and/or CD62L+CD8 fractions, such as using anti-CD8 and anti-CD62L antibodies.

In some embodiments, the enrichment for central memory T (TCM) cells is based on positive or high surface expression of CD45RO, CD62L, CCR7, CD28, CD3, and/or CD127; in some aspects, it is based on negative selection for cells expressing or highly expressing CD45RA and/or granzyme B. In some aspects, isolation of a CD8+ population enriched for TCM cells is carried out by depletion of cells expressing CD4, CD14, CD45RA, and positive selection or enrichment for cells expressing CD62L. In one aspect, enrichment for central memory T (TCM) cells is carried out starting with a negative fraction of cells selected based on CD4 expression, which is subjected to a negative selection based on expression of CD14 and CD45RA, and a positive selection based on CD62L. Such selections in some aspects are carried out simultaneously and in other aspects are carried out sequentially, in either order. In some aspects, the same CD4 expression-based selection step used in preparing the CD8+ cell population or subpopulation, also is used to generate the CD4+ cell population or sub-population, such that both the positive and negative fractions from the CD4-based separation are retained and used in subsequent steps of the methods, optionally following one or more further positive or negative selection steps.

In some aspects, the sample or composition of cells to be separated is incubated with small, magnetizable or magnetically responsive material, such as magnetically responsive particles or microparticles, such as paramagnetic beads (e.g., such as Dynalbeads or MACS beads). The magnetically responsive material, e.g., particle, generally is directly or indirectly attached to a binding partner, e.g., an antibody, that specifically binds to a molecule, e.g., surface marker, present on the cell, cells, or population of cells that it is desired to separate, e.g., that it is desired to negatively or positively select.

In some embodiments, the magnetic particle or bead comprises a magnetically responsive material bound to a specific binding member, such as an antibody or other binding partner. There are many well-known magnetically responsive materials used in magnetic separation methods. Suitable magnetic particles include those described in Molday, U.S. Pat. No. 4,452,773, and in European Patent Specification EP 452342 B, which are hereby incorporated by reference. Colloidal sized particles, such as those described in Owen U.S. Pat. No. 4,795,698, and Liberti et al., U.S. Pat. No. 5,200,084 are other examples.

The incubation generally is carried out under conditions whereby the antibodies or binding partners, or molecules, such as secondary antibodies or other reagents, which specifically bind to such antibodies or binding partners, which are attached to the magnetic particle or bead, specifically bind to cell surface molecules if present on cells within the sample.

In some aspects, the sample is placed in a magnetic field, and those cells having magnetically responsive or magnetizable particles attached thereto will be attracted to the magnet and separated from the unlabeled cells. For positive selection, cells that are attracted to the magnet are retained; for negative selection, cells that are not attracted (unlabeled cells) are retained. In some aspects, a combination of positive and negative selection is performed during the same selection step, where the positive and negative fractions are retained and further processed or subject to further separation steps.

In certain embodiments, the magnetically responsive particles are coated in primary antibodies or other binding partners, secondary antibodies, lectins, enzymes, or streptavidin. In certain embodiments, the magnetic particles are attached to cells via a coating of primary antibodies specific for one or more markers. In certain embodiments, the cells, rather than the beads, are labeled with a primary antibody or binding partner, and then cell-type specific secondary antibody- or other binding partner (e.g., streptavidin)-coated magnetic particles, are added. In certain embodiments, streptavidin-coated magnetic particles are used in conjunction with biotinylated primary or secondary antibodies.

In some embodiments, the magnetically responsive particles are left attached to the cells that are to be subsequently incubated, cultured and/or engineered; in some aspects, the particles are left attached to the cells for administration to a patient. In some embodiments, the magnetizable or magnetically responsive particles are removed from the cells. Methods for removing magnetizable particles from cells are known and include, e.g., the use of competing non-labeled antibodies, magnetizable particles or antibodies conjugated to cleavable linkers, etc. In some embodiments, the magnetizable particles are biodegradable.

In certain embodiments, the isolation or separation is carried out using a system, device, or apparatus that carries out one or more of the isolation, cell preparation, separation, processing, incubation, culture, and/or formulation steps of the methods. In some aspects, the system is used to carry out each of these steps in a closed or sterile environment, for example, to minimize error, user handling and/or contamination. In one example, the system is a system as described in International Patent Application, Publication Number WO2009/072003, or US 20110003380 A1.

In some embodiments, the system or apparatus carries out one or more, e.g., all, of the isolation, processing, engineering, and formulation steps in an integrated or self-contained system, and/or in an automated or programmable fashion. In some aspects, the system or apparatus includes a computer and/or computer program in communication with the system or apparatus, which allows a user to program, control, assess the outcome of, and/or adjust various aspects of the processing, isolation, engineering, and formulation steps.

In some embodiments, a cell population described herein is collected and enriched (or depleted) via flow cytometry, in which cells stained for multiple cell surface markers are carried in a fluidic stream. In some embodiments, a cell population described herein is collected and enriched (or depleted) via preparative scale (FACS)-sorting. In certain embodiments, a cell population described herein is collected and enriched (or depleted) by use of microelectromechanical systems (MEMS) chips in combination with a FACS-based detection system (see, e.g., WO 2010/033140, Cho et al. (2010) Lab Chip 10, 1567-1573; and Godin et al. (2008) J Biophoton. 1(5):355-376. In both cases, cells can be labeled with multiple markers, allowing for the isolation of well-defined T cell subsets at high purity.

In some embodiments, the antibodies or binding partners are labeled with one or more detectable marker, to facilitate separation for positive and/or negative selection. For example, separation may be based on binding to fluorescently labeled antibodies. In some examples, separation of cells based on binding of antibodies or other binding partners specific for one or more cell surface markers are carried in a fluidic stream, such as by fluorescence-activated cell sorting (FACS), including preparative scale (FACS) and/or microelectromechanical systems (MEMS) chips, e.g., in combination with a flow-cytometric detection system. Such methods allow for positive and negative selection based on multiple markers simultaneously.

In some embodiments, the methods include density-based cell separation methods, such as the preparation of white blood cells from peripheral blood by lysing the red blood cells and centrifugation through a Percoll or Ficoll gradient.

In any of the aforementioned separation steps, the separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

### Cell Preparation and Expansion

In some embodiments, the provided methods include cultivation, incubation, culture, and/or genetic engineering steps. For example, in some embodiments, provided are methods for incubating and/or engineering the depleted cell populations and culture-initiating compositions.

Thus, in some embodiments, the cell populations are incubated in a culture-initiating composition. The incubation and/or engineering may be carried out in a culture vessel, such as a unit, chamber, well, column, tube, tubing set, valve, vial, culture dish, bag, or other container for culture or cultivating cells.

In some embodiments, the cells are incubated and/or cultured prior to or in connection with genetic engineering. The incubation steps can include culture, cultivation, stimulation, activation, and/or propagation.

In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor. The cells of the invention can be activated and expanded, either prior to or after genetic modification of the cells, using methods as generally described, for example without limitation, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005. The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

Particularly in relation to CAR-expressing cells, T cells can be expanded in vitro or in vivo. Generally, the T cells of the invention can be expanded, for example, by contact with an agent that stimulates a CD3 TCR complex and a co-stimulatory molecule on the surface of the T cells to create an activation signal for the T cell. For example, chemicals such as calcium ionophore A23187, phorbol 12-myristate 13-acetate (PMA), or mitogenic lectins like phytohemagglutinin (PHA) can be used to create an activation signal for the T cell.

In some embodiments, T cell populations may be stimulated in vitro by contact with, for example, an anti-CD3 antibody, or antigen-binding fragment thereof, or an anti-CD2 antibody immobilized on a surface, or by contact with a protein kinase C activator (e.g., bryostatin) in conjunction with a calcium ionophore. In some embodiments, the T cell populations may be stimulated in vitro by contact with Muromonab-CD3 (OKT3). For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. Conditions appropriate for T cell culture include an appropriate media (e.g., Minimal Essential Media or RPMI Media 1640® or, X-vivo 5®, (Lonza)) that may contain factors necessary for proliferation and viability, including serum (e.g., fetal bovine or human serum), interleukin-2 (IL-2), insulin, IFN-γ, IL-4, IL-7, GM-CSF, IL-10, IL-2, IL-15, IL-21, TGF-β, and TNF, or any other additives for the growth of cells known to the skilled artisan. In a preferred embodiment, T cells are stimulated in vitro by exposure to OKT3 and IL-2. Other additives for the growth of cells include, but are not limited to, surfactant, Plasmanate, and reducing agents such as N-acetyl-cysteine and 2-mercaptoethanol. Media can include RPMI 16400, A1M-V, DMEM, MEM, a-MEM, F-12, X-Vivo 10, and X-Vivo 200, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° Celsius) and atmosphere (e.g., air plus 5% C02). T cells that have been exposed to varied stimulation times may exhibit different characteristics.

In some embodiments, the isolated cells of the invention can be expanded by co-culturing with tissue or cells. The cells can also be expanded in vivo, for example in the subject's blood after administrating the cell into the subject.

In some embodiments, when cells are expanded in vivo, at least one cell of the invention may be administered to a subject, and the administration may lead to an expansion of the cell in the subject, resulting in a population of cells. Alternatively, a nucleic acid sequence or vector of the invention may be administered to a subject. Once the nucleic acid sequence or vector is taken up by a cell within the subject and the cell proliferate or expand in the subject, this may result in a population of cells of the invention within the subject.

In certain embodiments, the resulting population of cells persists in the subject for at least three months, at least four months, at least five months, at least six months, at least seven months, at least eight months, at least nine months, at least ten months, at least eleven months, at least twelve months, at least eighteen months, at least two years, or at least three years after administration.

In some embodiments, the T cells are expanded by adding to the culture-initiating composition feeder cells, such as non-dividing peripheral blood mononuclear cells (PBMC), (e.g., such that the resulting population of cells contains at least about 5, 10, 20, or 40 or more PBMC feeder cells for each T lymphocyte in the initial population to be expanded); and incubating the culture (e.g. for a time sufficient to expand the numbers of T cells). In some aspects, the non-dividing feeder cells can comprise γ-irradiated PBMC feeder cells. In some embodiments, the PBMC are irradiated with γ rays in the range of about 3000 to 3600 rads to prevent cell division. In some aspects, the feeder cells are added to culture medium prior to the addition of the populations of T cells.

In some embodiments, the preparation methods include steps for freezing, e.g., cryopreserving, the cells, either before or after isolation, incubation, and/or engineering. In some embodiments, the freeze and subsequent thaw step removes granulocytes and, to some extent, monocytes in the cell population. In some embodiments, the cells are suspended in a freezing solution, e.g., following a washing step to remove plasma and platelets. Any of a variety of known freezing solutions and parameters in some aspects may be used. One example involves using PBS containing 20% DMSO and 8% human serum albumin (HSA), or other suitable cell freezing media. This is then diluted 1:1 with media so that the final concentration of DMSO and HSA are 10% and 4%, respectively. The cells are then frozen to −80° Celsius at a rate of 1 degree per minute and stored in the vapor phase of a liquid nitrogen storage tank.

Isolation of Ab or Antigen-Binding Ab Fragment from Cell Culture

Cells, such as hybridomas, that are producing Abs or antigen-binding Ab fragments of the present invention may be grown using standard methods, in suitable culture medium for this purpose (such as D-MEM or RPMI-1640), or in vivo as ascites. Abs or antigen-binding Ab fragments secreted by the cells can be separated from the culture medium, ascites fluid, or serum using conventional immunoglobulin purification procedures, such as, but not limited to, protein A-Sepharose, hydroxyapatite chromatography, gel electrophoresis, dialysis, or affinity chromatography (Ma H. et al., Methods. 2017 Mar. 1; 116:23-33. doi: 10.1016/j.ymeth.2016.11.008. Epub 2016 Nov. 18; Shukla A. A. et al. Trends Biotechnol. 2010 May; 28(5):253-61. doi: 10.1016/j.tibtech.2010.02.001. Epub 2010 Mar. 19; Arora S. et al., Methods. 2017 Mar. 1; 116:84-94. doi: 10.1016/j.ymeth.2016.12.010. Epub 2016 Dec. 22).

Methods for expressing, isolating, and evaluating multispecific and bispecific Abs and antigen-binding Ab fragments are also known in the art (for example, see Brinkmann U. et al., MAbs. 2017 February-March; 9(2): 182-212. Published online 2017 Jan. 10. doi: 10.1080/19420862.2016.1268307; Dimasi N. et al. Methods. 2018 Aug. 11. pii: S1046-2023(18)30149-X. doi: 10.1016/j.ymeth.2018.08.004).

### Therapeutic Applications

Anti-ADAM12 agents of the present invention (Abs, antigen-binding Ab fragments, multi-specific Abs, multi-specific antigen-binding Ab fragments, ADCs, or CARs that binds to ADAM12), nucleic acids encoding such an agent, vectors encoding such an agent, isolated cells obtained by the methods described above, or cell lines derived from such isolated cells, and/or pharmaceutical compositions comprising thereof can be used as a medicament in the treatment of a disease, disorder, or condition in a subject. In some embodiments, such a medicament can be used for treating an ADAM12-associated disease or condition.

### Target Diseases and Conditions

The ADAM12-associated condition may be, for example, but not limited to, cancer, fibrosis, autoimmunity, a cardiovascular condition, an allergic condition, a respiratory disease, a nephropathy, a neural disease, a muscular disease, a liver disease, metabolic syndrome, infection, and an inflammatory disorder.

In particular embodiments, the anti-ADAM12 agents of the present invention may be used to treat a cancer. ADAM12 is upregulated and/or plays a pathological role in a wide variety of cancers, such as, but not limited to bladder cancer, bone cancer, brain cancer, breast cancer, colon cancer, colorectal cancer, desmoid tumor, esophageal cancer, fibromatosis, glioblastoma, head and neck cancer, liver cancer, lung cancer, melanoma, oesophago-gastric adenocarcinoma, oligodendrioma, oral cancer, oral squamous cell carcinoma, osteosarcoma, ovarian cancer, pancreatic cancer, prostate cancer, skin cancer, small cell lung cancer, stomach cancer, and thyroid cancer (Kveiborg M. et al., Int J Biochem Cell Biol. 2008; 40(9):1685-702. doi: 10.1016/j.biocel.2008.01.025. Epub 2008 Feb. 1; Le Pabic H. et l., Hepatology. 2003 May; 37(5):1056-66; Skubitz K. M. et al., J Lab Clin Med. 2004 February; 143(2):89-98.; Carl-McGrath S. et al., Int J Oncol. 2005 January; 26(1):17-24; Mochizuki S. et al., Cancer Sci. 2007 May; 98(5):621-8. Epub 2007 Mar. 9; Colombo C. et al., J Pathol. 2011 December; 225(4):574-82. doi: 10.1002/path.2951. Epub 2011 Aug. 8; Sookprasert A. et al., Asian Pac J Cancer Prev. 2012; 13 Suppl:3-6; Uehara E. et al., Int J Oncol. 2012 May; 40(5):1414-22. doi: 10.3892/ijo.2012.1339. Epub 2012 Jan. 20; Baren J. P. et al., Br J Cancer. 2012 Jun. 26; 107(1):143-9. doi: 10.1038/bjc.2012.239. Epub 2012 Jun. 7; Rao V. H. et al., Oncogene. 2012 Jun. 7; 31(23):2888-98. doi: 10.1038/onc.2011.460. Epub 2011 Oct. 10; Kanakis D. et al., Dis Markers. 2013; 34(2):81-91. doi: 10.3233/DMA-120953; Georges S. et al., Eur J Cancer. 2013 June; 49(9):2253-63. doi: 10.1016/j.ejca.2013.02.020. Epub 2013 Mar. 13; Cireap N. et al., Pathol Oncol Res. 2013 October; 19(4):755-62. doi: 10.1007/s12253-013-9639-8. Epub 2013 May 6; Bilgin Dogru E. et al., Tumour Biol. 2014 November; 35(11):11647-53. doi: 10.1007/s13277-014-2514-8. Epub 2014 Aug. 20; Cheon D. J. Et al., Carcinogenesis. 2015 July; 36(7):739-47. doi: 10.1093/carcin/bgv059. Epub 2015 Apr. 29; Rao V. H. et al., Mol Carcinog. 2015 October; 54(10):1026-36. doi: 10.1002/mc.22171. Epub 2014 May 5; Li Z. et al., Oncol Rep. 2015 December; 34(6):3231-7; Liu G. et al., Oncol Rep. 2016 November; 36(5):3005-3013. doi: 10.3892/or.2016.5064. Epub 2016 Sep. 5; Fröhlich C. et al., Clin Cancer Res. 2006 Dec. 15; 12(24):7359-68; Roy R. et al., Mol Cancer Res. 2017 November; 15(11):1608-1622. doi: 10.1158/1541-7786.MCR-17-0188. Epub 2017 Aug. 1; Xiong L. et al., J Proteomics. 2018 Jun. 30; 182:34-44. doi: 10.1016/j.jprot.2018.04.033. Epub 2018 May 2; Veenstra V. L. et al., Oncogenesis. 2018 Nov. 16; 7(11):87. doi: 10.1038/s41389-018-0096-9). The anti-ADAM12 agents of the present invention may be used to treat any of the cancers above. The upregulation is particularly high in breast cancer, and interestingly, ADAM12 is known to induce overexpression of HER2/neu, the well-known breast cancer antigen (Nyren-Erickson E. K. Biochim Biophys Acta. 2013 October; 1830(10):4445-55. doi: 10.1016/j.bbagen.2013.05.011. Epub 2013 May 13). Therefore, breast cancer is one of the preferred target diseases of the present invention. The anti-ADAM12 agents of the present invention may also be used to treat any other cancers in which ADAM12 is upregulated or has a pathological role.

In certain embodiments, the anti-ADAM12 agents of the present invention may be used to treat a non-cancer disease or condition. ADAM12 is upregulated and/or plays a pathological role in many other diseases and conditions, such as, but not limited to, Alzheimer's disease, ostearthritis, muscular dystrophies, multiple sclerosis, fibrosis, cardiac hypertrophy, skin fibrosis and interstitial lung disease in systemic sclerosis, renal fibrosis, peripheral arterial disease, endometriosis, and Dupuytren's disease (Kveiborg M. et al., Int J Biochem Cell Biol. 2008; 40(9):1685-702. doi: 10.1016/j.biocel.2008.01.025. Epub 2008 Feb. 1; Harold D. et al., Am J Med Genet B Neuropsychiatr Genet. 2007 Jun. 5; 144B(4):448-52; Kerna I. et al., Rheumatol Int. 2012 February; 32(2):519-23. doi: 10.1007/s00296-010-1717-6. Epub 2011 Jan. 22; Dulauroy S. et al., Nat Med. 2012 August; 18(8):1262-70. doi: 10.1038/nm.2848. Epub 2012 Jul. 29; Berry E. et al., J Vasc Res. 2013; 50(1):52-68. doi: 10.1159/000345240. Epub 2012 Nov. 17; Taniguchi T. et al., J Eur Acad Dermatol Venereol. 2013 June; 27(6):747-53. doi: 10.1111/j.1468-3083.2012.04558.x. Epub 2012 Apr. 28; Ramdas V. et al., Am J Pathol. 2013 December; 183(6):1885-1896. doi: 10.1016/j.ajpath.2013.08.027. Epub 2013 Oct. 6; Dokun A. O. et al., Am J Physiol Heart Circ Physiol. 2015 September; 309(5):H790-803. doi: 10.1152/ajpheart.00803.2014. Epub 2015 Jul. 10; Miller M. A. et al., Sci Rep. 2015 Oct. 19; 5:15150. doi: 10.1038/srep15150; Sedic M. et al., (2012) Using Functional Genomics to Identify Drug Targets: A Dupuytren's Disease Example. In: Larson R. (eds) Bioinformatics and Drug Discovery. Methods in Molecular Biology (Methods and Protocols), vol 910. Humana Press, Totowa, NJ). Therefore, in some embodiments, anti-ADAM12 agents and compositions according to the present disclosure may be used to treat Alzheimer's disease, ostearthritis, muscular dystrophies, multiple sclerosis, fibrosis, cardiac hypertrophy, skin fibrosis and interstitial lung disease in systemic sclerosis, renal fibrosis, peripheral arterial disease, endometriosis, or Dupuytren's disease. In some embodiments, anti-ADAM12 agents and compositions according to the present disclosure may be used in the treatment of any condition characterized by increased ADAM12 expression.

### Subject

The subject referred to herein may be any living subject. In a preferred embodiment, the subject is a mammal. The mammal referred to herein can be any mammal. As used herein, the term “mammal” refers to any mammal, including, but not limited to, mammals of the order Rodentia, such as mice and hamsters, and mammals of the order Logomorpha, such as rabbits. The mammals may be from the order Carnivora, including Felines (cats) and Canines (dogs). The mammals may be from the order Artiodactyla, including Bovines (cows) and Swines (pigs) or of the order Perssodactyla, including Equines (horses). The mammals may be of the order Primates, Ceboids, or Simoids (monkeys) or of the order Anthropoids (humans and apes).

In some embodiments, the subject, to whom the Abs, antigen-binding Ab fragments, ADCs, CAR expressing cells, cells, cell populations, or compositions are administered is a primate, such as a human. In some embodiments, the primate is a monkey or an ape. The subject can be male or female and can be any suitable age, including infant, juvenile, adolescent, adult, and geriatric subjects. In some examples, the patient or subject is a validated animal model for disease, adoptive cell therapy, and/or for assessing toxic outcomes such as cytokine release syndrome (CRS).

In some embodiments, the subject has persistent or relapsed disease, e.g., following treatment with another immunotherapy and/or other therapy. In some embodiments, the administration effectively treats the subject despite the subject having become resistant to another therapy. In some embodiments, the subject has not relapsed but is determined to be at risk for relapse, such as at a high risk of relapse, and thus the compound or composition is administered prophylactically, e.g., to reduce the likelihood of or prevent relapse.

In some embodiments, the methods include administration of an Ab, Ab fragments, ADC, or CAR expressing cell or a composition containing such an anti-ADAM12 agent to a subject, tissue, or cell, such as one having, at risk for, or suspected of having a disease, condition or disorder associated with ADAM12, cancer, fibrosis, autoimmunity, a cardiovascular condition, an allergic condition, a respiratory disease, a nephropathy, a neural disease, a muscular disease, a liver disease, metabolic syndrome, infection, and an inflammatory disorder. In some embodiments, the anti-ADAM agents and/or compositions are administered to a subject having the particular disease or condition to be treated, e.g., via adoptive cell therapy, such as adoptive T cell therapy. In some embodiments, the anti-ADAM12 agents or compositions are administered to the subject, such as a subject having or at risk for the disease or condition. In some aspects, the methods thereby treat, e.g., ameliorate one or more symptom of the disease or condition, for example, by reducing, inhibiting, or inactivating ADAM12 and/or ADAM12-expressing cells.

### Cell Origin

For purposes of the methods of anti-ADAM12 CAR therapy, wherein host cells or populations of cells are administered, the cells can be cells that are xenogeneic, allogeneic or autologous to the subject.

In some embodiments, the cell therapy, e.g., adoptive cell therapy, e.g., adoptive T cell therapy, is carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the cell therapy, e.g., adoptive cell therapy, e.g., adoptive T cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

In certain embodiments, where cells are T cells and are not autologous to the subject, the expression of the cell's endogenous T cell receptors (TCRs) may be suppressed or disrupted. The TCR expression may be suppressed via any appropriate technique, for example, by silencing any compartment of the endogenous TCRs using tools such as, but not limited to, an siRNA, shRNA, micro RNA, or artificial microRNA. Alternatively, TCR gene may be disrupted or deleted via any appropriate technique, for example using the CRISPR/Cas system, transcription activator-like effector nucleases (e.g., TALEN®), or Zinc finger nucleases (ZFNs). The suppression or disruption of TCR may allow for reduction or prevention of undesired effects in which the TCRs recognize antigens in the subject as foreign and cause immune responses against the subject, an immune attack often called as graft-versus-host disease (GVHD).

In certain embodiments, where cells (donor cells) are not autologous to the subject, the expression of endogenous MHC or HLA gene(s) may be suppressed or disrupted, which may be achieved via any appropriate technique, such as but not limited to, an siRNA, shRNA, micro RNA, artificial microRNA, or gene editing using the CRISPR/Cas system, transcription activator-like effector nucleases (e.g., TALEN®), or Zinc finger nucleases (ZFNs). The suppression or disruption of MHC or HLA gene(s) may allow for reduction or prevention of undesired effects in which the subject's endogenous T cells recognize the donor cell's antigens presented on donor cell's MHC molecules as foreign and cause immune responses against the donor cells and increase the persistence of the administered cells within the subject. Cells expressing anti-ADAM12 Abs or antigen-binding Ab fragments or a composition comprising such may also be administered to a subject. In some embodiments, B cells or plasma cells expressing anti-ADAM12 Abs or antigen-binding Ab fragments may be adoptively transferred.

### Functional Activity

In one embodiment, the present invention includes a type of cellular therapy where isolated cells are genetically modified to express a CAR against ADAM12, and the CAR cell is infused into a subject in need thereof. Such administration can promote activation of the cells (e.g., T cell activation) in a target molecule specific manner, such that the cells of the disease or disorder are targeted for destruction. In the case where the cell is a T cell, cells, such as CAR T cells, are able to replicate in vivo resulting in long-term persistence that may lead to sustained control of diseases, disorders, or conditions associated with ADAM12, cancer, fibrotic conditions, cardiovascular conditions, inflammatory conditions, or autoimmune conditions.

In one embodiment, the isolated cells of the invention can undergo in vivo expansion and can persist for an extended amount of time. In another embodiment, where the isolated cell is a T cell, the isolated T cells of the invention evolve into specific memory T cells that can be reactivated to inhibit growth of any additional target molecule expressing cells. T cells may differentiate in vivo into a central memory-like state upon encounter and subsequent elimination of target cells expressing the surrogate antigen. Similarly, in certain embodiments, where the isolated cells is a B cells, the isolated B cells may evolve into memory B cells that can be reactivated to inhibit the growth of any additional target molecule expressing cells.

Without wishing to be bound by any particular theory, the immune response elicited by the isolated anti-ADAM12 agent-modified immune cells may be an active or a passive immune response. In addition, the anti-ADAM12 agent-mediated immune response may be part of an adoptive immunotherapy approach in which anti-ADAM12 agent-modified immune cells induce an immune response specific to the AB domain of the anti-ADAM12 agent.

In certain embodiments, anti-ADAM12 agent-expressing cells are modified in any number of ways, such that their therapeutic or prophylactic efficacy is increased. For example, the anti-ADAM12 agent may be conjugated either directly or indirectly through a linker to a targeting moiety. The practice of conjugating compounds, e.g., the CAR, to targeting moieties is known in the art. See, for instance, Wadwa et al., J. Drug Targeting 3: 1 1 1 (1995), and U.S. Pat. No. 5,087,616.

Once the cells are administered to a subject (e.g., a human), the biological activity of the engineered cell populations and/or antibodies in some aspects is measured by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable method known in the art, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells also can be measured by assaying expression and/or secretion of certain mediators, such as GM-CSF, IL-6, RANTES (CCL5), TNF-α, IL-4, IL-10, IL-13, IFN-γ, granzyme B, perforin, CD 107a, or IL-2.

In some aspects the biological activity is measured by assessing clinical outcome, such as the reduction in disease symptoms. In case of autoimmune diseases, decrease in autoreactive T cells, B cells, or Abs and reduced inflammation may represent successful biological activity. In case of cancer, improved efficacy may be shown by better infiltration of disease-resolving immune cells into the tumor, reduced tumor sizes, or reduced ascites. In some embodiments, gene expression profiles may be also investigated to evaluate the activity.

### Target Cells

Cells that may be targeted by any anti-ADAM12 agents of present invention include any ADAM12-expressing cells. The target cell may be present in any part of the body of a subject, including blood or lymphatic circulation, and disease-affected tissues. For example, when the target disease is solid cancer, the disease-affected tissues include, but are not limited to, bladder, bone, brain, breast, colon, rectum, connective tissue, esophagus, dermis, subcutaneous connective tissue, neuron, squamous cells, liver, lung, epidermis, esophagus, stomach, oligodendroma, oral tissue, oral squamous cells, ovary, pancreas, prostate, skin, lung, and thyroid. Alternatively, target cells may blood cells or hematopoietic cells.

Preferably, the anti-ADAM12 agent-expressing cells of the invention are used to treat cancer, wherein ADAM12 is upregulated. In particular, the cells of the invention may be used to treat breast cancer, lung cancer, brain cancer, stomach cancer, or skin cancer.

In general, cells that are positive for ADAM12 may be identified via known methods, for example, immunofluorescence or flow cytometry using specific antibodies, or alternatively, through cytotoxicity against target cells. Methods of testing an anti-ADAM12 agent for the ability to recognize target cells and for antigen specificity are known in the art. For instance, Clay et al., J. Immunol., 163: 507-513 (1999), teaches methods of measuring the release of cytokines (e.g., interferon-γ, granulocyte/monocyte colony stimulating factor (GM-CSF), tumor necrosis factor a (TNF-α) or interleukin 2 (IL-2)). In addition, CAR function can be evaluated by measurement of cellular cytotoxicity, as described in Zhao et al., J. Immunol., 174: 4415-4423 (2005).

A biopsy is the removal of tissue and/or cells from an individual. Such removal may be to collect tissue and/or cells from the individual in order to perform experimentation on the removed tissue and/or cells. This experimentation may include experiments to determine if the individual has and/or is suffering from a certain condition or disease-state. The condition or disease may be, e.g., cancer. With respect to detecting the presence of cells expressing anti-ADAM12 agent in a host, the sample comprising cells of the host can be a sample comprising whole cells, lysates thereof, or a fraction of the whole cell lysates, e.g., a nuclear or cytoplasmic fraction, a whole protein fraction, or a nucleic acid fraction. If the sample comprises whole cells, the cells can be any cells of the host, e.g., the cells of any organ or tissue, including blood cells or endothelial cells.

### Pharmaceutical Compositions

The compositions of the present invention may be administered in a number of ways depending upon whether local or systemic treatment is desired.

In general, administration may be topical, parenteral, or enteral.

As used herein, “parenteral administration” of a pharmaceutical composition includes any route of administration characterized by physical breaching of a tissue of a subject and administration of the pharmaceutical composition through the breach in the tissue, thus generally resulting in the direct administration into the blood stream, into muscle, or into an internal organ. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal, intravenous, intraarterial, intrathecal, intraventricular, intraurethral, intracranial, intrasynovial injection or infusions; and kidney dialytic infusion techniques. In a preferred embodiment, parenteral administration of the compositions of the present invention comprises subcutaneous or intraperitoneal administration.

The terms “oral”, “enteral”, “enterally”, “orally”, “non-parenteral”, “non-parenterally”, and the like, refer to administration of a compound or composition to an individual by a route or mode along the alimentary canal. Examples of “oral” routes of administration of a composition include, without limitation, swallowing liquid or solid forms of a composition from the mouth, administration of a composition through a nasojejunal or gastrostomy tube, intraduodenal administration of a composition, and rectal administration, e.g., using suppositories for the lower intestinal tract of the alimentary canal.

Compositions of the present invention may be suited for topical, parenteral, or enteral administration.

Preferably, formulated compositions comprising Abs, antigen-binding Ab fragments, ADCs, or CARs, polynucleotides or vectors encoding such, or cells expressing thereof are suitable for administration via parenteral administration for example, subcutaneous, intramuscular, intraperitoneal or intravenous injection.

Formulations of a pharmaceutical composition suitable for parenteral administration typically generally comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampoules or in multi-dose containers containing a preservative. Formulations for parenteral administration include, but are not limited to, suspensions, solutions, emulsions in oily or aqueous vehicles, pastes, and the like. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition. Parenteral formulations also include aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water. Exemplary parenteral administration forms include solutions or suspensions in sterile aqueous solutions, for example, aqueous propylene glycol or dextrose solutions. Such dosage forms can be suitably buffered, if desired. Other parentally-administrable formulations which are useful include those which comprise the active ingredient in microcrystalline form, or in a liposomal preparation. Formulations for parenteral administration may be formulated to be immediate and/or modified release. Modified release formulations include delayed-, sustained-, pulsed-, controlled-, targeted and programmed release. Such formulation may be, for example, made of a biodegradable, biocompatible polymer, such as, but not limited to, ethylene vinyl acetate, poly(alkyl cyanoacrylates), poly(anhydrides), poly(amindes), poly(ester), poly(ester amindes), poly(phosphoesters), polyglycolic acid (PGA), collagen, polyorthoester, polylactic acid (PLA), poly(lactic-co-glycolidc acid) (PLAGA), or naturally occurring biodegradable polymers such as chitosan and hyaluronic acid-based polymers (Kamaly N. et al, Chem Rev. Author manuscript; available in PMC 2017 Jul. 13).

Pharmaceutical compositions and formulations for topical administration may include transdermal patches, ointments, lotions, creams, gels, drops, suppositories, sprays, liquids, semi-solids, monophasic compositions, multiphasic compositions (e.g., oil-in-water, water-in-oil), foams, microsponges, liposomes, nanoemulsions, aerosol foams, polymers, fullerenes, and powders. Conventional pharmaceutical carriers, aqueous, powder or oily bases, thickeners and the like may be necessary or desirable.

Compositions and formulations for parenteral, intrathecal, or intraventricular administration may include sterile aqueous solutions that may also contain buffers, diluents and other suitable additives such as, but not limited to, penetration enhancers, carder compounds and other pharmaceutically acceptable carriers or excipients.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical compositions of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may be formulated to provide appropriate in vivo distribution of the active ingredient. In many cases, concentrating the distribution of an anti-tumor drug in the tumor site is challenging, and it can be so even when a drug has a specificity to a molecule expressed by cancer cells. Various strategies have been developed to address the issue and any appropriate strategies may be applied for the current invention (for example, reviewed in Rosenblum D. et al., Nat Commun. 2018 Apr. 12; 9(1):1410. doi: 10.1038/s41467-018-03705-y). For delivering a drug to the brain, the drug needs to cross the blood-brain barrier (BBB). Any appropriate strategies to enable BBB crossing may be utilized to for the delivery of any of the anti-ADAM12 agents of the agents (see for example, Dong X. et al., Theranostics. 2018; 8(6): 1481-1493, for exemplary strategies).

The compositions of the present invention may be formulated into any of many possible dosage forms such as, but not limited to, tablets, capsules, liquid syrups, soft gels, suppositories, aerosols, and enemas. The compositions of the present invention may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers.

In one embodiment of the present invention the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product. Agents that enhance uptake of oligonucleotides at the cellular level may also be added to the pharmaceutical and other compositions of the present invention. For example, cationic lipids, such as lipofectin (U.S. Pat. No. 5,705,188), cationic glycerol derivatives, and polycationic molecules, such as polylysine (WO 97/30731), also enhance the cellular uptake of oligonucleotides.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Formulations comprising any of the anti-ADAM12 agents of the present invention or populations of cells expressing any of the anti-ADAM12 agents such as anti-ADAM CARs of the present invention may include pharmaceutically acceptable excipient(s). Excipients included in the formulations will have different purposes depending, for example, on the CAR construct, the subpopulation of cells used, and the mode of administration. Examples of generally used excipients include, without limitation: saline, buffered saline, dextrose, water-for-infection, glycerol, ethanol, and combinations thereof, stabilizing agents, solubilizing agents and surfactants, buffers and preservatives, tonicity agents, bulking agents, and lubricating agents. The formulations comprising populations of the CAR-expressing cells of the present invention will typically have been prepared and cultured in the absence of any non-human components, such as animal serum (e.g., bovine serum albumin).

The formulation or composition may also contain more than one active ingredient useful for the particular indication, disease, or condition being treated with the binding molecules or cells, preferably those with activities complementary to the binding molecule or cell, where the respective activities do not adversely affect one another. Such active ingredients are suitably present in combination in amounts that are effective for the purpose intended. Thus, in some embodiments, the pharmaceutical composition further includes other pharmaceutically active agents or drugs. Such agents or drugs may be, but are not limited to, an anti-cancer drug, an anti-proliferative drug, a cytotoxic drug, an anti-angiogenic drug, an apoptotic drug, an immunostimulatory drug, an anti-microbial drug, an antibiotic drug, an antiviral drug, an anti-inflammatory drug, an anti-fibrotic drug, an immunosuppressive drug, a steroid, a bronchodilator, a beta blocker, a matrix metalloproteinase inhibitor, an ADAM12 inhibitor, an ADAM12 signaling inhibitor, an anti-ADAM12 agent of the present invention, an enzyme, a hormone, a neurotransmitter, a toxin, a compound, a small molecule, a small molecule inhibitor, a protein, a peptide, a vector, a plasmid, a viral particle, a nanoparticle, a DNA molecule, an RNA molecule, an siRNA, an shRNA, a micro RNA, an oligonucleotide, or an imaging drug. Specific examples are, for instance, but are not limited to, chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc.

The pharmaceutical composition in some aspects can employ time-released, delayed release, and sustained release delivery systems such that the delivery of the composition occurs prior to, and with sufficient time to cause, sensitization of the site to be treated. Many types of release delivery systems are available and known. Such systems can avoid repeated administrations of the composition, thereby increasing convenience to the subject and the physician.

### Kits

Also provided herein are kits comprising (a) one or more of anti-ADAM12 agents (Abs, antigen-binding Ab fragments, ADCs, CARs), polynucleotides encoding such, vectors encoding such, cells expressing such; and (b) for example an instruction for use in treating or diagnosing a disease or condition associated with ADAM12. The kit may include a label indicating the intended use of the contents of the kit. The term “label” as used herein includes any written materials, marketing materials, or recorded materials supplied on, with, in, or appended to the kit.

### Method of Administration

The administration route used in the method of the present invention may be any appropriate route, which depends upon whether local or systemic treatment is desired.

In general, administration may be topical, parenteral, or enteral.

Preferably, formulated compositions comprising Abs, antigen-binding Ab fragments, ADCs, or CARs, polynucleotides or vectors encoding such, cells expressing such may be administered parenterally, for example, via subcutaneous, intramuscular, intraperitoneal or intravenous injection.

In the case of adoptive cell therapy, methods for administration of cells for adoptive cell therapy are known and may be used in connection with the provided methods and compositions. For example, adoptive T cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338.

In some embodiments, the composition of the present invention may be administered using any appropriate medical devices (for example, reviewed in Richter B. B., J. BioDrugs (2018) 32: 425).

### Dosing

For administration of any of the anti-ADAM12 agents and compositions of the present invention, the dosage will vary and depend on, for example, the target disease, the severity of the disease, the route of administration, and pharmacokinetic factors. Dosing may be modified based on the response observed in the subject.

For administration of any of the anti-ADAM12 Abs, antigen-binding Ab fragments, or ADCs, or compositions comprising such, appropriate dosage regimen may be determined using any appropriate methodology (for example, Bai S. et al., Clin Pharmacokinet. 2012 Feb. 1; 51(2):119-35. doi: 10.2165/11596370-000000000-00000).

In some embodiments, the dosage may be from about 1 ng/kg to about 1 g/kg (of the body weight of a subject) per day. In some aspects, the dose may be from about 10 ng/kg/day to about 900 mg/kg/day, from about 20 ng/kg/day to about 800 mg/kg/day, from about 30 ng/kg/day to about 800 mg/kg/day, from about 40 ng/kg/day to about 700 mg/kg/day, from about 50 ng/kg/day to about 600 mg/kg/day, from about 60 ng/kg/day to about 500 mg/kg/day, from about 70 ng/kg/day to about 400 mg/kg/day, from about 80 ng/kg/day to about 300 mg/kg/day, from about 90 ng/kg/day to about 200 mg/kg/day, or from about 100 ng/kg/day to about 100 mg/kg/day. The treatment may be repeated or periodically given to a subject for days, months, or years, or until the desired effect is achieved. An exemplary dosing regimen include administering an initial dose of an anti-ADAM12 Abs, antigen-binding Ab fragments, or ADCs of about 2 mg/kg, followed by a weekly maintenance dose of about 1 mg/kg.

Dosing frequency may be, for example, three times per day, twice per day, once per day, every other day, once per week, every other week, once per three weeks, once per four weeks, once per five weeks, once per six weeks, once per seven weeks, once per eight weeks, once per nine weeks, once per ten weeks, once per three months, once per four months, once per six months, once per year, or even less frequent.

The pharmaceutical composition in some embodiments contains cells expressing the CAR of the present invention in amounts effective to treat or prevent the disease or condition, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

In certain embodiments, in the context of genetically engineered cells expressing an anti-ADAM12 agent such as a CAR, a subject is administered the range of about one million to about 100 billion cells, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges, and/or such a number of cells per kilogram of body weight of the subject. For example, in some embodiments the administration of the cells or population of cells can comprise administration of about 103 to about 109 cells per kg body weight including all integer values of cell numbers within those ranges.

The cells or population of cells can be administrated in one or more doses. In some embodiments, said effective amount of cells can be administrated as a single dose. In some embodiments, said effective amount of cells can be administrated as more than one dose over a period time. Timing of administration is within the judgment of managing physician and depends on the clinical condition of the patient. The cells or population of cells may be obtained from any source, such as a blood bank or a donor. While individual needs vary, determination of optimal ranges of effective amounts of a given cell type for a particular disease or conditions within the skill of the art. An effective amount means an amount which provides a therapeutic or prophylactic benefit. The dosage administrated will be dependent upon the age, health and weight of the recipient, kind of concurrent treatment, if any, frequency of treatment and the nature of the effect desired. In some embodiments, an effective amount of cells or composition comprising those cells are administrated parenterally. In some embodiments, administration can be an intravenous administration. In some embodiments, administration can be directly done by injection into the disease site.

For purposes of the invention, the amount or dose of the inventive anti-ADAM12 material administered should be sufficient to effect a therapeutic or prophylactic response in the subject or animal over a reasonable time frame. For example, the dose of the inventive anti-ADAM12 material should be sufficient to bind to antigen, or detect, treat or prevent disease in a period of from about 2 hours or longer, e.g., about 12 to about 24 or more hours, from the time of administration. In certain embodiments, the time period could be even longer. The dose will be determined by the efficacy of the particular inventive anti-ADAM12 material and the condition of the animal (e.g., human), as well as the body weight of the animal (e.g., human) to be treated.

For purposes of the invention, an assay, which comprises, for example, comparing the extent to which target cells are lysed or, in the context of CARs, IFN-γ is secreted by T cells expressing the inventive CAR, polypeptide, or protein upon administration of a given dose of such T cells to a mammal, among a set of mammals of which is each given a different dose of the T cells, could be used to determine a starting dose to be administered to a mammal. The extent to which target cells are lysed or IFN-γ is secreted upon administration of a certain dose can be assayed by methods known in the art.

In some embodiments, two or more of the anti-ADAM12 agents or compositions of the present invention may be administered to a subject in combination or separately.

In some embodiments, the anti-ADAM12 agents or compositions of the present invention are administered as part of a combination treatment, such as simultaneously with or sequentially with, in any order, another therapeutic intervention, such as an antibody or engineered cell or receptor or agent, such as a cytotoxic or therapeutic agent. The cells or antibodies in some embodiments are co-administered with one or more additional therapeutic agents or in connection with another therapeutic intervention, either simultaneously or sequentially in any order. In some contexts, the anti-ADAM12 agents or compositions are co-administered with another therapy sufficiently close in time such that the anti-ADAM12 agents or compositions enhance the effect of one or more additional therapeutic agents, or vice versa. In some embodiments, the cells or antibodies are administered prior to the one or more additional therapeutic agents. In some embodiments, the anti-ADAM12 agents, such as anti-ADAM12 CART cells or antibodies are administered after the one or more additional therapeutic agents. Furthermore, the compositions of the present invention may be given to a subject along with one or more of other therapies, which may be surgery, or a radiotherapy.

In some embodiments, in CAR T therapy, a lymphodepleting chemotherapy is administered to the subject prior to, concurrently with, or after administration (e.g., infusion) of CAR cells. In an example, the lymphodepleting chemotherapy is administered to the subject prior to administration of the cells. For example, the lymphodepleting chemotherapy ends 1-4 days (e.g., 1, 2, 3, or 4 days) prior to CAR cell infusion. In embodiments, multiple doses of CAR cells are administered, e.g., as described herein. In embodiments, a lymphodepleting chemotherapy is administered to the subject prior to, concurrently with, or after administration (e.g., infusion) of a CAR-expressing cell described herein. Examples of lymphodepletion include, but may not be limited to, nonmyeloablative lymphodepleting chemotherapy, myeloablative lymphodepleting chemotherapy, total body irradiation, etc. Examples of lymphodepleting agents include, but are not limited to, antithymocyte globulin, anti-CD3 antibodies, anti-CD4 antibodies, anti-CD8 antibodies, anti-CD52 antibodies, anti-CD2 antibodies, TCRαβ blockers, anti-CD20 antibodies, anti-CD19 antibodies, Bortezomib, rituximab, anti-CD154 antibodies, rapamycin, CD3 immunotoxin, fludarabine, cyclophosphamide, busulfan, melphalan, Mabthera, Tacrolimus, alefacept, alemtuzumab, OKT3, OKT4, OKT8, OKT11, fingolimod, anti-CD40 antibodies, anti-BR3 antibodies, Campath-1H, anti-CD25 antibodies, calcineurin inhibitors, mycophenolate, and steroids, which may be used alone or in combination.

### Use as a Diagnostic Tool

The anti-ADAM12 agents of the present invention, for example, anti-ADAM12 Abs and antigen-binding Ab fragments, can be also useful as a diagnostic tool that may be used in vivo, ex vivo, or in vitro.

For example, an anti-ADAM12 Abs or antigen-binding Ab fragment conjugated to an imaging agent may be administered to a subject or a patient to test if a diseased cell or tissue in the patient expresses ADAM12. The diagnoses may be done using any imaging tools that can detect the imaging agent. Alternatively, a biological sample such as, but is not limited to, blood or biopsy sample, may be obtained, and an anti-ADAM12 Abs or antigen-binding Ab fragment may be applied to the sample to test the expression of ADAM12.

These tests may determine whether the subject, or the cell or tissue of the subject, expresses ADAM12 or not. In some embodiments, the test may determine whether the subject, or the cell or tissue of the subject, expresses sufficient amount of ADAM12 to be targeted by the anti-ADAM12 therapeutic agent of the present invention. In some embodiments, the test may classify patients into different levels of ADAM12 expression. In one aspect, a subject may be classified as an expressor or a non-expressor. In another aspect, a subject may be classified as an over-expressor, mid-expressor, or low-expressor.

In some embodiments, an appropriate therapeutic approach may be determined depending on the ADAM12 expression. The expression may be determined using an anti-ADAM12 agent of the present invention as described herein, or alternatively using any other appropriate method, such as, but not limited to, by measuring RNA expression levels or by quantifying ADAM12 protein levels using an appropriate tool and/or technique. In one aspect, the anti-ADAM12 agent of the present invention may be given to an expressor but not to a non-expressor. In another aspect the anti-ADAM12 agent of the present invention may be given to an over-expressor but not to a mid-expressor or a low-expressor. In another aspect, the anti-ADAM12 agent of the present invention may be given to an over-expressor or a mid-expressor but not to a low-expressor. In yet another aspect, the anti-ADAM12 agent of the present invention may be given to a mid-expressor but not to a high-expressor or a low-expressor.

### Variations

Included in the scope of the invention are functional portions of the inventive anti-ADAM12 agents described herein. The term “functional portion,” when used in reference to an Ab, antigen-binding Ab fragment, ADC, or CAR, refers to any part or fragment of the Ab, antigen-binding Ab fragment, ADC, or CAR of the invention, which part or fragment retains the biological activity of the Ab, antigen-binding Ab fragment, ADC, or CAR of which it is a part (the parent). Functional portions encompass, for example, those parts of an Ab, antigen-binding Ab fragment, ADC, or CAR that retain the ability to recognize target cells, or detect, treat, or prevent a disease, to a similar extent, the same extent, or to a higher extent, as the parent. In reference to the parent Ab, antigen-binding Ab fragment, ADC, or CAR, the functional portion can comprise, for instance, about 10%, 25%, 30%, 50%, 68%, 80%, 90%, 95%, or more, of the parent.

The functional portion can comprise additional amino acids at the amino or carboxy terminus of the portion, or at both termini, which additional amino acids are not found in the amino acid sequence of the parent Ab, antigen-binding Ab fragment, ADC, or CAR. Desirably, the additional amino acids do not interfere with the biological function of the functional portion, e.g., recognize target cells, detect, treat, or prevent fibrosis and/or inflammation, etc. More desirably, the additional amino acids enhance the biological activity, as compared to the biological activity of the parent Ab, antigen-binding Ab fragment, ADC, or CAR.

Included in the scope of the invention are functional variants of the inventive Abs, antigen-binding Ab fragments, ADCs, or CARs described herein. The term “functional variant” as used herein refers to an Ab, antigen-binding Ab fragment, ADC, or CAR polypeptide, or protein having substantial or significant sequence identity or similarity to a parent, which functional variant retains the biological activity of the Ab, antigen-binding Ab fragment, ADC, or CAR of which it is a variant. Functional variants encompass, for example, those variants of the Ab, antigen-binding Ab fragment, ADC, or CAR described herein (the parent) that retain the ability to recognize target cells to a similar extent, the same extent, or to a higher extent, as the parent. In reference to the parent Ab, antigen-binding Ab fragment, ADC, or CAR, the functional variant can, for instance, be at least about 30%, 50%, 75%, 80%, 90%, 98% or more identical in amino acid sequence to the parent.

A functional variant can, for example, comprise the amino acid sequence of the parent with at least one conservative amino acid substitution. Alternatively or additionally, the functional variants can comprise the amino acid sequence of the parent with at least one non-conservative amino acid substitution. In this case, it is preferable for the non-conservative amino acid substitution to not interfere with or inhibit the biological activity of the functional variant. The non-conservative amino acid substitution may enhance the biological activity of the functional variant, such that the biological activity of the functional variant is increased as compared to the parent.

Amino acid substitutions of the inventive anti-ADAM12 agents are preferably conservative amino acid substitutions. Conservative amino acid substitutions are known in the art, and include amino acid substitutions in which one amino acid having certain physical and/or chemical properties is exchanged for another amino acid that has the same or similar chemical or physical properties. For instance, the conservative amino acid substitution can be an acidic/negatively charged polar amino acid substituted for another acidic/negatively charged polar amino acid (e.g., Asp or Glu), an amino acid with a nonpolar side chain substituted for another amino acid with a nonpolar side chain (e.g., Ala, Gly, Val, Ile, Leu, Met, Phe, Pro, Trp, Cys, Val, etc.), a basic/positively charged polar amino acid substituted for another basic/positively charged polar amino acid (e.g. Lys, His, Arg, etc.), an uncharged amino acid with a polar side chain substituted for another uncharged amino acid with a polar side chain (e.g., Asn, Gln, Ser, Thr, Tyr, etc.), an amino acid with a β-branched side-chain substituted for another amino acid with a β-branched side-chain (e.g., Ile, Thr, and Val), an amino acid with an aromatic side-chain substituted for another amino acid with an aromatic side chain (e.g., His, Phe, Trp, and Tyr), etc.

Also, amino acids may be added or removed from the sequence based on vector design.

The anti-ADAM12 agents can consist essentially of the specified amino acid sequence or sequences described herein, such that other components, e.g., other amino acids, do not materially change the biological activity of the functional variant.

The Abs, antigen-binding Ab fragments, ADCs, or CARs of embodiments of the invention (including functional portions and functional variants) can be of any length, i.e., can comprise any number of amino acids, provided that the Abs, antigen-binding Ab fragments, ADCs, or CARs (or functional portions or functional variants thereof) retain their biological activity, e.g., the ability to specifically bind to antigen, detect diseased cells in a mammal, or treat or prevent disease in a mammal, etc. For example, the Ab, antigen-binding Ab fragment, ADC, or CAR can be about 50 to about 5000 amino acids long, such as 50, 70, 75, 100, 125, 150, 175, 200, 300, 400, 500, 600, 700, 800, 900, 1000 or more amino acids in length.

The Abs, antigen-binding Ab fragments, ADCs, or CARs of embodiments of the invention (including functional portions and functional variants of the invention) can comprise synthetic amino acids in place of one or more naturally-occurring amino acids. Such synthetic amino acids are known in the art, and include, for example, aminocyclohexane carboxylic acid, norleucine, α-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, β-phenylserine β-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, α-aminocyclopentane carboxylic acid, α-aminocyclohexane carboxylic acid, α-aminocycloheptane carboxylic acid, α-(2-amino-2-norbornane)-carboxylic acid, α,γ-diaminobutyric acid, α,β-diaminopropionic acid, homophenylalanine, and α-tert-butylglycine.

The Abs, antigen-binding Ab fragments, ADCs, or CARs of embodiments of the invention (including functional portions and functional variants) can be glycosylated, amidated, carboxylated, phosphorylated, esterified, N-acylated, cyclized via, e.g., a disulfide bridge, or converted into an acid addition salt and/or optionally dimerized or polymerized, or conjugated.

The Abs, antigen-binding Ab fragments, ADCs, or CARs of embodiments of the invention (including functional portions and functional variants thereof) can be obtained by methods known in the art. The Abs, antigen-binding Ab fragments, ADCs, and CARs may be made by any suitable method of making polypeptides or proteins. Suitable methods of de novo synthesizing polypeptides and proteins are described in references, such as Chan et al., “Fmoc Solid Phase Peptide Synthesis”, Oxford University Press, Oxford, United Kingdom, 2000; “Peptide and Protein Drug Analysis”, ed. Reid, R., Marcel Dekker, Inc., 2000; “Epitope Mapping”, ed. Westwood et al., “Oxford University Press, Oxford, United Kingdom, 2001; and U.S. Pat. No. 5,449,752. Also, polypeptides and proteins can be recombinantly produced using the nucleic acids described herein using standard recombinant methods. See, for instance, Sambrook et al., “Molecular Cloning: A Laboratory Manual”, 3rd ed., Cold Spring Harbor Press, Cold Spring Harbor, N.Y. 2001; and Ausubel et al., “Current Protocols in Molecular Biology”, Greene Publishing Associates and John Wiley & Sons, N Y, 1994. Further, some of the Abs, antigen-binding Ab fragments, or CARs of the invention (including functional portions and functional variants thereof) can be isolated and/or purified from a source, such as a plant, a bacterium, an insect, a mammal, e.g., a rat, a human, etc. Methods of isolation and purification are well-known in the art. Alternatively, the Abs, antigen-binding Ab fragments, ADCs, or CARs described herein (including functional portions and functional variants thereof) can be commercially synthesized by companies. In this respect, the inventive Abs, antigen-binding Ab fragments, ADCs, or CARs can be synthetic, recombinant, isolated, and/or purified.

### Definitions

Although various embodiments and examples of the present invention have been described referring to certain molecules, compositions, methods, or protocols, it is to be understood that the present invention is not limited to the particular molecules, compositions, methods, or protocols described herein, as theses may vary. It is also to be understood that the terminology used in the description is for the purpose of describing the particular versions or embodiments only, and is not intended to limit the scope of the present invention which will be limited only by the appended claims.

All references cited herein, including patent documents and non-patent documents, are hereby incorporated by reference in their entirety. Nothing herein is to be construed as an admission that the present invention is not entitled to antedate such disclosure by virtue of prior invention.

In the specification above and in the appended claims, all transitional phrases such as “comprising,” “including,” “having,” “containing,” “involving,” “composed of,” and the like are to be understood to be open-ended, namely, to mean including but not limited to. Only the transitional phrases “consisting of” and “consisting essentially of” shall be closed or semi-closed transitional phrases, respectively.

It must also be noted that, unless the context clearly dictates otherwise, the singular forms “a,” “an,” and “the” as used herein and in the appended claims include plural reference. Thus, the reference to “a cell” refers to one or more cells and equivalents thereof known to those skilled in the art, and so forth. Unless defined otherwise, all technical and scientific terms used herein have the same meanings as commonly understood by a person of skilled in the art.

It should be understood that, unless clearly indicated otherwise, in any methods disclosed or claimed herein that comprise more than one step, the order of the steps to be performed is not restricted by the order of the steps cited.

The term “4-1BB,” “41BB,” or “BB” as used herein refers to a member of the TNFR superfamily with an amino acid sequence provided as GenBank Acc. No. AAA53133.1, or the equivalent residues from a non-human species, e.g., mouse, rodent, monkey, ape and the like. In one aspect, the “4-1BB costimulatory domain,” also referred to as “4-1BB CS domain” or “41BBCS,” may be derived from the cytoplasmic domain of 4-1BB. In some embodiments, “41BBCS” comprises the sequence provided as SEQ ID NO: 165 or the equivalent residues from a non-human species, e.g., mouse, rodent, monkey, ape and the like. In some embodiments, “41BBCS” may be encoded by a nucleic acid sequence provided as SEQ ID NO: 265.

The term “5′ cap” (also termed an RNA cap, an RNA 7-methylguanosine cap or an RNA m7G cap) as used herein is a modified guanine nucleotide that has been added to the “front” or 5′ end of a eukaryotic messenger RNA shortly after the start of transcription. The 5′ cap consists of a terminal group which is linked to the first transcribed nucleotide. Its presence is critical for recognition by the ribosome and protection from RNases. Cap addition is coupled to transcription, and occurs co-transcriptionally, such that each influences the other. Shortly after the start of transcription, the 5′ end of the mRNA being synthesized is bound by a cap-synthesizing complex associated with RNA polymerase. This enzymatic complex catalyzes the chemical reactions that are required for mRNA capping. Synthesis proceeds as a multi-step biochemical reaction. The capping moiety can be modified to modulate functionality of mRNA such as its stability or efficiency of translation.

The term “about” or “approximately” as used herein when referring to a numerical value, such as of weight, mass, volume, concentration, or time, should not be limited to the recited numerical value but rather encompasses variations of +/−10% of a given value.

“ADAM12” as used herein, also known as ADAM metallopeptidase domain 12, a disintegrin and metalloproteinase-12, a disintegrin and metalloproteinase domain-containing protein 12, ADAM12-OT1, CAR10, MCMP, MCMPMltna, Meltrin-a, MLTN, or MLTNA, is a member of the ADAM family (a disintegrin and metalloproteinase family). In humans, ADAM12 is encoded by the ADAM12 gene on chromosome 10, with gene location 10q26.2 (NCBI), and two naturally occurring ADAM12 splice variants named ADAM12-L (L is for long) and ADAM12-S(S is for short) exist (Kveiborg M. et al., Int J Biochem Cell Biol. 2008; 40(9):1685-702. doi: 10.1016/j.biocel.2008.01.025. Epub 2008 Feb. 1). Human ADAM12-L has an amino acid sequence provided as GenBank Accession: AAC08702.2, or the equivalent residues from a non-human species, e.g., mouse, rodent, monkey, ape and the like. In one aspect, human ADAM12-L has the amino acid sequence provided as SEQ ID NO: 101 or the equivalent residues from a non-human species, e.g., mouse, rodent, monkey, ape and the like. Human ADAM12-S has an amino acid sequence provided as GenBank Accession: AAC08703.2, or the equivalent residues from a non-human species, e.g., mouse, rodent, monkey, ape and the like. In one aspect, human ADAM12-S has the amino acid sequence provided as SEQ ID NO: 102 or the equivalent residues from a non-human species, e.g., mouse, rodent, monkey, ape and the like.

The term “allogeneic” as used herein refers to any material derived from a different animal of the same species as the individual to whom the material is introduced. Two or more individuals are said to be allogeneic to one another when the genes at one or more loci are not identical. In some aspects, allogeneic material from individuals of the same species may be sufficiently unlike genetically to interact antigenically.

The term “antibody” or “Ab,” or “immunoglobulin” as used herein, refers to an immunoglobulin molecule which specifically binds with an antigen. Typically, a full-size Ab comprises two pairs of chains, each pair comprising a heavy chain (HC) and a light chain (LC). A HC typically comprises a variable region and a constant region. A LC also typically comprises a variable region and constant region. The variable region of a heavy chain (VH) typically comprises three complementarity-determining regions (CDRs), which are referred to herein as CDR 1, CDR 2, and CDR 3 (or referred to as CDR-H1, CDR-H2, CDR-H3, respectively). The constant region of a HC typically comprises a fragment crystallizable region (Fc region), which dictates the isotype of the Ab, the type of Fc receptor the Ab binds to, and therefore the effector function of the Ab. Fc receptor types include, but are not limited to, FcaR (such as FcaRI), Fca/mR, FceR (such as FceRI, FceRII), and FcgR (such as FcgRI, FcgRIIA, FcgRIIB1, FcgRIIB2, FcgRIIIA, FcgRIIIB) and their associated downstream effects are well known in the art. The variable region of a light chain (VL) also typically comprises CDRs, which are CDR 1, CDR 2, and CDR 3 (or referred to as CDR-L1, CDR-L2, CDR-L3, respectively). In some embodiments, the antigen is ADAM12. Antibodies can be intact immunoglobulins derived from natural sources or from recombinant sources. A portion of an antibody that comprises a structure that enables specific binding to an antigen is referred to “antigen-binding fragment,” “AB domain,” “antigen-binding region,” or “AB region” of the Ab.

The term “antibody-drug conjugate” “Ab-drug conjugate”, or “ADC” as used herein refers to a conjugate of an Ab or antigen-binding Ab fragment and a drug. The drug may be attached to any part of the Ab or the antigen-binding Ab fragment via a direct or indirect attachment, such as via a linker. In some embodiments, an ADC may comprise an antibody (or antibody fragment such as a single-chain variable fragment (scFv)) linked to a payload drug (often cytotoxic). The antibody causes the ADC to bind to the target cancer cells. In some embodiments, the ADC is then internalized by the cell and the drug is released into the cell. Because of the targeting, the side effects may be lower and may provide a wider therapeutic window. Hydrophilic linkers (e.g., PEG4Mal) may prevent the drug being pumped out of resistant cancer cells through MDR (multiple drug resistance) transporters. The present disclosure is also related to immunoconjugates comprising an anti-ADAM12 binding agent conjugated to a therapeutic agent, such as a cytotoxin, a drug (e.g., an immunosuppressant) or a radiotoxin. Such conjugates may be referred to as “immunoconjugates”. Immunoconjugates that include one or more cytotoxins may also be referred to as “immunotoxins.” A cytotoxin or cytotoxic agent includes any agent that is detrimental to (e.g., kills) cells. Cytotoxins can be conjugated to antibodies according to at least some embodiments of the invention using linker technology available in the art. Examples of linker types that have been used to conjugate a cytotoxin to an antibody include, but are not limited to, hydrazones, thioethers, esters, disulfides and peptide-containing linkers. A linker can be chosen that is, for example, susceptible to cleavage by low pH within the lysosomal compartment or susceptible to cleavage by proteases, such as proteases preferentially expressed in tumor tissue such as cathepsins (e.g., cathepsins B, C, D). For further discussion of types of cytotoxins, linkers and methods for conjugating therapeutic agents to antibodies, see also Saito, G. et al. (2003) Adv. Drug Deliv. Rev. 55: 199-215; Trail, P. A. et al. (2003) Cancer Immunol. Immunother. 52:328-337; Payne, G. (2003) Cancer Cell 3:207-212; Allen, T. M. (2002) Nat. Rev. Cancer 2:750-763; Pastan, I. and Kreitman, R. J. (2002) Curr. Opin. Investig. Drugs 3: 1089-1091; Senter, P. D. and Springer, C. J. (2001) Adv. Drug Deliv. Rev. 53:247-264. Antibodies of the present invention also can be conjugated to a radioactive isotope to generate cytotoxic radiopharmaceuticals, also referred to as radioimmunoconjugates.

The term “antibody fragment” or “Ab fragment” as used herein refers to any portion or fragment of an Ab, including intact or full-length Abs that may be of any class or sub-class, including IgG and sub-classes thereof, IgM, IgE, IgA, and IgD. The term encompasses molecules constructed using one or more portions or fragments of one or more Abs. An Ab fragment can be immunoreactive portions of intact immunoglobulins. The term is used in the broadest sense and includes polyclonal and monoclonal antibodies, including intact antibodies and functional (antigen-binding) antibody fragments, including fragment antigen binding (Fab) fragments, F(ab′)2 fragments, Fab′ fragments, Fv fragments, recombinant IgG (rIgG) fragments, single chain antibody fragments, including single chain variable fragments (scFv), diabodies, and single domain antibodies (e.g., sdAb, sdFv, nanobody) fragments. The term also encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. In a specific embodiment, the antibody fragment is a scFv.

Unless otherwise stated, the term “Ab fragment” should be understood to encompass functional antibody fragments thereof. A portion of an Ab fragment that comprises a structure that enables specific binding to an antigen is referred to as “antigen-binding Ab fragment,” “AB domain,” “antigen-binding region,” or “antigen-binding region” of the Ab fragment.

A “heavy chain” or “HC” of an Ab, as used herein, refers to the larger of the two types of polypeptide chains present in all Ab molecules in their naturally occurring conformations.

A “light chain” or “LC” of an Ab, as used herein, refers to the smaller of the two types of polypeptide chains present in all Ab molecules in their naturally occurring conformations. Kappa and lambda light chains refer to the two major antibody light chain isotypes.

An “anti-ADAM12 agent” or “anti-ADAM12 material” as used herein refers to any agents that are able to target ADAM12 directly or indirectly. Anti-ADAM12 agents of the present invention include, but are not limited to, anti-ADAM12 Abs, anti-ADAM12 antigen-binding Ab fragments, anti-ADAM12 multi-specific Abs, anti-ADAM12 multi-specific antigen-binding Ab fragments, anti-ADAM12 ADCs, and anti-ADAM12 CARs, and nucleic acid sequences and vectors encoding the same, and cells expressing the same. In a broad sense, anti-ADAM12 agents may also encompass pharmaceutical compositions comprising any of the above-mentioned anti-ADAM12 agents.

The term “antigen” or “Ag” refers to a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one genes and that these nucleotide sequences are arranged in various combinations to encode polypeptides that elicit the desired immune response. Moreover, a skilled artisan will understand that an antigen need not be encoded by a “gene” at all. It is readily apparent that an antigen can be generated, synthesized, or can be derived from a biological sample, or might be macromolecule besides a polypeptide. Such a biological sample can include, but is not limited to a tissue sample, a cancer tissue sample, a tumor tissue sample, a leukemic cell sample, an inflamed tissue sample, and a cell or a fluid with other biological components. In some embodiments, the antigen is ADAM-12.

The term “antigen-binding domain” or “AB domain” refers to a portion of the anti-ADAM12 agents of the present invention and the portion comprises a structure that enables specific binding of the anti-ADAM12 agents to ADAM12. When the anti-ADAM12 agent is an Ab, the AB domain may comprise the variable region of the Ab or a portion of the variable region, such as the CDRs. When the anti-ADAM12 agent is an antigen-binding Ab fragment or an antibody-drug conjugate, the AB domain may comprise the variable region or a portion of the variable region, such as the CDRs, of the Ab that the anti-ADAM12 agent is derived from. When the anti-ADAM12 agent is a chimeric antigen receptor (CAR), the AB domain may be one or more extracellular domains of the CAR which have specificity for ADAM12. When the AB domain is derived from an Ab or antigen-binding Ab fragment, the AB domain may comprise the AB domain, such as the variable region or a portion of the variable region, such as the CDRs, of the Ab or antigen-binding Ab fragment that it is derived from. In some embodiments, the AB domain of an anti-ADAM12 agent of the present invention is scFv. In some embodiments, the AB domain may comprise or be derived from a naturally existing molecule that binds to ADAM12 or the ADAM12-binding portion of the molecule. Examples of such a molecule include, but are not limited to, alpha actinin 2 (ACTN2), insulin-like growth factor-binding protein 3 (IGFBP3), phosphatidylinositol 3 kinase regulatory subunit alpha (PIK3R1), IGFBP-3 (insulin-like growth factor binding protein-3), IGFBP-5, heparin-binding epidermal growth factor (HB-EGF), epidermal growth factor (EGF), Betacellulin, Delta-like 1, placental leucine aminopeptidase (P-LAP), and matrix metalloprotease 14 (MMP-14).

The term “apheresis” as used herein refers to the art-recognized extracorporeal process by which the blood of a donor or patient is removed from the donor or patient and passed through an apparatus that separates out selected particular constituent(s) and returns the remainder to the circulation of the donor or patient, e.g., by retransfusion. Thus, in the context of “an apheresis sample” refers to a sample obtained using apheresis.

The term “autologous” or “donor-derived” as used herein refers to any material derived from the same individual to whom it is later to be re-introduced.

The term “bind” refers to an attractive interaction between two molecules that results in a stable association in which the molecules are in close proximity to each other. The result of molecular binding is sometimes the formation of a molecular complex in which the attractive forces holding the components together are generally non-covalent, and thus are normally energetically weaker than covalent bonds.

The term “cancer” refers to a disease characterized by the uncontrolled growth of aberrant cells. Cancer cells can spread locally or through the bloodstream and lymphatic system to other parts of the body. Examples of various cancers relevant to the present invention include, but are not limited to bladder cancer, bone cancer, brain cancer, breast cancer, cervical cancer, colon cancer, colorectal cancer, desmoid tumor, esophageal cancer, fibromatosis, glioblastoma, head and neck cancer, leukemia, liver cancer, lung cancer, lymphoma, melanoma, oesophago-gastric adenocarcinoma, oligodendroma, oral cancer, oral squamous cell carcinoma, osteosarcoma, ovarian cancer, pancreatic cancer, prostate cancer, renal cancer, skin cancer, small cell lung cancer, stomach cancer, and thyroid cancer and the like.

The term “bispecific” as used herein refers to having two binding specificities. An anti-ADAM12 bispecific Ab or a bispecific antigen-binding Ab fragment, for example, of the present invention has at least one specificity for ADAM12. When the first specificity is for an epitope for ADAM12, the second specificity may be for another non-overlapping or non-competing epitope for ADAM12 or may be for a molecule other than ADAM12. The term “bispecific” is also used in the same manner for any other anti-ADAM12 agents of the present invention, such as anti-ADAM12 CARs.

The term “CD28” refers to the protein Cluster of Differentiation 28, one of the proteins expressed on T cells that provide co-stimulatory signals required for T cell activation and survival. Human CD28 protein may have at least 85, 90, 95, 96, 97, 98, 99 or 100% identity to NCBI Reference No: NP_006130 or a fragment thereof that has stimulatory activity. The term “CD28 transmembrane domain,” also referred to as “CD28 TM domain” or “CD28TM” refers to the amino acid residues derived from the transmembrane domain of CD28. In some embodiments, “CD28TM” comprises the sequence provided as SEQ ID NO: 161 or the equivalent residues from a non-human species, e.g., mouse, rodent, monkey, ape and the like. In some embodiments, “CD28 TM domain” may be encoded by the nucleic acid sequence provided as SEQ ID NO: 261. The term “CD28 hinge” as used herein refers to amino acid residues that may be used to join two domains or two portions within a domain in CARs of some of the embodiments. In some embodiments, “CD28 hinge” comprises the sequence provided as SEQ ID NO: 163 or the equivalent residues from a non-human species, e.g., mouse, rodent, monkey, ape and the like. In some embodiments, “CD28 hinge” may be encoded by the nucleic acid sequence provided as SEQ ID NO: 263. The term “CD28 costimulatory domain,” also referred to as “CD28 CS domain” or “CD28CS,” refers to the amino acid residues derived from the cytoplasmic domain of CD28. In some embodiments, “CD28CS” comprises the sequence provided as SEQ ID NO: 164 or the equivalent residues from a non-human species, e.g., mouse, rodent, monkey, ape and the like. In some embodiments, “CD28 CS domain” may be encoded by the nucleic acid sequence provided as SEQ ID NO: 264.

The term “CD3 zeta,” or alternatively, “zeta,” “zeta chain,” “CD3-zeta,” “CD3z,” “TCR-zeta,” or “CD247,” is a protein encoded by the CD247 gene on chromosome 1, with gene location 1q24.2, in humans. CD3 zeta, together with T cell receptor (TCR) and CD3 (a protein complex composed of a CD3 gamma, a CD3 delta, and two CD3 epsilon), forms the TCR complex. Human CD3 zeta may have an amino acid sequence provided as NP_000725 or NP_932170, or the equivalent residues from a non-human species, e.g., mouse, rodent, monkey, ape and the like. The term “CD3 zeta intracellular signaling domain,” or alternatively “CD3 zeta ICS domain” or a “CD3zICS,” is defined as the amino acid residues from the cytoplasmic domain of the CD3 zeta chain, or functional derivatives thereof, that are sufficient to functionally transmit an initial signal necessary for T cell activation. In one aspect, “CD3 zeta ICS domain” is the sequence provided as SEQ ID NO: 162. In one aspect, “CD3 zeta ICS domain” is encoded by the nucleic acid sequence provided as SEQ ID NO: 262.

The term “Chimeric Antigen Receptor” or alternatively a “CAR” refers to a set of polypeptides, typically two in the simplest embodiments, which when in an immune effector cell, provides the cell with specificity for a target cell, and with intracellular signal generation. In some embodiments, a CAR comprises at least an extracellular antigen binding domain (AB domain), a transmembrane domain (TM domain) and a cytoplasmic signaling domain (also referred to herein as “an intracellular signaling domain (ICS domain)”) comprising a functional signaling domain derived from a stimulatory molecule and/or costimulatory molecule as defined below. In some aspects, the set of polypeptides are contiguous with each other. In some embodiments, the set of polypeptides include a dimerization switch that, upon the presence of a dimerization molecule, can couple the polypeptides to one another, e.g., can couple an AB domain to an ICS domain. In one aspect, the stimulatory molecule is the zeta chain associated with the T cell receptor complex. In one aspect, the cytoplasmic portion of a CAR further comprises a costimulatory domain (CS domain) comprising one or more functional signaling domains derived from at least one costimulatory molecule as defined below. In one aspect, the costimulatory molecule is chosen from the costimulatory molecules described herein, e.g., 4-1BB (i.e., CD137), DAP10 and/or CD28. In one aspect, the CAR comprises a chimeric fusion protein comprising an extracellular AB domain, a TM domain and an ICS domain comprising a functional signaling domain derived from a stimulatory molecule. In one aspect, the CAR comprises a chimeric fusion protein comprising an extracellular AB domain, a TM domain, an ICS domain comprising a functional signaling domain derived from a stimulatory molecule, and a CS domain comprising a functional signaling domain derived from a costimulatory molecule. In one aspect, the CAR comprises a chimeric fusion protein comprising an extracellular AB domain, a TM domain, an ICS domain comprising a functional signaling domain derived from a stimulatory molecule, and two CS domains each of the two comprising a functional signaling domain derived from a costimulatory molecule(s) that is/are same with or different from each other. In one aspect, the CAR comprises a chimeric fusion protein comprising an extracellular AB domain, a TM domain, an ICS domain comprising a functional signaling domain derived from a stimulatory molecule, and at least two CS domains each comprising a functional signaling domain derived from a costimulatory molecule(s) that is/are same with or different from each other. In one aspect the CAR comprises an optional leader sequence at the amino-terminus (N-ter) of the CAR fusion protein. In one aspect, the CAR further comprises a leader sequence at the N-terminus of the extracellular antigen binding domain, wherein the leader sequence is optionally cleaved from the antigen binding domain (e.g., a scFv) during cellular processing and localization of the CAR to the cellular membrane. In some embodiments, the leader sequence (LS) comprises the amino acid sequence provided as SEQ ID NO: 160. In some embodiments, the LS may be encoded by a nucleic acid sequence provided as SEQ ID NO: 260.

The term “compete”, as used herein with regard to an Ab, antigen-binding Ab fragment, of AB domain of any of the anti-ADAM12 agents of the present invention, means that a first Ab, antigen-binding Ab fragment, or AB domain, binds to an epitope in a manner sufficiently similar to the binding of a second Ab, antigen-binding Ab fragment, or AB domain, such that the result of binding of the first Ab, antigen-binding Ab fragment, or AB domain with its cognate epitope is detectably decreased in the presence of the second Ab, antigen-binding Ab fragment, or AB domain compared to the binding of the first Ab, antigen-binding Ab fragment, or AB domain in the absence of the second Ab, antigen-binding Ab fragment, or AB domain. The alternative, where the binding of the second Ab, antigen-binding Ab fragment, or AB domain to its epitope is also detectably decreased in the presence of the first antibody, can, but need not be the case. That is, a first Ab, antigen-binding Ab fragment, or AB domain can inhibit the binding of a second Ab, antigen-binding Ab fragment, or AB domain to its epitope without that second Ab, antigen-binding Ab fragment, or AB domain inhibiting the binding of the first Ab, antigen-binding Ab fragment, or AB domain to its respective epitope. However, where each Ab, antigen-binding Ab fragment, or AB domain detectably inhibits the binding of the other Ab, antigen-binding Ab fragment, or AB domain with its cognate epitope or ligand, whether to the same, greater, or lesser extent, the two (Ab, antigen-binding Ab fragment, or AB domain) are said to “cross-compete” with each other for binding of their respective epitope(s). Both competing and cross-competing Abs, antigen-binding Ab fragments, or AB domains are encompassed by the invention. Regardless of the mechanism by which such competition or cross-competition occurs (e.g., steric hindrance, conformational change, or binding to a common epitope, or portion thereof), the skilled artisan would appreciate, based upon the teachings provided herein, that such competing and/or cross-competing Abs, antigen-binding Ab fragments, or AB domains are encompassed and can be useful for the methods disclosed herein.

The terms “complementarity determining region,” and “CDR,” synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3).

The term “conservative amino acid substitutions” herein are as commonly used in the art and include amino acid substitutions in which one amino acid having certain physical and/or chemical properties is exchanged for another amino acid that has the same or similar chemical or physical properties. For instance, the conservative amino acid substitution can be an acidic/negatively charged polar amino acid substituted for another acidic/negatively charged polar amino acid (e.g., Asp or Glu), an amino acid with a nonpolar side chain substituted for another amino acid with a nonpolar side chain (e.g., Ala, Gly, Val, Ile, Leu, Met, Phe, Pro, Trp, Cys, Val, etc.), a basic/positively charged polar amino acid substituted for another basic/positively charged polar amino acid (e.g. Lys, His, Arg, etc.), an uncharged amino acid with a polar side chain substituted for another uncharged amino acid with a polar side chain (e.g., Asn, Gin, Ser, Thr, Tyr, etc.), an amino acid with a β-branched side-chain substituted for another amino acid with a β-branched side-chain (e.g., Ile, Thr, and Val), an amino acid with an aromatic side-chain substituted for another amino acid with an aromatic side chain (e.g., His, Phe, Trp, and Tyr), etc. Non-conservative amino acid substitutions are amino acid substitutions that are not conservative amino acid substitutions.

The term “costimulatory molecule” refers to a cognate binding partner on a T cell that specifically binds with a costimulatory ligand, thereby mediating a costimulatory response by the T cell, such as, but not limited to, proliferation. Costimulatory molecules are cell surface molecules other than antigen receptors or their ligands that contribute to an efficient immune response. Costimulatory molecules include, but are not limited to a protein selected from the group consisting of an MHC class I molecule, TNF receptor proteins, Immunoglobulin-like proteins, cytokine receptors, integrins, signaling lymphocytic activation molecules (SLAM proteins), activating NK cell receptors, a Toll ligand receptor, B7-H3, BAFFR, BTLA, BLAME (SLAMF8), CD2, CD4, CD5, CD7, CD8alpha, CD8beta, CD11a, LFA-1 (CD11a/CD18), CD11b, CD11c, CD11d, CD18, CD19, CD19a, CD27, CD28, CD29, CD30, CD40, CD49a, CD49D, CD49f, CD69, CD84, CD96 (Tactile), CD100 (SEMA4D), CD103, OX40 (CD134), 4-1BB (CD137), SLAM (SLAMF1, CD150, IPO-3), CD160 (BYS5), SELPLG (CD162), DNAM1 (CD226), Ly9 (CD229), SLAMF4 (CD244, 2B4), ICOS (CD278), CEACAM1, CDS, CRTAM, DAP10, GADS, GITR, HVEM (LIGHTR), IA4, ICAM-1, IL2R beta, IL2R gamma, IL7R alpha, ITGA4, ITGA6, ITGAD, ITGAE, ITGAL, ITGAM, ITGAX, ITGB1, ITGB2, ITGB7, KIRDS2, LAT, LFA-1, LIGHT, LTBR, NKG2C, NKG2D, NKp30, NKp44, NKp46, NKp80 (KLRF1), PAG/Cbp, PD-1, PSGL1, SLAMF6 (NTB-A, Ly108), SLAMF7, SLP-76, TNFR2, TRANCE/RANKL, VLA1, VLA-6, and a ligand that specifically binds with CD83. In embodiments wherein a CAR comprises one or more CS domain, each CS domain comprises a functional signaling domain derived from a costimulatory molecule. In some embodiments, the encoded CS domain comprises 4-1BB, CD28, or DAP10. In one embodiment, the CS domain comprises the amino acid sequence of CD28CS, 41BBCS, or DAP10CS (SEQ ID NO: 164, 165, or 166), or is encoded by the nucleotide sequence encoding provided as SEQ ID NOs: 264, 265, or 266.

The term “cytokines” as used herein refers to a broad category of small proteins that are involved in cell signaling. Generally, their release has some effect on the behavior of cells around them. Cytokines may be involved in autocrine signaling, paracrine signaling and/or endocrine signaling as immunomodulating agents. Cytokines include chemokines, interferons, interleukins, lymphokines, and tumor necrosis factors. Cytokines are produced by a broad range of cells, including immune cells like macrophages, B lymphocytes, T lymphocytes and mast cells, as well as endothelial cells, fibroblasts, epithelial cells, and various stromal cells. “Chemokines” are a family of cytokines generally involved in mediating chemotaxis.

The term “cytotoxicity” generally refers to any cytocidal activity resulting from the exposure of the anti-ADAM12 agents of the invention or cells comprising the same to cells expressing ADAM12. This activity may be measured by known cytotoxicity assays, including IFN-γ production assays. When the target cell is a cancer or tumor cell, the term “anti-cancer cytotoxicity” or “anti-tumor cytotoxicity” may be used.

The term “DAP10” refers to a protein, which in humans is encoded by the HSCT gene. It may also be referred to as HSCT, KAP10, PIK3AP, or hematopoietic cell signal transducer. In some embodiments, DAP10 may have the sequence provided in Genbank Accession No.: Q9UBK5.1. The term “DAP10 costimulatory domain,” also referred to as “DAP10 CS domain” or “DAP10CS,” refers to the amino acid residues derived from the cytoplasmic domain of DAP10. In some embodiments, “DAP10CS” comprises the sequence provided as SEQ ID NO: 166 or the equivalent residues from a non-human species, e.g., mouse, rodent, monkey, ape and the like. In some embodiments, “DAP10 CS domain” may be encoded by the nucleic acid sequence provided as SEQ ID NO: 266.

The phrase “disease associated with expression of ADAM12” or “ADAM12-associated disease” includes, but is not limited to, a disease associated with expression of ADAM12 or condition associated with cells which express ADAM12 including, e.g., proliferative diseases such as a cancer or malignancy or a precancerous condition; or a noncancer related indication associated with cells which express ADAM12. Noncancer-related indications associated with ADAM12 include fibrosis, autoimmunity, a cardiovascular condition, an allergic condition, a respiratory disease, a nephropathy, a neural disease, a muscular disease, a liver disease, metabolic syndrome, infection, and an inflammatory disorder. Examples of various cancers that express ADAM12 include but are not limited to, bladder cancer, bone cancer, brain cancer, breast cancer, colon cancer, colorectal cancer, desmoid tumor, esophageal cancer, fibromatosis, glioblastoma, head and neck cancer, liver cancer, lung cancer, melanoma, oesophago-gastric adenocarcinoma, oligodendroma, oral cancer, oral squamous cell carcinoma, osteosarcoma, ovarian cancer, pancreatic cancer, prostate cancer, skin cancer, small cell lung cancer, stomach cancer, and thyroid cancer, and the like.

An “effective amount” or “an amount effective to treat” refers to a dose that is adequate to prevent or treat a disease, condition, or disorder in an individual. Amounts effective for a therapeutic or prophylactic use will depend on, for example, the stage and severity of the disease or disorder being treated, the age, weight, and general state of health of the patient, another pre-existing condition, and the judgment of the prescribing physician. The size of the dose will also be determined by the active ingredient selected, method of administration, timing and frequency of administration, the existence, nature, and extent of any adverse side effects that might accompany the administration of a particular active ingredient, and the desired physiological effect. It will be appreciated by one of skill in the art that various diseases or disorders could require prolonged treatment involving multiple administrations, perhaps using the inventive anti-ADAM12 agents, nucleic acids, vectors, cells, or compositions in each or various rounds of administration.

The terms “enteral,” “enterally,” “oral,” “orally,” “non-parenteral,” “non-parenterally,” and the like, refer to administration of a compound or composition to an individual by a route or mode along the alimentary canal. Examples of “oral” routes of administration of a composition include, without limitation, swallowing liquid or solid forms of a composition from the mouth, administration of a composition through a nasojejunal or gastrostomy tube, intraduodenal administration of a composition, and rectal administration, e.g., using suppositories for the lower intestinal tract of the alimentary canal.

The term “framework” as used herein refers to the non-CDR portions of the variable region of an Ab, or in some embodiments, Antigen-binding Ab fragment or an AB domain of a CAR. “Heavy chain (HC) framework” refers to the non-CDR portion of a HC variable region, and in general, there are four framework regions (FRs) in each full-length heavy chain variable region (FR-H1, FR-H2, FR-H3, and FR-H4). “Light chain (LC) framework” refers to the non-CDR portion of a LC variable region, and in general, there are four FRs in each full-length light chain variable region (FR-L1, FR-L2, FR-L3, and FR-L4). In some embodiments, “human-like HC framework” is at least 80%, at least 85%, at least 90%, at least 92%, at least 94%, at least 95%, at least 96%, at least 97%, at least 98%, at least 99%, or 100% identical to a human HC framework. In some embodiments, “human-like LC framework” is at least 80%, at least 85%, at least 90%, at least 92%, at least 94%, at least 95%, at least 96%, at least 97%, at least 98%, at least 99%, or 100% identical to a human LC framework.

The term “gene” is used broadly to refer to any segment of polynucleotide associated with a biological function. Thus, genes include introns and exons as in genomic sequence, or just the coding sequences as in cDNAs and/or the regulatory sequences required for their expression. For example, gene also refers to a nucleic acid fragment that expresses mRNA or functional RNA, or encodes a specific protein, and which includes regulatory sequences.

The term “hinge”, “spacer”, or “linker” refers to an amino acid sequence of variable length typically encoded between two or more domains or portions of a polypeptide construct to confer flexibility, improved spatial organization, proximity, etc.

As used herein, “human antibody” means an antibody having an amino acid sequence corresponding to that of an antibody produced by a human and/or which has been made using any of the techniques for making human antibodies known to those skilled in the art or disclosed herein. Human antibodies can be produced using various techniques known in the art. In one embodiment, the human antibody is selected from a phage library, where that phage library expresses human antibodies (Vaughan et al., Nature Biotechnology, 14:309-314, 1996; Sheets et al., Proc. Natl. Acad. Sci. (USA) 95:6157-6162, 1998; Hoogenboom and Winter, J. Mol. Biol., 227:381, 1991; Marks et al., J. Mol. Biol., 222:581, 1991). Human antibodies can also be made by immunization of animals into which human immunoglobulin loci have been transgenically introduced in place of the endogenous loci, e.g., mice in which the endogenous immunoglobulin genes have been partially or completely inactivated. This approach is described in U.S. Pat. Nos. 5,545,807; 5,545,806; 5,569,825; 5,625,126; 5,633,425; and 5,661,016. Alternatively, the human antibody may be prepared by immortalizing human B lymphocytes that produce an antibody directed against a target antigen (such B lymphocytes may be recovered from an individual or from single cell cloning of the cDNA, or may have been immunized in vitro). See, e.g., Cole et al. Monoclonal Antibodies and Cancer Therapy, Alan R. Liss, p. 77, 1985; Boerner et al., J. Immunol., 147 (1):86-95, 1991; and U.S. Pat. No. 5,750,373.

The term “humanization” of an Ab refers to modification of an Ab of a non-human origin to increase the sequence similarity to an Ab naturally produced in humans. The term “humanized antibody” as used herein refers to Abs generated via humanization of an Ab. Generally, a humanized or engineered antibody has one or more amino acid residues from a source which is non-human, e.g., but not limited to mouse, rat, rabbit, non-human primate or other mammal. These human amino acid residues are often referred to as “import” residues, which are typically taken from an “import” variable, constant or other domain of a known human sequence. Known human Ig sequences are disclosed, e.g., www.ncbi.nlm.nih.gov/entrez/query.fcgi; www.atcc.org/phage/hdb.html, each entirely incorporated herein by reference. Such imported sequences can be used to reduce immunogenicity or reduce, enhance or modify binding, affinity, avidity, specificity, half-life, or any other suitable characteristic, as known in the art. Generally part or all of the non-human or human CDR sequences are maintained while part or all of the non-human sequences of the framework and/or constant regions are replaced with human or other amino acids. Antibodies can also optionally be humanized with retention of high affinity for the antigen and other favorable biological properties using three-dimensional immunoglobulin models that are known to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, framework (FR) residues can be selected and combined from the consensus and import sequences so that the desired antibody characteristic, such as increased affinity for the target antigen(s), is achieved. In general, the CDR residues are directly and most substantially involved in influencing antigen binding. Humanization or engineering of antibodies of the present invention can be performed using any known method, such as but not limited to those described in, for example, Winter (Jones et al., Nature 321:522 (1986); Riechmann et al., Nature 332:323 (1988); Verhoeyen et al., Science 239:1534 (1988)), Sims et al., J. Immunol. 151: 2296 (1993); Chothia and Lesk, J. Mol. Biol. 196:901 (1987), Carter et al., Proc. Natl. Acad. Sci. U.S.A. 89:4285 (1992); Presta et al., J. Immunol. 151:2623 (1993), U.S. Pat. Nos. 5,723,323, 5,976,862, 5,824,514, 5,817,483, 5,814,476, 5,763,192, 5,723,323, 5,766,886, 5,714,352, 6,204,023, 6,180,370, 5,693,762, 5,530,101, 5,585,089, 5,225,539; 4,816,567, each entirely incorporated herein by reference, included references cited therein.

The term “iCAR” is a chimeric antigen receptor which contains inhibitory receptor signaling domains. These domains may be based, for example, on protectin D1 (PD1) or CTLA-4 (CD152). In some embodiments, the CAR expressing cells of the invention are further transduced to express an iCAR. In one aspect, this iCAR is added to restrict the CAR expressing cell's functional activity to tumor cells.

The term “immune cell” refers to a cell of hematopoietic origin functionally involved in the initiation and/or execution of innate and/or adaptive immune response.

The term “intracellular signaling domain” or “ICS domain” as used herein, refers to an intracellular portion of a molecule. The intracellular signaling domain generates a signal that promotes an immune effector function of the cell transduced with a nucleic acid sequence comprising a CAR, e.g., a CAR T cell. Examples of immune effector function, e.g., in a CAR T cell, include cytolytic activity and helper activity, including the secretion of cytokines. ICS domains include an ICS domain of a lymphocyte receptor chain, a TCR/CD3 complex protein, an Fc receptor subunit, an IL-2 receptor subunit, CD3 zeta, FcR gamma, FcR beta, CD3 gamma, CD3 delta, CD3 epsilon, CD5, CD22, CD79a, CD79b, CD66d, CD278(ICOS), Fc epsilon RI, DAP10, or DAP12.

An “isolated” biological component (such as an isolated protein, nucleic acid, vector, or cell) refers to a component that has been substantially separated or purified away from its environment or other biological components in the cell of the organism in which the component naturally occurs, for instance, other chromosomal and extra-chromosomal DNA and RNA, proteins, and organelles. Nucleic acids and proteins that have been “isolated” include nucleic acids and proteins purified by standard purification methods. The term also embraces nucleic acids and proteins prepared by recombinant technology as well as chemical synthesis. An isolated nucleic acid or protein can exist in substantially purified form, or can exist in a non-native environment such as, for example, a host cell.

A “leader sequence” or “LS” as used herein, also referred to as “signal peptide,” “signal sequence,” “targeting signal,” “localization signal,” “localization sequence,” “transit peptide,” or “leader peptide” in the art, is a short peptide present at the N-terminus of the majority of newly synthesized proteins that are destined towards the secretary pathway. The core of the signal peptide may contain a long stretch of hydrophobic amino acids. The signal peptide may or may not be cleaved from the mature polypeptide.

The term “linker” as used in the context of a scFv refers to a peptide linker that consists of amino acids such as glycine and/or serine residues used alone or in combination, to link variable heavy and variable light chain regions together. In one embodiment, the flexible polypeptide linker is a Gly/Ser linker and comprises one or more repeats of the amino acid sequence unit Gly-Gly-Gly-Gly-Ser (SEQ ID NO: 167). In one embodiment, the flexible polypeptide linker includes, but is not limited to, (Gly4Ser)3, which is also referred to as G4S X3 (SEQ ID NO: 168). Such a linker may be encoded for example, by the nucleic acid sequence as set forth in SEQ ID NO: 268.

The term “mammal” refers to any mammal, including, but not limited to, mammals of the order Rodentia, such as mice, rats, and hamsters, and mammals of the order Logomorpha, such as rabbits. The mammals may be from the order Carnivora, including Felines (cats) and Canines (dogs). The mammals may be from the order Artiodactyla, including Bovines (cows) and Swines (pigs) or of the order Perssodactyla, including Equines (horses). The mammals may be of the order Primates, Ceboids, or Simoids (monkeys) or of the order Anthropoids (humans and apes).

The term “masked CAR” refers to a CAR expressing cell that further comprises a masking peptide. This masking peptide may prevent off-target cell killing. The masking peptide is often N-terminal to the CAR construct and may block the cell's ability to bind to unintended targets. The masking peptide may be cleaved from the CAR expressing cell when it encounters a tumor thereby allowing the CAR expressing cell to attack its target without killing off-target cells. An anti-ADAM12 CARs of the present invention may be constructed to be a masked CAR.

The term “multispecific” as used herein refers to having two or more binding specificities. An anti-ADAM12 multispecific Ab or a multispecific antigen-binding Ab fragment, for example, of the present invention has at least one specificity for ADAM12. When the first specificity is for an epitope for ADAM12, the second (or third, fourth, and so forth) specificity may be for another epitope for ADAM12 or may be for a molecule other than ADAM12. The term “multicific” is also used in the same manner for any other anti-ADAM12 agents of the present invention, such as anti-ADAM12 CARs.

The term “nucleic acid” and “polynucleotide” refer to RNA or DNA that is linear or branched, single or double stranded, or a hybrid thereof. The term also encompasses RNA/DNA hybrids. The following are non-limiting examples of polynucleotides: a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes and primers. A polynucleotide may comprise modified nucleotides, such as methylated nucleotides and nucleotide analogs, uracil, other sugars and linking groups such as fluororibose and thiolate, and nucleotide branches. The sequence of nucleotides may be further modified after polymerization, such as by conjugation, with a labeling component. Other types of modifications included in this definition are caps, substitution of one or more of the naturally occurring nucleotides with an analog, and introduction of means for attaching the polynucleotide to proteins, metal ions, labeling components, other polynucleotides or solid support. The polynucleotides can be obtained by chemical synthesis or derived from a microorganism.

The term “OKT3” or “Muromonab-CD3” or “Orthoclone OKT3” refers to a monoclonal anti-CD3 Ab.

The term “parenteral” or “parenterally” as used herein includes any route of administration of a compound or composition, characterized by physical breaching of a tissue of a subject and administration of the pharmaceutical composition through the breach in the tissue, thus generally resulting in the direct administration into the blood stream, into muscle, or into an internal organ. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal, intravenous, intraarterial, intrathecal, intraventricular, intraurethral, intracranial, intrasynovial injection or infusions; and kidney dialytic infusion techniques. In a preferred embodiment, parenteral administration of the compositions of the present invention comprises subcutaneous or intraperitoneal administration.

The term “pharmaceutically acceptable excipient,” “pharmaceutical excipient,” “excipient,” “pharmaceutically acceptable carrier,” “pharmaceutical carrier,” or “carrier” as used herein refers to compounds or materials conventionally used in pharmaceutical compositions during formulation and/or to permit storage.

The term “promoter”, as used herein, is defined as a DNA sequence recognized by the synthetic machinery of the cell, or introduced synthetic machinery, required to initiate the specific transcription of a polynucleotide sequence

The “ribosome skip sequence” refers to an amino acid sequence that, when translated, causes cleavage of a nascent polyprotein on the ribosome, allowing for co-expression of multiple genes. In one aspect, the ribosome skip sequence may be the T2A sequence such as SEQ ID NO: 169, which may be encoded by SEQ ID NO: 269. Alternatively, any other 2A sequences may be used. Examples of other sequences may be found elsewhere in the literature of the relevant art (for example, see Kim, J. H., et al., High cleavage efficiency of a 2A peptide derived from porcine teschovirus-1 in human cell lines, zebrafish and mice. PLoS One. 2011; 6(4)).

The term “recombinant” means a polynucleotide, a protein, a cell, and so forth with semi-synthetic or synthetic origin which either does not occur in nature or is linked to another polynucleotide, a protein, a cell, and so forth in an arrangement not found in nature.

The term “scFv,” “single-chain Fv,” or “single-chain variable fragment” refers to a fusion protein comprising at least one antibody fragment comprising a variable region of a light chain and at least one antibody fragment comprising a variable region of a heavy chain, wherein the light and heavy chain variable regions are contiguously linked, e.g., via a synthetic linker, e.g., a short flexible polypeptide linker, and capable of being expressed as a single chain polypeptide, and wherein the scFv retains the specificity of the intact antibody from which it is derived. Unless specified, as used herein an scFv may have the VL and VH variable regions in either order, e.g., with respect to the N-terminal and C-terminal ends of the polypeptide, the scFv may comprise VL-linker-VH or may comprise VH-linker-VL. The linker may comprise portions of the framework sequences. In scFvs, the heavy chain variable domain (HC V, HCV, or VH) may be placed upstream of the light chain variable domain (LC V, LCV, or VL), and the two domains may optionally be linked via a linker (for example, the G4S X3 linker). In this case, when the scFv is for example derived from h6E6, the construct may be referred to as h6E6scFvHL, h6E6HL, h6E6scFvVHVL, or h6E6VHVL. Alternatively, the heavy chain variable domain may be placed downstream of the light chain variable domain, and the two domains may optionally be linked via a linker (for example, the G4S X3 linker). In this case, when the scFv is for example derived from h6E6, the construct may be referred to as h6E6scFvLH, h6E6LH, h6E6scFvVLVH, or h6E6VLVH. The same naming rules apply to other similar constructs herein.

The term “signaling domain” refers to the functional portion of a protein which acts by transmitting information within the cell to regulate cellular activity via defined signaling pathways by generating second messengers or functioning as effectors by responding to such messengers.

The term “stimulatory molecule,” refers to a molecule expressed by an immune cell (e.g., T cell, NK cell, B cell) that provides the cytoplasmic signaling sequence(s) that regulate activation of the immune cell in a stimulatory way for at least some aspect of the immune cell signaling pathway. In one aspect, the signal is a primary signal that is initiated by, for instance, binding of a TCR/CD3 complex with an MHC molecule loaded with peptide, and which leads to mediation of a T cell response, including, but not limited to, proliferation, activation, differentiation, and the like. A primary cytoplasmic signaling sequence (also referred to as a “primary signaling domain”) that acts in a stimulatory manner may contain a signaling motif which is known as an immunoreceptor tyrosine-based activation motif or ITAM. Examples of an ITAM containing cytoplasmic signaling sequence that are of particular use in the invention include, but are not limited to, those derived from CD3 zeta, common FcR gamma (FCER1G), Fc gamma RIIa, FcR beta (Fc epsilon R1b), CD3 gamma, CD3 delta, CD3 epsilon, CD79a, CD79b, DAP10, and DAP12. In a specific CAR of the invention, the intracellular signaling domain in any one or more CARS of the invention comprises an intracellular signaling sequence, e.g., a primary signaling sequence of CD3 zeta. In a specific CAR of the invention, the primary signaling sequence of human CD3 zeta, referred to as “CD3zICS” herein, is the amino acid sequence provided as SEQ ID NO: 162, and may be encoded by the nucleotide sequence SEQ ID NO: 262. Alternatively, equivalent residues from a non-human or mouse species, e.g., rodent, monkey, ape and the like, may be utilized.

The term “subject” as used herein may be any living organisms, preferably a mammal. In some embodiments, the subject is a primate such as a human. In some embodiments, the primate is a monkey or an ape. The subject can be male or female and can be any suitable age, including infant, juvenile, adolescent, adult, and geriatric subjects. In some examples, the patient or subject is a validated animal model for disease and/or for assessing toxic outcomes. The subject may also be referred to as “patient” in the art. The subject may have a disease or may be healthy.

The term “suicide mechanism” as used herein refers to a mechanism by which anti-ADAM12 agent-expressing cells of present invention may be eradicated from a subject administered with such cells. The suicide mechanism may be driven by, for example, inducible caspase 9 (Budde L. E. et al., PLoS One. 2013 Dec. 17; 8(12):e82742. doi: 10.1371/journal.pone.0082742. eCollection 2013), codon-optimized CD20 (Martin V. et al., Hum Gene Ther Methods. 2012 December; 23(6): 376-386), CD34, or polypeptide RQR8 (Philip et al, and WO2013153391A, which is hereby incorporated herein by reference). In some embodiments, the suicide mechanism may be included and utilized in CAR-expressing cells of present invention to optimize the length for the CAR-expressing cells to stay in the system of a subject or the amount of the CAR-expressing cells, to reduce or minimize the toxicity and/or to maximize the benefit of CAR-expressing cells.

The term “synthetic Ab” or “synthetic antigen-binding Ab fragment” as used herein, refers to an Ab or antigen-binding Ab fragment which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “target” as used herein refers to the molecule that an anti-ADAM12 agents of the present invention specifically binds to. The term also encompasses cells and tissues expressing the target molecule and also diseases that are associated with expression of the target.

The term “target cell” as used herein refers to a cell expressing the target molecule (such as ADAM12) of the anti-ADAM12 agents of the present invention on the cell surface. In some embodiments, the target cell is a cancer cell or tumor cell. In some embodiments, the target cell is a vascular cell. In some embodiments, the target cell is a fibroblast. In some embodiments, the target cell is an epithelial cell. In some embodiments, the target cell is a cell type that has a particular role in the pathology of cancer or inflammation. In some embodiments, the target cell is a cell type that has a particular role in the pathology of a disease such as but not limited to cancer, fibrosis, autoimmunity, an inflammatory disease, a cardiovascular condition, a metabolic disease, an allergic condition, a respiratory disease, a nephropathy, a neural disease, a muscular disease, a liver disease, metabolic syndrome, infection, and an inflammatory disorder.

The term “target molecule” as used herein refers to a molecule that is targeted by the anti-ADAM12 agents of the present invention. The AB domain of the anti-ADAM12 agents of the present invention has a binding affinity for the target molecule. In some embodiments, the target molecule is ADAM12.

The term “trCD19” refers to a truncated version of the CD19 protein, B-lymphocyte antigen CD19, also known as CD19 (Cluster of Differentiation 19), which is a protein that is encoded by the CD19 gene in humans and is found on the surface of B-cells. The trCD19 construct is any truncated version of said protein, such that a nucleic acid sequence encoding this construct may be transduced into a host cell and expressed on the surface of this cell for the purposes of detection, selection, and/or targeting. In one aspect, human trCD19 may comprise the amino acid sequence of SEQ ID NO: 170 or nucleotide sequence encoding such, such as SEQ ID NO: 270.

The term “transfected,” “transformed,” or “transduced” refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

By the term “transmembrane domain” or “TM domain”, what is implied is any three-dimensional protein structure which is thermodynamically stable in a membrane. This may be a single alpha helix, a transmembrane beta barrel, a beta-helix of gramicidin A, or any other structure. Transmembrane helices are usually about 20 amino acids in length. Typically, the transmembrane domain denotes a single transmembrane alpha helix of a transmembrane protein, also known as an integral protein.

As used herein, the term “treat,” “treatment,” or “treating” generally refers to the clinical procedure for reducing or ameliorating the progression, severity, and/or duration of a disease or of a condition, or for ameliorating one or more conditions or symptoms (preferably, one or more discernible ones) of a disease. The type of disease or condition to be treated may be, for example, but are not limited to, cancer, fibrosis or fibrotic disease, autoimmunity, a cardiovascular condition, an allergic condition, a respiratory disease, a nephropathy, a neural disease, a muscular disease, a liver disease, metabolic syndrome, infection, and an inflammatory disorder. Examples of cancer include, but are not limited to, bladder cancer, bone cancer, brain cancer, breast cancer, colon cancer, colorectal cancer, desmoid tumor, esophageal cancer, fibromatosis, glioblastoma, head and neck cancer, liver cancer, lung cancer, melanoma, oesophago-gastric adenocarcinoma, oligodendroma, oral cancer, oral squamous cell carcinoma, osteosarcoma, ovarian cancer, pancreatic cancer, prostate cancer, skin cancer, small cell lung cancer, stomach cancer, and thyroid cancer. Examples of a fibrotic disease include, but are not limited to, pulmonary fibrosis, an interstitial lung disease, cystic fibrosis, chronic obstructive pulmonary disease, sarcoidosis, an allergic airway disease, scleroderma, hepatic fibrosis, or cardiac fibrosis. The condition to be treated may be, for example, fibrosis, oxidative stress, or inflammation. In specific embodiments, the effect of the “treatment” may be evaluated by the amelioration of at least one measurable physical parameter of a disease, resulting from the administration of one or more therapies (e.g., an anti-ADAM12 Ab or antigen-binding Ab fragment, anti-ADAM12 ADC, or anti-ADAM12 CAR expressing cell). The parameter may be, for example, gene expression profiles, the mass of disease-affected tissues, inflammation-associated markers, cancer-associated markers, the number or frequency of disease-associated cells, tumor burden, the presence or absence of certain cytokines or chemokines or other disease-associated molecules, and may not necessarily discernible by the patient. In other embodiments “treat”, “treatment,” or “treating” may result in the inhibition of the progression of a disease, either physically by, e.g., stabilization of a discernible symptom, physiologically by, e.g., stabilization of a physical parameter, or both. In other embodiments the terms “treat”, “treatment” and “treating” refer to the reduction or stabilization of cancerous tissue or cells. Additionally, the terms “treat,” and “prevent” as well as words stemming therefrom, as used herein, do not necessarily imply 100% or complete cure or prevention. Rather, there are varying degrees of treatment effects or prevention effects of which one of ordinary skill in the art recognizes as having a potential benefit or therapeutic effect. In this respect, the inventive methods can provide any amount of any level of treatment or prevention effects of a disease in a mammal. Furthermore, the treatment or prevention provided by the inventive method can include treatment or prevention of one or more conditions or symptoms of the disease being treated or prevented. Also, for purposes herein, “prevention” can encompass delaying the onset of the disease, or a symptom or condition thereof.

The term “xenogeneic” or “xeno-” refers to a graft derived from an animal of a different species.

The experimental details of experiments are described in the following examples. These examples are offered to illustrate, but not to limit, the claimed invention.

### EXAMPLES

### Example 1: Humanization of Mouse Anti-ADAM12 Antibodies

### <Materials>

### Mouse Anti-ADAM12 Antibody (Clone 6E6) Sequence

Heavy chain (HC) variable domain (VH): SEQ ID NO: 111, encoded by SEQ ID NO: 211

CDR 1, CDR 2, and CDR 3 of VH: SEQ ID NOs: 112, 113, and 114, encoded by SEQ ID NOs: 212, 213, and 214

Light chain (LC) variable domain (VL): SEQ ID NO: 115, encoded by SEQ ID NO: 215

CDR 1, CDR 2, and CDR 3 of VL: SEQ ID NOs: 116, 117, and 118, encoded by SEQ ID NOs: 216, 217, and 218

### Mouse Anti-ADAM12 Antibody (Clone 6C10) Sequence

Heavy chain (HC) variable domain (VH): SEQ ID NO: 121, encoded by SEQ ID NO: 221

CDR 1, CDR 2, and CDR 3 of VH: SEQ ID NOs: 122, 123, and 124, encoded by SEQ ID NOs: 222, 223, and 224

Light chain (LC) variable domain (VL): SEQ ID NO: 125, encoded by SEQ ID NO: 225

CDR 1, CDR 2, and CDR 3 of VL: SEQ ID NOs: 126, 127, and 128, encoded by SEQ ID NOs: 226, 227, and 228

### <Methods>

The sequence of CDR 1, CDR 2, and CDR 3 from VH and VL of 6E6 and 6C10 were grafted onto a human framework sequence by the inventor and further humanized.

### <Results>

The humanized version of 6E6 and 6C10 were obtained, with the sequences below:

Humanized Anti-ADAM12 Antibody (h6E6) Sequence

VH: SEQ ID NO: 131, encoded by SEQ ID NO: 231

CDR 1, CDR 2, and CDR 3 of VH: SEQ ID NOs: 132, 133, and 134, encoded by SEQ ID NOs: 232, 233, and 234

VL: SEQ ID NO: 135, encoded by SEQ ID NO: 235

CDR 1, CDR 2, and CDR 3 of VL: SEQ ID NOs: 136, 137, and 138, encoded by SEQ ID NOs: 236, 237, and 238

Humanized Anti-ADAM12 Antibody (h6C10) Sequence

VH: SEQ ID NO: 141, encoded by SEQ ID NO: 241

CDR 1, CDR 2, and CDR 3 of VH: SEQ ID NOs: 142, 143, and 144, encoded by SEQ ID NOs: 242, 243, and 244

VL: SEQ ID NO: 145, encoded by SEQ ID NO: 245

CDR 1, CDR 2, and CDR 3 of VL: SEQ ID NOs: 146, 147, and 148, encoded by SEQ ID NOs: 246, 247, and 248

### Example 2: ADAM12 Expression on Cancer Cell Lines

### <Materials>

MCF7-ADAM12 cells (human breast cancer cell line MCF7 transfected with an ADAM12 expression vector) and U87-MG cells (human glioblastoma cell line)

Mouse anti-human ADAM12 IgG primary antibody (Clone 6C10)

Unpurified ascites samples harvested from mice harboring cells producing mouse anti-human ADAM12 antibody (Clone 7B8 or 8F8)

FITC-labeled anti-mouse IgG secondary antibody

### <Methods>

Cells were stained first with mouse anti-human ADAM12 IgG primary antibody (Clone 6C10) or with an unpurified ascites sample harvested from mice harboring cells producing anti-human ADAM12 antibody (Clone 7B8 or 8F8). Cells were then stained with FITC-labeled anti-mouse IgG and analyzed by flow cytometry.

### <Results>

The results from flow cytometry are shown in FIG. 5. All cells were stained positive for ADAM12 expression.

### Example 3: Generation and Expression of CARs

### <Materials>

Human T cells from Donor 1.

Empty vector (EV) encoding trCD19.

Vector encoding CAR and trCD19.

### <Methods>

Human T cells from Donor 1 were transduced with a vector encoding anti-ADAM12 CAR1, anti-ADAM12 CAR2, or an empty vector (EV, i.e., trCD19 only) and enriched for trCD19-positive cells. The sequences used in Examples 3 are SEQ ID NOs: 194 and 294 (amino acid sequence and nucleic acid sequence, respectively, for anti-ADAM12 CAR2, which is “LS-h6E6scFvLH-CD28H-CD28TM-CD28CS-CD3zICS-T2A-trCD19”) and SEQ ID NOs: 197 and 297 (amino acid sequence and nucleic acid sequence, respectively, for anti-ADAM12 CAR1, which is “LS-h6C10scFvHL-CD28H-CD28TM-CD28CS-CD3zICS-T2A-trCD19”).

### <Results>

Expression of both CARs was confirmed.

### Example 4: In Vitro Cytotoxicity by Anti-ADAM12 CAR-Expressing T Cells

### <Materials>

MCF7-ADAM12 cells

Luciferase expression vector JC73

Human T cells expressing trCD19 but no anti-ADAM12 (EV, generated as in Example 3)

Human T cells expressing anti-ADAM12 CAR1 (generated as in Example 3)

Human T cells expressing anti-ADAM12 CAR2 (generated as in Example 3)

### <Methods>

MCF7-ADAM12 cells were transduced with luciferase expression vector, and luciferase positive cells were selected using puromycin and used as target cells. Anti-ADAM12 CAR expressing human T cells from Example 3 were expanded and used as effector cells. T cells were plated with 5,000 luciferase-expressing MCF7-ADAM12 cells at effector:target (E:T) ratios of 10:1, 3:1, 1:1, and 0.3:1 in 96-well light-blocking luminometer plates. After 24 hrs of co-culture, remaining live tumor cells were detected by luciferase activity as measured by luminescence (FIG. 6 top). The plates were incubated an additional 24 hrs to allow for a 48 hr measurement (FIG. 6 bottom).

### <Results>

After 24 hrs of co-culture, a significant reduction in MCF7-ADAM12 cells was observed with anti-ADAM12 CAR1 T cells at the 1:1, 3:1, and 10:1 E:T ratio and with anti-ADAM12 CAR2 T cells at the 10:1 E:T ratio (FIG. 6 top). After 48 hrs, the signal from remaining MCF7-ADAM12 cells was significantly reduced at all E:T rations for both CAR1 and CAR2 (FIG. 6 bottom).

### Example 5: In Vitro Cytokine Production by Anti-ADAM12 CAR-Expressing T Cells

### <Methods>

Anti-ADAM12 CAR expressing human T cells and EV-transduced human T cells from Example 3 were expanded. 10\hat{ }5 MCF7-ADAM12 cells were cultured with 10≡CAR1 T cells, 10\hat{ }5 CAR2 T cells, 10\hat{ }5 EV T cells, or no T cells per well in a 96-well plate for 24 hours. Control wells only containing 10\hat{ }5 CAR1 T cells, CAR2 T cells, or EV T cells were also included. Supernatants were collected and IFN-g levels were measured by ELISA.

### <Results>

The IFN-g concentrations in the supernatant are shown in FIG. 8. Significantly higher levels of IFN-g were observed when CAR1 or CAR2 T cells were cocultured with MCF7-ADAM12 compared to when EV T cells were cocultured with MCF7-ADAM12. Only negligible amount of IFN-g was detected from wells of T cell only or MCF7-ADAM12 cell only. IFN-g production was negligible in the absence of ADAM12-expressing target cells.

### Example 6: In Vivo Efficacy by Anti-ADAM12 CAR-Expressing T Cells

### <Materials>

NOD scid gamma (NSG) mice

MCF7-ADAM12 cells transduced with luciferase (MCF7-ADAM12-Luc)

Human T cells expressing trCD19 but no anti-ADAM12 (EV T, generated as in Example 3)

Human T cells expressing anti-ADAM12 CAR1 (generated as in Example 3)

### <Methods>

2.5×10⁶ MCF7-ADAM12-Luc cells were injected intraperitoneally into NSG mice on Day 0. Mice were then administered with 5×10⁶ human T cells expressing trCD19 but no anti-ADAM12 (EV T) or 5×10⁶ human T cells expressing anti-ADAM12 CAR1 (CAR1 T) intraperitoneally on Day 7. The tumor burden of individual mice was monitored by bioluminescent imaging using the Xenogen-IVIS® Imaging System every week starting on Day 6. The weight of individual mice was recorded periodically starting on Day 0.

### <Results>

The changes in the tumor burden in the two treatment groups (EV T and CAR1 T) are shown in FIGS. 7A and 7B. In both groups, the tumor burden was detectable (both approximately 1.2×10⁹ photons/sec) on Day 6. The tumor burden dramatically decreased in CAR1 T group by Day 13, and the tumor burden remained low at Day 20, Day 27, and Day 34. The tumor burden in CAR1 T group was significantly lower than that in EV T group throughout the experiment. Mice in both groups continued to gain weight except for one mouse from EV T group, which lost weight after Day 34 and was euthanized on Day 44. No statistically significant difference was found in the average body weight between the treatment groups, as shown in FIG. 7C.

Although the foregoing invention has been described in some detail byway of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

### APPENDIX: AMINO ACID AND NUCLEIC ACID SEQUENCES

### ADAM12:

Human ADAM12-L (GenBank: AAC08702.2)
(SEQ ID NO: 101) Protein Sequence:
MAARPLPVSPARALLLALAGALLAPCEARGVSLWNQGRADEVVSASVRSGDLWIPVKSFDSKN
HPEVLNIRLQRESKELIINLERNEGLIASSFTETHYLQDGTDVSLARNYTVILGHCYYHGHVRGY
SDSAVSLSTCSGLRGLIVFENESYVLEPMKSATNRYKLFPAKKLKSVRGSCGSHHNTPNLAAKN
VFPPPSQTWARRHKRETLKATKYVELVIVADNREFQRQGKDLEKVKQRLIEIANHVDKFYRPL
NIRIVLVGVEVWNDMDKCSVSQDPFTSLHEFLDWRKMKLLPRKSHDNAQLVSGVYFQGTTIG
MAPIMSMCTADQSGGIVMDHSDNPLGAAVTLAHELGHNFGMNHDTLDRGCSCQMAVEKGGC
IMNASTGYPFPMVFSSCSRKDLETSLEKGMGVCLFNLPEVRESFGGQKCGNRFVEEGEECDCGE
PEECMNRCCNATTCTLKPDAVCAHGLCCEDCQLKPAGTACRDSSNSCDLPEFCTGASPHCPAN
VYLHDGHSCQDVDGYCYNGICQTHEQQCVTLWGPGAKPAPGICFERVNSAGDPYGNCGKVSKS
SFAKCEMRDAKCGKIQCQGGASRPVIGTNAVSIETNIPLQQGGRILCRGTHVYLGDDMPDPGLV
LAGTKCADGKICLNRQCQNISVFGVHECAMQCHGRGVCNNRKNCHCEAHWAPPFCDKFGFGG
STDSGPIRQADNQGLTIGILVTILCLLAAGFVVYLKRKTLIRLLFTNKKTTIEKLRCVRPSRPPRG
FQPCQAHLGHLGKGLMRKPPDSYPPKDNPRRLLQCQNVDISRPLNGLNVPQPQSTQRVLPPLH
RAPRAPSVPARPLPAKPALRQAQGTCKPNPPQKPLPADPLARTTRLTHALARTPGQWETGLR
LAPLRPAPQYPHQVPRSTHTAYIK
Human ADAM12-S (GenBank: AAC08703.2)
(SEQ ID NO: 102) Protein Sequence:
MAARPLPVSPARALLLALAGALLAPCEARGVSLWNQGRADEVVSASVRSGDLWIPVKSFDSKN
HPEVLNIRLQRESKELIINLERNEGLIASSFTETHYLQDGTDVSLARNYTVILGHCYYHGHVRGY
SDSAVSLSTCSGLRGLIVFENESYVLEPMKSATNRYKLFPAKKLKSVRGSCGSHHNTPNLAAKN
VFPPPSQTWARRHKRETLKATKYVELVIVADNREFQRQGKDLEKVKQRLIEIANHVDKFYRPL
NIRIVLVGVEVWNDMDKCSVSQDPFTSLHEFLDWRKMKLLPRKSHDNAQLVSGVYFQGTTIG
MAPIMSMCTADQSGGIVMDHSDNPLGAAVTLAHELGHNFGMNHDTLDRGCSCQMAVEKGGC
IMNASTGYPFPMVFSSCSRKDLETSLEKGMGVCLFNLPEVRESFGGQKCGNRFVEEGEECDCGE
PEECMNRCCNATTCTLKPDAVCAHGLCCEDCQLKPAGTACRDSSNSCDLPEFCTGASPHCPAN
VYLHDGHSCQDVDGYCYNGICQTHEQQCVTLWGPGAKPAPGICFERVNSAGDPYGNCGKVSKS
SFAKCEMRDAKCGKIQCQGGASRPVIGTNAVSIETNIPLQQGGRILCRGTHVYLGDDMPDPGLV
LAGTKCADGKICLNRQCQNISVFGVHECAMQCHGRGVCNNRKNCHCEAHWAPPFCDKFGFGG
STDSGPIRQAEARQEAAESNRERGQGQEPVGSQEHASTASLTLI
Mouse ADAM12 (NCBI Reference Sequence: NP_031426.2)
(SEQ ID NO: 103) Protein Sequence:
MAERPARRAPPARALLLALAGALLAPRAARGMSLWDQRGTYEVARASLLSKDPGIPGQSIPAK
DHPDVLTVQLQLESRDLILSLERNEGLIANGFTETHYLQDGTDVSLTRNHTDHCYYHGHVQGD
AASVVSLSTCSGLRGLIMFENKTYSLEPMKNTTDSYKLVPAESMTNIQGLCGSQHNKSNLTME
DVSPGTSQMRARRHKRETLKMTKYVELVIVADNREFQRQGKDLEKVKQRLIEIANHVDKFYR
PLNIRIVLVGVEVWNDIDKCSISQDPFTSLHEFLDWRKIKLLPRKSHDNAQLISGVYFQGTTIGM
APIMSMCTAEQSGGVVMDHSDSPLGAAVTLAHELGHNFGMNHDTLERGCSCRMAAEKGGCI
MNPSTGFPFPMVFSSCSRKDLEASLEKGMGMCLFNLPEVKQAFGGRKCGNGYVEEGEECDCGE
PEECTNRCCNATTCTLKPDAVCAHGQCCEDCQLKPPGTACRGSSNSCDLPEFCTGTAPHCPAN
VYLHDGHPCQGVDGYCYNGICQTHEQQCVTLWGPGAKPAPGICFERVNSAGDPYGNCGKDSK
SAFAKCELRDAKCGKIQCQGGASRPVIGTNAVSIETNIPQQEGGRILCRGTHVYLGDDMPDPGL
VLAGTKCAEGKICLNRRCQNISVFGVHKCAMQCHGRGVCNNRKNCHCEAHWAPPFCDKFGFG
GSTDSGPIRQADNQGLTVGILVSILCLLAAGFVVYLKRKTLMRLLFTHKKTTMEKLRCVHPSRT
PSGPHLGQAHHTPGKGLLMNRAPHFNTPKDRHSLKCQNMDISRPLDARAVPQLQSPQRVLLP
LHQTPRAPSGPARPLPASPAVRQAQGIRKPSPPQKPLPADPLSRTSRLTSALVRTPGQQEPGHR
PAPIRPAPKHQVPRPSHNAYIK

### Mouse Anti-ADAM12 Clone 6E6:

Mouse anti-ADAM12 Clone 6E6 Heavy chain variable (also referred to as 6E6 VH)
Protein Sequence:
(SEQ ID NO: 111)

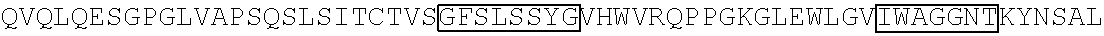

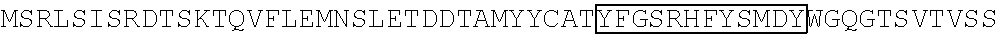

DNA Sequence:
(SEQ ID NO: 211)
CAGGTGCAGCTGCAGGAGTCTGGACCCGGCCTGGTGGCGCCCTCACAGAGCCTGTCCATCACT

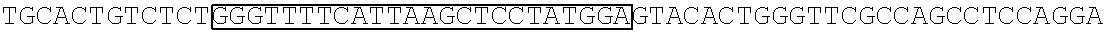

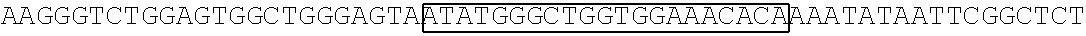

CATGTCCAGACTGAGCATCAGCAGAGACACCTCCAAGACCCAAGTCTTCTTAGAAATGAATAG

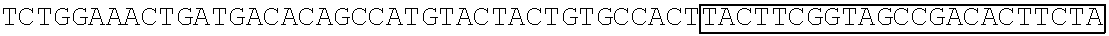

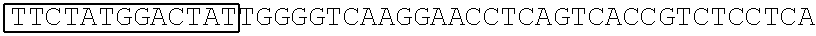

Mouse anti-ADAM12 6E6 VH CDR 1 (6E6 CDR-H1)
Protein Sequence:
(SEQ ID NO: 112)

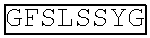

DNA Sequence:
(SEQ ID NO: 212)

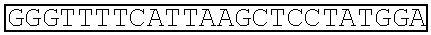

Mouse anti-ADAM12 6E6 VH CDR 2 (6E6 CDR-H2)
Protein Sequence:
(SEQ ID NO: 113)

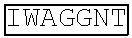

DNA Sequence:
(SEQ ID NO: 213)

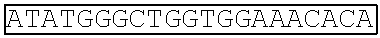

Mouse anti-ADAM12 6E6 VH CDR 3 (6E6 CDR-H3)
Protein Sequence:
(SEQ ID NO: 114)

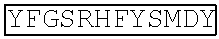

DNA Sequence:
(SEQ ID NO: 214)

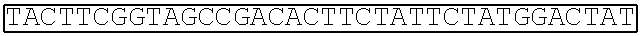

Mouse anti-ADAM12 Clont 6E6 Light chain variable (6E6 VL)
Protein Sequence:
(SEQ ID NO: 115)

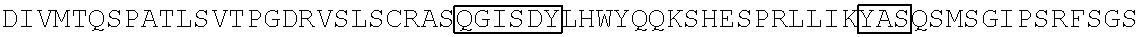

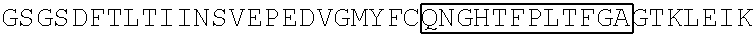

DNA Sequence:
(SEQ ID NO: 215)
GACATTGTGATGACTCAGTCTCCAGCCACCCTGTCTGTGACTCCAGGAGATAGAGTCTCTCTT

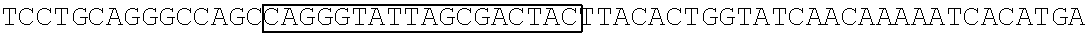

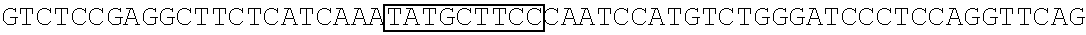

TGGCAGTGGATCAGGGTCAGATTTCACTCTCACTATCAACAGTGTGGAACCTGAAGATGTTG

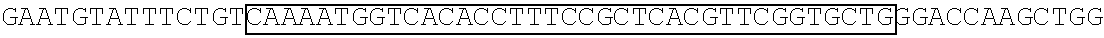

AAATAAAAC
Mouse anti-ADAM12 6E6 VL CDR 1 (6E6 CDR-L1)
Protein Sequence:
(SEQ ID NO: 116)

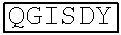

DNA Sequence:
(SEQ ID NO: 216)

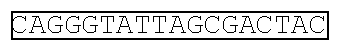

Mouse anti-ADAM12 6E6 VL CDR 2 (6E6 CDR-L2)
Protein Sequcnce:
(SEQ ID NO: 217)

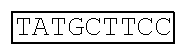

Mouse anti-ADAM12 6E6 VL CDR 3 (6E6 CDR-L3)
Protein Sequence:
(SEQ ID NO: 118)

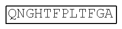

DNA Sequence:
(SEQ ID NO: 218)

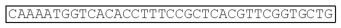

### Mouse Anti-ADAM12 Clone 6C10:

Mouse anti-ADAM12 Clone 6C10 Heavy chain variable (6C10 VH)
Protein Sequence:
(SEQ ID NO: 121)

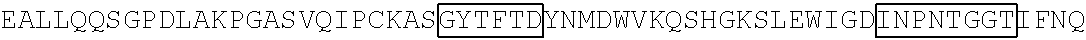

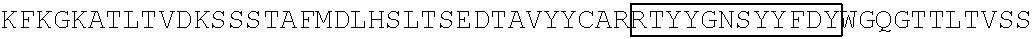

DNA Sequence:
(SEQ ID NO: 221)
GAGGCCCTGCTGCAGCAGTCTGGACCTGATCTGGCGAAGCCTGGGGCTTCAGTACAAA

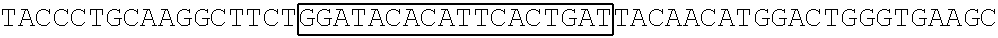

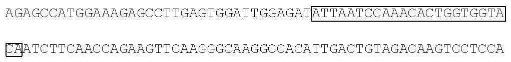

GCACAGCCTTCATGGACCTCCACAGCCTGACATCTGAGGACACTGCAGTCTATTACTG

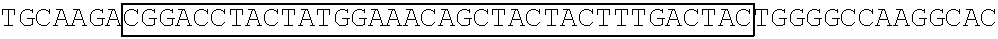

CACTCTCACAGTCTCCTCA
Mouse anti-ADAM12 6C10 VH CDR 1 (6C10 CDR-H1)
Protein Sequence:
(SEQ ID NO: 122)

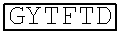

DNA Sequence:
(SEQ ID NO: 222)

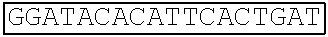

Mouse anti-ADAM12 6C10 VH CDR 2 (6C10 CDR-H2)
Protein Sequence:
(SEQ ID NO: 123)

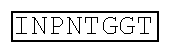

DNA Sequence:
(SEQ ID NO: 223)

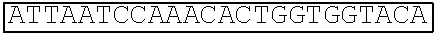

Mouse anti-ADAM12 6C10 VH CDR 3 (6C10 CDR-H3)
Protein Sequence:
(SEQ ID NO: 124)

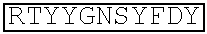

DNA Sequence:
(SEQ ID NO: 224)

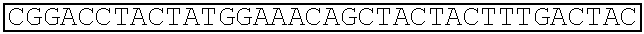

Mouse anti-ADAM12 Clone 6C10 Light chain variable (6C10 VL)
Protein Sequence:
(SEQ ID NO: 125)

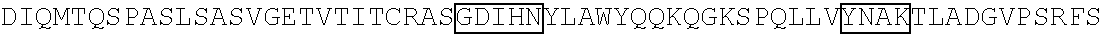

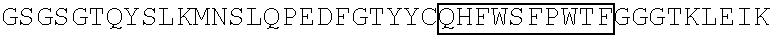

DNA Sequence:
(SEQ ID NO: 225)
GACATCCAGATGACTCAGTCTCCAGCCTCCCTATCTGCATCTGTGGGAGAAACTGTCA

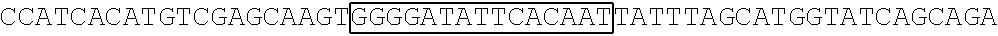

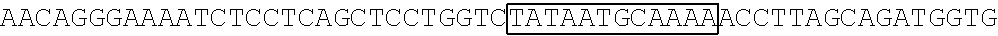

TGCCATCAAGGTTCAGTGGCAGTGGATCAGGAACACAATATTCTCTCAAGATGAACA

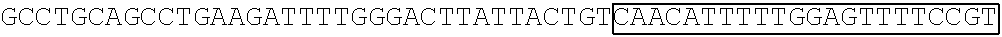

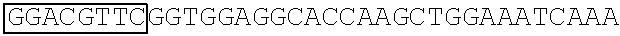

Mouse anti-ADAM12 6C10 VL CDR 1 (6C10 CDR-L1)
Protein Sequence:
(SEQ ID NO: 126)

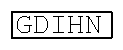

DNA Sequence:
(SEQ ID NO: 226)

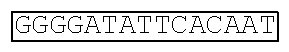

Mouse anti-ADAM12 6C10 VL CDR 2 (6C10 CDR-L2)
Protein Sequence:
(SEQ ID NO: 127)

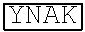

DNA Sequence:
(SEQ ID NO: 227)

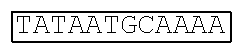

Mouse anti-ADAM12 6C10 VL CDR 3 (6C10 CDR-L3)
Protein Sequence:
(SEQ ID NO: 128)

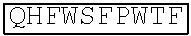

DNA Sequence:
(SEQ ID NO: 228)

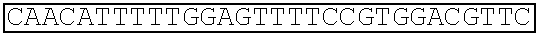

Humanized Anti-ADAM12 Clone 6E6 (h6E6):

Note: Boxed sequences correspond to CDRs (CDR1, CDR 2,
and CDR 3, in the order of appearance)
Humanized anti-ADAM12 Clone 6E6 Heavy chain variable (h6E6 VH)
Protein Sequence:
(SEQ ID NO: 131)

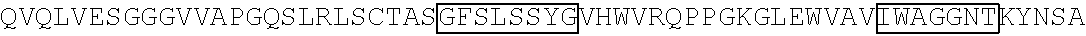

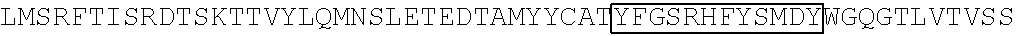

DNA Sequence:
(SEQ ID NO: 231)
CAGGTGCAGCTGGTGGAGAGCGGAGGAGGTGTAGTTGCTCCTGGACAGAGCCTGAGACTGAG

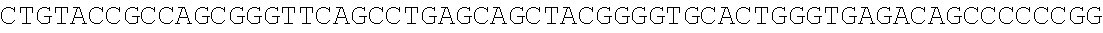

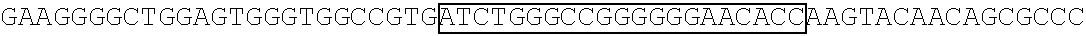

TGATGAGCAGATTCACCATCAGCAGAGATACCAGCAAGACCACCGTGTACCTGCAGATGAACA

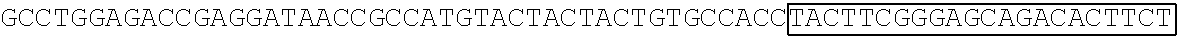

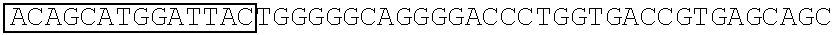

Humanized anti-ADAM12 6E6 VH CDR 1 (h6E6 CDR-H1)
Protein Sequence:
(SEQ ID NO: 132)

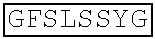

DNA Sequence:
(SEQ ID NO: 232)

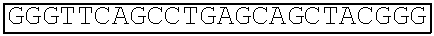

Humanized anti-ADAM12 6E6 VH CDR 2 (h6E6 CDR-H2)
Protein Sequence:
(SEQ ID NO: 133)

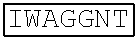

DNA Sequence:
(SEQ ID NO: 233)

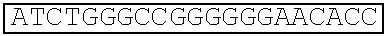

Humanized anti-ADAM12 6E6 VH CDR 3 (h6E6 CDR-H3)
Protein Sequence:
(SEQ ID NO: 134)

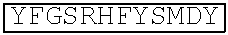

DNA Sequence:
(SEQ ID NO: 234)

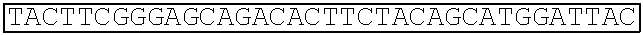

Humanized anti-ADAM12 Clone 6E6 Light chain variable (h6E6 VL)
Protein Sequence:
(SEQ ID NO: 135)

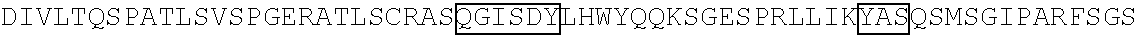

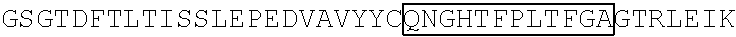

DNA Sequence:
(SEQ ID NO: 235)
GATATCGTGCTGACCCAGAGCCCCGCCACCCTGAGCGTGAGCCCCCGGGGAGAGAGCCACCCTG

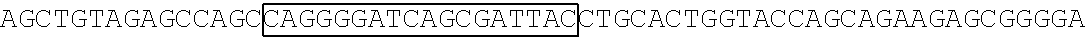

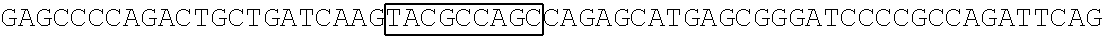

CGGGAGCGGGAGCGGGACCGATTTCACCCTGACCATCAGCAGCCTGGAGCCCGAGGATGTGGC

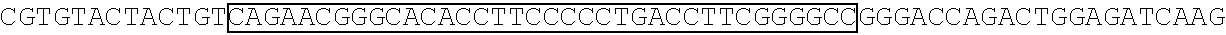

GATCAAG
Humanized anti-ADAM12 6E6 VL CDR 1 (h6E6 CDR-L1)
Protein Sequence:
(SEQ ID NO: 136)

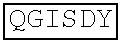

DNA Sequence:
(SEQ ID NO: 236)

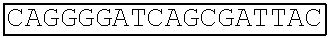

Humanized znti-ADAM12 6E6 VL CDR 2 (h6E6 CDR-L2)
Protein Sequence:
(SEQ ID NO: 137)

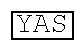

DNA Sequence:
(SEQ ID NO: 237)

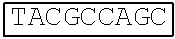

Humanized anti-ADAM12 6E6 VL CDR 3 (h6E6 CDR-L3)
Protein Sequence:
(SEQ ID NO: 138)

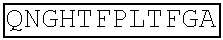

DNA Sequence:
(SEQ ID NO: 238)

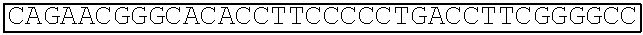

Humanized anti-ADAM12 6E6 scFv VH-G4S3-VL (h6E6 scFv HL, or h6E6scFvHL)
Protein Sequence:
(SEQ ID NO: 139)

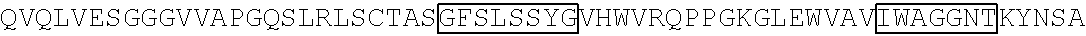

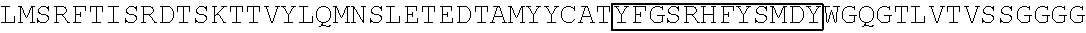

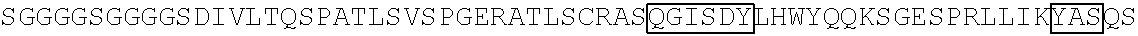

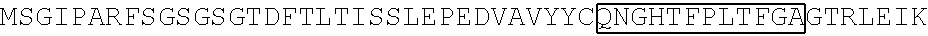

DNA Sequence:
(SEQ ID NO: 239)
CAGGTGCAGCTGGTGGAGAGCGGAGGAGGTGTAGTTGCTCCTGGACAGAGCCTGAGACTGAG

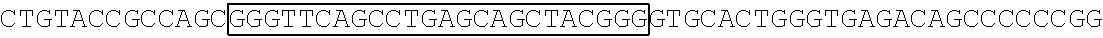

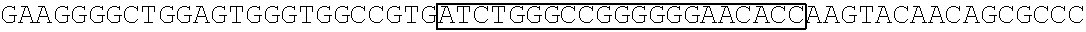

TGATGAGCAGATTCACCATCAGCAGAGATACCAGCAAGACCACCGTGTACCTGCAGATGAACA

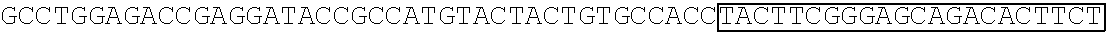

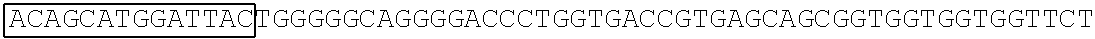

GGCGGCGGCGGCTCCGGTGGTGGTGGTTCCGATATCGTGCTGACCCAGAGCCCCGCCACCCTG

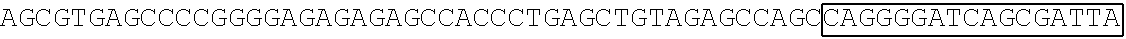

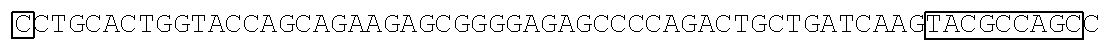

AGAGCATGAGCGGGATCCCCGCCAGATTCAGCGGAGCGGGAGCGGGACCGATTTCACCCTGA

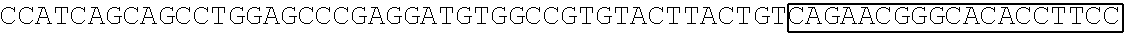

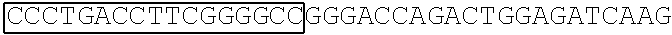

Humanized anti-ADAM12 6E6 scFv VL-G4S3-VH (h6E6 scFv LH, or H6E6 scFvLH)
Protein Sequence:
(SEQ ID NO: 140)

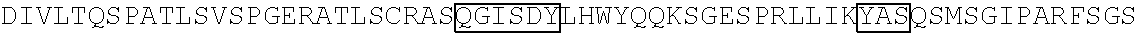

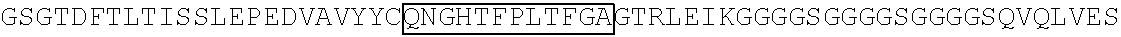

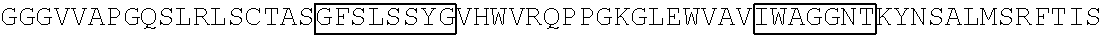

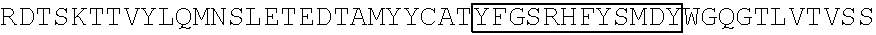

DNA Sequence:
(SEQ ID NO: 240)
GATATCGTGCTGACCCAGAGCCCCGCCACCCTGAGCGTGAGCCCCGGGGAGAGAGCCACCCTG

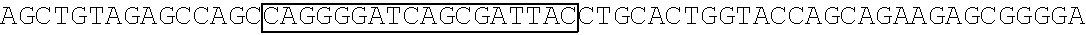

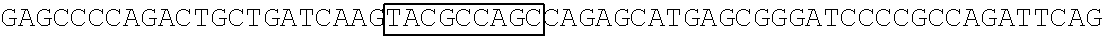

CGGGAGCGGGAGCGGGACCGATTTCACCCTGACCATCAGCAGCCTGGAGCCCGAGGATGTGGC

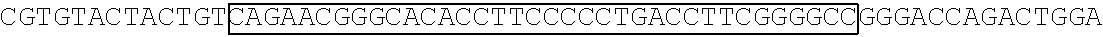

GATCAAGGGTGGTGGTGGTTCTGGCGGCGGCGGCTCCGGTGGTGGTGGTTCCCAGGTGCAGCT
GGTGGAGAGCGGAGGAGGTGTAGTTGCTCCTGGACAGAGCCTGAGACTGAGCTGTACCGCCA

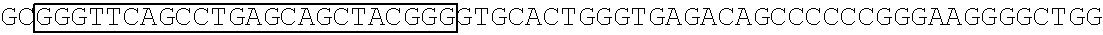

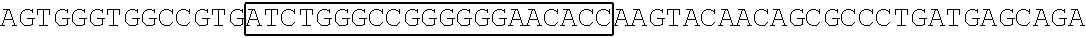

TTCACCATCAGCAGAGATACCAGCAAGACCACCGTGTACCTGCAGATGAACAGCCTGGAGACC

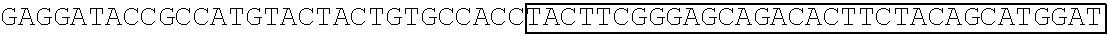

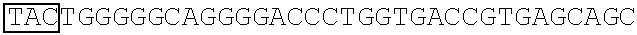

Humanized Anti-ADAM12 Clone 6C10 (h6C10):

Humanized anti-ADAM12 Clone 6C10 Heavy chain variable (h6C10 VH)
Protein Sequence:
(SEQ ID NO: 141)

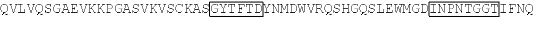

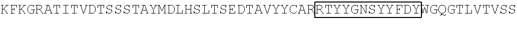

DNA Sequence:
(SEQ ID NO: 241)
CAGGTGCTGGTGCAGAGCGGGGCCGAGGTGAAGAAGCCCGGGGCCAGCGTGAAGGTGAGCTG

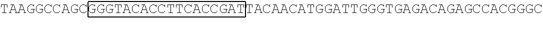

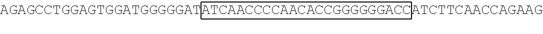

TTCAAGGGGAGAGCCACCATCACCGTGGATACCAGCAGCAGCACCGCCTACATGGATCTGCAC

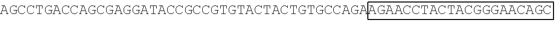

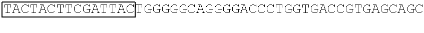

Humanized anti-ADAM12 6C10 VH CDR 1 (h6C10 CDR-H1)
Protein Sequence:
(SEQ ID NO: 142)

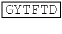

DNA Sequence:
(SEQ ID NO: 242)

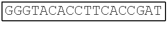

Humanized anti-ADAM12 6C10 VH CDR 2 (h6C10 CDR-H2)
(SEQ ID NO: 143)

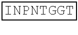

Protein Sequence:
(SEQ ID NO: 243)

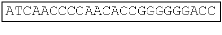

Humanized anti-ADAM12 6C10 VH CDR 3 (h6C10 CDR-H3)
Protein Sequence:
(SEQ ID NO: 144)

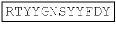

DNA Sequence:
(SEQ ID NO: 244)

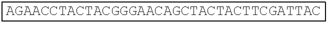

Humanized anti-ADAM12 Clone 6C10 Light chain variable (h6C10 VL)
Protein Sequence:
(SEQ ID NO: 145)

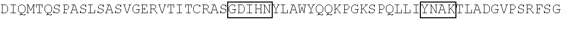

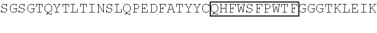

DNA Sequence:
(SEQ ID NO: 245)
GATATCCAGATGACCCAGAGCCCCGCCAGCCTGAGCGCCAGCGTGGGGAGAGAGTGACCATC

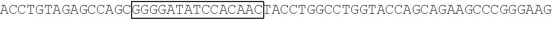

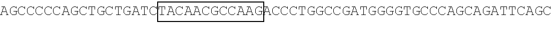

GGGAGCGGGAGCGGGACCCAGTACACCCTGACCATCAACAGCCTGCAGCCCGAGGATTTCGCC

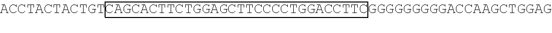

ATCAAG
Humanized anti-ADAM12 6C10 VL CDR 1 (h6C10 CDR-L1)
Protein Sequence:
(SEQ ID NO: 146)

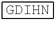

DNA Sequence:
(SEQ ID NO: 246)

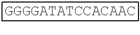

Humanized anti-ADAM12 6C10 VL CDR 2 (h6C10 CDR-L2)
Protein Sequence:
(SEQ ID NO: 147)

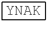

DNA Sequence:
(SEQ ID NO: 247)

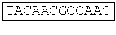

Humanized anti-ADAM12 6C10 VL CDR 3 (h6C10 CDR-L3)
Protein Sequence:
(SEQ ID NO: 148)

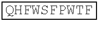

DNA Sequence:
(SEQ ID NO: 248)

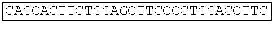

Humanized anti-ADAM12 6C10 scFv VH-G4S3-VL (h6C10 scFv HL,
or h6C10scFvHL)
Protein Sequence:
(SEQ ID NO: 149)

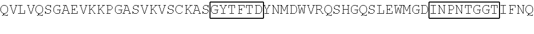

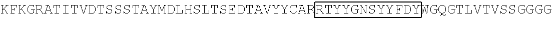

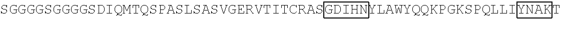

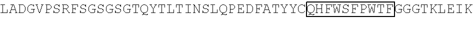

DNA Sequence:
(SEQ ID NO: 249)
CAGGTGCTGGTGCAGAGCGGGGCCGAGGTGAAGAAGCCCGGGGCCAGCGTGAAGGTGAGCTG

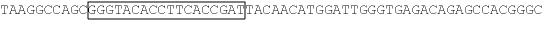

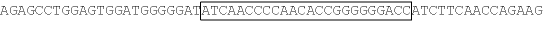

TTCAAGGGGAGAGCCACCATCACCGTGGATACCAGCAGCAGCACCGCCTACATGGATCTGCAC

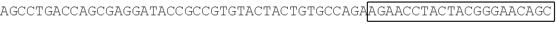

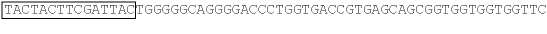

TGGCGGCGGCGGCTCCGGTGGTGGTGGTTCCGATATCCAGAATGACCCAGAGCCCCGCCAGCCT

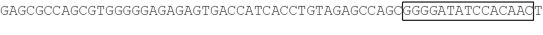

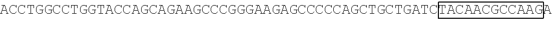

CCCTGGCCGATGGGGTGCCCAGCAGATTCAGCGGGAGCGGGAGCGGGACCCAGTACACCCTGA

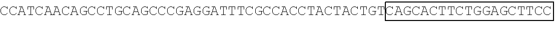

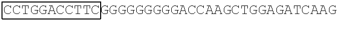

Humanized anti-ADAM12 6C10 scFv VL-G4S3-VH (h6C10 scFv, LH,
or h6C10scFvLH)
Protein Sequence:
(SEQ ID NO: 150)

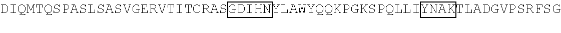

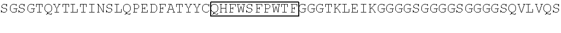

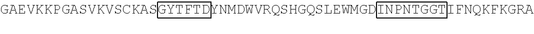

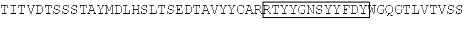

DNA Sequence:
(SEQ ID NO: 250)
GATATCCAGATGACCCAGAGCCCCGCCAGCCTGAGCGCCAGCGTGGGGAGAGAGTGACCATC

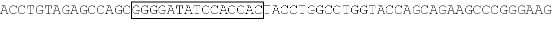

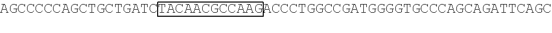

GGGAGCGGGAGCGGGACCCAGTACACCCTGACCATCAACAGCCTGCAGCCCGAGGATTTCGCC

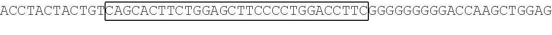

ATCAAGGGTGGTGGTGGTTCTGGCGGCGGCGGCTCCGGTGGTGGTGGTTCCCAGGTGCTGGTG

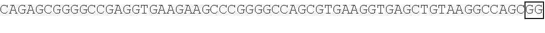

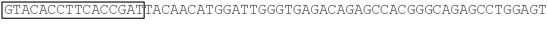

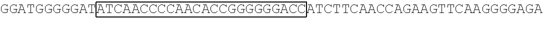

GCCACCATCACCGTGGATACCAGCAGCAGCACCGCCTACATGGATCTGCACAGCCTGACCAGC

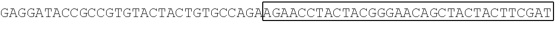

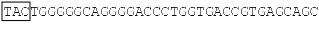

### Physiological Substrates of ADAM12:

Human alpha actinin 2 (ACTN2)
(SEQ ID NO: 151) Protein Sequence (NCBI Reference Sequence: NP_001094.1):
MNQIEPGVQYNYVYDEDEYMIQEEEWDRDLLLDPAWEKQQRKTFTAWCNSHLRKAGTQIEN
IEEDFRNGLKLMLLLEVISGERLPKPDRGKMRFHKIANVNKALDYIASKGVKLVSIGAEEIVDGN
VKMTLGMIWTIILRFAIQDISVEETSAKEGLLLWCQRKTAPYRNVNIQNFHTSWKDGLGLCALI
HRHRPDLIDYSKLNKDDPIGNINLAMEIAEKHLDIPKMLDAEDIVNTPKPDERAIMTYVSCFYH
AFAGAEQAETAANRICKVLAVNQENERLMEEYERLASELLEWIRRTIPWLENRTPEKTMQAM
QKKLEDFRDYRRKHKPPKVQEKCQLEINFNTLQTKLRISNRPAFMPSEGKMVSDIAGAWQRL
EQAEKGYEEWLLNEIRRLERLEHLAEKFRQKASTHETWAYGKEQILLQKDYESASLTEVRALL
RKHEAFESDLAAHQDRVEQIAAIAQELNELDYHDAVNVNDRCQKICDQWDRLGTLTQKRREA
LERMEKLLETIDQLHLEFAKRAAPFNNWMEGAMEDLQDMFIVHSIEEIQSLITAHEQFKATLP
EADGERQSIMAIQNEVEKVIQSYNIRISSSNPYSTVTMDELRTKWDKVKQLVPIRDQSLQEELA
RQHANERLRRQFAAQANAIGPWIQNKMEEIARSSIQITGALEDQMNQLKQYEHNIINYKNNID
KLEGDHQLIQEALVFDNKHTNYTMEHIRVGWELLLTTIARTINEVETQILTRDAKGITQEQMN
EFRASFNHFDRRKNGLMDHEDFRACLISMGYDLGEAEFARIMTLVDPNGQGTVTFQSFIDFMT
RETADTDTAEQVIASFRILASDKPYILAEELRRELPPDQAQYCIKRMPAYSGPGSVPGALDYAAF
SSALYGESDL
Human insulin-like growth factor-binding protein 3 (IGFBP3)
(SEQ ID NO: 152) Protein Sequence (NCBI Reference Sequence: NP_000589.2):
MQRARPTLWAAALTLLVLLRGPPVARAGASSAGLGPVVRCEPCDARALAQCAPPPAVCAELV
REPGCGCCLTCALSEGQPCGIYTERCGSGLRCQPSPDEARPLQALLDGRGLCVNASAVSRLRAYL
LPAPPAPGNASESEEDRSAGSVESPSVSSTHRVSDPKFHPLHSKIIIIKKGHAKDSQRYKVDYES
QSTDTQNFSSESKRETEYGPCRREMEDTLNHLKFLNVLSPRGVHIPNCDKKGFYKKKQCRPSK
GRKRGFCWCVDKYGQPLPGYTTKGKEDVHCYSMQSK
Human insulin-like growth factor-binding protein 5 (IGFBP5)
(SEQ ID NO: 153) Protein Sequence (NCBI Reference Sequence: NP_000590.1):
MVLLTAVLLLLAAYAGPAQSLGSFVHCEPCDEKALSMCPPSPLGCELVKEPGCGCCMTCALAEG
QSCGVYTERCAQGLRCLPRQDEEKPLHALLHGRGVCLNEKSYREQVKIERDSREHEEPTTSEM
AEETYSPKIFRPKHTRISELKAEAVKKDRRKKLTQSKFVGGAENTAHPRIISAPEMRQESEQGP
CRRHMEASLQELKASPRMVPRAVYLPNCDRKGFYKRKQCKPSRGRKRGICWCVDKYGMKLPG
MEYVDGDFQCHTFDSSNVE
Human phosphatidylinositol 3 kinase regulatory subunit alpha (PIK3R1)
(SEQ ID NO: 154) Protein Sequence (NCBI Reference Sequence: NP_852664.1):
MSAEGYQYRALYDYKKEREEDIDLHLGDILTVNKGSLVALGFSDGQEARPEEIGWLNGYNETT
GERGDFPGTYVEYIGRKKISPPTPKPRPPRPLPVAPGSSKTEADVEQQALTLPDLAEQFAPPDIA
PPLLIKLVEAIEKKGLECSTLYRTQSSSNLAELRQLLDCDTPSVDLEMIDVHVLADAFKRYLLDL
PNPVIPAAVYSEMISLAPEVQSSEEYIQLLKKLIRSPSIPHQYWLTLQYLLKHFFKLSQTSSKNLL
NARVLSEIFSPMLFRFSAASSDNTENLIKVIEILISTEWNERQPAPALPPKPPKPTTVANNGMN
NNMSLQDAEWYWGDISREEVNEKLRDTADGTFLVRDASTKMHGDYTLTLRKGGNNKLIKIF
HRDGKYGFSDPLTFSSVVELINHYRNESLAQYNPKLDVKLLYPVSKYQQDQVVKEDNIEAVGK
KLHEYNTQFQEKSREYDRLYEEYTRTSQEIQMKRTAIEAFNETIKIFEEQCQTQERYSKEYIEKF
KREGNEKEIQRIMHNYDKLKSRISEIIDSRRRLEEDLKKQAAEYREIDKRMNSIKPDLIQLRKTR
DQYLMWLTQKGVRQKKLNEWLGNENTEDQYSLVEDDEDLPHHDEKTWNVGSSNRNKAENL
LRGKRDGTFLVRESSKQGCYACSVVVDGEVKHCVINKTATGYGFAEPYNLYSSLKELVLHYQHT
SLVQHNDSLNVTLAYPVYAQQRR
Human heparin-binding epidermal growth factor (HB-EGF)
(SEQ ID NO: 155) Protein Sequence (NCBI Reference Sequence: NP_001936.1):
MKLLPSVVLKLFLAAVLSALVTGESLERLRRGLAAGTSNPDPPTVSTDQLLPLGGGRDRKVRDL
QEADLDLLRVTLSSKPQALATPNKEEHGKRKKKGKGLGKKRDPCLRKYKDFCIHGECKYVKEL
RAPSCICHPGYHGERCHGLSLPVENRLYTYDHTTILAVVAVVLSSVCLLVIVGLLMFRYHRRGGY
DVENEEKVKLGMTNSH
Human epidermal growth factor (EGF)
(SEQ ID NO: 156) Protein Sequence (GenBank: AFA26280.1):
NSDSECPLSHDGYCLHDGVCMYIEALDKYACNCVVGYIGERCQYRDLKWWELR
Human Betacellulin
(SEQ ID NO: 157) Protein Sequence (GenBank: AAH11618.1):
MDRAARCSGASSLPLLLALALGLVILHCVVADGNSTRSPETNGLLCGDPEENCAATTTQSKRKG
HFSRCPKQYKHYCIKGRCRFVVAEQTPSCVCDEGYIGARCERVDLFYLRGDRGQILVICMIAVM
VVFIILVIGVCTCCHPLRKRRKRKKKEEEMETLGKDITPINEDIEETNIA
Human Betacellulin Delta-like 1
(SEQ ID NO: 158) Protein Sequence (NCBI Reference Sequence: NP_005609.3):
MGSRCALALAVLSALLCQVWSSGVFELKLQEFVNKKGLLGNRNCCRGGAGPPPCACRTFFRVC
LKHYQASVSPEPPCTYGSAVTPVLGVDSFSLPDGGGADSAFSNPIRFPFGFTWPGTFSLIIEALH
TDSPDDLATENPERLISRLATQRHLTVGEEWSQDLHSSGRTDLKYSYRFVCDEHYYGEGCSVF
CRPRDDAFGHFTCGERGEKVCNPGWKGPYCTEPICLPGCDEQHGFCDKPGECKCRVGWQGRY
CDECIRYPGCLHGTCQQPWQCNCQEGWGGLFCNQDLNYCTHHKPCKNGATCTNTGQGSYTCS
CRPGYTGATCELGIDECDPSPCKNGGSCTDLENSYSCTCPPGFYGKICELSAMTCADGPCFNGG
RCSDSPDGGYSCRCPVGYSGFNCEKKIDYCSSSPCSNGAKCVDLGDAYLCRCQAGFSGRHCDDN
VDDCASSPCANGGTCRDGVNDFSCTCPPGYTGRNCSAPVSRCEHAPCHNGATCHERGHRYVCE
CARGYGGPNCQFLLPELPPGPAVVDLTEKLEGQGGPFPWVAVCAGVILVLMLLLGCAAVVVCV
RLRLQKHRPPADPCRGETETMNNLANCQREKDISVSIIGATQIKNTNKKADFHGDHSADKNGF
KARYPAVDYNLVQDLKGDDTAVRDAHSKRDTKCQPQGSSGEEKGTPTTLRGGEASERKRPDS
GCSTSKDTKYQSVYVISEEKDECVIATEV
Human placental leucine aminopeptidase (P-LAP)
(SEQ ID NO: 159) Protein Sequence (NCBI Reference Sequence: NP_005566.2):
MEPFTNDRLQLPRNMIENSMFEEEPDVVDLAKEPCLHPLEPDEVEYEPRGSRLLVRGLGEHE
MEEDEEDYESSAKLLGMSFMNRSSGLRNSATGYRQSPDGACSVPSARTMVVCAFVIVVAVSVI
MVIYLLPRCTFTKEGCHKKNQSIGLIQPFATNGKLFPWAQIRLPTAVVPLRYELSLHPNLTSMT
FRGSVTISVQALQVTWNIILHSTGHNISRVTFMSAVSSQEKQAEILEYAYHGQIAIVAPEALLAG
HNYTLKIEYSANISSSYYGFYGFSYTDESNEKKYFAATQFEPLAARSAFPCFDEPAFKATFIIKIIR
DEQYTALSNMPKKSSVVLDDGLVQDEFSESVKMSTYLVAFIVGEMKNLSQDVNGTLVSIYAVP
EKIGQVHYALETTVKLLEFFQNYFEIQYPLKKLDLVAIPDFEAGAMENWGLLTFREETLLYDSN
TSSMADRKLVTKIIAHELAHQWFGNLVTMKWWNDLWLNEGFATFMEYFSLEKIFKELSSYED
FLDARFKTMKKDSLNSSHPISSSVQSSEQIEEMFDSLSYFKGSSLLLMLKTYLSEDVFQHAVVLY
LHNHSYASIQSDDLWDSFNEVTNQTLDVKRMMKTWTLQKGFPLVTVQKKGKELFIQQERFFL
NMKPEIQPSDTSYLWHIPLSYVTEGRNYSKYQSVSLLDKKSGVINLTEEVLWVKVNINMNGYYI
VHYADDDWEALIHQLKINPYVLSDKDRANLINNIFELAGLGKVPLKRAFDLINYLGNENHTAPI
TEALFQTDLIYNLLEKLGYMDLASRLVTRVFKLLQNQIQQQTWTDEGTPSMRELRSALLEFAC
THNLGNCSTTAMKLFDDWMASNGTQSLPTDVMTTVFKVGAKTDKGWSFLLGKYISIGSEAEK
NKILEALASSEDVRKLYWLMKSSLNGDNFRTQKLSFIIRTVGRHFPGHLLAWDFVKENWNKL
VQKFPLGSYTIQNIVAGSTYLFSTKTHLSEVQAFFENQSEATFRLRCVQEALEVIQLNIQWMEK
NLKSLTWWL

### CAR Subparts:

Lead sequence (LS)
Protein Sequence:
(SEQ ID NO: 160)

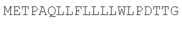

DNA Sequence:
(SEQ ID NO: 260)
ATGGAAACCCCAGCGCAGCTTCTCTTCCTCCTGCTACTCTGGCTCCCAGATACCACCGGA
Human CD28 transmembrane domain (CD28 TM domain, or CD28TM)
Protein Sequence:
(SEQ ID NO: 161)
FWVLVVVGGVLACYSLLVTVAFIIFW
DNA Sequence:
(SEQ ID NO: 261)
TTTTGGGTGCTGGTGGTGGTTGGTGGAGTCCTGGCTTGCTATAGCTTGCTAGTAACAGTGGCC
TTTATTATTTTCTGGGTG
Human CD3 zeta intracellular signaling domain (CD3z ICS domain,
CD3 ζ ICS, or CD3zICS)
Protein Sequence:
(SEQ ID NO: 162)
RVKFSRSADAPAYQQGQNQLYNELNLGRREEYDVLDKRRGRDPEMGGKPQRRKNPQEGLYN
ELQKDKMAEAYSEIGMKGERRRGKGHDGLYQGLSTATKDTYDALHMQALPPR
DNA Sequence:
(SEQ ID NO: 262)
AGAGTGAAGTTCAGCAGGAGCGCAGACGCCCCCGCGTACCAGCAGGGCCAGAACCAGCTCTAT
AACGAGCTCAATCTAGGACGAAGAGAGGAGTACGATGTTTTGGACAAGAGACGTGGCCGGGA
CCCTGAGATGGGGGGAAAGCCGCAGAGAAGGAAGAACCCTCAGGAAGGCCTGTACAATGAAC
TGCAGAAAGATAAGATGGCGGAGGCCTACAGTGAGATTGGGATGAAAGGCGAGCGCCGGAGG
GGCAAGGGGCACGATGGCCTTTACCAGGGTCTCAGTACAGCCACCAAGGACACCTACGACGCC
CTTCACATGCAGGCCCTGCCCCCTCGC
Human CD28 hinge (CD28 hinge, or CD28H)
Protein Sequence:
(SEQ ID NO: 163)
LEVKGKHLCPSPLFPGPSKP
DNA Sequence:
(SEQ ID NO: 263)
CTCCAGGTGAAAGGGAAACACCTTTGTCCAAGTCCCCTATTTCCCGGACCTTCTAAGCCC
Human CD28 costimulatory domain (CD28 CS domain, or CD28CS)
Protein Sequence:
(SEQ ID NO: 164)
RSKRSRLLHSDYMNMTPRRPGPTRKHYQPYAPPRDFAAYRSKL
DNA Sequence:
(SEQ ID NO: 264)
AGGAGTAAGAGGAGCAGGCTCCTGCACAGTGACTACATGAACATGACTCCCCGCCGCCCCGGG
CCCACCCGCAAGCATTACCAGCCCTATGCCCCACCACGCGACTTCGCAGCCTATCGCTCCAAGC
TT
Human 4-1BB costimulatory domain (4-1BB CS domain, 41BB CS domain, or 41BBCS)
Protein Sequence:
(SEQ ID NO: 165)
KRGRKKLLYIFKQPFMRPVQTTQEEDGCSCRFPEEEEGGCEL
DNA Sequence:
(SEQ ID NO: 265)
AAACGGGGCAGAAAGAAACTCCTGTATATATTCAAACAACCATTTATGAGACCAGTACAAAC
TACTCAAGAGGAAGATGGCTGTAGCTGCCGATTTCCAGAAGAAGAAGAAGGAGGATGTGAAC
TG
Human DAP10 costimulatory domain (DAP10 CS domain, or DAP10CS)
Protein Sequence:
(SEQ ID NO: 166)
LCARPRRSPAQEDGKVYINMPGRG
DNA Sequence:
(SEQ ID NO: 266)
CTGTGCGCACGCCCACGCCGCAGCCCCGCCCAAGAAGATGGCAAAGTCTACATCAACATGCCA
GGCAGGGGC
Glycine-Serine linker unite (Gly-Gly-Gly-Gly-Ser linker unit,
or GS linker unit)
Protein Sequence:
(SEQ ID NO: 167)
GGGGS
DNA Sequence:
(SEQ ID NO: 267)
GGTGGTGGTGGTTCT
(Gly-Gly-Gly-Gly-Ser)X3 linker ((Gly4Ser)3 linker, (G4S)3 linker, G4S X3
linker, or G4SX3 linker)
Protein Sequence:
(SEQ ID NO: 168)
GGGGSGGGGSGGGGS
DNA Sequence:
(SEQ ID NO: 268)
GGTGGTGGTGGTTCTGGCGGCGGCGGCTCCGGTGGTGGTGGTTCC
T2A ribosomal skip sequence (T2A)
Protein Sequence:
(SEQ ID NO: 169)
AGAKRSGSGEGRGSLLTCGDVEENPGPR
DNA Sequence:
(SEQ ID NO: 269)
GCCGGCGCCAAAAGGTCTGGCTCCGGTGAGGGCAGAGGAAGTCTTCTAACATGCGGTGACGTG
GAGGAGAATCCCGGCCCTAGA
truncated CD19 (trCD19)
Protein Sequence:
(SEQ ID NO: 170)
MPPPRLLFFLLFLTPMEVRPEEPLVVKVEEGDNAVLQCLKGTSDGPTQQLTWSRESPLKPFLK
LSLGLPGLGIHMRPLAIWLFIFNVSQQMGGFYLCQPGPPSEKAWQPGWTVNVEGSGELFRWN
VSDLGGLGCGLKNRSSEGPSSPSGKLMSPKLYWAKDRPEIWEGEPPCLPPRDSLNQSLSQDLT
MAPGSTLWLSCGVPPDSVSRGPLSWTHVHPKGPKSLLSLELKDDRPARDMWVMETGLLLPR
ATAQDAGKYYCHRGNLTMSFHLEITARPVLWHWLLRTGGWKVSAVTLAYIFCLCSLVGILHL
QRALVLRRKRKRMT
DNA Sequence:
(SEQ ID NO: 270)
ATGCCACCTCCTCGCCTCCTCTTCTTCCTCCTCTTCCTCACCCCCATGGAAGTCAGGCCCGAGG
AACCTCTAGTGGTGAAGGTGGAAGAGGGAGATAACGCTGTGCTGCAGTGCCTCAAGGGGACC
TCAGATGGCCCCACTCAGCAGCTGACCTGGTCTCGGGAGTCCCCGCTTAAACCCTTCTTAAAA
CTCAGCCTGGGGCTGCCAGGCCTGGGAATCCACATGAGGCCCCTGGCCATCTGGCTTTTCATC
TTCAACGTCTCTCAACAGATGGGGGGCTTCTACCTGTGCCAGCCGGGGCCCCCCTCTGAGAAG
GCCTGGCAGCCTGGCTGGACAGTCAATGTGGAGGGCAGCGGGGAGCTGTTCCCGGTGGAATGTT
TCGGACCTAGGTGGCCTGGGCTGTGGCCTGAAGAACAGGTCCTCAGAGGGCCCCAGCTCCCCT
TCCGGGAAGCTCATGAGCCCCAAGCTGTATGTGTGGGCCAAAGACCGCCCTGAGATCTGGGAG
GGAGAGCCTCCGTGTCTCCCACCGAGGGACAGCCTGAACCAGAGCCTCAGCCAGGACCTCACC
ATGGCCCCTGGCTCCACACTCTGGCTGTCCTGTGGGGTACCCCCTGACTCTGTGTCCAGGGGCC
CCCTCTCCTGGACCCATGTGCACCCCAAGGGGCCTAAGTCATTGCTGAGCCTAGAGCTGAAGG
ACGATCGCCCGGCCAGAGATATGTGGGTAATGGAGACGGGTCTGTTGTTGCCCCGGGCCACAG
CTCAAGACGCTGGAAAGTATTATTGTCACCGTGGCAACCTGACCATGTCATTCCACCTGGAGA
TCACTGCTCGGCCAGTACTATGGCACTGGCTGCTGAGGACTGGTGGCTGGAAGGTCTCAGCTG
TGACTTTGGCTTATCTGATCTTCTGCCTGTGTTCCCTTGTGGGCATTCTTCATCTTCAAAGAG
CCCTGGTCCTGAGGAGGAAAAGAAAGCGAATGACTTAA

### Anti-ADAM12 CARs:

h6E6scFvHL-CD28H-CD28TM-CD28CS-CD3zICS
Protein Sequence:
(SEQ ID NO: 171)

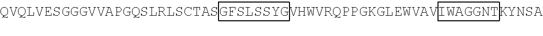

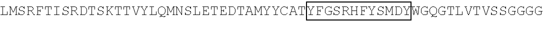

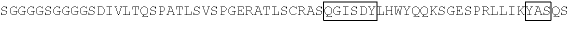

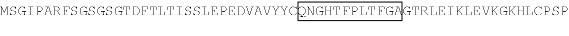

LFPGPSKPFWVLVVVGGVLACYSLLVTVAFIIFWVRSKRSRLLHSDYMNMTPRRPGPTRKHYQ
PYAPPRDFAAYRSKLRVKFSRSADAPAYQQGQNQLYNELNLGRREEYDVLDKRRGRDPEMGG
KPQRRKNPQQGLYNELQKDKMAEAYSEIGMKGERRRGKGHDGLYQGLSTATKDTYDALHMQ
ALPPR
DNA Sequence:
(SEQ ID NO: 271)
CAGGTGCAGCTGGTGGAGAGCGGAGGAGGTGTAGTTGCTCCTGGACAGAGCCTGAGACTGAG

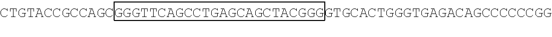

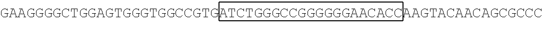

TGATGAGCAGATTCACCATCAGCAGAGATACCAGCAAGACCACCGTGTACCTGCAGATGAACA

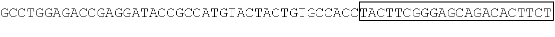

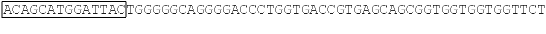

GGCGGCGGCGGCTCCGGTGGTGGTGGTTCCGATATCGTGCTGACCCAGAGCCCCGCCACCCTG

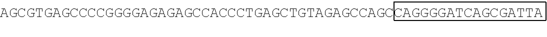

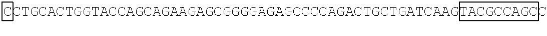

AGAGCATGAGCGGGATCCCCGCCAGATTCAGCGGGAGCGGGAGCGGGACCGATTTCACCCTGA

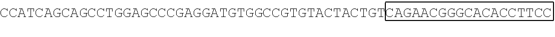

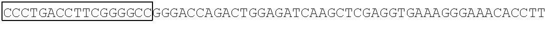

TGTCCAAGTCCCCTATTTCCCGGACCTTCTAAGCCCTTTTGGGTGCTGGTGGTGGTTGGTGGA
GTCCTGGCTTGCTATAGCTTGCTAGTAACAGTGGCCTTTATTATTTTCTGGGTGAGGAGTAAG
AGGAGCAGGCTCCTGCACAGTGACTACATGAACATGACTCCCCGCCGCCCCGGGCCCACCCGC
AAGCATTACCAGCCCTATGCCCCACCACGCGACTTCGCAGCCTATCGCTCCAAGCTTAGAGTG
AAGTTCAGCAGGAGCGCAGACGCCCCCGCGTACCAGCAGGGCCAGAACCAGCTCTATAACGAG
CTCAATCTAGGACGAAGAGAGGAGTACGATGTTTTGGACAAGAGACGTGGCCGGGACCCTGA
GATGGGGGGAAAGCCGCAGAGAAGGAAGAACCCTCAGGAAGGCCTGTACAATGAACTGCAGA
AAGATAAGATGGCGGAGGCCTACAGTGAGATTGGGATGAAAGGCGAGCGCCGGAGGGGCAAG
GGGCACGATGGCCTTTACCAGGGTCTCAGTACAGCCACCAAGGACACCTACGACGCCCTTCAC
ATGCAGGCCCTGCCCCCTCGC
h6E6scFvHL-CD28H-CD28TM-41BBCS-CD3zICS
Protein Sequence:
(SEQ ID NO: 172)

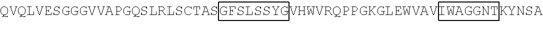

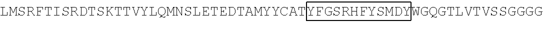

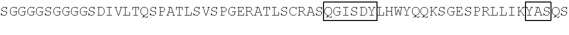

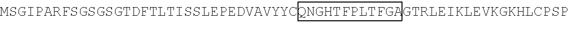

LFPGPSKPFWVLVVVGGVLACYSLLVTVAFIIFWVKRGRKKLLYIFKQPFMRPVQTTQEEDGCS
CRFPEEEEGGCELRVKFSRSADAPAYQQGQNQLYNELNLGRREEYDVLDKRRGRDPEMGGKP
QRRKNPQEGLYNELQKDKMAEAYSEIGMKGERRRGKGHDGLYQGLSTATKDTYDALHMQAL
PPR
DNA Sequence:
(SEQ ID NO: 272)
CAGGTGCAGCTGGTGGAGAGCGGAGGAGGTGTAGTTGCTCCTGGACAGAGCCTGAGACTGAG

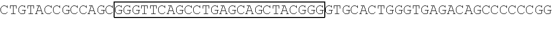

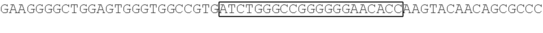

TGATGAGCAGATTCACCATCAGCAGAGATACCAGCAAGACCACCGTGTACCTGCAGATGAACA

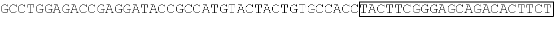

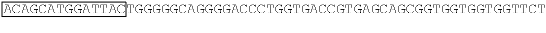

GGCGGCGGCGGCTCCGGTGGTGGTGGTTCCGATATCGTGCTGACCCAGAGCCCCGCCACCCTG

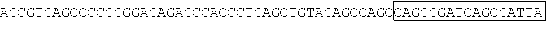

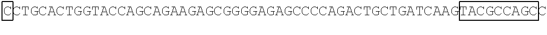

AGAGCATGAGCGGGATCCCCGCCAGATTCAGCGGGAGCGGGAGCGGGACCGATTTCACCCTGA

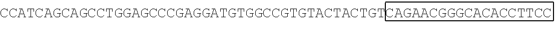

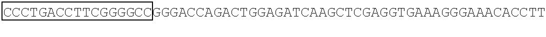

TGTCCAAGTCCCCTATTTCCCGGACCTTCTAAGCCCTTTTGGGTGCTGGTGGTGGTTGGTGGA
GTCCTGGCTTGCTATAGCTTGCTAGTAACAGTGGCCTTTATTATTTTCTGGGTGAAACGGGGC
AGAAAGAAACTCCTGTATATATTCAAACAACCATTTATGAGACCAGTACAAACTACTCAAGA
GGAAGATGGCTGTAGCTGCCGATTTCCAGAAGAAGAAGAAGGAGGATGTGAACTGAGAGTGA
AGTTCAGCAGGAGCGCAGACGCCCCCGCGTACCAGCAGGGCCAGAACCAGCTCTATAACGAGC
TCAATCTAGGACGAAGAGAGGAGTACGATGTTTTGGACAAGAGACGTGGCCGGGACCCTGAG
ATGGGGGGAAAGCCGCAGAGAAGGAAGAACCCTCAGGAAGGCCTGTACAATGAACTGCAGAA
AGATAAGATGGCGGAGGCCTACAGTGAGATTGGGATGAAAGGCGAGCGCCGGAGGGGCAAGG
GGCACGATGGCCTTTACCAGGGTCTCAGTACAGCCACCAAGGACACCTACGACGCCCTTCACA
TGCAGGCCCTGCCCCCTCGC
h6E6scFvHL-CD28H-CD28TM-DAP10CS-CD3zICS
Protein Sequence:
(SEQ ID NO: 173)

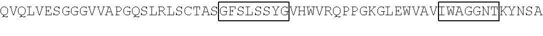

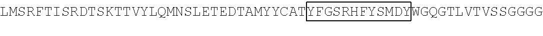

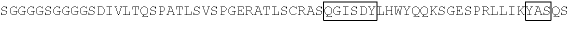

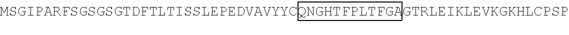

LFPGPSKPFWVLVVVGGVLACYSLLVTVAFIIFWVLCARPRRSPAQEDGKVYINMPGRGRVKFS
RSADAPAYQQGQNQLYNELNLGRREEYDVLDKRRGRDPEMGGKPQRRKNPQEGLYNELQKD
KMAEAYSEIGMKGERRRGKGHDGLYQGLSTATKDTYDALHMQALPPR
DNA Sequence:
(SEQ ID NO: 273)
CAGGTGCAGCTGGTGGAGAGCGGAGGAGGTGTAGTTGCTCCTGGACAGAGCCCTGAGACTGAG

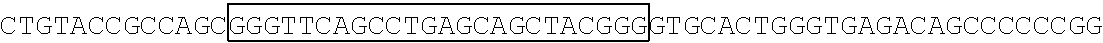

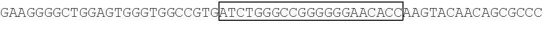

TGATGAGCAGATTCACCATCAGCAGAGATACCAGCAAGACCACCGTGTACCTGCAGATGAACA

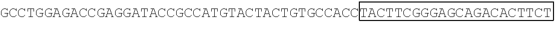

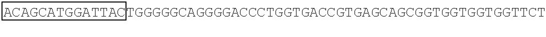

GGCGGCGGCGGCTCCGGTGGTGGTGGTTCCGATATCGTGCTGACCCAGAGCCCCCGCCACCCTG

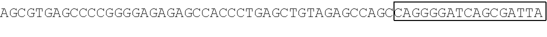

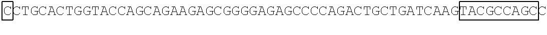

AGAGCATGAGCGGGATCCCCGCCAGATTCAGCGGGAGCGGGAGCGGGACCGATTTCACCCTGA

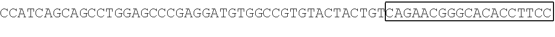

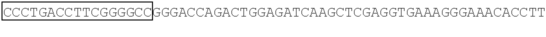

TGTCCAAGTCCCCTATTTCCCGGACCTTCTAAGCCCTTTTGGGTGCTGGTGGTGGTTGGTGGA
GTCCTGGCTTGCTATAGCTTGCTAGTAACAGTGGCCTTTATTATTTTCTGGGTGCTGTGCGCA
CGCCCACGCCGCAGCCCCGCCCAAGAAGATGGCAAAGTCTACATCAACATGCCAGGCAGGGGC
AGAGTGAAGTTCAGCAGGAGCGCAGACGCCCCCGCGTACCAGCAGGGCCAGAACCAGCTCTAT
AACGAGCTCAATCTAGGACGAAGAGAGGAGTACGATGTTTTGGACAAGAGACGTGGCCGGGA
CCCTGAGATGGGGGGAAAGCCGCAGAGAAGGAAGAACCCTCAGGAAGGCCTGTACAATGAAC
TGCAGAAAGATAAGATGGCGGAGGCCTACAGTGAGATTGGGATGAAAGGCGAGCGCCGGAGG
GGCAAGGGGCACGATGGCCTTTACCAGGGTCTCAGTACAGCCACCAAGGACACCTACGACGCC
CTTCACATGCAGGCCCTGCCCCCTCGC
h6E6scFvLH-CD28H-CD28TM-CD28CS-CD3zICS
Protein Sequence:
(SEQ ID NO: 174)

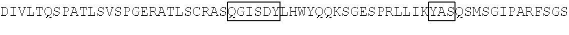

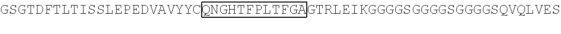

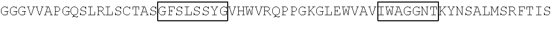

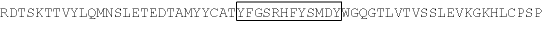

LFPGPSKPFWVLVVVGGVLACYSLLVTVAFIIFWVRSKRSRLLHSDYMNMTPRRPGPTRKHYQ
PYAPPRDFAAYRSKLRVKFSRSADAPAYQQGQNQLYNELNLGREEYDVLDKRRGRDPEMGG
KPQRRKNPQEGLYNELQKDKMAEAYSEIGMKGERRRGKGHDGLYQGLSTATKDTYDALHMQ
ALPPR
DNA Sequence:
(SEQ ID NO: 274)
GATATCGTGCTGACCCAGAGCCCCGCCACCCTGAGCGTGAGCCCCGGGGAGAGAGCCACCCTG

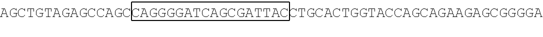

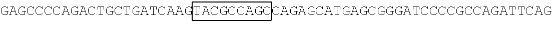

CGGGAGCGGGAGCGGGACCGATTTCACCCTGACCATCAGCAGCCTGGAGCCCGAGGATGTGGC

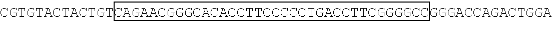

GATCAAGGGTGGTGGTGGTTCTGGCGGCGGCGGCTCCGGTGGTGGTGGTTCCCAGGTGCAGCT
GGTGGAGAGCGGAGGAGGTGTAGTTGCTCCTGGACAGAGCCTGAGACTGAGCTGTACCGCCA

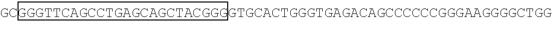

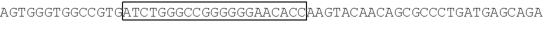

TTCACCATCAGCAGAGATACCAGCAAGACCACCGTGTACCTGCAGATGAACAGCCTGGAGACC

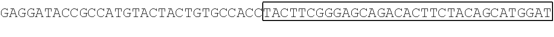

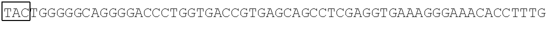

TCCAAGTCCCCTATTTCCCGGACCTTCTAAGCCCTTTTGGGTGCTGGTGGTGGTTGGTGGAGT
CCTGGCTTGCTATAGCTTGCTAGTAACAGTGGCCTTTATTATTTTCTGGGTGAGGAGTAAGA
GGAGCAGGCTCCTGCACAGTGACTACATGAACATGACTCCCCGCCGCCCCGGGCCCACCCGCA
AGCATTACCAGCCCTATGCCCCACCACGCGACTTCGCAGCCTATCGCTCCAAGCTTAGAGTGA
AGTTCAGCAGGAGCGCAGACGCCCCCGCGTACCAGCAGGGCCAGAACCAGCTCTATAACGACG
TCAATCTAGGACGAAGAGAGGAGTACGATGTTTTGGACAAGAGACGTGGCCGGGACCCTGAG
ATGGGGGGAAAGCCGCAGAGAAGGAAGAACCCTCAGGAAGGCCTGTACAATGAACTGCAGAA
AGATAAGATGGCGGAGGCCTACAGTGAGATTGGGATGAAAGGCGAGCGCCGGAGGGGCAAGG
GGCACGATGGCCTTTACCAGGGTCTCAGTACAGCCACCAAGGACACCTACGACGCCCTTCACA
TGCAGGCCCTGCCCCCTCGC
h6E6scFvLH-CD28H-CD28TM-41BBCS-CD3zICS
Protein Sequence:
(SEQ ID NO: 175)

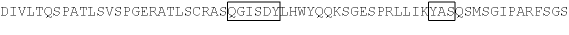

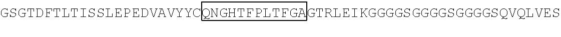

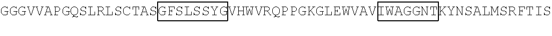

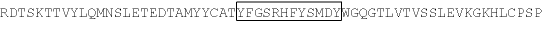

LFPGPSKPFWVLVVVGGVLACYSLLVTVAFIIFWVKRGRKKLLYIFKQPFMRPVQTTQEEDGCS
CRFPEEEEEGGCELRVKFSRSADAPAYQQGQNQLYNELNLGRREEYDVLDKRRGRDPEMGGKP
QRRKNPQEGLYNELQKDKMAEAYSEIGMKGERRRGKGHDGLYQGLSTATKDTYDALHMQAL
PPR
DNA Sequence:
(SEQ ID NO: 275)
GATATCGTGCTGACCCAGAGCCCCGCCACCCTGAGCGTGAGCCCCGGGGAGAGAGCCACCCTG

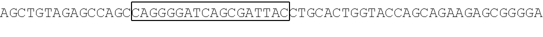

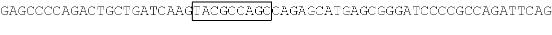

CGGGAGCGGGAGCGGGACCGATTTCACCCTGACCATCAGCAGCCTGGAGCCCGAGGATGTGGC

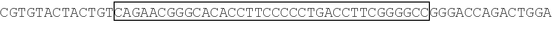

GATCAAGGGTGGTGGTGGTTCTGGCGGCGGCGGCTCCGGTGGTGGTGGTTCCCAGGTGCAGCT
GGTGGAGAGCGGAGGAGGTGTAGTTGCTCCTGGACAGAGCCTGAGACTGAGCTGTACCGCCA

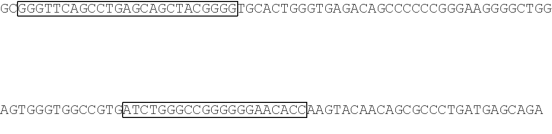

TTCACCATCAGCAGAGATACCAGCAAGACCACCGTGTACCTGCAGATGAACAGCCTGGAGACC

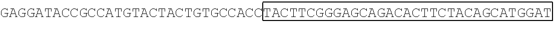

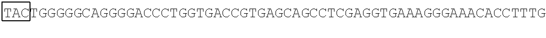

TCCAAGTCCCCTATTTCCCGGACCTTCTAAGCCCTTTTGGGTGCTGGTGGTGGTTGGTGGAGT
CCTGGCTTGCTATAGCTTGCTAGTAACAGTGGCCTTTATTATTTTCTGGGTGAAACGGGGCAG
AAAGAAACTCCTGTATATATTCAAACAACCATTTATGAGACCAGTACAAACTACTCAAGAGG
AAGATGGCTGTAGCTGCCGATTTCCAGAAGAAGAAGAAGGAGGATGTGAACTGAGAGTGAAG
TTCAGCAGGAGCGCAGACGCCCCCGCGTACCAGCAGGGCCAGAACCAGCTCTATAACGAGCTC
AATCTAGGACGAAGAGAGGAGTACGATGTTTTGGACAAGAGACGTGGCCGGGACCCTGAGAT
GGGGGGAAAGCCGCAGAGAAGGAAGAACCCTCAGGAAGGCCTGTACAATGAACTGCAGAAAG
ATAAGATGGCGGAGGCCTACAGTGAGATTGGGATGAAAGGCGAGCGCCGGAGGGGCAAGGGG
CACGATGGCCTTTACCAGGGTCTCAGTACAGCCACCAAGGACACCTACGACGCCCTTCACATG
CAGGCCCTGCCCCCTCGC
h6E6scFvLH-CD28H-CD28TM-DAP10CS-CD3zICS
Protein Sequence:
(SEQ ID NO: 176)

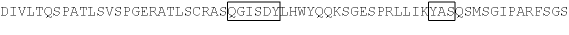

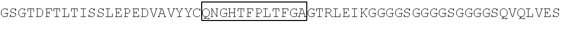

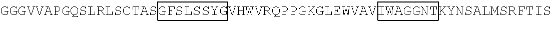

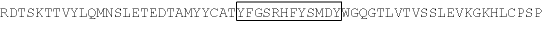

LFPGPSKPFWVLVVVGGVLACYSLLVTVAFIIFWVLCARPRRSPAQEDGKVYINMPGRGRVKFS
RSADAPAYQQGQNQLYNELNLGRREEYDVLDKRRGRDPEMGGKPQRRKNPQEGLYNELQKD
KMAEAYSEIGMKGERRRGKGHDGLYQGLSTATKDTYDALHMQALPPR
DNA Sequence:
(SEQ ID NO: 276)
GATATCGTGCTGACCCAGAGCCCCGCCACCCTGAGCGTGAGCCCCGGGGAGAGAGCCACCCTG

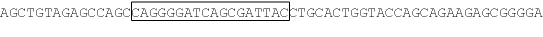

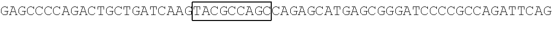

CGGGAGCGGGAGCGGGACCGATTTCACCCTGACCATCAGCAGCCTGGAGCCCGAGGATGTGGC

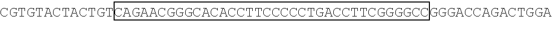

GATCAAGGGTGGTGGTGGTTCTGGCGGCGGCGGCTCCGGTGGTGGTGGTTCCCAGGTGCAGCT
GGTGGAGAGCGGAGGAGGTGTAGTTGCTCCTGGACAGAGCCTGAGACTGAGCTGTACCGCCA

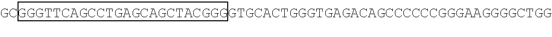

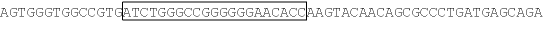

TTCACCATCAGCAGAGATACCAGCAAGACCACCGTGTACCTGCAGATGAACAGCCTGGAGACC

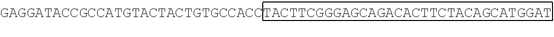

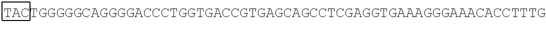

TCCAAGTCCCCTATTTCCCGGACCTTCTAAGCCCTTTTGGGTGCTGGTGGTGGTTGGTGGAGT
CCTGGCTTGCTATAGCTTGCTAGTAACAGTGGCCTTTATTATTTTCTGGGTGCTGTGCGCACG
CCCACGCCGCAGCCCCGCCCAAGAAGATGGCAAAGTCTACATCAACATGCCAGGCAGGGGCAG
AGTGAAGTTCAGCAGGAGCGCAGACGCCCCCGCGTACCAGCAGGGCCAGAACCAGCTCTATAA
CGAGCTCAATCTAGGACGAAGAGAGGAGTACGATGTTTTGGACAAGAGACGTGGCCGGGACC
CTGAGATGGGGGGAAAGCCGCAGAGAAGGAAGAACCCTCAGGAAGGCCTGTACAATGAACTG
CAGAAAGATAAGATGGCGGAGGCCTACAGTGAGATTGGGATGAAAGGCGAGCGCCGGAGGGG
CAAGGGGCACGATGGCCTTTACCAGGGTCTCAGTACAGCCACCAAGGACACCTACGACGCCCT
TCACATGCAGGCCCTGCCCCCTCGC
h6C10scFvHL-CD28H-CD28TM-CD28CS-CD3zICS
Protein Sequence:
(SEQ ID NO: 177)

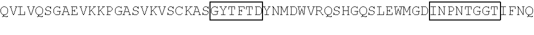

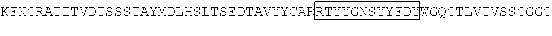

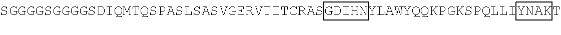

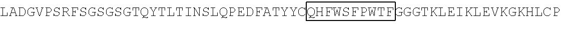

SPLFPGPSKPFWVLVVVGGVLACYSLLVTVAFIIFWVRSKRSRLLHSDYMNMTPRRPGPTRKH
YQPYAPPRDFAAYRSKLRVKFSRSADAPAYQQGQNQLYNELNLGRREEYDVLDKRRGRDPEM
GGKPQRRKNPQEGLYNELQKDKMAEAYSEIGMKGERRRGKGHDGLYQGLSTATKDTYDALH
MQALPPR
DNA Sequence:
(SEQ ID NO: 277)
CAGGTGCTGGTGCAGAGCGGGGCCGAGGTGAAGAAGCCCGGGCCAGCGTGAAGGTGAGCTG

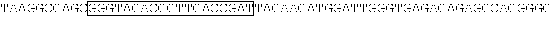

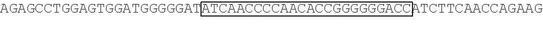

TTCAAGGGGAGAGCCACCATCACCGTGGATACCAGCAGCAGCACCGCCTACATGGATCTGCAC

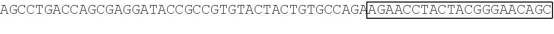

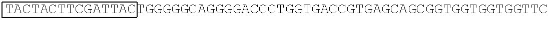

TGGCGGCGGCGGCTCCGGTGGTGGTGGTTCcGATATCCAGATGACCCAGAGCCCCGCCAGCCT
GAGCGCCAGCGTGGGGGAGAGAGTGACCATCACCTGTAGAGCCAGCGGGGATATCCACAACT
ACCTGGCCTGGTACCAGCAGAAGCCCGGGAAGAGCCCCCAGCTGCTGATCTACAACGCCAAGA
CCCTGGCCGATGGGGTGCCCAGCAGATTCAGCGGGAGCGGGAGCGGGACCCAGTACACCCTGA
CCATCAACAGCCTGCAGCCCGAGGATTTCGCCACCTACTACTGTCAGCACTTCTGGAGCTTCC
CCTGGACCTTCGGGGGGGGGACCAAGCTGGAGATCAAGCTCGAGGTGAAAGGGAAACACCTT
TGTCCAAGTCCCCTATTTCCCGGACCTTCTAAGCCCTTTTGGGTGCTGGTGGTGGTTGGTGGA
GTCCTGGCTTGCTATAGCTTGCTAGTAACAGTGGCCTTTATTATTTTCTGGGTGAGGAGTAAG
AGGAGCAGGCTCCTGCACAGTGACTACATGAACATGACTCCCCGCCGCCCCGGGCCCACCCGC
AAGCATTACCAGCCCTATGCCCCACCACGCGACTTCGCAGCCTATCGCTCCAAGCTTAGAGTG
AAGTTCAGCAGGAGCGCAGACGCCCCCGCGTACCAGCAGGGCCAGAACCAGCTCTATAACGAG
CTCAATCTAGGACGAAGAGAGGAGTACGATGTTTTGGACAAGAGACGTGGCCGGGACCCTGA
GATGGGGGGAAAGCCGCAGAGAAGGAAGAACCCTCAGGAAGGCCTGTACAATGAACTGCAGA
AAGATAAGATGGCGGAGGCCTACAGTGAGATTGGGATGAAAGGCGAGCGCCGGAGGGGCAAG
GGGCACGATGGCCTTTACCAGGGTCTCAGTACAGCCACCAAGGACACCTACGACGCCCTTCAC
ATGCAGGCCCTGCCCCCTCGC
h6C10scFvHL-CD28H-CD28TM-41BBCS-CD3zICS
Protein Sequence:
(SEQ ID NO: 178)

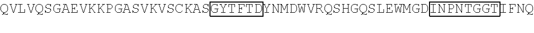

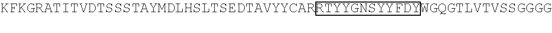

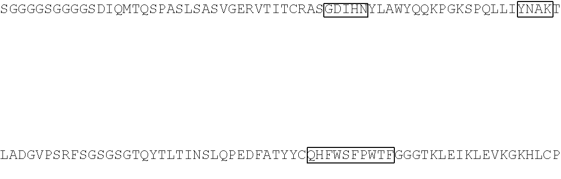

SPLFPGPSKPFWVLVVVGGVLACYSLLVTVAFIIFWVKRGRKKLLYIFKQPFMRPVQTTQEEDG
CSCRFPEEEEGGCELRVKFSRSADAPAYQQGQNQLYNELNLGRREEYDVLDKRRGRDPEMGG
KPQRRKNPQEGLYNELQKDKMAEAYSEIGMKGERRRGKGHDGLYQGLSTATKDTYDALHMQ
ALPPR
DNA Sequence:
(SEQ ID NO: 278)
CAGGTGCTGGTGCAGAGCGGGGCCGAGGTGAAGAAGCCCGGGGCCAGCGTGAAGGTGAGCTG

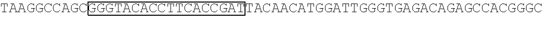

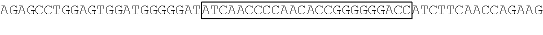

TTCAAGGGGAGAGCCACCATCACCGTGGATACCAGCAGCAGCACCGCCTACATGGATCTGCAC

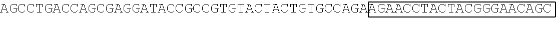

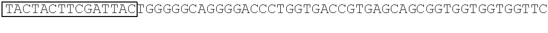

TGGCGGCGGCGGCTCCGGTGGTGGTGGTTCcGATATCCAGATGACCCAGAGCCCCGCCAGCCT
GAGCGCCAGCGTGGGGGAGAGAGTGACCATCACCTGTAGAGCCAGCGGGGATATCCACAACT
ACCTGGCCTGGTACCAGCAGAAGCCCGGGAAGAGCCCCCAGCTGCTGATCTACAACGCCAAGA
CCCTGGCCGATGGGGTGCCCAGCAGATTCAGCGGGAGCGGGAGCGGGACCCAGTACACCCTGA
CCATCAACAGCCTGCAGCCCGAGGATTTCGCCACCTACTACTGTCAGCACTTCTGGAGCTTCC
CCTGGACCTTCGGGGGGGGGACCAAGCTGGAGATCAAGCTCGAGGTGAAAGGGAAACACCTT
TGTCCAAGTCCCCTATTTCCCGGACCTTCTAAGCCCTTTTGGGTGCTGGTGGTGGTTGGTGGA
GTCCTGGCTTGCTATAGCTTGCTAGTAACAGTGGCCTTTATTATTTTCTGGGTGAAACGGGGC
AGAAAGAAACTCCTGTATATATTCAAACAACCATTTATGAGACCAGTACAAACTACTCAAGA
GGAAGATGGCTGTAGCTGCCGATTTCCAGAAGAAGAAGAAGGAGGATGTGAACTGAGAGTGA
AGTTCAGCAGGAGCGCAGACGCCCCCGCGTACCAGCAGGGCCAGAACCAGCTCTATAACGAGC
TCAATCTAGGACGAAGAGAGGAGTACGATGTTTTGGACAAGAGACGTGGCCGGGACCCTGAG
ATGGGGGGAAAGCCGCAGAGAAGGAAGAACCCTCAGGAAGGCCTGTACAATGAACTGCAGAA
AGATAAGATGGCGGAGGCCTACAGTGAGATTGGGATGAAAGGCGAGCGCCGGAGGGGCAAGG
GGCACGATGGCCTTTACCAGGGTCTCAGTACAGCCACCAAGGACACCTACGACGCCCTTCACA
TGCAGGCCCTGCCCCCTCGC
h6C10scFvHL-CD28H-CD28TM-DAP10CS-CD3zICS
Protein Sequence:
(SEQ ID NO: 179)

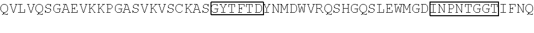

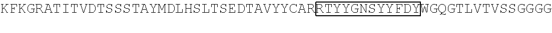

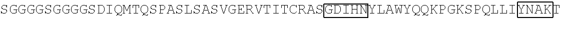

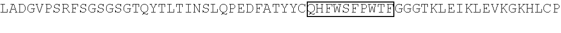

SPLFPGPSKPFWVLVVVGGVLACYSLLVTVAFIIFWVLCARPRRSPAQEDGKVYINMPGRGRVK
FSRSADAPAYQQGQNQLYNELNLGRREEYDVLDKRRGRDPEMGGKPQRRKNPQEGLYNELQ
KDKMAEAYSEIGMKGERRRGKGHDGLYQGLSTATKDTYDALHMQALPPR
DNA Sequence:
(SEQ ID NO: 279)
CAGGTGCTGGTGCAGAGCGGGGCCGAGGTGAAGAAGCCCGGGGCCAGCGTGAAGGTGAGCTG

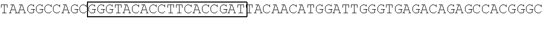

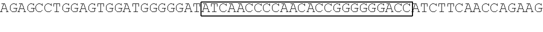

TTCAAGGGGAGAGCCACCATCACCGTGGATACCAGCAGCAGCACCGCCTACATGGATCTGCAC

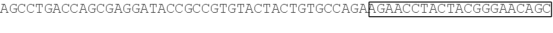

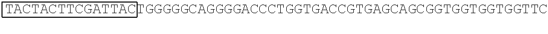

TGGCGGCGGCGGCTCCGGTGGTGGTGGTTCcGATATCCAGATGACCCAGAGCCCCCGCCAGCCT
GAGCGCCAGCGTGGGGGAGAGAGTGACCATCACCTGTAGAGCCAGCGGGGATATCCACAACT
ACCTGGCCTGGTACCAGCAGAAGCCCGGGAAGAGCCCCCAGCTGCTGATCTACAACGCCAAGA
CCCTGGCCGATGGGGTGCCCAGCAGATTCAGCGGGAGCGGGAGCGGGACCCAGTACACCCTGA
CCATCAACAGCCTGCAGCCCGAGGATTTCGCCACCTACTACTGTCAGCACTTCTGGAGCTTCC
CCTGGACCTTCGGGGGGGGGACCAAGCTGGAGATCAAGCTCGAGGTGAAAGGGAAACACCTT
TGTCCAAGTCCCCTATTTCCCGGACCTTCTAAGCCCTTTTGGGTGCTGGTGGTGGTTGGTGGA
GTCCTGGCTTGCTATAGCTTGCTAGTAACAGTGGCCTTTATTATTTTCTGGGTGCTGTGCGCA
CGCCCACGCCGCAGCCCCGCCCAAGAAGATGGCAAAGTCTACATCAACATGCCAGGCAGGGGC
AGAGTGAAGTTCAGCAGGAGCGCAGACGCCCCCGCGTACCAGCAGGGCCAGAACCAGCTCTAT
AACGAGCTCAATCTAGGACGAAGAGAGGAGTACGATGTTTTGGACAAGAGACGTGGCCGGGA
CCCTGAGATGGGGGGAAAGCCGCAGAGAAGGAAGAACCCTCAGGAAGGCCTGTACAATGAAC
TGCAGAAAGATAAGATGGCGGAGGCCTACAGTGAGATTGGGATGAAAGGCGAGCGCCGGAGG
GGCAAGGGGCACGATGGCCTTTACCAGGGTCTCAGTACAGCCACCAAGGACACCTACGACGCC
CTTCACATGCAGGCCCTGCCCCCTCGC
h6C10scFvLH-CD28H-CD28TM-CD28CS-CD3zICS
Protein Sequence:
(SEQ ID NO: 180)

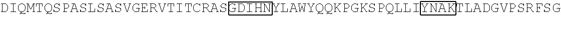

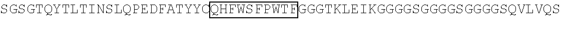

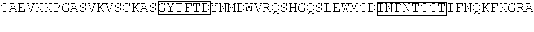

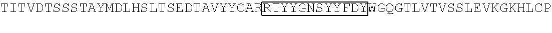

SPLFPGPSKPFWVLVVVGGVLACYSLLVTVAFIIFWVRSKRSRLLHSDYMNMTPRRPGPTRKH
YQPYAPPRDFAAYRSKLRVKFSRSADAPAYQQGQNQLYNELNLGRREEYDVLDKRRGRDPEM
GGKPQRRKNPQEGLYNELQKDKMAEAYSEIGMKGERRRGKGHDGLYQGLSTATKDTYDALH
MQALPPR
DNA Sequence:
(SEQ ID NO: 280)
GATATCCAGATGACCCAGAGCCCCGCCAGCCTGAGCGCCAGCGTGGGGGAGAGAGTGACCATC

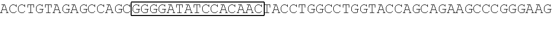

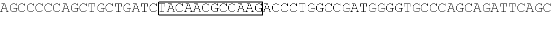

GGGAGCGGGAGCGGGACCCAGTACACCCTGACCATCAACAGCCTGCAGCCCGAGGATTTCGCC

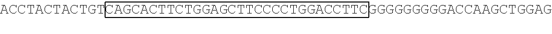

ATCAAGGGTGGTGGTGGTTCTGGCGGCGGCGGCTCCGGTGGTGGTGGTTCCCAGGTGCTGGTG

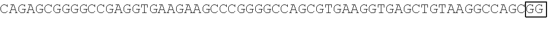

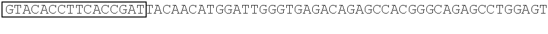

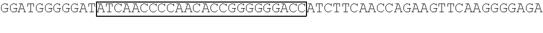

GCCACCATCACCGTGGATACCAGCAGCAGCACCGCCTACATGGATCTGCACAGCCTGACCAGC

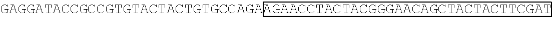

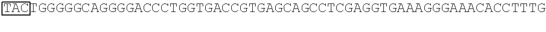

TCCAAGTCCCCTATTTCCCGGACCTTCTAAGCCCTTTTGGGTGCTGGTGGTGGTTGGTGGAGT
CCTGGCTTGCTATAGCTTGCTAGTAACAGTGGCCTTTATTATTTTCTGGGTGAGGAGTAAGA
GGAGCAGGCTCCTGCACAGTGACTACATGAACATGACTCCCCGCCGCCCCGGGCCCACCCGCA
AGCATTACCAGCCCTATGCCCCACCACGCGACTTCGCAGCCTATCGCTCCAAGCTTAGAGTGA
AGTTCAGCAGGAGCGCAGACGCCCCCGCGTACCAGCAGGGCCAGAACCAGCTCTATAACGAGC
TCAATCTAGGACGAAGAGAGGAGTACGATGTTTTGGACAAGAGACGTGGCCGGGACCCTGAG
ATGGGGGGAAAGCCGCAGAGAAGGAAGAACCCTCAGGAAGGCCTGTACAATGAACTGCAGAA
AGATAAGATGGCGGAGGCCTACAGTGAGATTGGGATGAAAGGCGAGCGCCGGAGGGGCAAGG
GGCACGATGGCCTTTACCAGGGTCTCAGTACAGCCACCAAGGACACCTACGACGCCCTTCACA
TGCAGGCCCTGCCCCCTCGC
h6C10scFvLH-CD28H-CD28TM-41BBCS-CD3zICS
Protein Sequence:
(SEQ ID NO: 181)

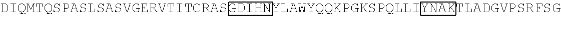

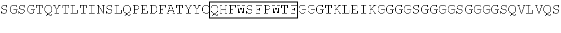

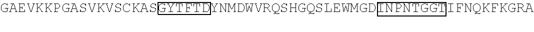

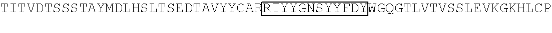

SPLFPGPSKPFWVLVVVGGVLACYSLLVTVAFIIFWVKRGRKKLLYIFKQPFMRPVQTTQEEDG
CSCRFPEEEEGGCELRVKFSRSADAPAYQQGQNQLYNELNLGRREEYDVLDKRRGRDPEMGG
KPQRRKNPQEGLYNELQKDKMAEAYSEIGMKGERRRGKGHDGLYQGLSTATKDTYDALHMQ
ALPPR
DNA Sequence:
(SEQ ID NO: 281)
GATATCCAGATGACCCAGAGCCCCGCCAGCCTGAGCGCCAGCGTGGGGGAGAGAGTGACCATC

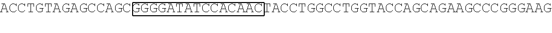

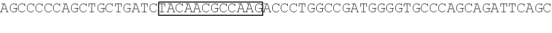

GGGAGCGGGAGCGGGACCCAGTACACCCTGACCATCAACAGCCTGCAGCCCGAGGATTTCGCC

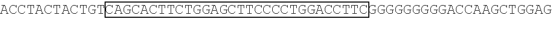

ATCAAGGGTGGTGGTGGTTCTGGCGGCGGCGGCTCCGGTGGTGGTGGTTCCCAGGTGCTGGTG

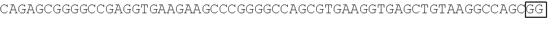

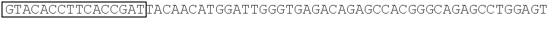

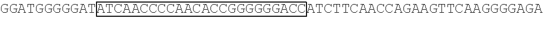

GCCACCATCACCGTGGATACCAGCAGCAGCACCGCCTACATGGATCTGCACAGCCTGACCAGC

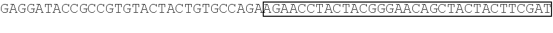

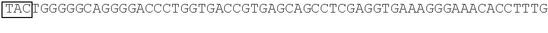

TCCAAGTCCCCTATTTCCCGGACCTTCTAAGCCCTTTTGGGTGCTGGTGGTGGTTGGTGGAGT
CCTGGCTTGCTATAGCTTGCTAGTAACAGTGGCCTTTATTATTTTCTGGGTGAAACGGGGCAG
AAAGAAACTCCTGTATATATTCAAACAACCATTTATGAGACCAGTACAAACTACTCAAGAGG
AAGATGGCTGTAGCTGCCGATTTCCAGAAGAAGAAGAAGGAGGATGTGAACTGAGAGTGAAG
TTCAGCAGGAGCGCAGACGCCCCCGCGTACCAGCAGGGCCAGAACCAGCTCTATAACGAGCTC
AATCTAGGACGAAGAGAGGAGTACGATGTTTTGGACAAGAGACGTGGCCGGGACCCTGAGAT
GGGGGGAAAGCCGCAGAGAAGGAAGAACCCTCAGGAAGGCCTCTACAATGAACTGCAGAAA
ATAAGATGGCGGAGGCCTACAGTGAGATTGGGATGAAAGGCGAGCGCCGGAGGGGCAAGGGG
CACGATGGCCTTTACCAGGGTCTCAGTACAGCCACCAAGGACACCTACGACGCCCTTCACATG
CAGGCCCTGCCCCCTCGC
6C10scFvLH-CD28H-CD28TM-DAP10CS-CD3zICS
Protein Sequence:
(SEQ ID NO: 182)

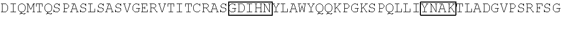

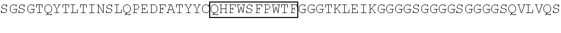

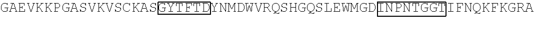

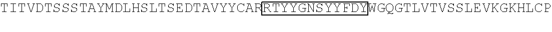

SPLFPGPSKPFWVLVVVGGVLACYSLLVTVAFIIFWVLCARPRRSPAQEDGKVYINMPGRGRVK
FSRSADAPAYQQGQNQLYNELNLGRREEYDVLDKRRGRDPEMGGKPQRRKNPQEGLYNELQ
KDKMAEAYSEIGMKGERRRGKGHDGLYQGLSTATKDTYDALHMQALPR
DNA Sequence:
(SEQ ID NO: 282)
GATATCCAGATGACCCAGAGCCCCGCCAGCCTGAGCGCCAGCGTGGGGGAGAGAGTGACCATC

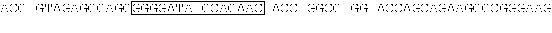

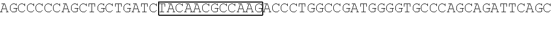

GGGAGCGGGAGCGGGACCCAGTACACCCTGACCATCAACAGCCTGCAGCCCGAGGATTTCGCC

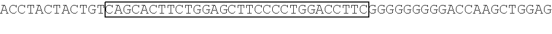

ATCAAGGGTGGTGGTGGTTCTGGCGGCGGCGGCTCCGGTGGTGGTGGTTCCCAGGTGCTGGTG

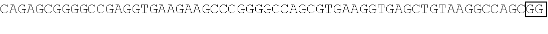

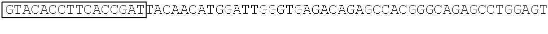

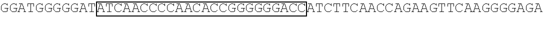

GCCACCATCACCGTGGATACCAGCAGCAGCACCGCCTACATGGATCTGCACAGCCTGACCAGC

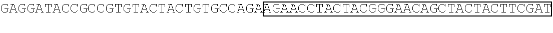

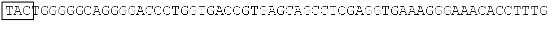

TCCAAGTCCCCTATTTCCCGGACCTTCTAAGCCCTTTTGGGTGCTGGTGGTGGTTGGTGGAGT
CCTGGCTTGCTATAGCTTGCTAGTAACAGTGGCCTTTATTATTTTCTGGGTGCTGTGCGCACG
CCCACGCCGCAGCCCCGCCCAAGAAGATGGCAAAGTCTACATCAACATGCCAGGCAGGGGCAG
AGTGAAGTTCAGCAGGAGCGCAGACGCCCCCGCGTACCAGCAGGGCCAGAACCAGCTCTATAA
CGAGCTCAATCTAGGACGAAGAGAGGAGTACGATGTTTTGGACAAGAGACGTGGCCGGGACC
CTGAGATGGGGGGAAAGCCGCAGAGAAGGAAGAACCCTCAGGAAGGCCTGTACAATGAACTG
CAGAAAGATAAGATGGCGGAGGCCTACAGTGAGATTGGGATGAAAGGCGAGCGCCGGAGGGG
CAAGGGGCACGATGGCCTTTACCAGGGTCTCAGTACAGCCACCAAGGACACCTACGACGCCCT
TCACATGCAGGCCCTGCCCCCTCGC

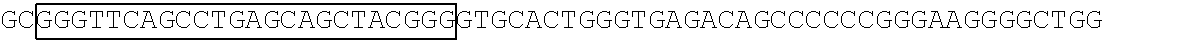

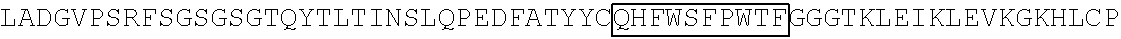

LS-h6E6scFvLH-CD28H-CD28TM-CD28CS-CD3zICS-T2A-trCD19
Protein Sequence:
(SEQ ID NO: 194)

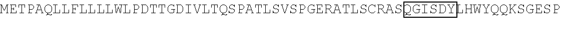

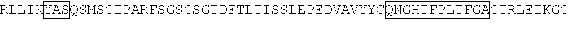

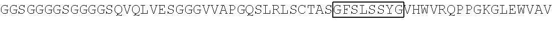

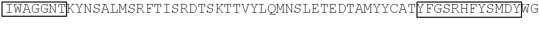

QGTLVTVSSLEVKGKHLCPSPLFPGPSKPFWVLVVVGGVLACYSLLVTVAFIIFWVRSKRSRLLH
SDYMNMTPRRPGPTRKGQPYAPPRDFAAYRSKLRVKFSRSADAPAYQQGQNQLYNELNLGR
REEYDVLDKRRGRDPEMGGKPQRRKNPQEGLYNELQKDKMAEAYSEIGMKGERRRGKGHDG
LYQGLSTATKDTYDALHMQALPPRAGAKRSGSGEGRGSLLTCGDVEENPGPRMPPPRLLFFLL
FLTPMEVRPEEPLVVKVEEGDNAVLQCLKGTSDGPTQQLTWSRESPLKPFLKLSLGLPGLGIH
MRPLAIWLFIFNVSQQMGGFYLCQPGPPSEKAWQPGWTVNVEGSGELFRWNVSDLGGLGCGL
KNRSSEGPSSPSGKLMSPKLYVWAKDRPEIWEGEPPCLPPRDSLNQSLSQDLTMAPGSTLWLS
CGVPPDSVSRGPLSWTHVHPKGPKSLLSLELKDDRPARDMWVMETGLLLPRATQADAGKYYC
HRGNLTMSFHLEITARPVLWHWLLRTGGWKVSAVTLAYLIFCLCSLVGILHLQRALVLRRKRK
RMT
DNA Sequence:
(SEQ ID NO: 294)
ATGGAAACCCCAGCGCAGCTTCTCTTCCTCCTGCTACTCTGGCTCCCAGATACCACCGGAGAT
ATCGTGCTGACCCAGAGCCCCGCCACCCTGAGCGTGAGCCCCGGGGAGAGAGCCACCCTGAGC

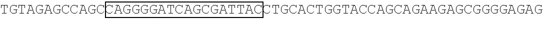

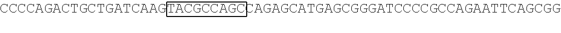

GAGCGGGAGCGGGACCGATTTCACCCTGACCATCAGCAGCCTGGAGCCCGAGGATGTGGCCGT

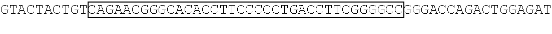

CAAGGGTGGTGGTGGTTCTGGCGGCGGCGGCTCCGGTGGTGGTGGTTCCCAGGTGCAGCTGGT

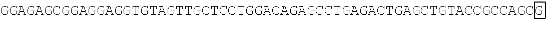

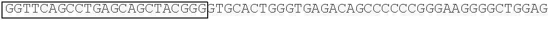

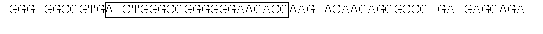

CACCATCAGCAGAGATACCAGCAAGACCACCGTGTACCTGCAGATGAACAGCCTGGAGACCGA

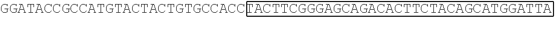

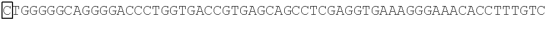

CAAGTCCCCTATTTCCCGGACCTTCTAAGCCCTTTTGGGTGCTGGTGGTGGTTGGTGGAGTCC
TGGCTTGCTATAGCTTGCTAGTAACAGTGGCCTTTATTATTTTCTGGGTGAGGAGTAAGAGG
AGCAGGCTCCTGCACAGTGACTACATGAACATGACTCCCCGCCGCCCCGGGCCCACCCGCAAG
CATTACCAGCCCTATGCCCCACCACGCGACTTCGCAGCCTATCGCTCCAAGCTTAGAGTGAAG
TTCAGCAGGAGCGCAGACGCCCCCGCGTACCAGCAGGGCCAGAACCAGCTCTATAACGAGCTC
AATCTAGGACGAAGAGAGGAGTACGATGTTTTGGACAAGAGACGTGGCCGGGACCCTGAGAT
GGGGGGAAAGCCGCAGAGAAGGAAGAACCCTCAGGAAGGCCTGTACAATGAACTGCAGAAAG
ATAAGATGGCGGAGGCCTACAGTGAGATTGGGATGAAAGGCGAGCGCCGGAGGGGCAAGGGG
CACGATGGCCTTTACCAGGGTCTCAGTACAGCCACCAAGGACACCTACGACGCCCTTCACATG
CAGGCCCTGCCCCCTCGCGCCGGCGCCAAAAGGTCTGGCTCCGGTGAGGGCAGAGGAAGTCTT
CTAACATGCGGTGACGTGGAGGAGAATCCCGGCCCTAGAATGCCACCTCCTCGCCTCCTCTTC
TTCCTCCTCTTCCTCACCCCCATGGAAGTCAGGCCCGAGGAACCTCTAGTGGTGAAGGTGGAA
GAGGGAGATAACGCTGTGCTGCAGTGCCTCAAGGGGACCTCAGATGGCCCCACTCAGCAGCTG
ACCTGGTCTCGGGAGTCCCCGCTTAAACCCTTCTTAAAACTCAGCCTGGGGCTGCCAGGCCTG
GGAATCCACATGAGGCCCCTGGCCATCTGGCTTTTCATCTTCAACGTCTCTCAACAGATGGGG
GGCTTCTACCTGTGCCAGCCGGGGCCCCCCTCTGAGAAGGCCTGGCAGCCTGGCTGGACAGTC
AATGTGGAGGGCAGCGGGGAGCTGTTCCGGTGGAATGTTTCGGACCTAGGTGGCCTGGGCTG
TGGCCTGAAGAACAGGTCCTCAGAGGGCCCCAGCTCCCCTTCCGGGAAGCTCATGAGCCCCAA
GCTGTATGTGTGGGCCAAAGACCGCCCTGAGATCTGGGAGGGAGAGCCTCCGTGTCTCCCACC
GAGGGACAGCCTGAACCAGAGCCTCAGCCAGGACCTCACCATGGCCCCTGGCTCCACACTCTG
GCTGTCCTGTGGGGTACCCCCTGACTCTGTGTCCAGGGGCCCCCTCTCCTGGACCCATGTGCAC
CCCAAGGGGCCTAAGTCATTGCTGAGCCTAGAGCTGAAGGACGATCGCCCGGCCAGAGATATG
TGGGTAATGGAGACGGGTCTGTTGTTGCCCCGGGCCACAGCTCAAGACGCTGGAAAGTATTAT
TGTCACCGTGGCAACCTGACCATGTCATTCCACCTGGAGATCACTGCTCGGCCAGTACTATGG
CACTGGCTGCTGAGGACTGGTGGCTGGAAGGTCTCAGCTGTGACTTTGGCTTATCTGATCTTC
TGCCTGTGTTCCCTTGTGGGCATTCTTCATCTTCAAAGAGCCCTGGTCCTGAGGAGGAAAAGA
AAGCGAATGACTTAA
LS-h6C10scFvHL-CD28H-CD28TM-CD28CS-CD3zICS-T2A-trCD19
Protein Sequence:
(SEQ ID NO: 197)

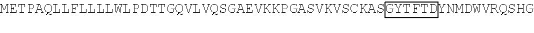

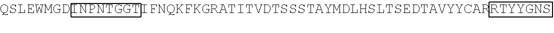

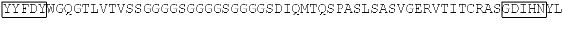

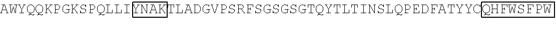

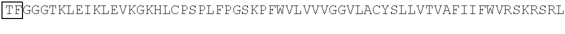

LHSDYMNMTPRRPGPTRKHYQPYAPPRDFAAYRSKLRVKFSRSADAPAPYQQGQNQLYNELNL
GRREEYDVLDKRRGRDPEMGGKPQRRKNPQEGLYNELQKDKMAEAYSEIGMKGERRRGKGH
DGLYQGLSTATKDTYDALHMQALPPRAGAKRSGSGEGRGSLLTCGDVEENPGPRMPPPRLLFF
LLFLTPMEVRPEEPLVVKVEEGDNAVLQCLKGTSDGPTQQLTWSRESPLKPFLKLSLGLPGLGI
HMRPLAIWLFIFNVSQQMGGFYLCQPGPPSEKAWQPGWTVNVEGSGELFRWNVSDLGGLGC
GLKNRSSEGPSSPSGKLMSPKLYVWAKDRPEIWEGEPPCLPPRDSLNQSLSQDLTMAPGSTLW
LSCGVPPDSVSRGPLSWTHVHPKGPKSLLSLELKDDRPARDMWVMETGLLLPRATAQDAGKY
YCHRGNLTMSFHLEITARPVLWHWLLRTGGWKVSAVTLAYLIFCLCSLVGILHLQRALVLRRK
RKRMT
DNA Sequence:
(SEQ ID NO: 297)
ATGGAAACCCCAGCGCAGCTTCTCTTCCTCCTGCTACTCTGGCTCCCAGATACCACCGGACAG
GTGCTGGTGCAGAGCGGGGCCGAGGTGAAGAAGCCCGGGGCCAGCGTGAAGGTGAGCTGTAA

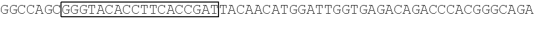

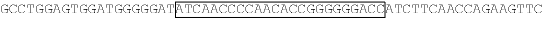

AAGGGGAGAGCCACCATCACCGTGGATACCAGCAGCAGCACCGCCTACATGGATCTGCACAGC

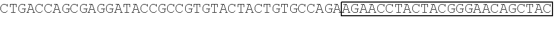

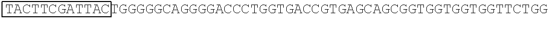

CGGCGGCGGCTCCGGTGGTGGTGGTTCcGATATCCAGATGACCCAGAGCCCCGCCAGCCTGAG
CGCCAGCGTGGGGGAGAGAGTGACCATCACCTGTAGAGCCAGCGGGGATATCCACAACTACCT
GGCCTGGTACCAGCAGAAGCCCGGGAAGAGCCCCCAGCTGCTGATCTACAACGCCAAGACCCT
GGCCGATGGGGTGCCCAGCAGATTCAGCGGGAGCGGGAGCGGGACCCAGTACACCCTGACCAT
CAACAGCCTGCAGCCCGAGGATTTCGCCACCTACTACTGTCAGCACTTCTGGAGCTTCCCCTG
GACCTTCGGGGGGGGGACCAAGCTGGAGATCAAGCTCGAGGTGAAAGGGAAACACCTTTGTC
CAAGTCCCCTATTTCCCGGACCTTCTAAGCCCTTTTGGGTGCTGGTGGTGGTTGGTGGAGTCC
TGGCTTGCTATAGCTTGCTAGTAACAGTGGCCTTTATTATTTTCTGGGTGAGGAGTAAGAGG
AGCAGGCTCCTGCACAGTGACTACATGAACATGACTCCCCGCCGCCCCGGGCCCACCCGCAAG
CATTACCAGCCCTATGCCCCACCACGCGACTTCGCAGCCTATCGCTCCAAGCTTAGAGTGAAG
TTCAGCAGGAGCGCAGACGCCCCCGCGTACCAGCAGGGCCAGAACCAGCTCTATAACGAGCTC
AATCTAGGACGAAGAGAGGAGTACGATGTTTTGGACAAGAGACGTGGCCGGGACCCTGAGAT
GGGGGGAAAGCCGCAGAGAAGGAAGAACCCTCAGGAAGGCCTGTACAATGAACTGCAGAAAG
ATAAGATGGCGGAGGCCTACAGTGAGATTGGGATGAAAGGCGAGCGCCGGAGGGGCAAGGGG
CACGATGGCCTTTACCAGGGTCTCAGTACAGCCACCAAGGACACCTACGACGCCCTTCACATG
CAGGCCCTGCCCCCTCGCGCCGGCGCCAAAAGGTCTGGCTCCGGTGAGGGCAGAGGAAGTCTT
CTAACATGCGGTGACGTGGAGGAGAATCCCGGCCCTAGAATGCCACCTCCTCGCCTCCTCTTC
TTCCTCCTCTTCCTCACCCCCATGGAAGTCAGGCCCGAGGAACCTCTAGTGGTGAAGGTGGAA
GAGGGAGATAACGCTGTGCTGCAGTGCCTCAAGGGGACCTCAGATGGCCCCACTCAGCAGCTG
ACCTGGTCTCGGGAGTCCCCGCTTAAACCCTTCTTAAAACTCAGCCTGGGGCTGCCAGGCCTG
GGAATCCACATGAGGCCCCTGGCCATCTGGCTTTTCATCTTCAACGTCTCTCAACAGATGGGG
GGCTTCTACCTGTGCCAGCCGGGGCCCCCCTCTGAGAAGGCCTGGCAGCCTGGCTGGACAGTC
AATGTGGAGGGCAGCGGGGAGCTGTTCCGGTGGAATGTTTCGGACCTAGGTGGCCTGGGCTG
TGGCCTGAAGAACAGGTCCTCAGAGGGCCCCAGCTCCCCTTCCGGGAAGCTCATGAGCCCCAA
GCTGTATGTGTGGGCCAAAGACCGCCCTGAGATCTGGGAGGGAGAGCCTCCGTGTCTCCCACC
GAGGGACAGCCTGAACCAGAGCCTCAGCCAGGACCTCACCATGGCCCCTGGCTCCACACTCTG
GCTGTCCTGTGGGGTACCCCCTGACTCTGTGTCCAGGGGCCCCCTCTCCTGGACCCATGTGCAC
CCCAAGGGGCCTAAGTCATTGCTGAGCCTAGAGCTGAAGGACGATCGCCCGGCCAGAGATATG
TGGGTAATGGAGACGGGTCTGTTGTTGCCCCGGGCCACAGCTCAAGACGCTGGAAAGTATTAT
TGTCACCGTGGCAACCTGACCATGTCATTCCACCTGGAGATCACTGCTCGGCCAGTACTATGG
CACTGGCTGCTGAGGACTGGTGGCTGGAAGGTCTCAGCTGTGACTTTGGCTTATCTGATCTTC
TGCCTGTGTTCCCTTGTGGGCATTCTTCATCTTCAAAGAGCCCTGGTCCTGAGGAGGAAAAGA
AAGCGAATGACTTAA

What is claimed is:

1. An antibody (Ab) or antigen-binding Ab fragment, wherein the Ab or antigen-binding Ab fragment binds to ADAM12 and comprises:
(a) a heavy chain (HC) variable domain comprising
a HC complementarity determining region 1 (CDR-H1),
a HC complementarity determining region 2 (CDR-H2),
a HC complementarity determining region 3 (CDR-H3), and
a human-like HC framework; and
(b) a light chain (LC) variable domain comprising
a LC complementarity determining region 1 (CDR-L1),
a LC complementarity determining region 2 (CDR-L2),
a LC complementarity determining region 3 (CDR-L3), and
a human-like LC framework,
optionally wherein said Ab or Ab fragment is selected from the group consisting of a monoclonal Ab, a monospecific Ab, a bispecific Ab, a multispecific Ab, a humanized Ab, a tetrameric Ab, a tetravalent Ab, a single chain Ab, a domain-specific Ab, a domain-deleted Ab, an scFc fusion protein, a chimeric Ab, a synthetic Ab, a recombinant Ab, a hybrid Ab, a mutated Ab, CDR-grafted Ab, a fragment antigen-binding (Fab), an F(ab′)2, an Fab′ fragment, a variable fragment (Fv), a single-chain Fv (scFv) fragment, an Fd fragment, a diabody, and a minibody.

2. The Ab or antigen-binding Ab fragment according to claim 1, wherein
(i) the CDR-H1, CDR-H2, and CDR-H3 comprise the amino acid sequences set forth in SEQ ID NOs: 132, 133, and 134, respectively, and the CDR-L1, CDR-L2, and CDR-L3 comprise the amino acid sequences set forth in SEQ ID NOs: 136, 137, and 138, respectively;
(ii) the CDR-H1, CDR-H2, and CDR-H3 comprise the amino acid sequences encoded by SEQ ID NOs: 232, 233, and 234, respectively, and the CDR-L1, CDR-L2, and CDR-L3 comprise the amino acid sequences encoded by SEQ ID NOs: 236, 237, and 238, respectively;
(iii) the CDR-H1, CDR-H2, and CDR-H3 comprise the amino acid sequences set forth in SEQ ID NOs: 142, 143, and 144, respectively, and the CDR-L1, CDR-L2, and CDR-L3 comprise the amino acid sequences set forth in SEQ ID NOs: 146, 147, and 148, respectively; or
(iv) the CDR-H1, CDR-H2, and CDR-H3 comprise the amino acid sequences encoded by SEQ ID NOs: 242, 243, and 244, respectively, and the CDR-L1, CDR-L2, and CDR-L3 comprise the amino acid sequences encoded by SEQ ID NOs: 246, 247, and 248, respectively,
and optionally,
(a) the human-like HC framework is at least 80%, at least 85%, at least 90%, at least 92%, at least 94%, at least 95%, at least 96%, at least 97%, at least 98%, at least 99%, or 100% identical to a human HC framework; and
(b) the human-like LC framework is at least 80%, at least 85%, at least 90%, at least 92%, at least 94%, at least 95%, at least 96%, at least 97%, at least 98%, at least 99%, or 100% identical to a human LC framework.

3. The Ab or antigen-binding Ab fragment according to claim 1, wherein
(i) the HC variable domain comprises an amino acid sequence at least 80%, at least 85%, at least 90%, at least 92%, at least 94%, at least 95%, at least 96%, at least 97%, at least 98%, at least 99%, or 100% identical to SEQ ID NO: 131, and the LC variable domain comprises an amino acid sequence at least 80%, at least 85%, at least 90%, at least 92%, at least 94%, at least 95%, at least 96%, at least 97%, at least 98%, at least 99%, or 100% identical to SEQ ID NO: 135;
(ii) the HC variable domain comprises an amino acid sequence at least 80%, at least 85%, at least 90%, at least 92%, at least 94%, at least 95%, at least 96%, at least 97%, at least 98%, at least 99%, or 100% identical to the amino acid sequence encoded by SEQ ID NO: 231, and the LC variable domain comprises an amino acid sequence at least 80%, at least 85%, at least 90%, at least 92%, at least 94%, at least 95%, at least 96%, at least 97%, at least 98%, at least 99%, or 100% identical to the amino acid sequence encoded by SEQ ID NO: 235;
(iii) the HC variable domain comprises an amino acid sequence at least 80%, at least 85%, at least 90%, at least 92%, at least 94%, at least 95%, at least 96%, at least 97%, at least 98%, at least 99%, or 100% identical to SEQ ID NO: 141, and the LC variable domain comprises an amino acid sequence at least 80%, at least 85%, at least 90%, at least 92%, at least 94%, at least 95%, at least 96%, at least 97%, at least 98%, at least 99%, or 100% identical to SEQ ID NO: 145; or
(iv) the HC variable domain comprises an amino acid sequence at least 80%, at least 85%, at least 90%, at least 92%, at least 94%, at least 95%, at least 96%, at least 97%, at least 98%, at least 99%, or 100% identical to the amino acid sequence encoded by SEQ ID NO: 241, and the LC variable domain comprises an amino acid sequence at least 80%, at least 85%, at least 90%, at least 92%, at least 94%, at least 95%, at least 96%, at least 97%, at least 98%, at least 99%, or 100% identical to the amino acid sequence encoded by SEQ ID NO: 245.

4. The Ab or antigen-binding Ab fragment according to claim 1, comprising an amino acid sequence at least 80%, at least 85%, at least 90%, at least 92%, at least 94%, at least 95%, at least 96%, at least 97%, at least 98%, at least 99%, or 100% identical to:
(i) the amino acid sequence set forth in SEQ ID NOs: 139, 140, 149, or 150; or
(ii) the amino acid sequence encoded by SEQ ID NOs: 239, 240, 249, or 250.

5. The Ab or antigen-binding Ab fragment according to claim 1, comprising two or more binding specificities, wherein the first specificity is for an epitope in ADAM12, wherein
(i) the second specificity is for another epitope in ADAM12, or
(ii) the second specificity is for an epitope in a second antigen other than ADAM12, optionally selected from the group consisting of CD3, NKG2D, 4-1BB, and Fc receptor (FcR).

6. The Ab or antigen-binding Ab fragment of claim 1, comprising a human-like fragment crystallizable (Fc) region,
(i) optionally wherein the human-like Fc region is at least 80%, at least 85%, at least 90%, at least 92%, at least 94%, at least 95%, at least 96%, at least 97%, at least 98%, at least 99%, or 100% identical to a human Fc region,
and
(ii) further optionally wherein the human-like Fc region binds to an FcR selected from the group consisting of Fc gamma receptor (FcgR), FcgRI, FcgRIIA, FcgRIIB1, FcgRIIB2, FcgRIIIA, FcgRIIIB, Fc epsilon receptor (FceR), FceRI, FceRII, Fc alpha receptor (FcaR), FcaRI, Fc alpha/mu receptor (Fca/mR), and neonatal Fc receptor (FcRn).

7. An antibody-drug conjugate (ADC) comprising:
(a) the Ab or antigen-binding Ab fragment of claim 1; and
(b) a drug conjugated to the Ab or antigen-binding Ab fragment,
(i) optionally wherein the drug is an anti-cancer drug, an anti-proliferative drug, a cytotoxic drug, an anti-angiogenic drug, an apoptotic drug, an immunostimulatory drug, an anti-microbial drug, an antibiotic drug, an antiviral drug, an anti-inflammatory drug, an anti-fibrotic drug, an immunosuppressive drug, a steroid, a bronchodilator, a beta blocker, a matrix metalloproteinase inhibitor, an ADAM12 inhibitor, an ADAM12 signaling inhibitor, an enzyme, a hormone, a neurotransmitter, a toxin, a radio isotope, a compound, a small molecule, a small molecule inhibitor, a protein, a peptide, a vector, a plasmid, a viral particle, a nanoparticle, a DNA molecule, an RNA molecule, an siRNA, an shRNA, a micro RNA, an oligonucleotide, or an imaging drug, and
(ii) further optionally wherein the drug is selected from the group consisting of doxorubicin, daunorubicin, cucurbitacin, chaetocin, chaetoglobosin, chlamydocin, calicheamicin, nemorubicin, cryptophyscin, mensacarcin, ansamitocin, mitomycin C, geldanamycin, mechercharmycin, rebeccamycin, safracin, okilactomycin, oligomycin, actinomycin, sandramycin, hypothemycin, polyketomycin, hydroxyellipticine, thiocolchicine, methotrexate, triptolide, taltobulin, lactacystin, dolastatin, auristatin, monomethyl auristatin E (MMAE), monomethyl auristatin F (MMAF), telomestatin, tubastatin A, combretastatin, maytansinoid, MMAD, MMAF, DM1, DM4, DTT, 16-GMB-APA-GA, 17-DMAP-GA, JW 55, pyrrolobenzodiazepine, SN-38, Ro 5-3335, puwainaphycin, duocarmycin, bafilomycin, taxoid, tubulysin, ferulenol, lusiol A, fumagillin, hygrolidin, glucopiericidin, amanitin, ansatrienin, cinerubin, phallacidin, phalloidin, phytosphongosine, piericidin, poronetin, phodophyllotoxin, gramicidin A, sanguinarine, sinefungin, herboxidiene, microcolin B, microcystin, muscotoxin A, tolytoxin, tripolin A, myoseverin, mytoxin B, nocuolin A, psuedolaric acid B, pseurotin A, cyclopamine, curvulin, colchicine, aphidicolin, englerin, cordycepin, apoptolidin, epothilone A, limaquinone, isatropolone, isofistularin, quinaldopeptin, ixabepilone, aeroplysinin, arruginosin, agrochelin, epothilone, and a derivative of any one of the foregoing.

8. A chimeric antigen receptor (CAR) comprising:
(a) an antigen-binding (AB) domain that binds to ADAM12;
(b) a transmembrane (TM) domain;
(c) an intracellular signaling (ICS) domain;
(d) optionally, a hinge that joins said AB domain and said TM domain; and
(e) optionally, one or more costimulatory (CS) domains.

9. The CAR according to claim 8, wherein:
(I) the AB domain comprises an amino acid sequence at least 80%, at least 85%, at least 90%, at least 92%, at least 94%, at least 95%, at least 96%, at least 97%, at least 98%, at least 99%, or 100% identical to the amino acid sequence
(i) set forth in SEQ ID NOs: 139, 140, 149, or 150, or
(ii) encoded by SEQ ID NOs: 239, 240, 249, or 250;
(II) the AB domain competes for binding to ADAM12 with an scFv comprising an amino acid sequence at least 80%, at least 85%, at least 90%, at least 92%, at least 94%, at least 95%, at least 96%, at least 97%, at least 98%, at least 99%, or 100% identical to the amino acid sequence
(i) set forth in SEQ ID NOs: 139, 140, 149, or 150, or
(ii) encoded by SEQ ID NOs: 239, 240, 249, or 250; or
(III) the AB domain comprises an amino acid sequence at least 80%, at least 85%, at least 90%, at least 92%, at least 94%, at least 95%, at least 96%, at least 97%, at least 98%, at least 99%, or 100% identical to the ADAM12-binding domain of a natural ADAM12-binding molecule,
and optionally wherein the natural ADAM12-binding molecule is selected from the group consisting of alpha actinin 2 (ACTN2), insulin-like growth factor-binding protein 3 (IGFBP3), IGFBP5, phosphatidylinositol 3 kinase regulatory subunit alpha (PIK3R1), heparin-binding epidermal growth factor (HB-EGF), epidermal growth factor (EGF), Betacellulin, Delta-like 1, placental leucine aminopeptidase (P-LAP), and matrix metalloprotease (MMP-14).

10. The CAR according to claim 8, wherein the TM domain is:
(I) derived from the TM region, or a membrane-spanning portion thereof, of a protein selected from the group consisting of CD28, CD3e, CD4, CD5, CD8, CD9, CD16, CD22, CD33, CD37, CD45, CD64, CD80, CD86, CD134, CD137, CD154, TCRa, TCRb, and CD3z,
(II) optionally derived from the TM region of CD28, or a membrane-spanning portion thereof, and
(III) further optionally comprising an amino acid sequence at least 80%, at least 85%, at least 90%, at least 95%, at least 98%, at least 99%, or 100% identical to the amino acid sequence:
(i) set forth in SEQ ID NO: 161; or
(ii) encoded by SEQ ID NO: 261.

11. The CAR according to claim 8, wherein the ICS domain is:
(I) derived from a cytoplasmic signaling sequence, or a functional fragment thereof, of a protein selected from the group consisting of CD3z, a lymphocyte receptor chain, a TCR/CD3 complex protein, an Fc receptor (FcR) subunit, an IL-2 receptor subunit, FcRg, FcRb, CD3g, CD3d, CD3e, CD5, CD22, CD66d, CD79a, CD79b, CD278 (ICOS), FceRI, DAP10, and DAP12,
(II) optionally derived from a cytoplasmic signaling sequence of CD3z, or a functional fragment thereof, and
(III) further optionally comprising an amino acid sequence at least 80%, at least 85%, at least 90%, at least 95%, at least 98%, at least 99%, or 100% identical to the amino acid sequence:
(i) set forth in SEQ ID NO: 162; or
(ii) encoded by SEQ ID NO: 262.

12. The CAR according to claim 8, wherein the hinge is
(I) derived from CD28, and
(II) optionally comprising an amino sequence at least 80%, at least 85%, at least 90%, at least 95%, at least 98%, at least 99%, or 100% identical to the amino acid sequence
(i) set forth in SEQ ID NO: 163; or
(ii) encoded by SEQ ID NO: 263.

13. The CAR according to claim 8, wherein at least one of said one or more CS domains is
(I) derived from a cytoplasmic signaling sequence, or functional fragment thereof, of a protein selected from the group consisting of CD28, DAP10, 4-1BB (CD137), CD2, CD4, CD5, CD7, CD8a, CD8b, CD11a, CD11b, CD11c, CD11d, CD18, CD19, CD27, CD29, CD30, CD40, CD49d, CD49f, CD69, CD84, CD96 (Tactile), CD100 (SEMA4D), CD103, OX40 (CD134), SLAM (SLAMFI, CD150, IPO-3), CD160 (BY55), SELPLG (CD162), DNAM1 (CD226), Ly9 (CD229), SLAMF4 (CD244, 2B4), ICOS (CD278), B7-H3, BAFFR, BTLA, BLAME (SLAMF8), CEACAMI, CDS, CRTAM, GADS, GITR, HVEM (LIGHTER), IA4, ICAM-1, IL2Rb, IL2Rg, IL7Ra, ITGA4, ITGA6, ITGAD, ITGAE, ITGAL, ITGAM, ITGAX, ITGB1, ITGB2, ITGB7, KIRDS2, LAT, LFA-1, LIGHT, LTBR, NKG2C, NKG2D, NKp30, NKp44, NKp46, NKp80 (KLRF1), PAG/Cbp, PD-1, PSGL1, SLAMF6 (NTB-A, Ly108), SLAMF7, SLP-76, TNFR2, TRANCE/RANKL, VLA1, VLA-6, and CD83 ligand,
(II) optionally derived from a cytoplasmic signaling sequence of CD28, 4-1BB, or DAP10, or functional fragment thereof, and
(III) further optionally comprising an amino sequence at least 80%, at least 85%, at least 90%, at least 95%, at least 98%, at least 99%, or 100% identical to the amino acid sequence:
(i) set forth in SEQ ID NO: 164;
(ii) encoded by SEQ ID NO: 264;
(iii) set forth in SEQ ID NO: 165;
(iv) encoded by SEQ ID NO: 265;
(v) set for the in SEQ ID NO: 166; or
(vi) encoded by SEQ ID NO: 266.

14. The CAR according to claim 8, comprising an amino acid sequence at least 80%, at least 85%, at least 90%, at least 95%, at least 98%, at least 99%, or 100% identical to the amino acid sequence of:
(i) h6E6scFvHL-CD28H-CD28TM-CD28CS-CD3zICS (SEQ ID NO: 171);
(ii) h6E6scFvHL-CD28H-CD28TM-41BBCS-CD3zICS (SEQ ID NO: 172);
(iii) h6E6scFvHL-CD28H-CD28TM-DAP10CS-CD3zICS (SEQ ID NO: 173);
(iv) h6E6scFvLH-CD28H-CD28TM-CD28CS-CD3zICS (SEQ ID NO: 174);
(v) h6E6scFvLH-CD28H-CD28TM-41BBCS-CD3zICS (SEQ ID NO: 175);
(vi) h6E6scFvLH-CD28H-CD28TM-DAP10CS-CD3zICS (SEQ ID NO: 176);
(vii) h6C10scFvHL-CD28H-CD28TM-CD28CS-CD3zICS (SEQ ID NO: 177);
(viii) h6C10scFvHL-CD28H-CD28TM-41BBCS-CD3zICS (SEQ ID NO: 178);
(ix) h6C10scFvHL-CD28H-CD28TM-DAP10CS-CD3zICS (SEQ ID NO: 179);
(x) h6C10scFvLH-CD28H-CD28TM-CD28CS-CD3zICS (SEQ ID NO: 180);
(xi) h6C10scFvLH-CD28H-CD28TM-41BBCS-CD3zICS (SEQ ID NO: 181);
(xii) h6C10scFvLH-CD28H-CD28TM-DAP10CS-CD3zICS (SEQ ID NO: 182); or
(xiii) to the amino acid sequence encoded by any one of SEQ ID NOs 271-282.

15. The CAR according to claim 8, further comprising a cytotoxic drug conjugated to the AB domain.

16. An isolated nucleic acid sequence encoding an antibody (Ab) or antigen-binding Ab fragment according to claim 1.

17. An isolated nucleic acid sequence encoding a CAR according to claim 8.

18. The isolated nucleic acid sequence according to claim 17, further comprising:
(I) a leader sequence (LS), wherein the LS is optionally at least 80%, at least 85%, at least 90%, at least 95%, at least 98%, at least 99%, or 100% identical to the amino acid sequence set forth in SEQ ID NO: 260, and optionally
(II) a T2A sequence and/or a sequence encoding truncated CD19 (trCD19), wherein the T2A sequence is optionally at least 80%, at least 85%, at least 90%, at least 95%, at least 98%, at least 99%, or 100% identical to SEQ ID NO: 269 and the trCD19 optionally comprises an amino acid sequence at least 85%, at least 90%, at least 95%, at least 98%, at least 99%, or 100% identical to the amino acid sequence set forth in SEQ ID NO: 170.

19. A vector comprising the nucleic acid sequence according to claim 16,
optionally wherein the vector is selected from a DNA, an RNA, a plasmid, a cosmid, a viral vector, a lentiviral vector, an adenoviral vector, or a retroviral vector.

20. A recombinant or isolated cell comprising:
(i) the Ab or antigen-binding Ab fragment according to claim 1,
(ii) an ADC containing said Ab or antigen-binding Ab fragment,
(iii) a CAR containing said Ab or antigen-binding Ab fragment,
(iv) a nucleic acid encoding (i), (ii) or (iii), or
(v) a vector comprising at least one nucleic acid according to (iv);
optionally wherein said cell is:
(I) a non-mammalian cell, optionally selected from the group consisting of a plant cell, a bacterial cell, a fungal cell, a yeast cell, a protozoan cell, and an insect cell;
(II) a mammalian cell, optionally selected from the group consisting of a human cell, a rat cell, and a mouse cell;
(III) a stem cell;
(IV) a primary cell, optionally a human primary cell or derived therefrom;
(V) a cell line, optionally a hybridoma cell line;
(VI) an immune cell;
(VII) MHC+ or MHC−; or
(VIII) selected from the group consisting of a cell line, a T cell, a T cell progenitor cell, a CD4+ T cell, a helper T cell, a regulatory T cell, a CD8+ T cell, a naïve T cell, an effector T cell, a memory T cell, a stem cell memory T (TSCM) cell, a central memory T (TCM) cell, an effector memory T (TEM) cell, a terminally differentiated effector memory T cell, a tumor-infiltrating lymphocyte (TIL), an immature T cell, a mature T cell, a cytotoxic T cell, a mucosa-associated invariant T (MAIT) cell, a TH1 cell, a TH2 cell, a TH3 cell, a TH17 cell, a TH9 cell, a TH22 cell, a follicular helper T cells, and a/b T cell, a g/d T cell, a Natural Killer T (NKT) cell, a cytokine-induced killer (CIK) cell, a lymphokine-activated killer (LAK) cell, a perforin-deficient cell, a granzyme-deficient cell, a B cell, a myeloid cell, a monocyte, a macrophage, and a dendritic cell.

21. The recombinant or isolated cell of claim 20, wherein the cell expresses a CAR comprising:
(a) an antigen-binding (AB) domain that binds to ADAM12;
(b) a transmembrane (TM) domain;
(c) an intracellular signaling (ICS) domain;
(d) optionally, a hinge that joins said AB domain and said TM domain; and
(e) optionally, one or more costimulatory (CS) domains;
further optionally wherein the cell is a T cell which has been modified such that its endogenous T cell receptor (TCR) is
(i) not expressed;
(ii) not functionally expressed; or
(iii) expressed at reduced levels compared to a wild-type T cell,
and further optionally wherein, when the CAR binds to its target molecule:
(I) the cell is activated or stimulated to proliferate;
(II) the cell exhibits cytotoxicity against cells expressing the target molecule;
(III) administration of the cell ameliorates a disease, a cancer, a cardiac condition, an autoimmune condition, an inflammatory condition, or a fibrotic condition;
(IV) the cell increases expression of cytokines and/or chemokines, optionally wherein the cytokine is IFN-g; or
(V) the cell decreases expression of cytokines and/or chemokines, optionally wherein the cytokine is TGF-b.

22. A population of cells comprising at least one first recombinant or isolated cell according to claim 20, and optionally at least one second recombinant or isolated cell which is engineered to express a different CAR, ADC or antibody or antigen binding antibody fragment than the first recombinant or isolated cell.

23. A pharmaceutical composition comprising at least one of:
(a) an Ab or antigen-binding Ab fragment according to claim 1,
(ii) an ADC containing said Ab or antigen-binding Ab fragment,
(iii) a CAR containing said Ab or antigen-binding Ab fragment,
(iv) a nucleic acid encoding (i), (ii) or (iii),
(v) a vector comprising a nucleic acid according to (iv),
(vi) a cell or population of cells comprising one or more of (i) to (vi); and
(b) optionally, a pharmaceutically acceptable excipient or carrier.

24. A method of treating a subject, the method comprising administering to the subject in need thereof a therapeutically effective amount of at least one of:
(i) an Ab or antigen-binding Ab fragment according to claim 1,
(ii) an ADC containing said Ab or antigen-binding Ab fragment,
(iii) a CAR containing said Ab or antigen-binding Ab fragment,
(iv) a nucleic acid encoding (i), (ii) or (iii),
(v) a vector comprising a nucleic acid according to (iv),
(vi) a cell or population of cells comprising one or more of (i) to (vi); or
(vii) a pharmaceutical composition comprising any of (i) to (vii) and a pharmaceutically acceptable carrier,
wherein the method is used in the treatment of:
(I) cancer, fibrosis, autoimmunity, a cardiovascular condition, an allergic condition, a respiratory disease, a nephropathy, a neural disease, a muscular disease, a liver disease, metabolic syndrome, infection, or an inflammatory disorder, or
(II) cancer, wherein the cancer is selected from the group consisting of bladder cancer, bone cancer, brain cancer, breast cancer, colon cancer, colorectal cancer, desmoid tumor, esophageal cancer, fibromatosis, glioblastoma, head and neck cancer, liver cancer, lung cancer, melanoma, oesophago-gastric adenocarcinoma, oligodendroma, oral cancer, oral squamous cell carcinoma, osteosarcoma, ovarian cancer, pancreatic cancer, prostate cancer, skin cancer, small cell lung cancer, stomach cancer, and thyroid cancer.

25. A method of treating a subject with an anti-ADAM12 agent, the method comprising the steps of:
(a) obtaining or having obtained a biological sample from the subject;
(b) measuring the expression level of ADAM12 in the biological sample;
(c) determining whether the subject is an ADAM12 over-expresser; and
(d) if the subject is an ADAM12 over-expresser, administering to the subject a therapeutically effective amount of at least one of:
(i) an Ab or antigen-binding Ab fragment according to claim 1,
(ii) an ADC containing said Ab or antigen-binding Ab fragment,
(iii) a CAR containing said Ab or antigen-binding Ab fragment,
(iv) a nucleic acid encoding (i), (ii) or (iii),
(v) a vector comprising a nucleic acid according to (iv),
(vi) a cell or population of cells comprising one or more of (i) to (vi); or
(vii) a pharmaceutical composition comprising any of (i) to (vii) and a pharmaceutically acceptable carrier;
wherein the subject is suffering from cancer, wherein the cancer is optionally selected from the group consisting of bladder cancer, bone cancer, brain cancer, breast cancer, colon cancer, colorectal cancer, desmoid tumor, esophageal cancer, fibromatosis, glioblastoma, head and neck cancer, liver cancer, lung cancer, melanoma, oesophago-gastric adenocarcinoma, oligodendroma, oral cancer, oral squamous cell carcinoma, osteosarcoma, ovarian cancer, pancreatic cancer, prostate cancer, skin cancer, small cell lung cancer, stomach cancer, and thyroid cancer.

26. A method for stimulating an immune response in a subject, comprising administering to the subject a therapeutically effective amount of at least one of:
(i) an Ab or antigen-binding Ab fragment according to claim 1,
(ii) an ADC containing said Ab or antigen-binding Ab fragment,
(iii) a CAR containing said Ab or antigen-binding Ab fragment,
(iv) a nucleic acid encoding (i), (ii) or (iii),
(v) a vector comprising a nucleic acid according to (iv),
(vi) a cell or population of cells comprising one or more of (i) to (vi); or
(vii) a pharmaceutical composition comprising any of (i) to (vii) and a pharmaceutically acceptable carrier.

27. A method of treating a disease in a subject, comprising administering to the subject in need thereof a therapeutically effective amount of at least one of:
(i) an Ab or antigen-binding Ab fragment according to claim 1,
(ii) an ADC containing said Ab or antigen-binding Ab fragment,
(iii) a CAR containing said Ab or antigen-binding Ab fragment,
(iv) a nucleic acid encoding (i), (ii) or (iii),
(v) a vector comprising a nucleic acid according to (iv),
(vi) a cell or population of cells comprising one or more of (i) to (vi); or
(vii) a pharmaceutical composition comprising any of (i) to (vii) and a pharmaceutically acceptable carrier
wherein the disease is
(I) cancer, fibrosis, autoimmunity, a cardiovascular condition, an allergic condition, a respiratory disease, a nephropathy, a neural disease, a muscular disease, a liver disease, metabolic syndrome, infection, or an inflammatory disorder, or
(II) a cancer selected from the group consisting of bladder cancer, bone cancer, brain cancer, breast cancer, colon cancer, colorectal cancer, desmoid tumor, esophageal cancer, fibromatosis, glioblastoma, head and neck cancer, liver cancer, lung cancer, melanoma, oesophago-gastric adenocarcinoma, oligodendroma, oral cancer, oral squamous cell carcinoma, osteosarcoma, ovarian cancer, pancreatic cancer, prostate cancer, skin cancer, small cell lung cancer, stomach cancer, and thyroid cancer;
(III) or a combination of any of the foregoing.

28. A method of expanding a population of cells in a subject in need thereof, the method comprising administering to the subject:
(i) an Ab or antigen-binding Ab fragment according to claim 1,
(ii) an ADC containing said Ab or antigen-binding Ab fragment,
(iii) a CAR containing said Ab or antigen-binding Ab fragment,
(iv) a nucleic acid encoding (i), (ii) or (iii),
(v) a vector comprising a nucleic acid according to (iv),
(vi) a cell or population of cells comprising one or more of (i) to (vi); or
(vii) a pharmaceutical composition comprising any of (i) to (vii) and a pharmaceutically acceptable carrier;
(I) optionally wherein the resulting population of cells persists in the subject for at least three months, at least four months, at least five months, at least six months, at least seven months, at least eight months, at least nine months, at least ten months, at least eleven months, at least twelve months, at least eighteen months, at least two years, or at least three years after administration, and
(II) further optionally wherein the subject is suffering from cancer, wherein the cancer is further optionally selected from the group consisting of bladder cancer, bone cancer, brain cancer, breast cancer, colon cancer, colorectal cancer, desmoid tumor, esophageal cancer, fibromatosis, glioblastoma, head and neck cancer, liver cancer, lung cancer, melanoma, oesophago-gastric adenocarcinoma, oligodendroma, oral cancer, oral squamous cell carcinoma, osteosarcoma, ovarian cancer, pancreatic cancer, prostate cancer, skin cancer, small cell lung cancer, stomach cancer, and thyroid cancer.

29. The method according to claim 24, further comprising administering a second agent, wherein the second agent is optionally an anti-cancer drug, an anti-proliferative drug, a cytotoxic drug, an anti-angiogenic drug, an apoptotic drug, an immunostimulatory drug, an anti-microbial drug, an antibiotic drug, an antiviral drug, an anti-inflammatory drug, an anti-fibrotic drug, an immunosuppressive drug, a steroid, a bronchodilator, a beta blocker, a matrix metalloproteinase inhibitor, an ADAM12 inhibitor, an ADAM12 signaling inhibitor, an enzyme, a hormone, a neurotransmitter, a toxin, a radio isotope, a compound, a small molecule, a small molecule inhibitor, a protein, a peptide, a vector, a plasmid, a viral particle, a nanoparticle, a DNA molecule, an RNA molecule, an siRNA, an shRNA, a micro RNA, an oligonucleotide, an imaging drug, or a CAR, ADC or antibody or antigen binding antibody fragment that targets an antigen other than ADAM12 or a recombinant or isolated cell which is engineered to express a CAR, ADC or antibody or antigen binding antibody fragment that targets an antigen other than ADAM12.

30. A method of generating a cell comprising the CAR according to claim 8, the method comprising:
introducing into a cell (i-a) a nucleic acid sequence encoding at least one CAR according to claim 8 or a vector containing
optionally further comprising:
(iii) isolating the cell based on expression of said CAR and/or a selectable marker as determined via flow cytometry or immunofluorescence assays.


SEQUENCE LISTING (297 sequences)

SEQ ID NO: 1-100 - skipped (100 entries)

SEQ ID NO: 101 - protein, 909 aa, Homo sapiens
MAARPLPVSPARALLLALAGALLAPCEARGVSLWNQGRADEVVSASVRSGDLWIPVKSFDSKNHPEVLNIRLQRESKELIINLERNEGLIASSFTETHYLQDGTDVSLARNYTVILGHCYYHGHVRGYSDSAVSLSTCSGLRGLIVFENESYVLEPMKSATNRYKLFPAKKLKSVRGSCGSHHNTPNLAAKNVFPPPSQTWARRHKRETLKATKYVELVIVADNREFQRQGKDLEKVKQRLIEIANHVDKFYRPLNIRIVLVGVEVWNDMDKCSVSQDPFTSLHEFLDWRKMKLLPRKSHDNAQLVSGVYFQGTTIGMAPIMSMCTADQSGGIVMDHSDNPLGAAVTLAHELGHNFGMNHDTLDRGCSCQMAVEKGGCIMNASTGYPFPMVFSSCSRKDLETSLEKGMGVCLFNLPEVRESFGGQKCGNRFVEEGEECDCGEPEECMNRCCNATTCTLKPDAVCAHGLCCEDCQLKPAGTACRDSSNSCDLPEFCTGASPHCPANVYLHDGHSCQDVDGYCYNGICQTHEQQCVTLWGPGAKPAPGICFERVNSAGDPYGNCGKVSKSSFAKCEMRDAKCGKIQCQGGASRPVIGTNAVSIETNIPLQQGGRILCRGTHVYLGDDMPDPGLVLAGTKCADGKICLNRQCQNISVFGVHECAMQCHGRGVCNNRKNCHCEAHWAPPFCDKFGFGGSTDSGPIRQADNQGLTIGILVTILCLLAAGFVVYLKRKTLIRLLFTNKKTTIEKLRCVRPSRPPRGFQPCQAHLGHLGKGLMRKPPDSYPPKDNPRRLLQCQNVDISRPLNGLNVPQPQSTQRVLPPLHRAPRAPSVPARPLPAKPALRQAQGTCKPNPPQKPLPADPLARTTRLTHALARTPGQWETGLRLAPLRPAPQYPHQVPRSTHTAYIK

SEQ ID NO: 102 - protein, 738 aa, Homo sapiens [sequence omitted]

SEQ ID NO: 103 - protein, 903 aa, Mus musculus
MAERPARRAPPARALLLALAGALLAPRAARGMSLWDQRGTYEVARASLLSKDPGIPGQSIPAKDHPDVLTVQLQLESRDLILSLERNEGLIANGFTETHYLQDGTDVSLTRNHTDHCYYHGHVQGDAASVVSLSTCSGLRGLIMFENKTYSLEPMKNTTDSYKLVPAESMTNIQGLCGSQHNKSNLTMEDVSPGTSQMRARRHKRETLKMTKYVELVIVADNREFQRQGKDLEKVKQRLIEIANHVDKFYRPLNIRIVLVGVEVWNDIDKCSISQDPFTSLHEFLDWRKIKLLPRKSHDNAQLISGVYFQGTTIGMAPIMSMCTAEQSGGVVMDHSDSPLGAAVTLAHELGHNFGMNHDTLERGCSCRMAAEKGGCIMNPSTGFPFPMVFSSCSRKDLEASLEKGMGMCLFNLPEVKQAFGGRKCGNGYVEEGEECDCGEPEECTNRCCNATTCTLKPDAVCAHGQCCEDCQLKPPGTACRGSSNSCDLPEFCTGTAPHCPANVYLHDGHPCQGVDGYCYNGICQTHEQQCVTLWGPGAKPAPGICFERVNSAGDPYGNCGKDSKSAFAKCELRDAKCGKIQCQGGASRPVIGTNAVSIETNIPQQEGGRILCRGTHVYLGDDMPDPGLVLAGTKCAEGKICLNRRCQNISVFGVHKCAMQCHGRGVCNNRKNCHCEAHWAPPFCDKFGFGGSTDSGPIRQADNQGLTVGILVSILCLLAAGFVVYLKRKTLMRLLFTHKKTTMEKLRCVHPSRTPSGPHLGQAHHTPGKGLLMNRAPHFNTPKDRHSLKCQNMDISRPLDARAVPQLQSPQRVLLPLHQTPRAPSGPARPLPASPAVRQAQGIRKPSPPQKPLPADPLSRTSRLTSALVRTPGQQEPGHRPAPIRPAPKHQVPRPSHNAYIK

SEQ ID NO: 104-110 - skipped (7 entries)

SEQ ID NO: 111 - protein, 120 aa, Artificial Sequence
  Mouse anti-ADAM12 Clone 6E6 Heavy chain variable
QVQLQESGPGLVAPSQSLSITCTVSGFSLSSYGVHWVRQPPGKGLEWLGVIWAGGNTKYNSALMSRLSISRDTSKTQVFLEMNSLETDDTAMYYCATYFGSRHFYSMDYWGQGTSVTVSS

SEQ ID NO: 112 - protein, 8 aa, Artificial Sequence
  Mouse anti-ADAM12 6E6 VH CDR 1
GFSLSSYG

SEQ ID NO: 113 - protein, 7 aa, Artificial Sequence
  Mouse anti-ADAM12 6E6 VH CDR 2
IWAGGNT

SEQ ID NO: 114 - protein, 12 aa, Artificial Sequence
  Mouse anti-ADAM12 6E6 VH CDR 3
YFGSRHFYSMDY

SEQ ID NO: 115 - protein, 107 aa, Artificial Sequence
  Mouse anti-ADAM12 Clone 6E6 Light chain variable
DIVMTQSPATLSVTPGDRVSLSCRASQGISDYLHWYQQKSHESPRLLIKYASQSMSGIPSRFSGSGSGSDFTLTINSVEPEDVGMYFCQNGHTFPLTFGAGTKLEIK

SEQ ID NO: 116 - protein, 6 aa, Artificial Sequence
  Mouse anti-ADAM12 6E6 VL CDR 1
QGISDY

SEQ ID NO: 117 - protein, 3 aa, Artificial Sequence
  Mouse anti-ADAM12 6E6 VL CDR 2
YAS

SEQ ID NO: 118 - protein, 12 aa, Artificial Sequence
  Mouse anti-ADAM12 6E6 VL CDR 3
QNGHTFPLTFGA

SEQ ID NO: 119-120 - skipped (2 entries)

SEQ ID NO: 121 - protein, 121 aa, Artificial Sequence
  Mouse anti-ADAM12 Clone 6C10 Heavy chain variable
EALLQQSGPDLAKPGASVQIPCKASGYTFTDYNMDWVKQSHGKSLEWIGDINPNTGGTIFNQKFKGKATLTVDKSSSTAFMDLHSLTSEDTAVYYCARRTYYGNSYYFDYWGQGTTLTVSS

SEQ ID NO: 122 - protein, 6 aa, Artificial Sequence
  Mouse anti-ADAM12 6C10 VH CDR 1
GYTFTD

SEQ ID NO: 123 - protein, 8 aa, Artificial Sequence
  Mouse anti-ADAM12 6C10 VH CDR 2
INPNTGGT

SEQ ID NO: 124 - protein, 12 aa, Artificial Sequence
  Mouse anti-ADAM12 6C10 VH CDR 3
RTYYGNSYYFDY

SEQ ID NO: 125 - protein, 107 aa, Artificial Sequence
  Mouse anti-ADAM12 Clone 6C10 Light chain variable
DIQMTQSPASLSASVGETVTITCRASGDIHNYLAWYQQKQGKSPQLLVYNAKTLADGVPSRFSGSGSGTQYSLKMNSLQPEDFGTYYCQHFWSFPWTFGGGTKLEIK

SEQ ID NO: 126 - protein, 5 aa, Artificial Sequence
  Mouse anti-ADAM12 6C10 VL CDR 1
GDIHN

SEQ ID NO: 127 - protein, 4 aa, Artificial Sequence
  Mouse anti-ADAM12 6C10 VL CDR 2
YNAK

SEQ ID NO: 128 - protein, 10 aa, Artificial Sequence
  Mouse anti-ADAM12 6C10 VL CDR 3
QHFWSFPWTF

SEQ ID NO: 129-130 - skipped (2 entries)

SEQ ID NO: 131 - protein, 120 aa, Artificial Sequence
  Humanized anti-ADAM12 Clone 6E6 Heavy chain variable
QVQLVESGGGVVAPGQSLRLSCTASGFSLSSYGVHWVRQPPGKGLEWVAVIWAGGNTKYNSALMSRFTISRDTSKTTVYLQMNSLETEDTAMYYCATYFGSRHFYSMDYWGQGTLVTVSS

SEQ ID NO: 132 - protein, 8 aa, Artificial Sequence
  Humanized anti-ADAM12 6E6 VH CDR 1
GFSLSSYG

SEQ ID NO: 133 - protein, 7 aa, Artificial Sequence
  Humanized anti-ADAM12 6E6 VH CDR 2
IWAGGNT

SEQ ID NO: 134 - protein, 12 aa, Artificial Sequence
  Humanized anti-ADAM12 6E6 VH CDR 3
YFGSRHFYSMDY

SEQ ID NO: 135 - protein, 107 aa, Artificial Sequence
  Humanized anti-ADAM12 Clone 6E6 Light chain variable
DIVLTQSPATLSVSPGERATLSCRASQGISDYLHWYQQKSGESPRLLIKYASQSMSGIPARFSGSGSGTDFTLTISSLEPEDVAVYYCQNGHTFPLTFGAGTRLEIK

SEQ ID NO: 136 - protein, 6 aa, Artificial Sequence
  Humanized anti-ADAM12 6E6 VL CDR 1
QGISDY

SEQ ID NO: 137 - protein, 3 aa, Artificial Sequence
  Humanized anti-ADAM12 6E6 VL CDR 2
YAS

SEQ ID NO: 138 - protein, 12 aa, Artificial Sequence
  Humanized anti-ADAM12 6E6 VL CDR 3
QNGHTFPLTFGA

SEQ ID NO: 139 - protein, 242 aa, Artificial Sequence
  Humanized anti-ADAM12 6E6 scFv VH-G4S3-VL
QVQLVESGGGVVAPGQSLRLSCTASGFSLSSYGVHWVRQPPGKGLEWVAVIWAGGNTKYNSALMSRFTISRDTSKTTVYLQMNSLETEDTAMYYCATYFGSRHFYSMDYWGQGTLVTVSSGGGGSGGGGSGGGGSDIVLTQSPATLSVSPGERATLSCRASQGISDYLHWYQQKSGESPRLLIKYASQSMSGIPARFSGSGSGTDFTLTISSLEPEDVAVYYCQNGHTFPLTFGAGTRLEIK

SEQ ID NO: 140 - protein, 242 aa, Artificial Sequence
  Humanized anti-ADAM12 6E6 scFv VL-G4S3-VH
DIVLTQSPATLSVSPGERATLSCRASQGISDYLHWYQQKSGESPRLLIKYASQSMSGIPARFSGSGSGTDFTLTISSLEPEDVAVYYCQNGHTFPLTFGAGTRLEIKGGGGSGGGGSGGGGSQVQLVESGGGVVAPGQSLRLSCTASGFSLSSYGVHWVRQPPGKGLEWVAVIWAGGNTKYNSALMSRFTISRDTSKTTVYLQMNSLETEDTAMYYCATYFGSRHFYSMDYWGQGTLVTVSS

SEQ ID NO: 141 - protein, 120 aa, Artificial Sequence
  Humanized anti-ADAM12 Clone 6C10 Heavy chain variable
QVLVQSGAEVKKPGASVKVSCKASGYTFTDYNMDWVRQSHGQSLEWMGDINPNTGGTIFNQKFKGRATITVDTSSSTAYMDLHSLTSEDTAVYYCARRTYYGNSYYFDYWGQGTLVTVSS

SEQ ID NO: 142 - protein, 6 aa, Artificial Sequence
  Humanized anti-ADAM12 6C10 VH CDR 1
GYTFTD

SEQ ID NO: 143 - protein, 8 aa, Artificial Sequence
  Humanized anti-ADAM12 6C10 VH CDR 2
INPNTGGT

SEQ ID NO: 144 - protein, 12 aa, Artificial Sequence
  Humanized anti-ADAM12 6C10 VH CDR 3
RTYYGNSYYFDY

SEQ ID NO: 145 - protein, 107 aa, Artificial Sequence
  Humanized anti-ADAM12 Clone 6C10 Light chain variable
DIQMTQSPASLSASVGERVTITCRASGDIHNYLAWYQQKPGKSPQLLIYNAKTLADGVPSRFSGSGSGTQYTLTINSLQPEDFATYYCQHFWSFPWTFGGGTKLEIK

SEQ ID NO: 146 - protein, 5 aa, Artificial Sequence
  Humanized anti-ADAM12 6C10 VL CDR 1
GDIHN

SEQ ID NO: 147 - protein, 4 aa, Artificial Sequence
  Humanized anti-ADAM12 6C10 VL CDR 2
YNAK

SEQ ID NO: 148 - protein, 10 aa, Artificial Sequence
  Humanized anti-ADAM12 6C10 VL CDR 3
QHFWSFPWTF

SEQ ID NO: 149 - protein, 242 aa, Artificial Sequence
  Humanized anti-ADAM12 6C10 scFv VH-G4S3-VL
QVLVQSGAEVKKPGASVKVSCKASGYTFTDYNMDWVRQSHGQSLEWMGDINPNTGGTIFNQKFKGRATITVDTSSSTAYMDLHSLTSEDTAVYYCARRTYYGNSYYFDYWGQGTLVTVSSGGGGSGGGGSGGGGSDIQMTQSPASLSASVGERVTITCRASGDIHNYLAWYQQKPGKSPQLLIYNAKTLADGVPSRFSGSGSGTQYTLTINSLQPEDFATYYCQHFWSFPWTFGGGTKLEIK

SEQ ID NO: 150 - protein, 242 aa, Artificial Sequence
  Humanized anti-ADAM12 6C10 scFv VL-G4S3-VH
DIQMTQSPASLSASVGERVTITCRASGDIHNYLAWYQQKPGKSPQLLIYNAKTLADGVPSRFSGSGSGTQYTLTINSLQPEDFATYYCQHFWSFPWTFGGGTKLEIKGGGGSGGGGSGGGGSQVLVQSGAEVKKPGASVKVSCKASGYTFTDYNMDWVRQSHGQSLEWMGDINPNTGGTIFNQKFKGRATITVDTSSSTAYMDLHSLTSEDTAVYYCARRTYYGNSYYFDYWGQGTLVTVSS

SEQ ID NO: 151 - protein, 894 aa, Homo sapiens
MNQIEPGVQYNYVYDEDEYMIQEEEWDRDLLLDPAWEKQQRKTFTAWCNSHLRKAGTQIENIEEDFRNGLKLMLLLEVISGERLPKPDRGKMRFHKIANVNKALDYIASKGVKLVSIGAEEIVDGNVKMTLGMIWTIILRFAIQDISVEETSAKEGLLLWCQRKTAPYRNVNIQNFHTSWKDGLGLCALIHRHRPDLIDYSKLNKDDPIGNINLAMEIAEKHLDIPKMLDAEDIVNTPKPDERAIMTYVSCFYHAFAGAEQAETAANRICKVLAVNQENERLMEEYERLASELLEWIRRTIPWLENRTPEKTMQAMQKKLEDFRDYRRKHKPPKVQEKCQLEINFNTLQTKLRISNRPAFMPSEGKMVSDIAGAWQRLEQAEKGYEEWLLNEIRRLERLEHLAEKFRQKASTHETWAYGKEQILLQKDYESASLTEVRALLRKHEAFESDLAAHQDRVEQIAAIAQELNELDYHDAVNVNDRCQKICDQWDRLGTLTQKRREALERMEKLLETIDQLHLEFAKRAAPFNNWMEGAMEDLQDMFIVHSIEEIQSLITAHEQFKATLPEADGERQSIMAIQNEVEKVIQSYNIRISSSNPYSTVTMDELRTKWDKVKQLVPIRDQSLQEELARQHANERLRRQFAAQANAIGPWIQNKMEEIARSSIQITGALEDQMNQLKQYEHNIINYKNNIDKLEGDHQLIQEALVFDNKHTNYTMEHIRVGWELLLTTIARTINEVETQILTRDAKGITQEQMNEFRASFNHFDRRKNGLMDHEDFRACLISMGYDLGEAEFARIMTLVDPNGQGTVTFQSFIDFMTRETADTDTAEQVIASFRILASDKPYILAEELRRELPPDQAQYCIKRMPAYSGPGSVPGALDYAAFSSALYGESDL

SEQ ID NO: 152 - protein, 291 aa, Homo sapiens
MQRARPTLWAAALTLLVLLRGPPVARAGASSAGLGPVVRCEPCDARALAQCAPPPAVCAELVREPGCGCCLTCALSEGQPCGIYTERCGSGLRCQPSPDEARPLQALLDGRGLCVNASAVSRLRAYLLPAPPAPGNASESEEDRSAGSVESPSVSSTHRVSDPKFHPLHSKIIIIKKGHAKDSQRYKVDYESQSTDTQNFSSESKRETEYGPCRREMEDTLNHLKFLNVLSPRGVHIPNCDKKGFYKKKQCRPSKGRKRGFCWCVDKYGQPLPGYTTKGKEDVHCYSMQSK

SEQ ID NO: 153 - protein, 272 aa, Homo sapiens
MVLLTAVLLLLAAYAGPAQSLGSFVHCEPCDEKALSMCPPSPLGCELVKEPGCGCCMTCALAEGQSCGVYTERCAQGLRCLPRQDEEKPLHALLHGRGVCLNEKSYREQVKIERDSREHEEPTTSEMAEETYSPKIFRPKHTRISELKAEAVKKDRRKKLTQSKFVGGAENTAHPRIISAPEMRQESEQGPCRRHMEASLQELKASPRMVPRAVYLPNCDRKGFYKRKQCKPSRGRKRGICWCVDKYGMKLPGMEYVDGDFQCHTFDSSNVE

SEQ ID NO: 154 - protein, 724 aa, Homo sapiens
MSAEGYQYRALYDYKKEREEDIDLHLGDILTVNKGSLVALGFSDGQEARPEEIGWLNGYNETTGERGDFPGTYVEYIGRKKISPPTPKPRPPRPLPVAPGSSKTEADVEQQALTLPDLAEQFAPPDIAPPLLIKLVEAIEKKGLECSTLYRTQSSSNLAELRQLLDCDTPSVDLEMIDVHVLADAFKRYLLDLPNPVIPAAVYSEMISLAPEVQSSEEYIQLLKKLIRSPSIPHQYWLTLQYLLKHFFKLSQTSSKNLLNARVLSEIFSPMLFRFSAASSDNTENLIKVIEILISTEWNERQPAPALPPKPPKPTTVANNGMNNNMSLQDAEWYWGDISREEVNEKLRDTADGTFLVRDASTKMHGDYTLTLRKGGNNKLIKIFHRDGKYGFSDPLTFSSVVELINHYRNESLAQYNPKLDVKLLYPVSKYQQDQVVKEDNIEAVGKKLHEYNTQFQEKSREYDRLYEEYTRTSQEIQMKRTAIEAFNETIKIFEEQCQTQERYSKEYIEKFKREGNEKEIQRIMHNYDKLKSRISEIIDSRRRLEEDLKKQAAEYREIDKRMNSIKPDLIQLRKTRDQYLMWLTQKGVRQKKLNEWLGNENTEDQYSLVEDDEDLPHHDEKTWNVGSSNRNKAENLLRGKRDGTFLVRESSKQGCYACSVVVDGEVKHCVINKTATGYGFAEPYNLYSSLKELVLHYQHTSLVQHNDSLNVTLAYPVYAQQRR

SEQ ID NO: 155 - protein, 208 aa, Homo sapiens
MKLLPSVVLKLFLAAVLSALVTGESLERLRRGLAAGTSNPDPPTVSTDQLLPLGGGRDRKVRDLQEADLDLLRVTLSSKPQALATPNKEEHGKRKKKGKGLGKKRDPCLRKYKDFCIHGECKYVKELRAPSCICHPGYHGERCHGLSLPVENRLYTYDHTTILAVVAVVLSSVCLLVIVGLLMFRYHRRGGYDVENEEKVKLGMTNSH

SEQ ID NO: 156 - protein, 53 aa, Homo sapiens
NSDSECPLSHDGYCLHDGVCMYIEALDKYACNCVVGYIGERCQYRDLKWWELR

SEQ ID NO: 157 - protein, 178 aa, Homo sapiens
MDRAARCSGASSLPLLLALALGLVILHCVVADGNSTRSPETNGLLCGDPEENCAATTTQSKRKGHFSRCPKQYKHYCIKGRCRFVVAEQTPSCVCDEGYIGARCERVDLFYLRGDRGQILVICMIAVMVVFIILVIGVCTCCHPLRKRRKRKKKEEEMETLGKDITPINEDIEETNIA

SEQ ID NO: 158 - protein, 723 aa, Homo sapiens
MGSRCALALAVLSALLCQVWSSGVFELKLQEFVNKKGLLGNRNCCRGGAGPPPCACRTFFRVCLKHYQASVSPEPPCTYGSAVTPVLGVDSFSLPDGGGADSAFSNPIRFPFGFTWPGTFSLIIEALHTDSPDDLATENPERLISRLATQRHLTVGEEWSQDLHSSGRTDLKYSYRFVCDEHYYGEGCSVFCRPRDDAFGHFTCGERGEKVCNPGWKGPYCTEPICLPGCDEQHGFCDKPGECKCRVGWQGRYCDECIRYPGCLHGTCQQPWQCNCQEGWGGLFCNQDLNYCTHHKPCKNGATCTNTGQGSYTCSCRPGYTGATCELGIDECDPSPCKNGGSCTDLENSYSCTCPPGFYGKICELSAMTCADGPCFNGGRCSDSPDGGYSCRCPVGYSGFNCEKKIDYCSSSPCSNGAKCVDLGDAYLCRCQAGFSGRHCDDNVDDCASSPCANGGTCRDGVNDFSCTCPPGYTGRNCSAPVSRCEHAPCHNGATCHERGHRYVCECARGYGGPNCQFLLPELPPGPAVVDLTEKLEGQGGPFPWVAVCAGVILVLMLLLGCAAVVVCVRLRLQKHRPPADPCRGETETMNNLANCQREKDISVSIIGATQIKNTNKKADFHGDHSADKNGFKARYPAVDYNLVQDLKGDDTAVRDAHSKRDTKCQPQGSSGEEKGTPTTLRGGEASERKRPDSGCSTSKDTKYQSVYVISEEKDECVIATEV

SEQ ID NO: 159 - protein, 1025 aa, Homo sapiens
MEPFTNDRLQLPRNMIENSMFEEEPDVVDLAKEPCLHPLEPDEVEYEPRGSRLLVRGLGEHEMEEDEEDYESSAKLLGMSFMNRSSGLRNSATGYRQSPDGACSVPSARTMVVCAFVIVVAVSVIMVIYLLPRCTFTKEGCHKKNQSIGLIQPFATNGKLFPWAQIRLPTAVVPLRYELSLHPNLTSMTFRGSVTISVQALQVTWNIILHSTGHNISRVTFMSAVSSQEKQAEILEYAYHGQIAIVAPEALLAGHNYTLKIEYSANISSSYYGFYGFSYTDESNEKKYFAATQFEPLAARSAFPCFDEPAFKATFIIKIIRDEQYTALSNMPKKSSVVLDDGLVQDEFSESVKMSTYLVAFIVGEMKNLSQDVNGTLVSIYAVPEKIGQVHYALETTVKLLEFFQNYFEIQYPLKKLDLVAIPDFEAGAMENWGLLTFREETLLYDSNTSSMADRKLVTKIIAHELAHQWFGNLVTMKWWNDLWLNEGFATFMEYFSLEKIFKELSSYEDFLDARFKTMKKDSLNSSHPISSSVQSSEQIEEMFDSLSYFKGSSLLLMLKTYLSEDVFQHAVVLYLHNHSYASIQSDDLWDSFNEVTNQTLDVKRMMKTWTLQKGFPLVTVQKKGKELFIQQERFFLNMKPEIQPSDTSYLWHIPLSYVTEGRNYSKYQSVSLLDKKSGVINLTEEVLWVKVNINMNGYYIVHYADDDWEALIHQLKINPYVLSDKDRANLINNIFELAGLGKVPLKRAFDLINYLGNENHTAPITEALFQTDLIYNLLEKLGYMDLASRLVTRVFKLLQNQIQQQTWTDEGTPSMRELRSALLEFACTHNLGNCSTTAMKLFDDWMASNGTQSLPTDVMTTVFKVGAKTDKGWSFLLGKYISIGSEAEKNKILEALASSEDVRKLYWLMKSSLNGDNFRTQKLSFIIRTVGRHFPGHLLAWDFVKENWNKLVQKFPLGSYTIQNIVAGSTYLFSTKTHLSEVQAFFENQSEATFRLRCVQEALEVIQLNIQWMEKNLKSLTWWL

SEQ ID NO: 160 - protein, 20 aa, Artificial Sequence
  Lead sequence
METPAQLLFLLLLWLPDTTG

SEQ ID NO: 161 - protein, 27 aa, Artificial Sequence
  Human CD28 transmembrane domain
FWVLVVVGGVLACYSLLVTVAFIIFWV

SEQ ID NO: 162 - protein, 113 aa, Artificial Sequence
  Human CD3 zeta intracellular signaling domain
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 163 - protein, 20 aa, Artificial Sequence
  Human CD28 hinge
LEVKGKHLCPSPLFPGPSKP

SEQ ID NO: 164 - protein, 43 aa, Artificial Sequence
  Human CD28 costimulatory domain
RSKRSRLLHSDYMNMTPRRPGPTRKHYQPYAPPRDFAAYRSKL

SEQ ID NO: 165 - protein, 42 aa, Artificial Sequence
  Human 4-1BB costimulatory domain
KRGRKKLLYIFKQPFMRPVQTTQEEDGCSCRFPEEEEGGCEL

SEQ ID NO: 166 - protein, 24 aa, Artificial Sequence
  Human DAP10 costimulatory domain
LCARPRRSPAQEDGKVYINMPGRG

SEQ ID NO: 167 - protein, 5 aa, Artificial Sequence
  Glycine-Serine linker unite
GGGGS

SEQ ID NO: 168 - protein, 15 aa, Artificial Sequence
  (Gly-Gly-Gly-Gly-Ser)X3 linker ((Gly4Ser)3 linker, (G4S)3 linker, G4S X3 linker, or G4SX3 linker)
GGGGSGGGGSGGGGS

SEQ ID NO: 169 - protein, 28 aa, Artificial Sequence
  T2A ribosomal skip sequence
AGAKRSGSGEGRGSLLTCGDVEENPGPR

SEQ ID NO: 170 - protein, 327 aa, Artificial Sequence
  truncated CD19
MPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKRKRMT

SEQ ID NO: 171 - protein, 445 aa, Artificial Sequence
  h6E6scFvHL-CD28H-CD28TM-CD28CS-CD3zICS
QVQLVESGGGVVAPGQSLRLSCTASGFSLSSYGVHWVRQPPGKGLEWVAVIWAGGNTKYNSALMSRFTISRDTSKTTVYLQMNSLETEDTAMYYCATYFGSRHFYSMDYWGQGTLVTVSSGGGGSGGGGSGGGGSDIVLTQSPATLSVSPGERATLSCRASQGISDYLHWYQQKSGESPRLLIKYASQSMSGIPARFSGSGSGTDFTLTISSLEPEDVAVYYCQNGHTFPLTFGAGTRLEIKLEVKGKHLCPSPLFPGPSKPFWVLVVVGGVLACYSLLVTVAFIIFWVRSKRSRLLHSDYMNMTPRRPGPTRKHYQPYAPPRDFAAYRSKLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 172 - protein, 444 aa, Artificial Sequence
  h6E6scFvHL-CD28H-CD28TM-41BBCS-CD3zICS
QVQLVESGGGVVAPGQSLRLSCTASGFSLSSYGVHWVRQPPGKGLEWVAVIWAGGNTKYNSALMSRFTISRDTSKTTVYLQMNSLETEDTAMYYCATYFGSRHFYSMDYWGQGTLVTVSSGGGGSGGGGSGGGGSDIVLTQSPATLSVSPGERATLSCRASQGISDYLHWYQQKSGESPRLLIKYASQSMSGIPARFSGSGSGTDFTLTISSLEPEDVAVYYCQNGHTFPLTFGAGTRLEIKLEVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 173 - protein, 426 aa, Artificial Sequence
  h6E6scFvHL-CD28H-CD28TM-DAP10CS-CD3zICS
QVQLVESGGGVVAPGQSLRLSCTASGFSLSSYGVHWVRQPPGKGLEWVAVIWAGGNTKYNSALMSRFTISRDTSKTTVYLQMNSLETEDTAMYYCATYFGSRHFYSMDYWGQGTLVTVSSGGGGSGGGGSGGGGSDIVLTQSPATLSVSPGERATLSCRASQGISDYLHWYQQKSGESPRLLIKYASQSMSGIPARFSGSGSGTDFTLTISSLEPEDVAVYYCQNGHTFPLTFGAGTRLEIKLEVKGKHLCPSPLFPGPSKPFWVLVVVGGVLACYSLLVTVAFIIFWVLCARPRRSPAQEDGKVYINMPGRGRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 174 - protein, 445 aa, Artificial Sequence
  h6E6scFvLH-CD28H-CD28TM-CD28CS-CD3zICS
DIVLTQSPATLSVSPGERATLSCRASQGISDYLHWYQQKSGESPRLLIKYASQSMSGIPARFSGSGSGTDFTLTISSLEPEDVAVYYCQNGHTFPLTFGAGTRLEIKGGGGSGGGGSGGGGSQVQLVESGGGVVAPGQSLRLSCTASGFSLSSYGVHWVRQPPGKGLEWVAVIWAGGNTKYNSALMSRFTISRDTSKTTVYLQMNSLETEDTAMYYCATYFGSRHFYSMDYWGQGTLVTVSSLEVKGKHLCPSPLFPGPSKPFWVLVVVGGVLACYSLLVTVAFIIFWVRSKRSRLLHSDYMNMTPRRPGPTRKHYQPYAPPRDFAAYRSKLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 175 - protein, 444 aa, Artificial Sequence
  h6E6scFvLH-CD28H-CD28TM-41BBCS-CD3zICS
DIVLTQSPATLSVSPGERATLSCRASQGISDYLHWYQQKSGESPRLLIKYASQSMSGIPARFSGSGSGTDFTLTISSLEPEDVAVYYCQNGHTFPLTFGAGTRLEIKGGGGSGGGGSGGGGSQVQLVESGGGVVAPGQSLRLSCTASGFSLSSYGVHWVRQPPGKGLEWVAVIWAGGNTKYNSALMSRFTISRDTSKTTVYLQMNSLETEDTAMYYCATYFGSRHFYSMDYWGQGTLVTVSSLEVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 176 - protein, 426 aa, Artificial Sequence
  h6E6scFvLH-CD28H-CD28TM-DAP10CS-CD3zICS
DIVLTQSPATLSVSPGERATLSCRASQGISDYLHWYQQKSGESPRLLIKYASQSMSGIPARFSGSGSGTDFTLTISSLEPEDVAVYYCQNGHTFPLTFGAGTRLEIKGGGGSGGGGSGGGGSQVQLVESGGGVVAPGQSLRLSCTASGFSLSSYGVHWVRQPPGKGLEWVAVIWAGGNTKYNSALMSRFTISRDTSKTTVYLQMNSLETEDTAMYYCATYFGSRHFYSMDYWGQGTLVTVSSLEVKGKHLCPSPLFPGPSKPFWVLVVVGGVLACYSLLVTVAFIIFWVLCARPRRSPAQEDGKVYINMPGRGRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 177 - protein, 445 aa, Artificial Sequence
  h6C10scFvHL-CD28H-CD28TM-CD28CS-CD3zICS
QVLVQSGAEVKKPGASVKVSCKASGYTFTDYNMDWVRQSHGQSLEWMGDINPNTGGTIFNQKFKGRATITVDTSSSTAYMDLHSLTSEDTAVYYCARRTYYGNSYYFDYWGQGTLVTVSSGGGGSGGGGSGGGGSDIQMTQSPASLSASVGERVTITCRASGDIHNYLAWYQQKPGKSPQLLIYNAKTLADGVPSRFSGSGSGTQYTLTINSLQPEDFATYYCQHFWSFPWTFGGGTKLEIKLEVKGKHLCPSPLFPGPSKPFWVLVVVGGVLACYSLLVTVAFIIFWVRSKRSRLLHSDYMNMTPRRPGPTRKHYQPYAPPRDFAAYRSKLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 178 - protein, 444 aa, Artificial Sequence
  h6C10scFvHL-CD28H-CD28TM-41BBCS-CD3zICS
QVLVQSGAEVKKPGASVKVSCKASGYTFTDYNMDWVRQSHGQSLEWMGDINPNTGGTIFNQKFKGRATITVDTSSSTAYMDLHSLTSEDTAVYYCARRTYYGNSYYFDYWGQGTLVTVSSGGGGSGGGGSGGGGSDIQMTQSPASLSASVGERVTITCRASGDIHNYLAWYQQKPGKSPQLLIYNAKTLADGVPSRFSGSGSGTQYTLTINSLQPEDFATYYCQHFWSFPWTFGGGTKLEIKLEVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 179 - protein, 426 aa, Artificial Sequence
  h6C10scFvHL-CD28H-CD28TM-DAP10CS-CD3zICS
QVLVQSGAEVKKPGASVKVSCKASGYTFTDYNMDWVRQSHGQSLEWMGDINPNTGGTIFNQKFKGRATITVDTSSSTAYMDLHSLTSEDTAVYYCARRTYYGNSYYFDYWGQGTLVTVSSGGGGSGGGGSGGGGSDIQMTQSPASLSASVGERVTITCRASGDIHNYLAWYQQKPGKSPQLLIYNAKTLADGVPSRFSGSGSGTQYTLTINSLQPEDFATYYCQHFWSFPWTFGGGTKLEIKLEVKGKHLCPSPLFPGPSKPFWVLVVVGGVLACYSLLVTVAFIIFWVLCARPRRSPAQEDGKVYINMPGRGRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 180 - protein, 445 aa, Artificial Sequence
  h6C10scFvLH-CD28H-CD28TM-CD28CS-CD3zICS
DIQMTQSPASLSASVGERVTITCRASGDIHNYLAWYQQKPGKSPQLLIYNAKTLADGVPSRFSGSGSGTQYTLTINSLQPEDFATYYCQHFWSFPWTFGGGTKLEIKGGGGSGGGGSGGGGSQVLVQSGAEVKKPGASVKVSCKASGYTFTDYNMDWVRQSHGQSLEWMGDINPNTGGTIFNQKFKGRATITVDTSSSTAYMDLHSLTSEDTAVYYCARRTYYGNSYYFDYWGQGTLVTVSSLEVKGKHLCPSPLFPGPSKPFWVLVVVGGVLACYSLLVTVAFIIFWVRSKRSRLLHSDYMNMTPRRPGPTRKHYQPYAPPRDFAAYRSKLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 181 - protein, 444 aa, Artificial Sequence
  h6C10scFvLH-CD28H-CD28TM-41BBCS-CD3zICS
DIQMTQSPASLSASVGERVTITCRASGDIHNYLAWYQQKPGKSPQLLIYNAKTLADGVPSRFSGSGSGTQYTLTINSLQPEDFATYYCQHFWSFPWTFGGGTKLEIKGGGGSGGGGSGGGGSQVLVQSGAEVKKPGASVKVSCKASGYTFTDYNMDWVRQSHGQSLEWMGDINPNTGGTIFNQKFKGRATITVDTSSSTAYMDLHSLTSEDTAVYYCARRTYYGNSYYFDYWGQGTLVTVSSLEVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 182 - protein, 426 aa, Artificial Sequence
  6C10scFvLH-CD28H-CD28TM-DAP10CS-CD3zICS
DIQMTQSPASLSASVGERVTITCRASGDIHNYLAWYQQKPGKSPQLLIYNAKTLADGVPSRFSGSGSGTQYTLTINSLQPEDFATYYCQHFWSFPWTFGGGTKLEIKGGGGSGGGGSGGGGSQVLVQSGAEVKKPGASVKVSCKASGYTFTDYNMDWVRQSHGQSLEWMGDINPNTGGTIFNQKFKGRATITVDTSSSTAYMDLHSLTSEDTAVYYCARRTYYGNSYYFDYWGQGTLVTVSSLEVKGKHLCPSPLFPGPSKPFWVLVVVGGVLACYSLLVTVAFIIFWVLCARPRRSPAQEDGKVYINMPGRGRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 183-193 - skipped (11 entries)

SEQ ID NO: 194 - protein, 820 aa, Artificial Sequence
  LS-h6E6scFvLH-CD28H-CD28TM-CD28CS-CD3zICS-T2A-trCD19
METPAQLLFLLLLWLPDTTGDIVLTQSPATLSVSPGERATLSCRASQGISDYLHWYQQKSGESPRLLIKYASQSMSGIPARFSGSGSGTDFTLTISSLEPEDVAVYYCQNGHTFPLTFGAGTRLEIKGGGGSGGGGSGGGGSQVQLVESGGGVVAPGQSLRLSCTASGFSLSSYGVHWVRQPPGKGLEWVAVIWAGGNTKYNSALMSRFTISRDTSKTTVYLQMNSLETEDTAMYYCATYFGSRHFYSMDYWGQGTLVTVSSLEVKGKHLCPSPLFPGPSKPFWVLVVVGGVLACYSLLVTVAFIIFWVRSKRSRLLHSDYMNMTPRRPGPTRKHYQPYAPPRDFAAYRSKLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRAGAKRSGSGEGRGSLLTCGDVEENPGPRMPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKRKRMT

SEQ ID NO: 195-196 - skipped (2 entries)

SEQ ID NO: 197 - protein, 820 aa, Artificial Sequence
  LS-h6C10scFvHL-CD28H-CD28TM-CD28CS-CD3zICS-T2A-trCD19
METPAQLLFLLLLWLPDTTGQVLVQSGAEVKKPGASVKVSCKASGYTFTDYNMDWVRQSHGQSLEWMGDINPNTGGTIFNQKFKGRATITVDTSSSTAYMDLHSLTSEDTAVYYCARRTYYGNSYYFDYWGQGTLVTVSSGGGGSGGGGSGGGGSDIQMTQSPASLSASVGERVTITCRASGDIHNYLAWYQQKPGKSPQLLIYNAKTLADGVPSRFSGSGSGTQYTLTINSLQPEDFATYYCQHFWSFPWTFGGGTKLEIKLEVKGKHLCPSPLFPGPSKPFWVLVVVGGVLACYSLLVTVAFIIFWVRSKRSRLLHSDYMNMTPRRPGPTRKHYQPYAPPRDFAAYRSKLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRAGAKRSGSGEGRGSLLTCGDVEENPGPRMPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKRKRMT

SEQ ID NO: 198-210 - skipped (13 entries)

SEQ ID NO: 211 - DNA, 360 bp, Artificial Sequence
  Mouse anti-ADAM12 Clone 6E6 Heavy chain variable
caggtgcagctgcaggagtctggacccggcctggtggcgccctcacagagcctgtccatcacttgcactgtctctgggttttcattaagctcctatggagtacactgggttcgccagcctccaggaaagggtctggagtggctgggagtaatatgggctggtggaaacacaaaatataattcggctctcatgtccagactgagcatcagcagagacacctccaagacccaagtcttcttagaaatgaatagtctggaaactgatgacacagccatgtactactgtgccacttacttcggtagccgacacttctattctatggactattggggtcaaggaacctcagtcaccgtctcctca

SEQ ID NO: 212 - DNA, 24 bp, Artificial Sequence
  Mouse anti-ADAM12 6E6 VH CDR 1
gggttttcattaagctcctatgga

SEQ ID NO: 213 - DNA, 21 bp, Artificial Sequence
  Mouse anti-ADAM12 6E6 VH CDR 2
atatgggctggtggaaacaca

SEQ ID NO: 214 - DNA, 36 bp, Artificial Sequence
  Mouse anti-ADAM12 6E6 VH CDR 3
tacttcggtagccgacacttctattctatggactat

SEQ ID NO: 215 - DNA, 322 bp, Artificial Sequence
  Mouse anti-ADAM12 Clone 6E6 Light chain variable
gacattgtgatgactcagtctccagccaccctgtctgtgactccaggagatagagtctctctttcctgcagggccagccagggtattagcgactacttacactggtatcaacaaaaatcacatgagtctccgaggcttctcatcaaatatgcttcccaatccatgtctgggatcccctccaggttcagtggcagtggatcagggtcagatttcactctcactatcaacagtgtggaacctgaagatgttggaatgtatttctgtcaaaatggtcacacctttccgctcacgttcggtgctgggaccaagctggaaataaaac

SEQ ID NO: 216 - DNA, 18 bp, Artificial Sequence
  Mouse anti-ADAM12 6E6 VL CDR 1
cagggtattagcgactac

SEQ ID NO: 217 - DNA, 9 bp, Artificial Sequence
  Mouse anti-ADAM12 6E6 VL CDR 2
tatgcttcc

SEQ ID NO: 218 - DNA, 37 bp, Artificial Sequence
  Mouse anti-ADAM12 6E6 VL CDR 3
caaaatggtcacacctttccgctcacgttcggtgctg

SEQ ID NO: 219-220 - skipped (2 entries)

SEQ ID NO: 221 - DNA, 363 bp, Artificial Sequence
  Mouse anti-ADAM12 Clone 6C10 Heavy chain variable
gaggccctgctgcagcagtctggacctgatctggcgaagcctggggcttcagtacaaataccctgcaaggcttctggatacacattcactgattacaacatggactgggtgaagcagagccatggaaagagccttgagtggattggagatattaatccaaacactggtggtacaatcttcaaccagaagttcaagggcaaggccacattgactgtagacaagtcctccagcacagccttcatggacctccacagcctgacatctgaggacactgcagtctattactgtgcaagacggacctactatggaaacagctactactttgactactggggccaaggcaccactctcacagtctcctca

SEQ ID NO: 222 - DNA, 18 bp, Artificial Sequence
  Mouse anti-ADAM12 6C10 VH CDR 1
ggatacacattcactgat

SEQ ID NO: 223 - DNA, 24 bp, Artificial Sequence
  Mouse anti-ADAM12 6C10 VH CDR 2
attaatccaaacactggtggtaca

SEQ ID NO: 224 - DNA, 36 bp, Artificial Sequence
  Mouse anti-ADAM12 6C10 VH CDR 3
cggacctactatggaaacagctactactttgactac

SEQ ID NO: 225 - DNA, 321 bp, Artificial Sequence
  Mouse anti-ADAM12 Clone 6C10 Light chain variable
gacatccagatgactcagtctccagcctccctatctgcatctgtgggagaaactgtcaccatcacatgtcgagcaagtggggatattcacaattatttagcatggtatcagcagaaacagggaaaatctcctcagctcctggtctataatgcaaaaaccttagcagatggtgtgccatcaaggttcagtggcagtggatcaggaacacaatattctctcaagatgaacagcctgcagcctgaagattttgggacttattactgtcaacatttttggagttttccgtggacgttcggtggaggcaccaagctggaaatcaaa

SEQ ID NO: 226 - DNA, 15 bp, Artificial Sequence
  Mouse anti-ADAM12 6C10 VL CDR 1
ggggatattcacaat

SEQ ID NO: 227 - DNA, 12 bp, Artificial Sequence
  Mouse anti-ADAM12 6C10 VL CDR 2
tataatgcaaaa

SEQ ID NO: 228 - DNA, 30 bp, Artificial Sequence
  Mouse anti-ADAM12 6C10 VL CDR 3
caacatttttggagttttccgtggacgttc

SEQ ID NO: 229-230 - skipped (2 entries)

SEQ ID NO: 231 - DNA, 360 bp, Artificial Sequence
  Humanized anti-ADAM12 Clone 6E6 Heavy chain variable
caggtgcagctggtggagagcggaggaggtgtagttgctcctggacagagcctgagactgagctgtaccgccagcgggttcagcctgagcagctacggggtgcactgggtgagacagccccccgggaaggggctggagtgggtggccgtgatctgggccggggggaacaccaagtacaacagcgccctgatgagcagattcaccatcagcagagataccagcaagaccaccgtgtacctgcagatgaacagcctggagaccgaggataccgccatgtactactgtgccacctacttcgggagcagacacttctacagcatggattactgggggcaggggaccctggtgaccgtgagcagc

SEQ ID NO: 232 - DNA, 24 bp, Artificial Sequence
  Humanized anti-ADAM12 6E6 VH CDR 1
gggttcagcctgagcagctacggg

SEQ ID NO: 233 - DNA, 21 bp, Artificial Sequence
  Humanized anti-ADAM12 6E6 VH CDR 2
atctgggccggggggaacacc

SEQ ID NO: 234 - DNA, 36 bp, Artificial Sequence
  Humanized anti-ADAM12 6E6 VH CDR 3
tacttcgggagcagacacttctacagcatggattac

SEQ ID NO: 235 - DNA, 321 bp, Artificial Sequence
  Humanized anti-ADAM12 Clone 6E6 Light chain variable
gatatcgtgctgacccagagccccgccaccctgagcgtgagccccggggagagagccaccctgagctgtagagccagccaggggatcagcgattacctgcactggtaccagcagaagagcggggagagccccagactgctgatcaagtacgccagccagagcatgagcgggatccccgccagattcagcgggagcgggagcgggaccgatttcaccctgaccatcagcagcctggagcccgaggatgtggccgtgtactactgtcagaacgggcacaccttccccctgaccttcggggccgggaccagactggagatcaag

SEQ ID NO: 236 - DNA, 18 bp, Artificial Sequence
  Humanized anti-ADAM12 6E6 VL CDR 1
caggggatcagcgattac

SEQ ID NO: 237 - DNA, 9 bp, Artificial Sequence
  Humanized anti-ADAM12 6E6 VL CDR 2
tacgccagc

SEQ ID NO: 238 - DNA, 36 bp, Artificial Sequence
  Humanized anti-ADAM12 6E6 VL CDR 3
cagaacgggcacaccttccccctgaccttcggggcc

SEQ ID NO: 239 - DNA, 726 bp, Artificial Sequence
  Humanized anti-ADAM12 6E6 scFv VH-G4S3-VL
caggtgcagctggtggagagcggaggaggtgtagttgctcctggacagagcctgagactgagctgtaccgccagcgggttcagcctgagcagctacggggtgcactgggtgagacagccccccgggaaggggctggagtgggtggccgtgatctgggccggggggaacaccaagtacaacagcgccctgatgagcagattcaccatcagcagagataccagcaagaccaccgtgtacctgcagatgaacagcctggagaccgaggataccgccatgtactactgtgccacctacttcgggagcagacacttctacagcatggattactgggggcaggggaccctggtgaccgtgagcagcggtggtggtggttctggcggcggcggctccggtggtggtggttccgatatcgtgctgacccagagccccgccaccctgagcgtgagccccggggagagagccaccctgagctgtagagccagccaggggatcagcgattacctgcactggtaccagcagaagagcggggagagccccagactgctgatcaagtacgccagccagagcatgagcgggatccccgccagattcagcgggagcgggagcgggaccgatttcaccctgaccatcagcagcctggagcccgaggatgtggccgtgtactactgtcagaacgggcacaccttccccctgaccttcggggccgggaccagactggagatcaag

SEQ ID NO: 240 - DNA, 726 bp, Artificial Sequence
  Humanized anti-ADAM12 6E6 scFv VL-G4S3-VH
gatatcgtgctgacccagagccccgccaccctgagcgtgagccccggggagagagccaccctgagctgtagagccagccaggggatcagcgattacctgcactggtaccagcagaagagcggggagagccccagactgctgatcaagtacgccagccagagcatgagcgggatccccgccagattcagcgggagcgggagcgggaccgatttcaccctgaccatcagcagcctggagcccgaggatgtggccgtgtactactgtcagaacgggcacaccttccccctgaccttcggggccgggaccagactggagatcaagggtggtggtggttctggcggcggcggctccggtggtggtggttcccaggtgcagctggtggagagcggaggaggtgtagttgctcctggacagagcctgagactgagctgtaccgccagcgggttcagcctgagcagctacggggtgcactgggtgagacagccccccgggaaggggctggagtgggtggccgtgatctgggccggggggaacaccaagtacaacagcgccctgatgagcagattcaccatcagcagagataccagcaagaccaccgtgtacctgcagatgaacagcctggagaccgaggataccgccatgtactactgtgccacctacttcgggagcagacacttctacagcatggattactgggggcaggggaccctggtgaccgtgagcagc

SEQ ID NO: 241 - DNA, 360 bp, Artificial Sequence
  Humanized anti-ADAM12 Clone 6C10 Heavy chain variable
caggtgctggtgcagagcggggccgaggtgaagaagcccggggccagcgtgaaggtgagctgtaaggccagcgggtacaccttcaccgattacaacatggattgggtgagacagagccacgggcagagcctggagtggatgggggatatcaaccccaacaccggggggaccatcttcaaccagaagttcaaggggagagccaccatcaccgtggataccagcagcagcaccgcctacatggatctgcacagcctgaccagcgaggataccgccgtgtactactgtgccagaagaacctactacgggaacagctactacttcgattactgggggcaggggaccctggtgaccgtgagcagc

SEQ ID NO: 242 - DNA, 18 bp, Artificial Sequence
  Humanized anti-ADAM12 6C10 VH CDR 1
gggtacaccttcaccgat

SEQ ID NO: 243 - DNA, 24 bp, Artificial Sequence
  Humanized anti-ADAM12 6C10 VH CDR 2
atcaaccccaacaccggggggacc

SEQ ID NO: 244 - DNA, 36 bp, Artificial Sequence
  Humanized anti-ADAM12 6C10 VH CDR 3
agaacctactacgggaacagctactacttcgattac

SEQ ID NO: 245 - DNA, 321 bp, Artificial Sequence
  Humanized anti-ADAM12 Clone 6C10 Light chain variable
gatatccagatgacccagagccccgccagcctgagcgccagcgtgggggagagagtgaccatcacctgtagagccagcggggatatccacaactacctggcctggtaccagcagaagcccgggaagagcccccagctgctgatctacaacgccaagaccctggccgatggggtgcccagcagattcagcgggagcgggagcgggacccagtacaccctgaccatcaacagcctgcagcccgaggatttcgccacctactactgtcagcacttctggagcttcccctggaccttcgggggggggaccaagctggagatcaag

SEQ ID NO: 246 - DNA, 15 bp, Artificial Sequence
  Humanized anti-ADAM12 6C10 VL CDR 1
ggggatatccacaac

SEQ ID NO: 247 - DNA, 12 bp, Artificial Sequence
  Humanized anti-ADAM12 6C10 VL CDR 2
tacaacgccaag

SEQ ID NO: 248 - DNA, 30 bp, Artificial Sequence
  Humanized anti-ADAM12 6C10 VL CDR 3
cagcacttctggagcttcccctggaccttc

SEQ ID NO: 249 - DNA, 726 bp, Artificial Sequence
  Humanized anti-ADAM12 6C10 scFv VH-G4S3-VL
caggtgctggtgcagagcggggccgaggtgaagaagcccggggccagcgtgaaggtgagctgtaaggccagcgggtacaccttcaccgattacaacatggattgggtgagacagagccacgggcagagcctggagtggatgggggatatcaaccccaacaccggggggaccatcttcaaccagaagttcaaggggagagccaccatcaccgtggataccagcagcagcaccgcctacatggatctgcacagcctgaccagcgaggataccgccgtgtactactgtgccagaagaacctactacgggaacagctactacttcgattactgggggcaggggaccctggtgaccgtgagcagcggtggtggtggttctggcggcggcggctccggtggtggtggttccgatatccagatgacccagagccccgccagcctgagcgccagcgtgggggagagagtgaccatcacctgtagagccagcggggatatccacaactacctggcctggtaccagcagaagcccgggaagagcccccagctgctgatctacaacgccaagaccctggccgatggggtgcccagcagattcagcgggagcgggagcgggacccagtacaccctgaccatcaacagcctgcagcccgaggatttcgccacctactactgtcagcacttctggagcttcccctggaccttcgggggggggaccaagctggagatcaag

SEQ ID NO: 250 - DNA, 726 bp, Artificial Sequence
  Humanized anti-ADAM12 6C10 scFv VL-G4S3-VH
gatatccagatgacccagagccccgccagcctgagcgccagcgtgggggagagagtgaccatcacctgtagagccagcggggatatccacaactacctggcctggtaccagcagaagcccgggaagagcccccagctgctgatctacaacgccaagaccctggccgatggggtgcccagcagattcagcgggagcgggagcgggacccagtacaccctgaccatcaacagcctgcagcccgaggatttcgccacctactactgtcagcacttctggagcttcccctggaccttcgggggggggaccaagctggagatcaagggtggtggtggttctggcggcggcggctccggtggtggtggttcccaggtgctggtgcagagcggggccgaggtgaagaagcccggggccagcgtgaaggtgagctgtaaggccagcgggtacaccttcaccgattacaacatggattgggtgagacagagccacgggcagagcctggagtggatgggggatatcaaccccaacaccggggggaccatcttcaaccagaagttcaaggggagagccaccatcaccgtggataccagcagcagcaccgcctacatggatctgcacagcctgaccagcgaggataccgccgtgtactactgtgccagaagaacctactacgggaacagctactacttcgattactgggggcaggggaccctggtgaccgtgagcagc

SEQ ID NO: 251-259 - skipped (9 entries)

SEQ ID NO: 260 - DNA, 60 bp, Artificial Sequence
  Lead sequence
atggaaaccccagcgcagcttctcttcctcctgctactctggctcccagataccaccgga

SEQ ID NO: 261 - DNA, 81 bp, Artificial Sequence
  Human CD28 transmembrane domain
ttttgggtgctggtggtggttggtggagtcctggcttgctatagcttgctagtaacagtggcctttattattttctgggtg

SEQ ID NO: 262 - DNA, 339 bp, Artificial Sequence
  Human CD3 zeta intracellular signaling domain
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 263 - DNA, 60 bp, Artificial Sequence
  Human CD28 hinge
ctcgaggtgaaagggaaacacctttgtccaagtcccctatttcccggaccttctaagccc

SEQ ID NO: 264 - DNA, 129 bp, Artificial Sequence
  Human CD28 costimulatory domain
aggagtaagaggagcaggctcctgcacagtgactacatgaacatgactccccgccgccccgggcccacccgcaagcattaccagccctatgccccaccacgcgacttcgcagcctatcgctccaagctt

SEQ ID NO: 265 - DNA, 126 bp, Artificial Sequence
  Human 4-1BB costimulatory domain
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 266 - DNA, 72 bp, Artificial Sequence
  Human DAP10 costimulatory domain
ctgtgcgcacgcccacgccgcagccccgcccaagaagatggcaaagtctacatcaacatgccaggcaggggc

SEQ ID NO: 267 - DNA, 15 bp, Artificial Sequence
  Glycine-Serine linker unite
ggtggtggtggttct

SEQ ID NO: 268 - DNA, 45 bp, Artificial Sequence
  (Gly-Gly-Gly-Gly-Ser)X3 linker ((Gly4Ser)3 linker, (G4S)3 linker, G4S X3 linker, or G4SX3 linker)
ggtggtggtggttctggcggcggcggctccggtggtggtggttcc

SEQ ID NO: 269 - DNA, 84 bp, Artificial Sequence
  T2A ribosomal skip sequence
gccggcgccaaaaggtctggctccggtgagggcagaggaagtcttctaacatgcggtgacgtggaggagaatcccggccctaga

SEQ ID NO: 270 - DNA, 984 bp, Artificial Sequence
  truncated CD19
atgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagaaagcgaatgacttaa

SEQ ID NO: 271 - DNA, 1335 bp, Artificial Sequence
  h6E6scFvHL-CD28H-CD28TM-CD28CS-CD3zICS
caggtgcagctggtggagagcggaggaggtgtagttgctcctggacagagcctgagactgagctgtaccgccagcgggttcagcctgagcagctacggggtgcactgggtgagacagccccccgggaaggggctggagtgggtggccgtgatctgggccggggggaacaccaagtacaacagcgccctgatgagcagattcaccatcagcagagataccagcaagaccaccgtgtacctgcagatgaacagcctggagaccgaggataccgccatgtactactgtgccacctacttcgggagcagacacttctacagcatggattactgggggcaggggaccctggtgaccgtgagcagcggtggtggtggttctggcggcggcggctccggtggtggtggttccgatatcgtgctgacccagagccccgccaccctgagcgtgagccccggggagagagccaccctgagctgtagagccagccaggggatcagcgattacctgcactggtaccagcagaagagcggggagagccccagactgctgatcaagtacgccagccagagcatgagcgggatccccgccagattcagcgggagcgggagcgggaccgatttcaccctgaccatcagcagcctggagcccgaggatgtggccgtgtactactgtcagaacgggcacaccttccccctgaccttcggggccgggaccagactggagatcaagctcgaggtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccaagcttagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 272 - DNA, 1332 bp, Artificial Sequence
  h6E6scFvHL-CD28H-CD28TM-41BBCS-CD3zICS
caggtgcagctggtggagagcggaggaggtgtagttgctcctggacagagcctgagactgagctgtaccgccagcgggttcagcctgagcagctacggggtgcactgggtgagacagccccccgggaaggggctggagtgggtggccgtgatctgggccggggggaacaccaagtacaacagcgccctgatgagcagattcaccatcagcagagataccagcaagaccaccgtgtacctgcagatgaacagcctggagaccgaggataccgccatgtactactgtgccacctacttcgggagcagacacttctacagcatggattactgggggcaggggaccctggtgaccgtgagcagcggtggtggtggttctggcggcggcggctccggtggtggtggttccgatatcgtgctgacccagagccccgccaccctgagcgtgagccccggggagagagccaccctgagctgtagagccagccaggggatcagcgattacctgcactggtaccagcagaagagcggggagagccccagactgctgatcaagtacgccagccagagcatgagcgggatccccgccagattcagcgggagcgggagcgggaccgatttcaccctgaccatcagcagcctggagcccgaggatgtggccgtgtactactgtcagaacgggcacaccttccccctgaccttcggggccgggaccagactggagatcaagctcgaggtgaaagggaaacacctttgtccaagtcccctatttcccggaccttctaagcccttttgggtgctggtggtggttggtggagtcctggcttgctatagcttgctagtaacagtggcctttattattttctgggtgaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 273 - DNA, 1278 bp, Artificial Sequence
  h6E6scFvHL-CD28H-CD28TM-DAP10CS-CD3zICS
caggtgcagctggtggagagcggaggaggtgtagttgctcctggacagagcctgagactgagctgtaccgccagcgggttcagcctgagcagctacggggtgcactgggtgagacagccccccgggaaggggctggagtgggtggccgtgatctgggccggggggaacaccaagtacaacagcgccctgatgagcagattcaccatcagcagagataccagcaagaccaccgtgtacctgcagatgaacagcctggagaccgaggataccgccatgtactactgtgccacctacttcgggagcagacacttctacagcatggattactgggggcaggggaccctggtgaccgtgagcagcggtggtggtggttctggcggcggcggctccggtggtggtggttccgatatcgtgctgacccagagccccgccaccctgagcgtgagccccggggagagagccaccctgagctgtagagccagccaggggatcagcgattacctgcactggtaccagcagaagagcggggagagccccagactgctgatcaagtacgccagccagagcatgagcgggatccccgccagattcagcgggagcgggagcgggaccgatttcaccctgaccatcagcagcctggagcccgaggatgtggccgtgtactactgtcagaacgggcacaccttccccctgaccttcggggccgggaccagactggagatcaagctcgaggtgaaagggaaacacctttgtccaagtcccctatttcccggaccttctaagcccttttgggtgctggtggtggttggtggagtcctggcttgctatagcttgctagtaacagtggcctttattattttctgggtgctgtgcgcacgcccacgccgcagccccgcccaagaagatggcaaagtctacatcaacatgccaggcaggggcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 274 - DNA, 1335 bp, Artificial Sequence
  h6E6scFvLH-CD28H-CD28TM-CD28CS-CD3zICS
gatatcgtgctgacccagagccccgccaccctgagcgtgagccccggggagagagccaccctgagctgtagagccagccaggggatcagcgattacctgcactggtaccagcagaagagcggggagagccccagactgctgatcaagtacgccagccagagcatgagcgggatccccgccagattcagcgggagcgggagcgggaccgatttcaccctgaccatcagcagcctggagcccgaggatgtggccgtgtactactgtcagaacgggcacaccttccccctgaccttcggggccgggaccagactggagatcaagggtggtggtggttctggcggcggcggctccggtggtggtggttcccaggtgcagctggtggagagcggaggaggtgtagttgctcctggacagagcctgagactgagctgtaccgccagcgggttcagcctgagcagctacggggtgcactgggtgagacagccccccgggaaggggctggagtgggtggccgtgatctgggccggggggaacaccaagtacaacagcgccctgatgagcagattcaccatcagcagagataccagcaagaccaccgtgtacctgcagatgaacagcctggagaccgaggataccgccatgtactactgtgccacctacttcgggagcagacacttctacagcatggattactgggggcaggggaccctggtgaccgtgagcagcctcgaggtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccaagcttagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 275 - DNA, 1332 bp, Artificial Sequence
  h6E6scFvLH-CD28H-CD28TM-41BBCS-CD3zICS
gatatcgtgctgacccagagccccgccaccctgagcgtgagccccggggagagagccaccctgagctgtagagccagccaggggatcagcgattacctgcactggtaccagcagaagagcggggagagccccagactgctgatcaagtacgccagccagagcatgagcgggatccccgccagattcagcgggagcgggagcgggaccgatttcaccctgaccatcagcagcctggagcccgaggatgtggccgtgtactactgtcagaacgggcacaccttccccctgaccttcggggccgggaccagactggagatcaagggtggtggtggttctggcggcggcggctccggtggtggtggttcccaggtgcagctggtggagagcggaggaggtgtagttgctcctggacagagcctgagactgagctgtaccgccagcgggttcagcctgagcagctacggggtgcactgggtgagacagccccccgggaaggggctggagtgggtggccgtgatctgggccggggggaacaccaagtacaacagcgccctgatgagcagattcaccatcagcagagataccagcaagaccaccgtgtacctgcagatgaacagcctggagaccgaggataccgccatgtactactgtgccacctacttcgggagcagacacttctacagcatggattactgggggcaggggaccctggtgaccgtgagcagcctcgaggtgaaagggaaacacctttgtccaagtcccctatttcccggaccttctaagcccttttgggtgctggtggtggttggtggagtcctggcttgctatagcttgctagtaacagtggcctttattattttctgggtgaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 276 - DNA, 1278 bp, Artificial Sequence
  h6E6scFvLH-CD28H-CD28TM-DAP10CS-CD3zICS
gatatcgtgctgacccagagccccgccaccctgagcgtgagccccggggagagagccaccctgagctgtagagccagccaggggatcagcgattacctgcactggtaccagcagaagagcggggagagccccagactgctgatcaagtacgccagccagagcatgagcgggatccccgccagattcagcgggagcgggagcgggaccgatttcaccctgaccatcagcagcctggagcccgaggatgtggccgtgtactactgtcagaacgggcacaccttccccctgaccttcggggccgggaccagactggagatcaagggtggtggtggttctggcggcggcggctccggtggtggtggttcccaggtgcagctggtggagagcggaggaggtgtagttgctcctggacagagcctgagactgagctgtaccgccagcgggttcagcctgagcagctacggggtgcactgggtgagacagccccccgggaaggggctggagtgggtggccgtgatctgggccggggggaacaccaagtacaacagcgccctgatgagcagattcaccatcagcagagataccagcaagaccaccgtgtacctgcagatgaacagcctggagaccgaggataccgccatgtactactgtgccacctacttcgggagcagacacttctacagcatggattactgggggcaggggaccctggtgaccgtgagcagcctcgaggtgaaagggaaacacctttgtccaagtcccctatttcccggaccttctaagcccttttgggtgctggtggtggttggtggagtcctggcttgctatagcttgctagtaacagtggcctttattattttctgggtgctgtgcgcacgcccacgccgcagccccgcccaagaagatggcaaagtctacatcaacatgccaggcaggggcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 277 - DNA, 1335 bp, Artificial Sequence
  h6C10scFvHL-CD28H-CD28TM-CD28CS-CD3zICS
caggtgctggtgcagagcggggccgaggtgaagaagcccggggccagcgtgaaggtgagctgtaaggccagcgggtacaccttcaccgattacaacatggattgggtgagacagagccacgggcagagcctggagtggatgggggatatcaaccccaacaccggggggaccatcttcaaccagaagttcaaggggagagccaccatcaccgtggataccagcagcagcaccgcctacatggatctgcacagcctgaccagcgaggataccgccgtgtactactgtgccagaagaacctactacgggaacagctactacttcgattactgggggcaggggaccctggtgaccgtgagcagcggtggtggtggttctggcggcggcggctccggtggtggtggttccgatatccagatgacccagagccccgccagcctgagcgccagcgtgggggagagagtgaccatcacctgtagagccagcggggatatccacaactacctggcctggtaccagcagaagcccgggaagagcccccagctgctgatctacaacgccaagaccctggccgatggggtgcccagcagattcagcgggagcgggagcgggacccagtacaccctgaccatcaacagcctgcagcccgaggatttcgccacctactactgtcagcacttctggagcttcccctggaccttcgggggggggaccaagctggagatcaagctcgaggtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccaagcttagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 278 - DNA, 1332 bp, Artificial Sequence
  h6C10scFvHL-CD28H-CD28TM-41BBCS-CD3zICS
caggtgctggtgcagagcggggccgaggtgaagaagcccggggccagcgtgaaggtgagctgtaaggccagcgggtacaccttcaccgattacaacatggattgggtgagacagagccacgggcagagcctggagtggatgggggatatcaaccccaacaccggggggaccatcttcaaccagaagttcaaggggagagccaccatcaccgtggataccagcagcagcaccgcctacatggatctgcacagcctgaccagcgaggataccgccgtgtactactgtgccagaagaacctactacgggaacagctactacttcgattactgggggcaggggaccctggtgaccgtgagcagcggtggtggtggttctggcggcggcggctccggtggtggtggttccgatatccagatgacccagagccccgccagcctgagcgccagcgtgggggagagagtgaccatcacctgtagagccagcggggatatccacaactacctggcctggtaccagcagaagcccgggaagagcccccagctgctgatctacaacgccaagaccctggccgatggggtgcccagcagattcagcgggagcgggagcgggacccagtacaccctgaccatcaacagcctgcagcccgaggatttcgccacctactactgtcagcacttctggagcttcccctggaccttcgggggggggaccaagctggagatcaagctcgaggtgaaagggaaacacctttgtccaagtcccctatttcccggaccttctaagcccttttgggtgctggtggtggttggtggagtcctggcttgctatagcttgctagtaacagtggcctttattattttctgggtgaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 279 - DNA, 1278 bp, Artificial Sequence
  h6C10scFvHL-CD28H-CD28TM-DAP10CS-CD3zICS
caggtgctggtgcagagcggggccgaggtgaagaagcccggggccagcgtgaaggtgagctgtaaggccagcgggtacaccttcaccgattacaacatggattgggtgagacagagccacgggcagagcctggagtggatgggggatatcaaccccaacaccggggggaccatcttcaaccagaagttcaaggggagagccaccatcaccgtggataccagcagcagcaccgcctacatggatctgcacagcctgaccagcgaggataccgccgtgtactactgtgccagaagaacctactacgggaacagctactacttcgattactgggggcaggggaccctggtgaccgtgagcagcggtggtggtggttctggcggcggcggctccggtggtggtggttccgatatccagatgacccagagccccgccagcctgagcgccagcgtgggggagagagtgaccatcacctgtagagccagcggggatatccacaactacctggcctggtaccagcagaagcccgggaagagcccccagctgctgatctacaacgccaagaccctggccgatggggtgcccagcagattcagcgggagcgggagcgggacccagtacaccctgaccatcaacagcctgcagcccgaggatttcgccacctactactgtcagcacttctggagcttcccctggaccttcgggggggggaccaagctggagatcaagctcgaggtgaaagggaaacacctttgtccaagtcccctatttcccggaccttctaagcccttttgggtgctggtggtggttggtggagtcctggcttgctatagcttgctagtaacagtggcctttattattttctgggtgctgtgcgcacgcccacgccgcagccccgcccaagaagatggcaaagtctacatcaacatgccaggcaggggcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 280 - DNA, 1335 bp, Artificial Sequence
  h6C10scFvLH-CD28H-CD28TM-CD28CS-CD3zICS
gatatccagatgacccagagccccgccagcctgagcgccagcgtgggggagagagtgaccatcacctgtagagccagcggggatatccacaactacctggcctggtaccagcagaagcccgggaagagcccccagctgctgatctacaacgccaagaccctggccgatggggtgcccagcagattcagcgggagcgggagcgggacccagtacaccctgaccatcaacagcctgcagcccgaggatttcgccacctactactgtcagcacttctggagcttcccctggaccttcgggggggggaccaagctggagatcaagggtggtggtggttctggcggcggcggctccggtggtggtggttcccaggtgctggtgcagagcggggccgaggtgaagaagcccggggccagcgtgaaggtgagctgtaaggccagcgggtacaccttcaccgattacaacatggattgggtgagacagagccacgggcagagcctggagtggatgggggatatcaaccccaacaccggggggaccatcttcaaccagaagttcaaggggagagccaccatcaccgtggataccagcagcagcaccgcctacatggatctgcacagcctgaccagcgaggataccgccgtgtactactgtgccagaagaacctactacgggaacagctactacttcgattactgggggcaggggaccctggtgaccgtgagcagcctcgaggtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccaagcttagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 281 - DNA, 1332 bp, Artificial Sequence
  h6C10scFvLH-CD28H-CD28TM-41BBCS-CD3zICS
gatatccagatgacccagagccccgccagcctgagcgccagcgtgggggagagagtgaccatcacctgtagagccagcggggatatccacaactacctggcctggtaccagcagaagcccgggaagagcccccagctgctgatctacaacgccaagaccctggccgatggggtgcccagcagattcagcgggagcgggagcgggacccagtacaccctgaccatcaacagcctgcagcccgaggatttcgccacctactactgtcagcacttctggagcttcccctggaccttcgggggggggaccaagctggagatcaagggtggtggtggttctggcggcggcggctccggtggtggtggttcccaggtgctggtgcagagcggggccgaggtgaagaagcccggggccagcgtgaaggtgagctgtaaggccagcgggtacaccttcaccgattacaacatggattgggtgagacagagccacgggcagagcctggagtggatgggggatatcaaccccaacaccggggggaccatcttcaaccagaagttcaaggggagagccaccatcaccgtggataccagcagcagcaccgcctacatggatctgcacagcctgaccagcgaggataccgccgtgtactactgtgccagaagaacctactacgggaacagctactacttcgattactgggggcaggggaccctggtgaccgtgagcagcctcgaggtgaaagggaaacacctttgtccaagtcccctatttcccggaccttctaagcccttttgggtgctggtggtggttggtggagtcctggcttgctatagcttgctagtaacagtggcctttattattttctgggtgaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 282 - DNA, 1278 bp, Artificial Sequence
  6C10scFvLH-CD28H-CD28TM-DAP10CS-CD3zICS
gatatccagatgacccagagccccgccagcctgagcgccagcgtgggggagagagtgaccatcacctgtagagccagcggggatatccacaactacctggcctggtaccagcagaagcccgggaagagcccccagctgctgatctacaacgccaagaccctggccgatggggtgcccagcagattcagcgggagcgggagcgggacccagtacaccctgaccatcaacagcctgcagcccgaggatttcgccacctactactgtcagcacttctggagcttcccctggaccttcgggggggggaccaagctggagatcaagggtggtggtggttctggcggcggcggctccggtggtggtggttcccaggtgctggtgcagagcggggccgaggtgaagaagcccggggccagcgtgaaggtgagctgtaaggccagcgggtacaccttcaccgattacaacatggattgggtgagacagagccacgggcagagcctggagtggatgggggatatcaaccccaacaccggggggaccatcttcaaccagaagttcaaggggagagccaccatcaccgtggataccagcagcagcaccgcctacatggatctgcacagcctgaccagcgaggataccgccgtgtactactgtgccagaagaacctactacgggaacagctactacttcgattactgggggcaggggaccctggtgaccgtgagcagcctcgaggtgaaagggaaacacctttgtccaagtcccctatttcccggaccttctaagcccttttgggtgctggtggtggttggtggagtcctggcttgctatagcttgctagtaacagtggcctttattattttctgggtgctgtgcgcacgcccacgccgcagccccgcccaagaagatggcaaagtctacatcaacatgccaggcaggggcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 283-293 - skipped (11 entries)

SEQ ID NO: 294 - DNA, 2463 bp, Artificial Sequence
  LS-h6E6scFvLH-CD28H-CD28TM-CD28CS-CD3zICS-T2A-trCD19
atggaaaccccagcgcagcttctcttcctcctgctactctggctcccagataccaccggagatatcgtgctgacccagagccccgccaccctgagcgtgagccccggggagagagccaccctgagctgtagagccagccaggggatcagcgattacctgcactggtaccagcagaagagcggggagagccccagactgctgatcaagtacgccagccagagcatgagcgggatccccgccagattcagcgggagcgggagcgggaccgatttcaccctgaccatcagcagcctggagcccgaggatgtggccgtgtactactgtcagaacgggcacaccttccccctgaccttcggggccgggaccagactggagatcaagggtggtggtggttctggcggcggcggctccggtggtggtggttcccaggtgcagctggtggagagcggaggaggtgtagttgctcctggacagagcctgagactgagctgtaccgccagcgggttcagcctgagcagctacggggtgcactgggtgagacagccccccgggaaggggctggagtgggtggccgtgatctgggccggggggaacaccaagtacaacagcgccctgatgagcagattcaccatcagcagagataccagcaagaccaccgtgtacctgcagatgaacagcctggagaccgaggataccgccatgtactactgtgccacctacttcgggagcagacacttctacagcatggattactgggggcaggggaccctggtgaccgtgagcagcctcgaggtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccaagcttagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgccggcgccaaaaggtctggctccggtgagggcagaggaagtcttctaacatgcggtgacgtggaggagaatcccggccctagaatgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagaaagcgaatgacttaa

SEQ ID NO: 295-296 - skipped (2 entries)

SEQ ID NO: 297 - DNA, 2463 bp, Artificial Sequence
  LS-h6C10scFvHL-CD28H-CD28TM-CD28CS-CD3zICS-T2A-trCD19
atggaaaccccagcgcagcttctcttcctcctgctactctggctcccagataccaccggacaggtgctggtgcagagcggggccgaggtgaagaagcccggggccagcgtgaaggtgagctgtaaggccagcgggtacaccttcaccgattacaacatggattgggtgagacagagccacgggcagagcctggagtggatgggggatatcaaccccaacaccggggggaccatcttcaaccagaagttcaaggggagagccaccatcaccgtggataccagcagcagcaccgcctacatggatctgcacagcctgaccagcgaggataccgccgtgtactactgtgccagaagaacctactacgggaacagctactacttcgattactgggggcaggggaccctggtgaccgtgagcagcggtggtggtggttctggcggcggcggctccggtggtggtggttccgatatccagatgacccagagccccgccagcctgagcgccagcgtgggggagagagtgaccatcacctgtagagccagcggggatatccacaactacctggcctggtaccagcagaagcccgggaagagcccccagctgctgatctacaacgccaagaccctggccgatggggtgcccagcagattcagcgggagcgggagcgggacccagtacaccctgaccatcaacagcctgcagcccgaggatttcgccacctactactgtcagcacttctggagcttcccctggaccttcgggggggggaccaagctggagatcaagctcgaggtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccaagcttagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgccggcgccaaaaggtctggctccggtgagggcagaggaagtcttctaacatgcggtgacgtggaggagaatcccggccctagaatgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagaaagcgaatgacttaa